(19) United States
(12) Patent Application Publication
(10) Pub. No.: US 2024/0316106 A1
(43) Pub. Date: Sep. 26, 2024

(54) METHODS FOR GENERATING PRIMARY IMMUNE CELLS

(71) Applicant: AstraZeneca AB, Sodertalje (SE)

(72) Inventors: Deepali Malhotra, Wilmington, DE (US); Michael Overstreet, Wilmington, DE (US); Gordon Moody, Wilmington, DE (US); Mark Cobbold, Wilmington, DE (US)

(21) Appl. No.: 18/583,213

(22) Filed: Feb. 21, 2024

Related U.S. Application Data
(60) Provisional application No. 63/486,398, filed on Feb. 22, 2023.

(51) Int. Cl. A61K 35/17; A61K 39/00; A61P 35/00; C07K 14/05; C07K 14/47; C07K 14/82; C12N 5/0783; C12N 9/22; C12N 15/11; C12N 15/86

(52) U.S. Cl. CPC A61K 35/17; A61K 39/4611; A61K 39/4631; A61K 39/464452; A61K 39/464457; A61K 39/464464; A61P 35/00; C07K 14/05; C07K 14/4703; C07K 14/82; C12N 5/0636; C12N 9/22; C12N 15/111; C12N 15/86; C12N 2310/20; C12N 2740/15043

(57) ABSTRACT

The disclosure relates to methods, cells, and compositions for preparing cell populations and compositions for adoptive cell therapy. In particular, provided herein are methods for expansion and proliferation of primary immune cells including T cell populations.

### CROSS REFERENCE TO RELATED APPLICATIONS

This application claims priority to U.S. Provisional Application No. 63/486,398, filed Feb. 22, 2023, the disclosure of which is incorporated by reference in its entirety.

### FIELD OF THE DISCLOSURE

The disclosure relates to methods, cells, and compositions for preparing cell populations and compositions for adoptive cell therapy. In particular, provided herein are methods for expansion and proliferation of primary immune cells including T cell populations.

### BACKGROUND

Engineered adoptive cellular therapies have been transformative for patients with hematological malignancies in recent years with the first approval for a chimeric antigen receptor (CAR)-based therapy by the FDA in 2017 (Larson & Maus, Nat Rev Cancer 21, 145-161 (2021); Yu, et al., Nature Reviews Drug Discovery 19, 583-584 (2020)). Since 2017, the number of clinical trials investigating adoptive cell therapies such as CAR-T cells, CAR-Natural Killer (NK) and CAR-NKT cells, T cell receptor (TCR)-T cells, tumor infiltrating lymphocytes (TILs), tumor-specific antigen-targeting T cells, and other cellular therapies has grown rapidly. More recently, the first CAR-macrophages (CAR-M) have entered the clinic for the treatment of solid tumors (Mukhopadhyay, Nat Methods 17, 561 (2020); Klichinsky, et al., Nat Biotechnol., 8, 947-953 (2020); Villanueva, Nature Reviews Drug Discovery 19, 308 (2020); ClinicalTrials.gov Identifier: NCT04660929)).

While there is much potential for cellular therapies to be curative for patients, a number of factors limit the widespread development and administration of these drugs. Most cellular therapies are currently produced in an autologous fashion and are associated with variable cell product quality, cytokine release syndrome and other toxicities, extended manufacturing times, high costs, and a limited period in which these therapies may be genetically modified to enhance their efficacy (Larson &Maus, Nat Rev Cancer 21, 145-161 (2021)).

The majority of cellular therapies currently being tested in the clinic utilize CAR-T or CAR-NK cells, as these subsets of immune cells demonstrate potent cytotoxicity. Mature primary human T cells that are used for these therapies are found in the blood and secondary lymphoid organs of humans where they act to protect individuals against infectious diseases and cancer. T cells are comprised of αβ (“classic” T cells) and γδ subsets. αβ T cells consist of CD4+ helper T cells and CD8⁺ cytotoxic T cells. CD4⁺ T cells can be further subdivided into TH1 cells, TH2 cells, TH9 cells, TH17 cells, TFH cells, and regulatory T cells. Many αβ T cell subsets exhibit potent cytotoxic function which has been harnessed for the development of cellular therapies.

Similarly, mature primary human NK cells that can be used for cellular therapies are found in the blood, secondary lymphoid organs, liver, and mucosal associated lymphoid tissues, sites that NK cells patrol for the presence of pathogens or transformed cells (Jianhua, et al., Trends in Immunology 34, 573-582 (2013). Like T cells, NK cells demonstrate potent cytotoxic function and are of interest for the development of cellular therapies.

However, primary human immune cells, such as T cells and NK cells, also possess a finite potential for proliferation in vitro and in vivo, limiting their ability to be used for the generation of widespread off-the-shelf cellular therapies. Further, this limited proliferative capacity of mature primary human immune cells impairs their ability to be genetically edited to mitigate cytokine release syndrome and other potential cellular-therapy-associated toxicities, to overcome tumor microenvironment-associated challenges, and to prevent the rejection of allogeneic cellular therapy products in patients.

Patient-derived leukemic cell lines have been studied for decades in cell culture, where their transformed status confers a long-term proliferative capacity that enables their use in a variety of cellular assays. This in turn has facilitated the development of numerous therapies. These cells, however, generally lack the potent cytotoxic function of mature primary human T and NK cells, as they are often immature or derived from dysfunctional T cell clones. The transformed nature of these cells can be mapped on to a collection of mutations that are also frequently found in patients with T cell acute lymphoblastic leukemia. Furthermore, mature T cells from non-human primates can be transformed by herpes viruses through pathways that converge on some of the same mechanisms involved in the transformation of primary human T cells in patients (Biesinger, et al., Proc Natl Acad Sci USA 89, 3116-3119 (1992); Weber, et al., Proc Natl Acad Sci USA 90, 11049-11053 (1993); Fickenscher H, Fleckenstein B., Philos Trans R Soc Lond B Biol Sci. 356(1408):545-67 (2001); Tsygankov, J Cell Physiol. 203(2):305-18 (2005).

While previous studies suggest that primary human T cells may be immortalized through the over-expression of factors such as telomerase-reverse transcriptase (TERT) (Barsov, Methods Mol Biol. 511, 143-58 (2009); Rufer, et al., Blood 98, 597-603 (2001); Hooijberg, et al., J Immunol. 165, 4239-45 (2000)) and human T cell leukemia virus type 1 or human T cell leukemia virus type 2 (HTLV-1/HTLV-2) transcriptional trans-activator protein Tax (Akagi, et. al., Oncogene 14, 2071-2080 (1997); Grassmann, et al., Proc Natl Acad Sci USA 86, 3351-3355 (1989); Ren, et al., J. Biol. Chem. 287, 34683-34693 (2012), or by viruses such as Herpesvirus saimiri (Biesinger, et al., Proc Natl Acad Sci USA 89, 3116-3119 (1992); Weber, et al., Proc Natl Acad Sci USA 90, 11049-11053 (1993)) and HTLV-1/HTLV-2, these approaches are not highly reproducible and can result in reprogramming of modified or infected cells. In addition, cells whose proliferative longevity has been enhanced through the overexpression of TERT still require the use of feeder cells or extensive exogenous stimulation through their T cell receptors to drive proliferation (Rufer, et al., Blood 98, 597-603 (2001); Hooijberg, et al., J Immunol. 165, 4239-45 (2000)). The use of allogeneic feeder cells and extensive repeat stimulation is undesirable when establishing a bank of mature primary human T or NK cells as these methodologies are challenging to scale and may ultimately drive the cells to a dysfunctional state. Further, the use of infectious agents with the ability to transform mature primary human T or NK cells limits the use of these cells in the development of cellular therapies as patients are often immunocompromised.

In light of these challenges, there is a significant need to establish alternative methods by which to extend the proliferative longevity of primary human immune cells to enable large-scale manufacturing of allogeneic cytotoxic cells. The disclosure describes methods and cells that address this unmet need.

### SUMMARY

In one aspect, this disclosure provides a method of generating a population of primary immune cells resistant to replicative senescence (RRS), comprising:
(a) inhibiting the expression of cyclin-dependent kinase inhibitor 2A (CDKN2A), cyclin-dependent kinase inhibitor 2B (CDKN2B), and S-methyl-5′-thioadenosine phosphorylase (MTAP) in the population of primary immune cells;
(b) introducing a transgene encoding one or more STAT5A mutants and/or one or more STAT5B mutants in the population of primary immune cells; and
(c) culturing the primary immune cells in a culture medium;
wherein the culturing induces proliferation of the primary immune cells to yield the population of primary immune cells resistant to replicative senescence (RRS).

In another aspect, this disclosure provides a method of generating a population of primary immune cells resistant to replicative senescence (RRS), comprising:
(a) inhibiting the expression of cyclin-dependent kinase inhibitor 2A (CDKN2A), cyclin-dependent kinase inhibitor 2B (CDKN2B), and S-methyl-5′-thioadenosine phosphorylase (MTAP) in the population of primary immune cells;
(b) introducing a transgene encoding MYC in the population of primary immune cells; and
(c) culturing the primary immune cells in a culture medium;
wherein the culturing induces proliferation of the primary immune cells to yield the population of primary immune cells resistant to replicative senescence (RRS).

In yet another aspect, this disclosure provides a method of generating a population of primary immune cells resistant to replicative senescence (RRS), comprising:
(a) inhibiting the expression of cyclin-dependent kinase inhibitor 2A (CDKN2A), cyclin-dependent kinase inhibitor 2B (CDKN2B), and S-methyl-5′-thioadenosine phosphorylase (MTAP) in the population of primary immune cells;
(b) introducing a transgene encoding TERT in the population of primary immune cells; and
(c) culturing the primary immune cells in a culture medium;
wherein the culturing induces proliferation of the primary immune cells to yield the population of primary immune cells resistant to replicative senescence (RRS).

In yet another aspect, this disclosure provides a method of generating a population of primary immune cells resistant to replicative senescence (RRS), comprising:
(a) inhibiting the expression of one or more endogenous regulatory factors in the population of primary immune cells, wherein the endogenous regulatory factor is cyclin-dependent kinase inhibitor 2A (CDKN2A), cyclin-dependent kinase inhibitor 2B (CDKN2B), or S-methyl-5′-thioadenosine phosphorylase (MTAP);
(b) inhibiting the expression of one or more endogenous immune related genes in the population of primary immune cells, wherein the endogenous immune related gene is beta-2 microglobulin (B2M) and/or T-cell receptor α constant (TRAC);
(c) introducing a transgene encoding one or more STAT5A mutants and/or one or more STAT5B mutants in the population of primary immune cells; and
(d) culturing the population of primary immune cells in a culture medium;
wherein the culturing induces proliferation of the primary immune cells to yield the population of primary immune cells resistant to replicative senescence (RRS).

In yet another aspect, this disclosure provides a method of generating a population of primary immune cells resistant to replicative senescence (RRS), comprising:
(a) inhibiting the expression of cyclin-dependent kinase inhibitor 2A (CDKN2A), cyclin-dependent kinase inhibitor 2B (CDKN2B), and S-methyl-5′-thioadenosine phosphorylase (MTAP) in the population of primary immune cells;
(b) inhibiting the expression of one or more endogenous immune related genes in the population of primary immune cells, wherein the endogenous immune related gene is beta-2 microglobulin (B2M) and/or T-cell receptor α constant (TRAC);
(c) introducing a transgene encoding MYC in the population of primary immune cells; and
(d) culturing the primary immune cells in a culture medium;
wherein the culturing induces proliferation of the primary immune cells to yield the population of primary immune cells resistant to replicative senescence (RRS).

In yet another aspect, this disclosure provides a method of generating a population of primary immune cells resistant to replicative senescence (RRS), comprising:
(a) inhibiting the expression of cyclin-dependent kinase inhibitor 2A (CDKN2A), cyclin-dependent kinase inhibitor 2B (CDKN2B), and S-methyl-5′-thioadenosine phosphorylase (MTAP) in the population of primary immune cells;
(b) inhibiting the expression of one or more endogenous immune related genes in the population of primary immune cells, wherein the endogenous immune related gene is beta-2 microglobulin (B2M) and/or T-cell receptor α constant (TRAC);
(c) introducing a transgene encoding TERT in the population of primary immune cells; and
(d) culturing the primary immune cells in a culture medium;
wherein the culturing induces proliferation of the primary immune cells to yield the population of primary immune cells resistant to replicative senescence (RRS).

In yet another aspect, this disclosure provides a method of generating a population of primary immune cells resistant to replicative senescence (RRS), comprising:
(a) inhibiting expression of cyclin-dependent kinase inhibitor 2A (CDKN2A), cyclin-dependent kinase inhibitor 2B (CDKN2B), or S-methyl-5′-thioadenosine phosphorylase (MTAP) in the population of primary immune cells;
(b) introducing a transgene encoding one or more STAT5A mutants and/or one or more STAT5B mutants in the population of primary immune cells;
(c) introducing a transgene encoding TERT in the population of primary immune cells; and
(d) culturing the population of primary immune cells in a culture medium;
wherein the culturing induces proliferation of the primary immune cells to yield the population of primary immune cells resistant to replicative senescence (RRS).

In yet another aspect, this disclosure provides a method of generating a population of primary immune cells resistant to replicative senescence (RRS), comprising:
(a) inhibiting the expression of cyclin-dependent kinase inhibitor 2A (CDKN2A), cyclin-dependent kinase inhibitor 2B (CDKN2B), and S-methyl-5′-thioadenosine phosphorylase (MTAP) in the population of primary immune cells;
(b) introducing a transgene encoding MYC in the population of primary immune cells;
(c) introducing a transgene encoding TERT in the population of primary immune cells; and
(d) culturing the primary immune cells in a culture medium;
wherein the culturing induces proliferation of the primary immune cells to yield the population of primary immune cells resistant to replicative senescence (RRS).

In some embodiments of the methods disclosed herein, the one or more STAT5A mutants can be H299R, N642H, Y665F, S711F, and combinations thereof, and/or wherein the one or more STAT5B mutants can be H298R, R430C, E433K, N642H, Y665F, P702A, V712E, S715F, and combinations thereof.

In some embodiments of the methods disclosed herein, the method further comprises introducing a transgene encoding TERT in the population of primary immune cells.

In some embodiments of the methods disclosed herein, the method further comprises inhibiting the expression of one or more endogenous immune related genes in the population of primary immune cells. In some embodiments, the endogenous immune related gene is beta-2 microglobulin (B2M) and/or T-cell receptor α constant (TRAC).

In some embodiments of the methods disclosed herein, the method comprises introducing one or more transgenes encoding an anti-apoptotic factor or a virally-derived factor into the primary immune cells. In some embodiments, the anti-apoptotic factor is either B-cell lymphoma-extra large (Bcl-xL) or B-cell lymphoma 2 (Bcl-2). In some embodiments, the virally-derived factor is any one of Saimiriine gammaherpesvirus 2 StpA A11, Herpesvirus saimiri StpC, Herpesvirus saimiri Tip, or a modified Herpesvirus Ateles-Epstein-Barr virus Tio-LMP1.

In some embodiments of the methods disclosed herein, the method further comprises inhibiting the expression of cluster of differentiation 38 (CD38) in the population of primary immune cells.

In some embodiments of the methods disclosed herein, the method further comprises inhibiting the expression of phosphatase and tensin homolog (PTEN) in the population of primary immune cells.

In some embodiments of the methods disclosed herein, the method further comprises, inhibiting the expression of p53 in the population of primary immune cells.

In some embodiments of the methods disclosed herein, the method further comprises introducing a transgene encoding MYC in the population of primary immune cells.

In some embodiments of the methods disclosed herein, the method further comprises introducing a transgene encoding KRAS in the population of primary immune cells. In some embodiments, KRAS is a mutant KRAS A146V.

In some embodiments of the methods disclosed herein, the method further comprises introducing a polynucleotide that encodes a chimeric antigen receptor (CAR) in the population of primary immune cells.

In some embodiments of the methods disclosed herein, the population of primary immune cells comprises total T cells.

In some embodiments of the methods disclosed herein, the population of primary immune cells comprises CD8+ T cells.

In some embodiments of the methods disclosed herein, the population of primary immune cells comprises CD4+ T cells.

In some embodiments of the methods disclosed herein, the population of primary immune cells comprises gamma-delta T cells, mucosal associated invariant T (MAIT) T cells, natural killer (NK) cells, and/or natural killer T (NKT) cells.

In some embodiments of the methods disclosed herein, the population of primary immune cells is human.

In an aspect, this disclosure provides an engineered immune cell population produced according to the methods as disclosed herein.

In another aspect, this disclosure provides a pharmaceutical composition comprising the engineered immune cell population as disclosed herein and a pharmaceutically acceptable carrier.

In yet another aspect, this disclosure provides a method of treating a cancer in a subject in need thereof, comprising administering to the subject a therapeutically effective amount of the pharmaceutical composition as disclosed herein.

In an aspect, this disclosure provides an engineered T cell that does not express cyclin-dependent kinase inhibitor 2A (CDKN2A), cyclin-dependent kinase inhibitor 2B (CDKN2B), and/or S-methyl-5′-thioadenosine phosphorylase (MTAP), and comprises an optional transgene encoding B-cell lymphoma-extra large (Bcl-xL) and a transgene encoding MYC.

In another aspect, this disclosure provides an engineered T cell that does not express cyclin-dependent kinase inhibitor 2A (CDKN2A), cyclin-dependent kinase inhibitor 2B (CDKN2B), and/or S-methyl-5′-thioadenosine phosphorylase (MTAP), and comprises a transgene encoding TERT.

In yet another aspect, this disclosure provides an engineered T cell that does not express cyclin-dependent kinase inhibitor 2A (CDKN2A), cyclin-dependent kinase inhibitor 2B (CDKN2B), S-methyl-5′-thioadenosine phosphorylase (MTAP), beta-2 microglobulin (B2M), and/or T-cell receptor α constant (TRAC), wherein the engineered T cell comprises a transgene encoding one or more STAT5A mutants and/or one or more STAT5B mutants, and wherein the engineered T cell comprises a transgene encoding TERT.

In yet another aspect, this disclosure provides an engineered T cell expressing a transgene encoding a B-cell lymphoma-extra large (Bcl-XL), wherein the engineered T cell does not express cyclin-dependent kinase inhibitor 2A (CDKN2A), cyclin-dependent kinase inhibitor 2B (CDKN2B), S-methyl-5′-thioadenosine phosphorylase (MTAP), and/or phosphatase and tensin homolog (PTEN), and wherein the engineered T cell comprises a transgene encoding one or more STAT5A mutants and/or one or more STAT5B mutants.

In some embodiments of the engineered T cells disclosed herein, the one or more STAT5A mutants can be H299R, N642H, Y665F, S711F, and combinations thereof, and/or wherein the one or more STAT5B mutants can be H298R, R430C, E433K, N642H, Y665F, P702A, V712E, S715F, and combinations thereof.

In some embodiments of the engineered T cells disclosed herein, the engineered T cells further comprise a transgene encoding TERT in the population of primary immune cells.

In some embodiments of the engineered T cells disclosed herein, the engineered T cells further comprise inhibiting the expression of one or more endogenous immune related genes in the population of primary immune cells. In some embodiments, the endogenous immune related gene is beta-2 microglobulin (B2M) and/or T-cell receptor α constant (TRAC).

In some embodiments of the engineered T cells disclosed herein, the engineered T cells comprises one or more transgenes encoding an anti-apoptotic factor or a virally-derived factor into the primary immune cells. In some embodiments, the anti-apoptotic factor is either B-cell lymphoma-extra large (Bcl-xL) or B-cell lymphoma 2 (Bcl-2). In some embodiments, the virally-derived factor is any one of Saimiriine gammaherpesvirus 2 StpA A11, Herpesvirus saimiri StpC, Herpesvirus saimiri Tip, or a modified Herpesvirus Ateles-Epstein-Barr virus Tio-LMP1.

In some embodiments of the engineered T cells disclosed herein, the engineered T cells further comprise inhibited expression of cluster of differentiation 38 (CD38) in the population of primary immune cells.

In some embodiments of the engineered T cells disclosed herein, the engineered T cells further comprise inhibited expression of phosphatase and tensin homolog (PTEN) in the population of primary immune cells.

In some embodiments of the engineered T cells disclosed herein, the engineered T cells further comprise inhibited expression of p53 in the population of primary immune cells.

In some embodiments of the engineered T cells disclosed herein, the engineered T cells further comprise a transgene encoding MYC in the population of primary immune cells.

In some embodiments of the engineered T cells disclosed herein, the engineered T cells further comprise a transgene encoding KRAS in the population of primary immune cells. In some embodiments, KRAS is a mutant KRAS A146V.

In some embodiments of the engineered T cells disclosed herein, the engineered T cells further comprise a polynucleotide that encodes a chimeric antigen receptor (CAR) in the population of primary immune cells.

In an aspect, this disclosure provides for the use of an engineered T cell for the manufacture of a medicament for treating cancer in a patient, wherein the engineered T cell does not express cyclin-dependent kinase inhibitor 2A (CDKN2A), cyclin-dependent kinase inhibitor 2B (CDKN2B), and/or S-methyl-5′-thioadenosine phosphorylase (MTAP), and wherein the engineered T cell comprises a transgene encoding one or more STAT5A mutants and/or one or more STAT5B mutants.

In another aspect, this disclosure provides for the use of an engineered T cell for the manufacture of a medicament for treating cancer in a patient, wherein the engineered T cell does not express cyclin-dependent kinase inhibitor 2A (CDKN2A), cyclin-dependent kinase inhibitor 2B (CDKN2B), and/or S-methyl-5′-thioadenosine phosphorylase (MTAP), and wherein the engineered T cell comprises an optional transgene encoding B-cell lymphoma-extra large (Bcl-xL) and a transgene encoding MYC.

In yet another aspect, this disclosure provides for the use of an engineered T cell for the manufacture of a medicament for treating cancer in a patient, wherein the engineered T cell does not express cyclin-dependent kinase inhibitor 2A (CDKN2A), cyclin-dependent kinase inhibitor 2B (CDKN2B), and/or S-methyl-5′-thioadenosine phosphorylase (MTAP), and wherein the engineered T cell comprises a transgene encoding TERT.

In some embodiments of the use of the engineered T cells disclosed herein, the one or more STAT5A mutants can be H299R, N642H, Y665F, S711F, and combinations thereof, and/or wherein the one or more STAT5B mutants can be H298R, R430C, E433K, N642H, Y665F, P702A, V712E, S715F, and combinations thereof.

In some embodiments of the use of the engineered T cells disclosed herein, the engineered T cells further comprise a transgene encoding TERT in the population of primary immune cells.

In some embodiments of the use of the engineered T cells disclosed herein, the engineered T cells further comprise inhibited expression of one or more endogenous immune related genes in the population of primary immune cells. In some embodiments, the endogenous immune related gene is beta-2 microglobulin (B2M) and/or T-cell receptor α constant (TRAC).

In some embodiments of the use of the engineered T cells disclosed herein, the engineered T cells comprises one or more transgenes encoding an anti-apoptotic factor or a virally-derived factor into the primary immune cells. In some embodiments, the anti-apoptotic factor is either B-cell lymphoma-extra large (Bcl-xL) or B-cell lymphoma 2 (Bcl-2). In some embodiments, the virally-derived factor is any one of Saimiriine gammaherpesvirus 2 StpA A11, Herpesvirus saimiri StpC, Herpesvirus saimiri Tip, or a modified Herpesvirus Ateles-Epstein-Barr virus Tio-LMP1.

In some embodiments of the use of the engineered T cells disclosed herein, the engineered T cells further comprise inhibited expression of cluster of differentiation 38 (CD38) in the population of primary immune cells.

In some embodiments of the use of the engineered T cells disclosed herein, the engineered T cells further comprise inhibited expression of phosphatase and tensin homolog (PTEN) in the population of primary immune cells.

In some embodiments of the use of the engineered T cells disclosed herein, the engineered T cells further comprise inhibited expression of p53 in the population of primary immune cells.

In some embodiments of the use of the engineered T cells disclosed herein, the engineered T cells further comprise a transgene encoding MYC in the population of primary immune cells.

In some embodiments of the use of the engineered T cells disclosed herein, the engineered T cells further comprise a transgene encoding KRAS in the population of primary immune cells. In some embodiments, KRAS is a mutant KRAS A146V.

In some embodiments of the use of the engineered T cells disclosed herein, the engineered T cells further comprise a polynucleotide that encodes a chimeric antigen receptor (CAR) in the population of primary immune cells.

In an aspect, this disclosure provides an engineered T cell for the treatment of cancer in a patient, wherein the engineered T cell does not express cyclin-dependent kinase inhibitor 2A (CDKN2A), cyclin-dependent kinase inhibitor 2B (CDKN2B), and/or S-methyl-5′-thioadenosine phosphorylase (MTAP), and wherein the engineered T cell comprises a transgene encoding one or more STAT5A mutants and/or one or more STAT5B mutants.

In another aspect, this disclosure provides an engineered T cell for the treatment of cancer in a patient, wherein the engineered T cell does not express cyclin-dependent kinase inhibitor 2A (CDKN2A), cyclin-dependent kinase inhibitor 2B (CDKN2B), and/or S-methyl-5′-thioadenosine phosphorylase (MTAP), and comprises an optional transgene encoding B-cell lymphoma-extra large (Bcl-xL) and a transgene encoding MYC.

In yet another aspect, this disclosure provides an engineered T cell for the treatment of cancer in a patient that does not express cyclin-dependent kinase inhibitor 2A (CDKN2A), cyclin-dependent kinase inhibitor 2B (CDKN2B), and/or S-methyl-5′-thioadenosine phosphorylase (MTAP), and comprises a transgene encoding TERT.

In some embodiments of the engineered T cells disclosed herein, the one or more STAT5A mutants can be H299R, N642H, Y665F, S711F, and combinations thereof, and/or wherein the one or more STAT5B mutants can be H298R, R430C, E433K, N642H, Y665F, P702A, V712E, S715F, and combinations thereof.

In some embodiments of the engineered T cells disclosed herein, the engineered T cells further comprise a transgene encoding TERT in the population of primary immune cells.

In some embodiments of the engineered T cells disclosed herein, the engineered T cells further comprise inhibited expression of one or more endogenous immune related genes in the population of primary immune cells. In some embodiments, the endogenous immune related gene is beta-2 microglobulin (B2M) and/or T-cell receptor α constant (TRAC).

In some embodiments of the engineered T cells disclosed herein, the engineered T cells comprises one or more transgenes encoding an anti-apoptotic factor or a virally-derived factor into the primary immune cells. In some embodiments, the anti-apoptotic factor is either B-cell lymphoma-extra large (Bcl-xL) or B-cell lymphoma 2 (Bcl-2). In some embodiments, the virally-derived factor is any one of Saimiriine gammaherpesvirus 2 StpA A11, Herpesvirus saimiri StpC, Herpesvirus saimiri Tip, or a modified Herpesvirus Ateles-Epstein-Barr virus Tio-LMP1.

In some embodiments of the engineered T cells disclosed herein, the engineered T cells further comprise inhibited expression of cluster of differentiation 38 (CD38) in the population of primary immune cells.

In some embodiments of the engineered T cells disclosed herein, the engineered T cells further comprise inhibited expression of phosphatase and tensin homolog (PTEN) in the population of primary immune cells.

In some embodiments of the engineered T cells disclosed herein, the engineered T cells further comprise inhibited expression of p53 in the population of primary immune cells.

In some embodiments of the engineered T cells disclosed herein, the engineered T cells further comprise a transgene encoding MYC in the population of primary immune cells.

In some embodiments of the engineered T cells disclosed herein, the engineered T cells further comprise a transgene encoding KRAS in the population of primary immune cells. In some embodiments, KRAS is a mutant KRAS A146V.

In some embodiments of the engineered T cells disclosed herein, the engineered T cells further comprise a polynucleotide that encodes a chimeric antigen receptor (CAR) in the population of primary immune cells.

### BRIEF DESCRIPTION OF THE DRAWINGS

FIGS. 1A-1B illustrate that Bel-xL insertion conferred a selective advantage for T cell survival in long-term culture. Total primary human T cells (FIG. 1A) or purified primary human CD8⁺ T cells (FIG. 1B) were isolated, stimulated, transfected, and restimulated as described in FIG. 2.

FIG. 2 illustrates the method for identifying survival-enhancing transgenes in primary human T cells.

FIGS. 3A-3B illustrate that ablation of expression of cell cycle regulatory molecules enhanced the proliferative capacity of T cells in long-term culture.

FIGS. 4A-4D illustrate that restimulation of T_{REX}+Bcl-xL cells can enhance their proliferation in long-term culture. FIG. 4A shows total fold expansion of T_{REX}+Bcl-xL cells over time. FIGS. 4B-4D show total fold expansion of T_{REX}+Bcl-xL cells and PTEN-deficient T_{REX}+Bcl-xL cells that were restimulated with αCD3 or αCD3/αCD28 Dynabeads for 3 days after which cells were debeaded. Total fold expansion was tracked and graphed for resting and treated cells over time. Arrows indicate periods of restimulation. Black arrow indicates timepoint of evaluation of additional restimulation modalities. FIGS. 4A-4D show log scale.

FIGS. 5A-5B illustrate that T_{REX}+Bcl-xL cells are dependent on IL-2 for expansion and survival in cell culture. Total fold expansion (FIG. 5A) and cell viability (FIG. 5B) were assessed for 3 T_{REX}+Bcl-xL lines established as in FIG. 3 from two different donors grown in the presence of increasing quantities of recombinant human interleukin 2 (IL-2) for 6 days.

FIGS. 6A-6K illustrate that T_{REX}+Bcl-xL cells phenotypically resemble normal primary human CD8⁺ T cells. T_{REX}+Bcl-xL cells were stained with a fixable viability dye as well as a panel of antibodies to CD3, CD4, CD8, CD28, CD45RO, CCR7, PD1, and TIGIT. T_{REX}+Bcl-xL lines demonstrate expression of CD3 (FIG. 6A) and comprise a high frequency of CD8+ cells (FIG. 6B). T_{REX}+Bcl-xL lines exhibited donor or cell-line specific attributes as exemplified by expression of markers such as PD1 and TIGIT (FIG. 6C), CD28 (FIG. 6D), and CCR7 and CD45RO (FIG. 6E) regardless of Bcl-xL overexpression (GFP⁺ and GFP⁻ cells). T_{REX}+Bcl-xL lines demonstrate expression of CCR2 (FIG. 6F), CCR5 (FIG. 6G), and CXCR3 (FIG. 6J). Expression of CCR6 (FIG. 6H) was heterogenous, while expression of CCR7 (FIG. 6I) and CXCR5 (FIG. 6K) was low to absent.

FIGS. 7A-7F illustrate that T_{REX}+Bcl-xL cells are cytotoxic. Percent cytolysis was computed 12 hours (FIG. 7A) and 24 hours (FIG. 7B) post-addition of effector cells and a T cell engager or control antibody. Supernatants were collected from co-cultures 72 hours post-addition of effector cells and the T cell engager and analyzed for the presence of interferon γ (IFN-γ) (FIG. 7C), IL-2 (FIG. 7D), tumor necrosis factor α (TNF-α) (FIG. 7E), and granzyme B (FIG. 7F).

FIGS. 8A-8G illustrate that T_{REX}+Bcl-xL cells can produce functional CAR-T_{REX}+Bcl-xL cells. Surface CAR expression was assessed 22-days post-transduction using flow cytometry (FIG. 8A). Percent cytolysis was computed 12 hours (FIG. 8B) and 24 hours (FIG. 8C) after addition of effector cells. CAR-T_{REX} activity was benchmarked against that of CAR-T cells and CAR-CD8⁺ T cells. Supernatants were collected from co-cultures 72 hours post-addition of effector cells and analyzed for the presence of IFN-γ (FIG. 8D), IL-2 (FIG. 8E), TNF-α (FIG. 8F), and granzyme B (FIG. 8G).

FIGS. 9A-9C show that T_{REX} cells traffic to similar locations as primary CD8⁺ T cells and are responsive to IL-2 in vivo.

FIGS. 10A-10B show that CAR-T_{REX} cells respond to IL-2 and IL-15 in vivo.

FIGS. 11A-11D show that CAR-T_{REX} cells target solid tumors in vivo.

FIG. 12 shows REX edits reproducibility confer enhanced in vitro proliferation relative to unmodified donor-matched CD8+ T cells. Fold expansion of T_{REX} cells or donor-matched primary (unedited) CD8+ T cells was tracked over time for 4 additional healthy donors.

FIGS. 13A and 13B show that CAR-T_{REX} cells target BCMA+ tumor cells similarly to unmodified CAR-T cells.

FIG. 14 shows that anti-BCMA-T_{REX} and anti-HER2-T_{REX} cells produce lower levels of inflammatory cytokines than anti-BCMA-CAR-T cells and anti-HER2-CAR-T cells following CAR engagement.

FIG. 15 shows that CAR-T_{REX} cells target BCMA+ tumor cells, persist in a serial kill assay, and respond to IL-2.

FIG. 16 shows that the T_{REX} cell phenotype can be generated using different combinations of edits.

FIG. 17 shows that the T_{REX} cells are edited at the expected loci.

FIGS. 18A, 18B, and 18C show that T_{REX} cells demonstrate enrichment in cell cycle-associated gene signatures.

FIG. 19 shows T_{REX} cells are dependent on IL-2 for survival and proliferation.

FIGS. 20A and 20B show that CAR-T_{REX} cells target HER2hi tumor cells similarly to unmodified CAR-T cells with less overall cytokine production.

FIG. 21 shows that REX edits bolster the proliferative capacity of CD4+ T_{REX} cells.

FIG. 22 shows that γδ T_{REX} cells can be generated using REX edits.

FIG. 23 shows that γδ T_{REX} cells are active in a T cell engager (TCE) assay in vitro.

FIG. 24 shows that γδ T_{REX} cells can be generated from multiple γδ cell subsets and diversity is maintained following CAR transduction.

FIGS. 25A and 25B show that γδ-T_{REX} cells target BCMA+ tumor cells similarly to unmodified CAR-T cells.

FIG. 26 shows that REX edits in NK cells support an NK_{REX} cell phenotype.

FIG. 27 shows that NK_{REX} cells are dependent on cytokines for proliferation and survival.

FIG. 28 shows that NK_{REX} cells maintain CAR expression over time.

FIGS. 29A-29C show that NK_{REX} cells are cytotoxic in vitro and CAR expression can further enhance potency.

FIG. 30 shows that T_{REX} cells are sensitive to T cell depleting agents and chemotherapies.

FIG. 31 shows that B2MKO T_{REX} cells are sensitive to NK cell mediated depletion and this can be modulated using anti-CD38 antibodies.

FIG. 32 shows that STAT5A and STAT5B mutants enrich in REX edited CD8⁺ T cells in vitro. STAT mutants were overexpressed with Bcl-XL in T_{REX} cells (REX edit containing CD8⁺ T cells) as per the timeline shown (top). Enrichment of STAT mutants was followed over time using a fluorescent reporter (bottom).

FIG. 33 shows that STAT5A and STAT5B mutants exhibit varying degrees of IL-2 independence in vitro.

FIG. 34 shows that STAT mutant expressing T_{REX} cells retain functionality in a T cell engager assay in accordance with observed surface CD3 expression. T_{REX} cells were co-cultured with two different antigen-expressing tumor lines (left and right). STAT5B N642H mutant containing T_{REX} cells demonstrated low cytotoxicity in this assay due to low surface expression of CD3.

FIG. 35 shows that STAT5 mutant CAR-T_{REX} cells maintain cytotoxic function in a CAR-directed manner in vitro. Percent cytolysis was determined 12 hours post initiation of co-culture of T_{REX} cells and target cells at various effector:target (E:T) cell ratios. STAT5A and STAT5B mutant T_{REX} cells retained functionality as indicated by their capacity to specifically lyse antigen-expressing tumor cells following engagement of the CAR.

FIGS. 36A and 36B show that additional STAT5A and STAT5B mutants enrich in REX edited CD8⁺ T cells in vitro. Two STAT5A mutants and five STAT5B mutants were overexpressed in T_{REX} cells (REX edit containing CD8⁺ T cells) as per the timeline shown (FIG. 36A). STAT mutants were introduced using transposons (T) or lentiviruses (L) as indicated. Enrichment of STAT mutants was followed over time using a fluorescent reporter (FIG. 36B). STAT5A and STAT5B mutant expressing T_{REX} cells enriched during the cell culture process.

FIGS. 37A and 37B show that expression of STAT5A and STAT5B mutants enhances REX edited CD8⁺ T cell expansion in vitro. Proliferation of STAT5A mutant T_{REX} cells, STAT5B mutant T_{REX} cells, and control T_{REX} cells generated in FIG. 36 was tracked over time. STAT5A (FIG. 37A) and STAT5B (FIG. 37B) mutant T_{REX} cells demonstrated enhanced expansion relative to unedited control T_{REX} cells.

FIGS. 38A and 38B show that STAT5A and STAT5B mutants are functional in T_{REX} cells, leading to upregulation of CD25 expression. Surface expression of CD25 was assessed in control T_{REX} cells (UT) and STAT5A/STAT5B mutant containing T_{REX} cells over extended cell culture. STAT mutants were introduced using transposons (FIG. 38A), or lentiviruses (FIG. 38B).

FIG. 39 shows an exemplary workflow for the generation of alternative T_{REX} cell chassis.

FIGS. 40A and 40B show specific edit combinations reproducibly enrich in T_{REX} cells. Engineered T_{REX} cells were assessed for enrichment of edit combinations over time (FIG. 40A is round 1, and FIG. 40B is round 2).

FIG. 41 shows TERT expressing T_{REX}0, T_{REX}3B, and T_{REX}3C cells enrich in culture. T_{REX}0 cells (REX edits), T_{REX}3B cells (REX edits; MYC; Bcl-xL), and T_{REX}3C cells (REX edits; KRAS A146V; MYC; Bcl-xL) were generated and then further modified to overexpress TERT. Enrichment of TERT overexpressing T_{REX}0 cells (T_{REX}0T), T_{REX}3B cells (T_{REX}3BT), and T_{REX}3C cells (T_{REX}3CT) was tracked over time.

FIG. 42 shows certain T_{REX} variants demonstrate enhanced expansion and longevity relative to REX edited CD8⁺ T cells. T_{REX} cell variants were generated as described above and cell expansion was tracked over time. Specific edit combinations (3B, 3C, 3BP, 3CN, 0T, 3BT, and 3CT) enhanced the longevity and proliferative capacity of T_{REX} cell variants relative to T_{REX} (T_{REX}0) cells. However, in some instances edit combinations (2A, 2B, 3A, 3BN) impaired T_{REX} cell longevity and proliferation in cell culture.

FIG. 43 shows that T_{REX} cell variants show increased potency relative to T_{REX}0 in a T cell engager assay. Cytotoxic potential of T_{REX} cells (T_{REX}0) and T_{REX} cell variants was assessed using a control (non-targeting) or active (tumor targeting) T cell engager using the impedance-based xCELLigence platform. Percent cytolysis was determined 12 and 72 hours post addition of T cell engagers. Effector cells were co-cultured with target cells at different effector:target (E:T) cell ratios for T_{REX}0 cells (REX edits), T_{REX}0T cells (REX edits; TERT), T_{REX}3C cells (REX edits; KRAS A146V; MYC, Bcl-xL), T_{REX}3C_3 cells (REX edits; KRAS A146V; MYC; Bcl-XL), T_{REX}3CN cells (REX edits; KRAS A146V; MYC; Bcl-xL; PTEN CRISPR), T_{REX}3B cells (REX edits; MYC; Bcl-xL), T_{REX}3B_3 cells (REX edits; MYC; Bcl-xL), T_{REX}3BT cells (REX edits; MYC; Bcl-xL; TERT), T_{REX}3BP cells (REX edits; MYC; Bcl-xL; TP53 CRISPR), and T_{REX}3BN cells (REX edits; MYC; Bcl-xL; PTEN CRISPR). Dashed line signifies cytotoxicity of T_{REX} (T_{REX}0) cells at 72 hours.

FIGS. 44A and 44B shows that T_{REX} cell variants can express a CAR and CAR-T_{REX} cell variants expand robustly during the culture process. A BCMA targeting CAR was introduced into young T_{REX} cells (T_{REX}0 D60) and T_{REX} cell variants (T_{REX}0T, T_{REX}3C_3, T_{REX}3CN, T_{REX}3B, T_{REX}3B_3, T_{REX}3BT, and T_{REX}3BP). CAR-expressing cells were further enriched to high purity prior to functional assessment (FIG. 44A). Expansion of T_{REX} cells, T_{REX} cell variants, CAR-T_{REX} cells, and CAR-T_{REX} cell variants was tracked throughout the editing, transduction, and enrichment process for each group (FIG. 44B).

FIG. 45 shows CAR-T_{REX} cell variants remain functional even after more than 200 days in culture.

FIG. 46 shows CAR-T_{REX} cell variants persist similarly to Primary CAR-T cells in a serial kill assay. 221-day old CAR-T_{REX} cell variants are at least as functional as young CAR-T_{REX} cell benchmarks.

FIG. 47 shows that TERT overexpression confers an advantage to NK_{REX} and CAR-NK_{REX} cells. NK_{REX} cells (REX edit containing NK cells) were generated and in some instances modified to express a BCMA-targeting CAR (top). NK_{REX} and CAR-NK_{REX} were then further engineered to overexpress TERT. All groups were monitored for enrichment of TERT-expressing NK_{REX} and CAR-NK_{REX} cells over time (top). Proliferation of all groups was also measured throughout the culture process (bottom). Cells were grown under indicated cytokine conditions (IL-2 or IL-2+IL-15).

FIG. 48 shows that TERT overexpressing CAR-NK_{REX} cells show improved function in vitro. Percent cytolysis was determined 6, 12, 48, and 96 hours post initiation of co-culture of effector cells and target cells at various effector:target (E:T) cell ratios.

FIG. 49 shows a workflow for generation of an alternative T_{REX} cell chassis. T_{REX} cells (REX edit containing CD8⁺ T cells) were generated and then further modified to overexpress specific genes of interest (transposon insertion) prior to an enrichment screen. In some instances, these cells were further modified to overexpress other genes of interest (further edits). Engineered T_{REX} cells were assessed for long term growth potential and functionality using different assays.

FIG. 50 shows that additional edit combinations enrich in T_{REX} cells. T_{REX} cells (REX edit containing CD8⁺ T cells) were generated and then further modified as in FIG. 49 to produce T_{REX}0T cells, T_{REX}3B′ cells, T_{REX}3B′T cells, T_{REX}3B cells, T_{REX}3C cells, T_{REX}3C′ cells, and T_{REX}3C′T cells. Engineered T_{REX} cells were assessed for enrichment of edit combinations over time.

FIG. 51 shows that T_{REX} cell variants demonstrate enhanced expansion and longevity relative to REX edited CD8⁺ T cells. T_{REX} cell variants were generated as described in FIG. 49. T_{REX} cell variants (T_{REX}0T, T_{REX}3B′, T_{REX}3B′T, T_{REX}3B, T_{REX}3C*, T_{REX}3C′, and T_{REX}3C′T*) exhibited enhanced longevity and proliferative capacity relative to T_{REX} (T_{REX}0) cells. a: REX edits; b: Transposon insertion; c: REX3B′ sort; d: REX3C′ sort; e: REX3B sort 1; f: REX0 sort; g: REX3B sort 2; h: REX3B re-thaw (for growth curves); and i: TERT insertion.

FIG. 52 shows that T_{REX} cell variants can be single-cell cloned. T_{REX} cells (REX edit containing CD8⁺ T cells) were generated and then further modified as in FIG. 49 to produce T_{REX}3B′ cells, T_{REX}3B cells, and T_{REX}3C′ cells. Single-cell clonability of engineered T_{REX} cells was assessed using flow cytometry based cell sorting or limiting dilution analysis. Engineered cells were seeded at 1, 10, or 100 cells per well and cultured in the presence of IL-2 containing media for 2 weeks. In contrast to T_{REX}0 cells, which could not be single cell cloned, T_{REX}3B′ cells, T_{REX}3B cells, and T_{REX}3C′ cells could produce colonies at varying frequencies. Similar results were obtained for T_{REX}0T, T_{REX}3B, and T_{REX}3CN cells.

FIG. 53 shows that CAR-T_{REX} cell variants expand robustly during the culture process. A BCMA targeting CAR was introduced into young T_{REX} cells (T_{REX}0) and T_{REX} cell variants (T_{REX}0T, T_{REX}3B′, T_{REX}3B′T, T_{REX}3B, T_{REX}3C′, and T_{REX}3C′T). Expansion of CAR-T_{REX} cells and CAR-T_{REX} cell variants was tracked throughout the editing, transduction, and enrichment process for each group (a: REX edits; b: Transposon insertion; c: REX3B′ sort; d: REX3C′ sort; c: REX3B sort 1; f: REX3B sort 2; g: REX3B re-thaw; 1: REX0 purification; 2: REX3C′ and REX3B′ purification; 3: REX0 cryopreservation; 4: REX3B purification; 5: REX3C′T purification; 6: REX3C′ cryopreservation; 7: REX3C′T cryopreservation; 8: REX3B′T cryopreservation).

FIGS. 54A and 54B show that CAR-T_{REX} cell variants are cytotoxic and persist in serial kill assays. A BCMA targeting CAR was introduced into total primary T cells, young T_{REX} cells (T_{REX}0-D88) and T_{REX} cell variants (T_{REX}0T, T_{REX}3B′, T_{REX}3B′T, T_{REX}3B, T_{REX}3C′, and T_{REX}3C′T-D126-182). Cytotoxicity (FIG. 54A) and persistence (FIG. 54B) of CAR-expressing cells were measured over multiple rounds of co-culture with BCMA-expressing JJN3 target cells at an effector:target cell ratio of 1:1. Following each round of co-culture, percent cytolysis (FIG. 54A) and effector cell expansion (FIG. 54B) were assessed.

FIG. 55 shows that CAR-T_{REX} cell variants are at least as functional as young CAR-T_{REX} cell benchmarks in vivo. NSG mice were inoculated with 10E6 MMIS-luciferase tumor cells and 3 days later, primary CAR-T cells (D14), CAR-T_{REX} cells (T_{REX}0, D99-102), or CAR-T_{REX} cell variants (T_{REX}0T, T_{REX}3B′, T_{REX}3B′T, T_{REX}3B, and T_{REX}3C′-D137-200) were dosed at 2E6, 10E6, or 20E6 cells per mouse. All CAR-T_{REX} cell variants demonstrated in vivo functionality, despite higher degrees of in vitro expansion.

FIG. 56 shows that cryo-recovered TERT overexpressing CAR-NK_{REX} cells are cytotoxic and persist better in serial kill assays than younger cryo-recovered CAR-NK_{REX} cells. BCMA targeting CAR-NK_{REX} cells and TERT overexpressing CAR-NK_{REX} cells were generated as described in FIG. 47. Cytotoxicity (left) and persistence (right) of CAR-expressing cells were measured over multiple rounds of co-culture with BCMA-expressing JJN3 target cells at an effector:target cell ratio of 2:1 in the presence of IL-2. Following each round of co-culture, percent cytolysis (left) and effector cell expansion (right) were assessed. TERT overexpressing CAR-NK_{REX} cells demonstrated enhanced functionality and persistence in this assay.

FIG. 57 shows that TERT overexpressing CAR-NK_{REX} cells maintain cytotoxic potential following cryo-recovery and expansion to more than 400 days in culture. TERT overexpressing CAR-NK_{REX} cells were cryo-preserved on day 256 and then cryo-recovered and expanded to day 414 before assay initiation. Percent cytolysis was computed at the end of the co-culture.

FIG. 58 shows a schematic overview of the generation of TERT overexpressing NK_{REX} and CAR-NK_{REX} cells.

FIG. 59 shows that TERT overexpression reproducibly confers an advantage to NK_{REX} and CAR-NK_{REX} cells. NK_{REX} cells were generated and modified to express a BCMA-targeting CAR. All groups were monitored for expansion throughout the engineering and culture process (top). Enrichment of TERT-expressing NK_{REX} cells was also assessed over time (bottom).

FIG. 60 shows that TERT overexpressing CAR-NK_{REX} cells are cytotoxic and expand better in serial kill assays than younger CAR-NK_{REX} cells. Cytotoxicity (left) and persistence (right) of effector cells were measured over multiple rounds of co-culture with BCMA-expressing JJN3 target cells at an effector:target cell ratio of 2:1 in the presence of IL-2.

FIG. 61 shows a timeline for generation of STAT mutant expressing T_{REX} cells.

FIG. 62 shows STAT5A and STAT5B mutants enrich in REX edited CD8⁺ T cells in vitro enrichment.

FIG. 63A shows that STAT5A and STAT5B mutants are functional in T_{REX} cells, leading to upregulation of CD25 expression 5 days after transduction. Surface expression of CD25 was assessed in control T_{REX} cells (UT) and STAT5A, STAT5B, and STAT3 mutant containing T_{REX} cells over extended cell culture.

FIG. 63B shows that STAT5A and STAT5B mutants are functional in T_{REX} cells, leading to upregulation of CD25 expression 20 days after transduction. Surface expression of CD25 was assessed in control T_{REX} cells (UT) and STAT5A, STAT5B, and STAT3 mutant containing T_{REX} cells over extended cell culture.

FIG. 63C shows that STAT5A and STAT5B mutants are functional in T_{REX} cells, leading to upregulation of CD25 expression 35 days after transduction. Surface expression of CD25 was assessed in control T_{REX} cells (UT) and STAT5A, STAT5B, and STAT3 mutant containing T_{REX} cells over extended cell culture.

FIG. 63D shows that STAT5A and STAT5B mutants are functional in T_{REX} cells, leading to upregulation of CD25 expression 42 days after transduction. Surface expression of CD25 was assessed in control T_{REX} cells (UT) and STAT5A, STAT5B, and STAT3 mutant containing T_{REX} cells over extended cell culture.

FIG. 64 shows that STAT5A and STAT5B mutant T_{REX} cells can express a CAR. A BCMA targeting CAR was introduced into unmodified T_{REX} cells (T_{REX} (UT)) and STAT mutant T_{REX} cells (STAT MU1, STAT MU2, STAT MU3, STAT MU4, STAT MU5, STAT MU6, STAT MU7, STAT MU8, STAT MU9, STAT MU10, and STAT MU11). CAR-expressing cells were further enriched to high purity prior to functional assessment.

FIG. 65 shows STAT mutant CAR-T_{REX} cell expand robustly during the culture process. Expansion of STAT mutant CAR-T_{REX} cells was tracked throughout the editing, transduction, and enrichment process.

FIG. 66 shows that STAT5A and STAT5B mutants confer varying degrees of IL-2 independence in vitro. STAT5A mutant, STAT5B mutant, and STAT3 mutant T_{REX} cells and control T_{REX} cells were cultured in media in the absence of IL-2 and expansion was followed over time (top). In the absence of a CAR, multiple groups showed IL-2 dependence (control T_{REX} cells, STAT MU1, STAT MU3, STAT MU4, STAT MU6, STAT MU7, STAT MU8, STAT MU10, STAT MU12, STAT MU13) while others grew independently of IL-2 (STAT MU2, STAT MU5, STAT MU9, and STAT MU11). Cells were grown in the presence of decreasing amounts of IL-2 and expansion was followed over time (bottom). STAT MU6 and STAT MU7 CAR-T_{REX} cells maintained dependence on IL-2 (though these groups proliferated under low-IL-2 conditions). STAT MU8 and STAT MU9 CAR-T_{REX} cells demonstrated a capacity to proliferate in the absence of IL-2, while the greatest degree of IL-2 independence was observed in STAT MU4, STAT MU5, STAT MU10 and STAT MU11 CAR-T_{REX} cells.

FIG. 67 shows that STAT5B mutant CAR-T_{REX} cell variants remain functional even after more than 150 days in culture. Cytotoxicity of CAR-expressing cells was measured using the impedance-based xCELLigence platform. Percent cytolysis was determined 12 hours and 72 hours post initiation of co-culture of effector cells and target cells at various effector:target (E:T) cell ratios.

FIG. 68 shows that STAT5B mutant CAR-T_{REX} cells demonstrate enhanced persistence and functionality relative to young CAR-T_{REX} cell benchmarks in a serial kill assay. Cytotoxicity and persistence of CAR-expressing cells were measured over multiple rounds of co-culture with BCMA-expressing JJN3 target cells at an effector:target cell ratio of 0.3:1. Following each round of co-culture, percent cytolysis (top) and effector cell expansion (bottom) were assessed. All STAT5B mutants enhanced CAR-T_{REX} cell persistence and cytotoxicity to varying degrees.

FIG. 69 shows that STAT5B mutant CAR-T_{REX} cells are at least as functional as young CAR-T_{REX} cell benchmarks in vivo. NSG mice were inoculated with 2E6 MMIS-luciferase tumor cells. Six days later, BCMA-T_{REX} cells (T_{REX}, day 99) or STAT mutant BCMA-T_{REX} cells (STAT MU5, STAT MU6, STAT MU7, STAT MU9, and STAT MU11-all day 112) were dosed at 2E6 or 10E6 cells per mouse. All STAT mutant T_{REX} cells demonstrated in vivo functionality, despite higher degrees of in vitro expansion (numbers shown under group title).

FIG. 70 shows a schematic overview of the generation of STAT mutant overexpressing NK_{REX} cells. Eight STAT5B mutants and two STAT3 mutants were overexpressed in NK_{REX} cells (REX edit containing NK cells) as per the timeline shown.

FIG. 71 shows that STAT5B and STAT3 mutants enrich in REX edited NK cells in vitro. Enrichment of STAT mutants was followed over time using a fluorescent reporter. STAT5B and STAT3 mutant expressing NK_{REX} cells enriched during the cell culture process.

FIG. 72 shows STAT5B and STAT3 mutant expression enhances proliferation of NK_{REX} cells. STAT5B mutants and STAT3 mutants were overexpressed in NK_{REX} cells (REX edit containing NK cells) as per FIG. 70. STAT5B mutant NK_{REX} cells (top) and STAT3 mutant NK_{REX} cells (bottom) proliferated at a faster rate than donor matched unmodified NK_{REX} cells as evidenced by steeper growth curves following introduction of the STAT mutants.

FIG. 73 shows that STAT mutant expressing NK_{REX} cells retain functionality in an in vitro cytotoxicity assay. Cytotoxic function of STAT mutant containing NK_{REX} cells was assessed through co-culture of control (unmodified) NK_{REX} and STAT mutant NK_{REX} cells with K562-luciferase cells and percent cytolysis was determined 24 hours post initiation of the co-cultures. Effector cells were co-cultured with K562 cells at 2 different effector:target cell ratios (1:1-top and 2:1-bottom). STAT5B mutant and STAT3 mutant expressing NK_{REX} cells demonstrated similar cytotoxic activity to unmodified NK_{REX} cells.

### DETAILED DESCRIPTION

The disclosure relates to methods, cells, and compositions for preparing cell populations and compositions for adoptive cell therapy. In particular, provided herein are methods for expansion and proliferation of primary immune cells including T cell populations.

As utilized in accordance with the present disclosure, unless otherwise indicated, all technical and scientific terms shall be understood to have the same meaning as commonly understood by one of ordinary skill in the art. Unless otherwise required by context, singular terms shall include pluralities and plural terms shall include the singular.

As used herein, the terms “comprise” and “include” and variations thereof (e.g., “comprises,” “comprising,” “includes,” and “including”) will be understood to indicate the inclusion of a stated component, feature, element, or step or group of components, features, elements or steps but not the exclusion of any other component, feature, element, or step or group of components, features, elements, or steps. Any of the terms “comprising,” “consisting essentially of,” and “consisting of” may be replaced with either of the other two terms, while retaining their ordinary meanings.

As used herein, the singular forms “a,” “an” and “the” include plural referents unless the context clearly indicates otherwise.

Percentages disclosed herein can vary in amount by ±10, 20, or 30% from values disclosed and remain within the scope of the contemplated disclosure.

Unless otherwise indicated or otherwise evident from the context and understanding of one of ordinary skill in the art, values herein that are expressed as ranges can assume any specific value or sub-range within the stated ranges in different aspects of the disclosure, to the tenth of the unit of the lower limit of the range, unless the context clearly dictates otherwise.

As used herein, ranges and amounts can be expressed as “about” a particular value or range. The term “about” also includes the exact amount. For example, “about 5%” means “about 5%” and also “5%.” The term “about” can also refer to +10% of a given value or range of values. Therefore, about 5% also means 4.5%-5.5%, for example. Unless otherwise clear from context, all numerical values provided herein are modified by the term “about.”

As used herein, the terms “or” and “and/or” can describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” Resistant to replicative senescence (RRS) refers to primary immune cells that are resistant to replicative senescence (RS) that leads to a finite number of population doublings. As a result, the population of primary immune cells described herein advantageously have extended proliferative capacity.

In one aspect, this disclosure provides a method of generating a population of primary immune cells resistant to replicative senescence (RRS), comprising: (a) inhibiting the expression of cyclin-dependent kinase inhibitor 2A (CDKN2A), cyclin-dependent kinase inhibitor 2B (CDKN2B), and S-methyl-5′-thioadenosine phosphorylase (MTAP) in the population of primary immune cells; (b) introducing a transgene encoding one or more STAT5A mutants and/or one or more STAT5B mutants in the population of primary immune cells; and (c) culturing the primary immune cells in a culture medium; wherein the culturing induces proliferation of the primary immune cells to yield the population of primary immune cells resistant to replicative senescence (RRS).

In one aspect, this disclosure provides a method of generating a population of primary immune cells resistant to replicative senescence (RRS), comprising: (a) inhibiting the expression of cyclin-dependent kinase inhibitor 2A (CDKN2A), cyclin-dependent kinase inhibitor 2B (CDKN2B), and S-methyl-5′-thioadenosine phosphorylase (MTAP) in the population of primary immune cells; and (b) introducing a transgene encoding MYC in the population of primary immune cells; and (c) culturing the primary immune cells in a culture medium; wherein the culturing induces proliferation of the primary immune cells to yield the population of primary immune cells resistant to replicative senescence (RRS).

In one aspect, this disclosure provides a method of generating a population of primary immune cells resistant to replicative senescence (RRS), comprising: (a) inhibiting the expression of cyclin-dependent kinase inhibitor 2A (CDKN2A), cyclin-dependent kinase inhibitor 2B (CDKN2B), and S-methyl-5′-thioadenosine phosphorylase (MTAP) in the population of primary immune cells; (b) introducing a transgene encoding TERT in the population of primary immune cells; and (c) culturing the primary immune cells in a culture medium; wherein the culturing induces proliferation of the primary immune cells to yield the population of primary immune cells resistant to replicative senescence (RRS).

In one aspect, this disclosure provides a method of generating a population of primary immune cells resistant to replicative senescence (RRS), comprising: (a) inhibiting the expression of one or more endogenous regulatory factors in the population of primary immune cells, wherein the endogenous regulatory factor is cyclin-dependent kinase inhibitor 2A (CDKN2A), cyclin-dependent kinase inhibitor 2B (CDKN2B), or S-methyl-5′-thioadenosine phosphorylase (MTAP); (b) inhibiting the expression of one or more endogenous immune related genes in the population of primary immune cells, wherein the endogenous immune related gene is beta-2 microglobulin (B2M) and/or T-cell receptor α constant (TRAC); (c) introducing a transgene encoding one or more STAT5A mutants and/or one or more STAT5B mutants in the population of primary immune cells; and (d) culturing the population of primary immune cells in a culture medium; wherein the culturing induces proliferation of the primary immune cells to yield the population of primary immune cells resistant to replicative senescence (RRS).

In one aspect, this disclosure provides a method of generating a population of primary immune cells resistant to replicative senescence (RRS), comprising: (a) inhibiting the expression of cyclin-dependent kinase inhibitor 2A (CDKN2A), cyclin-dependent kinase inhibitor 2B (CDKN2B), and S-methyl-5′-thioadenosine phosphorylase (MTAP) in the population of primary immune cells; (b) inhibiting the expression of one or more endogenous immune related genes in the population of primary immune cells, wherein the endogenous immune related gene is beta-2 microglobulin (B2M) and/or T-cell receptor α constant (TRAC); and (c) introducing a transgene encoding MYC in the population of primary immune cells; and (d) culturing the primary immune cells in a culture medium; wherein the culturing induces proliferation of the primary immune cells to yield the population of primary immune cells resistant to replicative senescence (RRS).

In one aspect, this disclosure provides a method of generating a population of primary immune cells resistant to replicative senescence (RRS), comprising: (a) inhibiting the expression of cyclin-dependent kinase inhibitor 2A (CDKN2A), cyclin-dependent kinase inhibitor 2B (CDKN2B), and S-methyl-5′-thioadenosine phosphorylase (MTAP) in the population of primary immune cells; (b) inhibiting the expression of one or more endogenous immune related genes in the population of primary immune cells, wherein the endogenous immune related gene is beta-2 microglobulin (B2M) and/or T-cell receptor α constant (TRAC); (c) introducing a transgene encoding TERT in the population of primary immune cells; and (d) culturing the primary immune cells in a culture medium; wherein the culturing induces proliferation of the primary immune cells to yield the population of primary immune cells resistant to replicative senescence (RRS).

In one aspect, this disclosure provides a method of generating a population of primary immune cells resistant to replicative senescence (RRS), comprising: (a) inhibiting expression of cyclin-dependent kinase inhibitor 2A (CDKN2A), cyclin-dependent kinase inhibitor 2B (CDKN2B), or S-methyl-5′-thioadenosine phosphorylase (MTAP) in the population of primary immune cells; (b) introducing a transgene encoding one or more STAT5A mutants and/or one or more STAT5B mutants in the population of primary immune cells; (c) introducing a transgene encoding TERT in the population of primary immune cells; and (d) culturing the population of primary immune cells in a culture medium; wherein the culturing induces proliferation of the primary immune cells to yield the population of primary immune cells resistant to replicative senescence (RRS).

In one aspect, this disclosure provides a method of generating a population of primary immune cells resistant to replicative senescence (RRS), comprising: (a) inhibiting the expression of cyclin-dependent kinase inhibitor 2A (CDKN2A), cyclin-dependent kinase inhibitor 2B (CDKN2B), and S-methyl-5′-thioadenosine phosphorylase (MTAP) in the population of primary immune cells; (b) introducing a transgene encoding MYC in the population of primary immune cells; (c) introducing a transgene encoding TERT in the population of primary immune cells; and (d) culturing the primary immune cells in a culture medium; wherein the culturing induces proliferation of the primary immune cells to yield the population of primary immune cells resistant to replicative senescence (RRS).

In some embodiments of the methods disclosed herein, the one or more STAT5A mutants can be H299R, N642H, Y665F, S711F, and combinations thereof, and/or wherein the one or more STAT5B mutants can be H298R, R430C, E433K, N642H, Y665F, P702A, V712E, S715F, and combinations thereof.

In some embodiments of the methods disclosed herein, the method further comprises introducing a transgene encoding TERT in the population of primary immune cells. In some embodiments of the methods disclosed herein, the method further comprises inhibiting the expression of one or more endogenous immune related genes in the population of primary immune cells. In some embodiments, the endogenous immune related gene is beta-2 microglobulin (B2M) and/or T-cell receptor α constant (TRAC). In some embodiments of the methods disclosed herein, the method comprises introducing one or more transgenes encoding an anti-apoptotic factor or a virally-derived factor into the primary immune cells. In some embodiments, the anti-apoptotic factor is either B-cell lymphoma-extra large (Bcl-xL) or B-cell lymphoma 2 (Bcl-2). In some embodiments, the anti-apoptotic factor is B-cell lymphoma-extra large (Bcl-XL). In some embodiments, the virally-derived factor is any one of Saimiriine gammaherpesvirus 2 StpA A11, Herpesvirus saimiri StpC, Herpesvirus saimiri Tip, or a modified Herpesvirus Ateles-Epstein-Barr virus Tio-LMP1.

In some embodiments of the methods disclosed herein, the method further comprises inhibiting the expression of cluster of differentiation 38 (CD38) in the population of primary immune cells. In some embodiments of the methods disclosed herein, the method further comprises inhibiting the expression of phosphatase and tensin homolog (PTEN) in the population of primary immune cells. In some embodiments of the methods disclosed herein, the method further comprises, inhibiting the expression of p53 in the population of primary immune cells.

In some embodiments of the methods disclosed herein, the method further comprises introducing a transgene encoding MYC in the population of primary immune cells.

In some embodiments of the methods disclosed herein, the method further comprises introducing a transgene encoding KRAS in the population of primary immune cells. In some embodiments, KRAS is a mutant KRAS A146V.

In some embodiments of the methods disclosed herein, the method further comprises introducing a polynucleotide that encodes a chimeric antigen receptor (CAR) in the population of primary immune cells.

In some embodiments of the methods disclosed herein, the population of primary immune cells comprises total T cells. In some embodiments of the methods disclosed herein, the population of primary immune cells comprises CD8⁺ T cells. In some embodiments of the methods disclosed herein, the population of primary immune cells comprises CD4⁺ T cells.

In some embodiments of the methods disclosed herein, the population of primary immune cells comprises gamma-delta T cells, mucosal associated invariant T (MAIT) T cells, natural killer (NK) cells, and/or natural killer T (NKT) cells. In some embodiments of the methods disclosed herein, the population of primary immune cells is human.

A genetic edit refers to a change to the genetic material of the primary immune cell. A genetic edit includes genetic material to be added, removed, or altered. In particular aspects, genetic edits comprise introducing a transgene into the primary immune cells and/or inhibiting the expression of a gene in the primary immune cell. In particular aspects, introducing one or more genetic edits comprise introducing one or more transgenes. In some aspects, the one or more transgenes encode an anti-apoptotic factor, a virally-derived factor, or oncogene or proto-oncogene into the primary immune cells. In some embodiments, a genetic edit can refer to one or more of: (1) inhibiting the expression of cyclin-dependent kinase inhibitor 2A (CDKN2A), cyclin-dependent kinase inhibitor 2B (CDKN2B), and S-methyl-5′-thioadenosine phosphorylase (MTAP); (2) introducing a transgene encoding one or more STAT5A mutants and/or one or more STAT5B mutants; (3) introducing a transgene encoding B-cell lymphoma-extra large (Bcl-xL); (4) introducing a transgene encoding MYC; (5) introducing a transgene encoding TERT; (6) inhibiting the expression of phosphatase and tensin homolog (PTEN); (7) introducing a transgene encoding KRAS or a mutant thereof; (8) inhibiting the expression of p53; (9) inhibiting the expression of cluster of differentiation 38 (CD38); and/or (10) inhibiting the expression of beta-2 microglobulin (B2M) and/or T-cell receptor α constant (TRAC).

The term “Primary immune cell(s)” can refer to any cell(s) involved in a primary immune response such as T cells, B-cells and NK cells, neutrophils, and monocytes/macrophages/dendritic cells. In some aspects, primary immune cells can comprise total T cells, CD4-positive T cells, CD8-positive T cells, regulatory T cells, gamma-delta T cells, mucosal associated invariant T (MAIT) T cells, natural killer (NK) cells, or natural killer T (NKT) cells.

The term “transgene” refers to any nucleic acid sequence that is introduced into the cell by experimental manipulations. A transgene may be an “endogenous DNA sequence” or a “heterologous DNA sequence.” The transgene may be isolated and obtained in suitable quantity using one or more methods that are well known in the art. These methods and others useful for isolating a transgene are set forth, for example, in Sambrook et al. (supra) and in Berger and Kimmel (Methods in Enzymology: Guide to Molecular Cloning Techniques, vol. 152, Academic Press, Inc., San Diego, CA (1987)).

The transgene can be incorporated into a “transgene construct” that comprises the gene of interest along with other regulatory DNA sequences needed either for temporal, or cell specific, or enhanced expression of the transgenes of interest.

The transgene may be introduced into the cells by any suitable method or technique known in the art. In particular aspects, the transgene is introduced using a plasmid-based DNA transposon, lentivirus platform, or site-specific integration via CRISPR. The transgene expression in the cell can be constitutive or inducible.

In particular aspects, the transgene encodes an anti-apoptotic factor. An “anti-apoptotic factor” refers to a protein or an oligonucleotide (which may be an oligonucleotide encoding for a protein or a silencing nucleotide) which acts to prevent apoptosis of a cell, in particular a cell experiencing stress, a cell received signal to undergo apoptosis or a cell undergoing abnormal cell proliferation. In particular aspects, the anti-apoptotic factor is B-cell lymphoma-extra large (Bcl-xL) or B-cell lymphoma 2 (Bcl-2).

In particular aspects, the transgene encodes one or more oncogenes or proto-oncogenes selected from MYC, KRAS, a KRAS mutant, NRAS, and a NRAS mutant. In particular aspects, the oncogene is a KRAS mutant selected from G12C and/or A146V. In particular aspects, the anti-apoptotic factor is a NRAS mutant selected from G12D.

In particular aspects, the transgene encodes a virally-derived factor. A “virally-derived factor” refers to both naturally-occurring viral peptides, polypeptides, or proteins, as well as peptides, polypeptides, or proteins displaying a degree of sequence identity and/or similarity to a viral protein and/or maintaining one or more structural, mechanistic, or antigenic qualities of the viral protein. In particular aspects, the virally-derived factor is from Saimiriine gammaherpesvirus 2 StpA A11, Herpesvirus saimiri StpC, Herpesvirus saimiri Tip, or a modified Herpesvirus Ateles-Epstein-Barr virus Tio-LMP1.

In other aspects, the transgene encodes a protein relating to activating signals in the cell.

In some aspects, methods of the disclosure further include inhibiting the expression of one or more endogenous regulatory factors in the primary immune cells such that the activity of the endogenous regulatory factor is eliminated or reduced. As used herein a “regulatory factor” refers to a gene that encodes a protein involved in regulating the cell cycle arrest, cell death, or signal suppression. The endogenous regulatory factor may be down regulated or blocked by any suitable method or technique known in the art. Known methods for down regulation of gene expression or decreasing the activity of a factor include, but are not limited to, CRISPR/Cas (including cytosine and adenine base editors), microRNA, shRNA, RNAi, TALENs, zinc finger nucleases, meganucleases, neutralizing antibodies, small molecule inhibitors, chemical inhibitors blocking downstream signaling pathways, and the like. The inhibition of the endogenous regulatory factor can be complete inhibition, partial inhibition, down regulation of gene expression or decreasing the activity of a factor. In some aspects, endogenous regulatory factor activity or gene expression is reduced by between 1%-100% (i.e., 1%, 5%, 10%, 15%, 20%, 25%, 30%, 35%, 40%, 45%, 50%, 55%, 60%, 65%, 70%, 75%, 80%, 85%, 90%, 95%, 98%, 99%, 100%). A regulatory factor includes a gene that encodes a protein involved in regulating the cell cycle arrest, cell death or signal suppression. In particular aspects, the one or more endogenous regulatory factors are cyclin-dependent kinase inhibitor 2A (CDKN2A), cyclin-dependent kinase inhibitor 2B (CDKN2B), and/or S-methyl-5′-thioadenosine phosphorylase (MTAP). In particular aspects, the one or more endogenous regulatory factors are RB Transcriptional Corepressor 1 (RB1), TP53, Autophagy and Beclin 1 Regulator 1 (AMBRA1), Neurofibromatosis type 1 (NF1), Tyrosine-protein phosphatase non-receptor type 2 (PTPN2), or Suppressor of Cytokine Signaling 1 (SOCS1).

In some aspects, methods of the disclosure further comprise introducing one or more transgenes encoding one or more regulatory factors in the primary immune cells. In some aspects, methods of the disclosure further comprise introducing one or more transgenes encoding one or more regulatory factors in the primary immune cells, wherein the one or more regulatory factors is overexpressed in the primary immune cells. In particular aspects, the transgene is introduced using a plasmid-based DNA transposon, lentivirus platform, or site-specific integration via CRISPR. In particular aspects, the one or more regulatory factors can include a signal transducer and activator of transcription 5A (STAT5A) mutant, a signal transducer and activator of transcription 5B (STA5B) mutant, or a MYC proto-oncogene bHLH transcription factor (c-MYC, MYC).

In particular aspects, a transgene encodes a signal transducer and activator of transcription 5A (STAT5A) mutant and/or a signal transducer and activator of transcription 5B (STA5B) mutant. STAT5A and STAT5B are members of the STAT family of transcription factors. STAT family members act as transcription activators that mediate the signal transduction pathways triggered by various cell ligands, such as IL2, IL4, CSF1, and different growth hormones. In particular aspects, the transgene is introduced using a plasmid-based DNA transposon, lentivirus platform, or site-specific integration via CRISPR. In some embodiments, a STAT5A mutant can include, but is not limited to, H299R, N642H, Y665F, S711F, and combinations thereof. In some embodiments, a STAT5B mutant can include, but is not limited to, H298R, R430C, E433K, N642H, Y665F, P702A, V712E, S715F, and combinations thereof.

In particular aspects, a transgene encodes telomerase reverse transcriptase (TERT).

The term “endogenous” refers to developing or originating within a cell, a tissue, or an organism or part of a cell, a tissue or an organism.

In some aspects, methods of the disclosure further include inhibiting the expression of one or more endogenous immune related genes in the primary immune cells such that the activity of the immune related genes is eliminated or reduced. As used herein an “immune related gene” refers to a gene that encodes a protein involved in effecting an immune response. In certain aspects, the immune related gene encodes a protein that is involved in host-versus-graft (HvG) and graft-versus-host (GvH) allogeneic immune responses. The immune related gene may be down regulated or blocked by any suitable method or technique known in the art. Known methods for down regulation of gene expression or decreasing the activity of an immune related gene include, but are not limited to, CRISPR/Cas (including cytosine and adenine base editors), microRNA, shRNA, RNAi, TALENs, zinc finger nucleases, meganucleases, neutralizing antibodies, small molecule inhibitors, chemical inhibitors blocking downstream signaling pathways, and the like. The inhibition of the endogenous immune related gene can be complete inhibition, partial inhibition, down regulation of gene expression or decreasing the activity of a factor. In some aspects, endogenous immune related gene activity or gene expression is reduced by between 1%-100% (i.e., 1%, 5%, 10%, 15%, 20%, 25%, 30%, 35%, 40%, 45%, 50%, 55%, 60%, 65%, 70%, 75%, 80%, 85%, 90%, 95%, 98%, 99%, 100%). An immune related gene includes a gene that encodes a protein involved in effecting an immune response. An immune related gene can encode a protein that is involved in host-versus-graft (HvG) and graft-versus-host (GvH) allogeneic immune responses. In particular aspects, the one or more endogenous immune related genes are beta-2 microglobulin (B2M) or T-cell receptor α constant (TRAC). In particular aspects, the one or more endogenous immune related genes are genes of the major histocompatibility complex (MHC), human leukocyte antigen class I genes (e.g. HLA-A, HLA-B, HLA-C), human leukocyte antigen class II genes (HLA-DR, HLA-DQ, and HLA-DP), T cell receptors (e.g. αβ T cell receptor), interleukin 1 (IL-1), interleukin 2 (IL-2), interleukin 4 (IL-4), interleukin 6 (IL-6), interleukin 10 (IL-10), interleukin 23 (IL-23), interferon-γ (IFNγ), CCL2, CCL3, CCL4, CCL5, CXCL2, CXCL9-11, CCL17, CCL27, programmed death-1 (PD-1), TIM3, or TIGIT.

In further aspects, the methods disclosed herein include inhibiting the expression of cluster of differentiation 38 (CD38) in the primary immune cells such that the activity of CD38 is eliminated or reduced. CD38 may be down regulated or blocked by any suitable method or technique known in the art. Known methods for down regulation of gene expression or decreasing the activity of CD38 include, but are not limited to, CRISPR/Cas (including cytosine and adenine base editors), microRNA, shRNA, RNAi, TALENs, zinc finger nucleases, meganucleases, neutralizing antibodies, small molecule inhibitors, chemical inhibitors blocking downstream signaling pathways, and the like. In some aspects, CD38 activity or gene expression is reduced by between 1%-100% (i.e., 1%, 5%, 10%, 15%, 20%, 25%, 30%, 35%, 40%, 45%, 50%, 55%, 60%, 65%, 70%, 75%, 80%, 85%, 90%, 95%, 98%, 99%, 100%).

In further aspects, the methods disclosed herein include inhibiting the expression of p53 in the primary immune cells such that the activity of p53 is eliminated or reduced. In some embodiments, p53 may be down regulated or blocked by any suitable method or technique known in the art. Known methods for down regulation of gene expression or decreasing the activity of p53 include, but are not limited to, CRISPR/Cas (including cytosine and adenine base editors), microRNA, shRNA, RNAi, TALENs, zinc finger nucleases, meganucleases, neutralizing antibodies, small molecule inhibitors, chemical inhibitors blocking downstream signaling pathways, and the like. In some aspects, p53 activity or gene expression is reduced by between 1%-100% (i.e., 1%, 5%, 10%, 15%, 20%, 25%, 30%, 35%, 40%, 45%, 50%, 55%, 60%, 65%, 70%, 75%, 80%, 85%, 90%, 95%, 98%, 99%, 100%).

In further aspects, the methods disclosed herein include inhibiting the expression of phosphatase and tensin homolog (PTEN) in the primary immune cells such that the activity of PTEN is eliminated or reduced. PTEN may be down regulated or blocked by any suitable method or technique known in the art. Known methods for down regulation of gene expression or decreasing the activity of PTEN include, but are not limited to, CRISPR/Cas (including cytosine and adenine base editors), microRNA, shRNA, RNAi, TALENs, zinc finger nucleases, meganucleases, neutralizing antibodies, small molecule inhibitors, chemical inhibitors blocking downstream signaling pathways, and the like. In some aspects, PTEN activity or gene expression is reduced by between 1%-100% (i.e., 1%, 5%, 10%, 15%, 20%, 25%, 30%, 35%, 40%, 45%, 50%, 55%, 60%, 65%, 70%, 75%, 80%, 85%, 90%, 95%, 98%, 99%, 100%).

The term “T_{REX}” refers to a “T cell that is Renewably Expandable” using e.g., the techniques and genetic modifications provided herein. More specifically, T_{REX} cells refer to cells with decreased or ablated expression of some or all of cyclin-dependent kinase inhibitor 2A (CDKN2A), cyclin-dependent kinase inhibitor 2B CDKN2B, and S-methyl-5′-thioadenosine phosphorylase (MTAP).

In some aspects, inhibiting the expression of one or more endogenous regulatory factors (e.g., CDKN2A, CDKN2B, or MTAP) occurs after introduction of the one or more transgenes into the cells. In some aspects, primary immune cells in which one or more transgene has been introduced are cultured for at least 2 days, at least 3 days, at least 4 days, at least 5 days, at least 10 days, at least 11 days, at least 12 days, at least 13 days, at least 14 days, at least 15 days, at least 16 days, at least 17 days, at least 18 days, at least 19 days, at least 20 days before inhibition of one or more endogenous regulatory factor is performed. In further aspects, inhibiting the expression of PTEN occurs after introduction of the one or more transgenes into the cells. In further aspects, inhibiting the expression of P53 occurs after introduction of the one or more transgenes into the cells. In further aspects, inhibiting the expression of CD38 occurs after introduction of the one or more transgenes into the cells. In further aspects, introducing a transgene encoding TERT occurs after introduction of the one or more transgenes into the cells. In some aspects, the method comprises the following sequential steps i) introducing one or more transgenes into the immune cells and then culturing the cells for at least 2 days, 5 days, at least 10 days, at least 11 days, at least 12 days, at least 13 days, at least 14 days, at least 15 days, at least 16 days, at least 17 days, at least 18 days, at least 19 days, at least 20 days; ii) inhibiting one or more endogenous regulatory factor culturing the cell for at least 2 days, 5 days, at least 10 days, at least 11 days, at least 12 days, at least 13 days, at least 14 days, at least 15 days, at least 16 days, at least 17 days, at least 18 days, at least 19 days, at least 20 days; and iii) inhibiting PTEN expression, and/or P53 expression, and/or CD38 expression, and/or introducing TERT.

In some aspects, inhibiting the expression of one or more endogenous regulatory factors (e.g., CDKN2A, CDKN2B, or MTAP) occurs before introduction of one or more transgenes into the cells. In some aspects, primary immune cells in which one or more endogenous regulatory factors (e.g., CDKN2A, CDKN2B, MTAP) have been inhibited or ablated are cultured for at least 2 days, at least 3 days, at least 4 days, at least 5 days, at least 10 days, at least 11 days, at least 12 days, at least 13 days, at least 14 days, at least 15 days, at least 16 days, at least 17 days, at least 18 days, at least 19 days, at least 20 days before introduction of one or more transgenes is performed. In certain embodiments, one or more transgenes encoding Bcl-xL, MYC, KRAS, STAT5A, STAT5B, or any combination or mutant thereof, can be introduced at least 2 days, at least 3 days, at least 4 days, at least 5 days, at least 10 days, at least 11 days, at least 12 days, at least 13 days, at least 14 days, at least 15 days, at least 16 days, at least 17 days, at least 18 days, at least 19 days, at least 20 days after inhibiting the expression of one or more endogenous regulatory factors (e.g., CDKN2A, CDKN2B, or MTAP). In further aspects, inhibiting the expression of PTEN occurs after introduction of the one or more transgenes into the cells. In further aspects, inhibiting the expression of P53 occurs after introduction of the one or more transgenes into the cells. In further aspects, inhibiting the expression of CD38 occurs after introduction of the one or more transgenes into the cells. In further aspects, introducing a transgene encoding TERT occurs after introduction of the one or more transgenes into the cells. In some aspects, the method comprises the following sequential steps: i) inhibiting one or more endogenous regulatory factor culturing the cell for at least 2 days, 5 days, at least 10 days, at least 11 days, at least 12 days, at least 13 days, at least 14 days, at least 15 days, at least 16 days, at least 17 days, at least 18 days, at least 19 days, at least 20 days; ii) introducing one or more transgenes into the immune cells and then culturing the cells for at least 2 days, 5 days, at least 10 days, at least 11 days, at least 12 days, at least 13 days, at least 14 days, at least 15 days, at least 16 days, at least 17 days, at least 18 days, at least 19 days, at least 20 days; and iii) inhibiting PTEN expression, and/or P53 expression, and/or CD38 expression, and/or introducing TERT.

Primary immune cells are cultured under conditions appropriate for promoting proliferation and expansion. In vitro expansion using the culture step activates and induces proliferation of the primary immune cells to yield an expanded population comprising primary immune cells sufficient in numbers for use in therapy.

The methods disclosed herein are performed ex vivo meaning that the methods take place outside an organism. Treatment of immune cells ex vivo means exposing cells to certain biological molecules in vitro preferably under sterile conditions. In some cases, ex vivo methods additionally include culturing immune cells that have been isolated from a human prior to administration back into the same or different human subject.

The primary immune cells including the expanded populations, and/or the engineered T cells of this disclosure can comprise total T cells, CD4-positive T cells, CD8-positive T cells, regulatory T cells, gamma-delta T cells, mucosal associated invariant T (MAIT) T cells, natural killer (NK) cells, or natural killer T (NKT) cells. T cells are broadly divided into cells expressing CD4 on their surface (also referred to as CD4-positive cells) and cells expressing CD8 on their surface (also referred to as CD8-positive cells). T cells appropriate for use according to the methods provided herein are mononuclear lymphocytes derived from bone marrow (BM), peripheral blood (PB), or cord blood (CB) of a human donor. These cells could be collected directly from BM, PB, or CB or after mobilization or stimulation via administration of growth factors and/or cytokines such as granulocyte-colony stimulating factor (G-CSF) or granulocyte-macrophage colony-stimulating factor (GM-CSF) to allogeneic or autologous donors. Those skilled in the art would appreciate that there are many established protocols for isolating peripheral blood mononuclear cells (PBMC) from peripheral blood. Isolation of PBMC can be aided by density-gradient separation protocols, usually employing a density-gradient centrifugation technique using Ficoll®-Hypaque or Histopaque® for separating lymphocytes from other elements in the blood. Preferably, PBMC isolation is performed under sterile conditions. Isolation of PBMC can also use negative selection kits. Alternatively, cell elutriation methods may be employed to separate mononuclear cell populations. In some aspects, the primary immune cells are human.

In some cases, methods of this disclosure further include introducing a genetically engineered or chimeric antigen receptor into activated T cells, wherein the method thereby generates an expanded population comprising of T cells expressing the genetically engineered or chimeric antigen receptor. Chimeric antigen receptors (CARs), also known as chimeric T cell receptors, artificial T cell receptors, and chimeric immunoreceptors, are engineered receptors, which graft specificity onto an immune effector cell. In general, a chimeric antigen receptor is a transmembrane protein having a target-antigen binding domain that is fused via a spacer and a transmembrane domain to a signaling endodomain. When the CAR binds its target antigen, an activating signal is transmitted to the T cell. In one embodiment, a polynucleotide that encodes a chimeric antigen receptor is introduced to the primary cells. In one embodiment, a nucleic acid vector encoding the chimeric antigen receptor or genetically engineered receptor is introduced into the T cells whereby the T cells express the chimeric antigen receptor. In some aspects, the CAR binds glypican 3 (GPC3), human epidermal growth factor receptor 2 ((HER2); also known as Erb-B2 Receptor Tyrosine Kinase 2 (ERBB2)), B-cell maturation antigen (BCMA). In certain aspects, the CAR can bind any target for use in immunotherapy.

CAR construct design: CAR constructs of the present disclosure can have several components, many of which can be selected based upon a desired or refined function of the resultant CAR construct. In addition to an antigen binding domain, CAR constructs can have a spacer domain, a hinge domain, a signal peptide domain, a transmembrane domain, and one or more costimulatory domains. Selection of one component over another (i.e., selection of a specific co-stimulatory domain from one receptor versus a co-stimulatory domain from a different receptor) can influence clinical efficacy and safety profiles.

Antigen binding domain: Antigen binding domains contemplated herein can include antibodies or one or more antigen-binding fragments thereof. In an embodiment, a CAR construct targets GPC3. In an embodiment, a CAR construct targets BCMA. In an embodiment, a CAR construct targets HER2. In an embodiment, a CAR construct targets any molecule useful in an immunotherapy. In certain aspects, the antigen binding domain comprises a single chain variable fragment (scFv) containing light and heavy chain variable regions from one or more antibodies specific for GPC3, BCMA, or HER2 that are either directly linked together or linked together via a flexible linker (e.g., a repeat of G4S having 1, 2, 3 or more repeats).

Spacer domain: A CAR construct can have a spacer domain to provide conformational freedom to facilitate binding to the target antigen on the target cell. The optimal length of a spacer domain may depend on the proximity of the binding epitope to the target cell surface. For example, proximal epitopes can require longer spacers and distal epitopes can require shorter ones. Besides promoting binding of the CAR to the target antigen, achieving an optimal distance between a CAR cell and a cancer cell may also help to sterically occlude large inhibitory molecules from the immunological synapse formed between the CAR cell and the target cancer cell. A CAR can have a long spacer, an intermediate spacer, or a shorter spacer. Long spacers can include a CH2CH3 domain (˜220 amino acids) of immunoglobulin G1 (IgG1) or IgG4 (either native or with modifications common in therapeutic antibodies, such as a S228P mutation), whereas the CH3 region can be used on its own to construct an intermediate spacer (˜120 amino acids). Shorter spacers can be derived from segments (<60 amino acids) of CD28, CD8a, CD3 or CD4. Short spacers can also be derived from the hinge regions of IgG molecules. These hinge regions may be derived from any IgG isotype and may or may not contain mutations common in therapeutic antibodies such as the S228P mutation mentioned above.

Hinge domain: A CAR can also have a hinge domain. The flexible hinge domain is a short peptide fragment that provides conformational freedom to facilitate binding to the target antigen on the tumor cell. It may be used alone or in conjunction with a spacer sequence. The terms “hinge” and “spacer” are often used interchangeably—for example, IgG4 sequences can be considered both “hinge” and “spacer” sequences (i.e., hinge/spacer sequences).

Signal peptide: A CAR construct can further include a sequence comprising a signal peptide. Signal peptides function to prompt a cell to translocate the CAR to the cellular membrane. Examples include an IgG1 heavy chain signal polypeptide, Ig kappa or lambda light chain signal peptides, granulocyte-macrophage colony stimulating factor receptor 2 (GM-CSFR2 or CSFR2) signal peptide, a CD8a signal polypeptide, or a CD33 signal peptide.

Transmembrane domain: A CAR construct can further include a sequence comprising a transmembrane domain. The transmembrane domain can include a hydrophobic a helix that spans the cell membrane. The properties of the transmembrane domain have not been as meticulously studied as other aspects of CAR constructs, but they can potentially affect CAR expression and association with endogenous membrane proteins. Transmembrane domains can be derived, for example, from CD4, CD8a, or CD28.

Costimulatory domain: A CAR construct can further include one or more sequences that form a co-stimulatory domain. A co-stimulatory domain is a domain capable of potentiating or modulating the response of immune effector cells. Co-stimulatory domains can include sequences, for example, from one or more of CD3zeta (or CD32), CD28, 4-1BB, OX-40, ICOS, CD27, GITR, CD2, IL-2RB and MyD88/CD40. The choice of co-stimulatory domain influences the phenotype and metabolic signature of CAR cells. For example, CD28 co-stimulation yields a potent, yet short-lived, effector-like phenotype, with high levels of cytolytic capacity, interleukin-2 (IL-2) secretion, and glycolysis. By contrast, T cells modified with CARs bearing 4-1BB costimulatory domains tend to expand and persist longer in vivo, have increased oxidative metabolism, are less prone to exhaustion, and have an increased capacity to generate central memory T cells.

In particular aspects, the methods disclosed herein compromise early stimulation of the primary immune cells to ensure the cells are in cycle prior to introduction of the one or more genetic edits to the cells. In other aspects, the methods disclosed herein compromise late stimulation of the primary immune cells (also referred to as “restimulation”). Once the primary immune cells have exited cell cycle, the cells are restimulated causing the cells to re-enter into cell cycle (i.e., proliferation).

In particular aspects, the methods disclosed herein further comprise stimulating the population of primary immune cells before introducing one or more genetic edits to the population of primary immune cells. In some embodiments, the one or more genetic edits can refer to one or more of: (1) inhibiting the expression of cyclin-dependent kinase inhibitor 2A (CDKN2A), cyclin-dependent kinase inhibitor 2B (CDKN2B), and S-methyl-5′-thioadenosine phosphorylase (MTAP); (2) introducing a transgene encoding one or more STAT5A mutants and/or one or more STAT5B mutants; (3) introducing a transgene encoding B-cell lymphoma-extra large (Bcl-xL); (4) introducing a transgene encoding MYC; (5) introducing a transgene encoding TERT; (6) inhibiting the expression of phosphatase and tensin homolog (PTEN); (7) introducing a transgene encoding KRAS or a mutant thereof; (8) inhibiting the expression of p53; (9) inhibiting the expression of cluster of differentiation 38 (CD38); and/or (10) inhibiting the expression of beta-2 microglobulin (B2M) and/or T-cell receptor α constant (TRAC). In particular aspects, the population of primary immune cells is stimulated before at least 1 day, at least 2 days, at least 5 days, at least 10 days, at least 15 days, at least 20 days, or at least 30 days before the introduction of the one or more genetic edits to the population of primary immune cells. Thus, in particular aspects, provided herein is a method of generating a population of primary immune cells resistant to replicative senescence (RRS), comprising: i) stimulating the population of primary immune cells; ii) introducing one or more genetic edits into the population of primary immune cells; iii) culturing the population of primary immune cells in a culture medium; wherein the culturing induces proliferation of the primary immune cells to yield the population of primary immune cells resistant to replicative senescence (RRS).

In particular aspects, the methods disclosed herein further comprise stimulating the population of primary immune cells following introducing one or more genetic edits to the primary immune cells. In some embodiments, the one or more genetic edits can refer to one or more of: (1) inhibiting the expression of cyclin-dependent kinase inhibitor 2A (CDKN2A), cyclin-dependent kinase inhibitor 2B (CDKN2B), and S-methyl-5′-thioadenosine phosphorylase (MTAP); (2) introducing a transgene encoding one or more STAT5A mutants and/or one or more STAT5B mutants; (3) introducing a transgene encoding B-cell lymphoma-extra large (Bcl-xL); (4) introducing a transgene encoding MYC; (5) introducing a transgene encoding TERT; (6) inhibiting the expression of phosphatase and tensin homolog (PTEN); (7) introducing a transgene encoding KRAS or a mutant thereof; (8) inhibiting the expression of p53; (9) inhibiting the expression of cluster of differentiation 38 (CD38); and/or (10) inhibiting the expression of beta-2 microglobulin (B2M) and/or T-cell receptor α constant (TRAC). In particular aspects, the population of primary immune cells is stimulated after at least 1 day, at least 2 days, at least 5 days, at least 10 days, at least 15 days, at least 20 days, or at least 30 days following the introduction of the one or more genetic edits to the population of primary immune cells. Thus, in particular aspects, provided herein is a method of generating a population of primary immune cells resistant to replicative senescence (RRS), comprising: i) introducing one or more genetic edits into the population of primary immune cells; ii) culturing the population of primary immune cells in a culture medium; iii) stimulating the population of primary immune cells; and iv) culturing the population of primary immune cells in the culture medium; wherein the culturing induces proliferation of the primary immune cells to yield the population of primary immune cells resistant to replicative senescence (RRS).

In further aspects, the primary immune cells are re-stimulated at least one time, at least two times, at least three time, at least four times, or at least five times. Thus, in particular aspects, provided herein is a method of generating a population of primary immune cells resistant to replicative senescence (RRS), comprising: i) introducing one or more genetic edits into the population of primary immune cells; ii) culturing the population of primary immune cells in a culture medium; iii) stimulating the population of primary immune cells; iv) culturing the population of primary immune cells in the culture medium; v) re-stimulating the population of primary immune cells; and vi) culturing the population of primary immune cells in the culture medium wherein the culturing induces proliferation of the primary immune cells to yield the population of primary immune cells resistant to replicative senescence (RRS). Any suitable stimulus known in the art can be used stimulate the immune cells.

In particular aspects, the primary immune cells undergo at least about a 50-fold expansion, at least about a 500-fold expansion, at least about a 5000-fold expansion, at least about a 250,000-fold expansion, at least about a 500,000-fold expansion, at least about a 106 fold expansion, at least about a 10⁷ fold expansion, at least about a 10⁸ fold expansion, at least about a 10⁹ fold expansion, or at least about a 10¹⁰ fold expansion during culturing. In particular aspects, the population of expanded primary immune cells is resistant to replicative senescence. Furthermore, these cells are not functionally exhausted following long-term expansion and can be directed to carry out cytotoxic function through engagement of their TCRs by a T cell engager antibody or through engagement of a chimeric antigen receptor (CAR), (or through a natural or genetically-introduced TCR).

In particular aspects, the primary immune cells are cultured in a culture medium that includes supportive cytokine(s) but does not include a primary immune cell stimulus. In particular aspects, the primary immune cells undergo expansion during culturing in the absence of feeder cells or stimulation through CD3 and/or their antigen receptor. The ability of the disclosed methods to generate immune cells in the absence of extensive T cell re-stimulation or feeder cells advantageously eliminates the issues of scaling up the methods and producing dysfunctional populations of immune cells.

The methods disclosed herein advantageously provide populations of expanded primary immune cells including human CD8⁺ T cells, human CD4⁺ T cells, human regulatory T cells human gamma-delta T cells, or human natural killer T cells that have the ability to proliferate for substantial periods of time in the absence of re-stimulation through their T cell receptors (TCRs), expanding millions of fold in long-term culture. In particular aspects, the population of primary immune cells are cultured for at least 20 days, at least 30 days, at least 40 days, at least 50 days, at least 60 days, at least 70 days, at least 80 days, at least 90 days, at least 100 days, at least 150 days, at least 200 days, at least 300 days, or at least 400 days.

In further aspects provided herein is an engineered T cell expressing a transgene encoding a B-cell lymphoma-extra large (Bcl-xL) that does not express cyclin-dependent kinase inhibitor 2A (CDKN2A), cyclin-dependent kinase inhibitor 2B (CDKN2B), and/or S-methyl-5′-thioadenosine phosphorylase (MTAP).

In further aspects provided herein is an engineered T cell that does not express cyclin-dependent kinase inhibitor 2A (CDKN2A), cyclin-dependent kinase inhibitor 2B (CDKN2B), and/or S-methyl-5′-thioadenosine phosphorylase (MTAP).

In further aspects provided herein is an engineered T cell expressing a transgene encoding a B-cell lymphoma-extra large (Bcl-XL) that does not express cyclin-dependent kinase inhibitor 2A (CDKN2A), cyclin-dependent kinase inhibitor 2B (CDKN2B), S-methyl-5′-thioadenosine phosphorylase (MTAP) and/or phosphatase and tensin homolog (PTEN).

In further aspects provided herein is an engineered T cell that does not express cyclin-dependent kinase inhibitor 2A (CDKN2A), cyclin-dependent kinase inhibitor 2B (CDKN2B), and/or S-methyl-5′-thioadenosine phosphorylase (MTAP), and comprises a transgene encoding a signal transducer and activator of transcription 5A (STAT5A) mutant and/or a signal transducer and activator of transcription 5B (STA5B) mutant. In some embodiments, a STAT5A mutant can include, but is not limited to, H299R, N642H, Y665F, S711F, and combinations thereof. In some embodiments, a STAT5B mutant can include, but is not limited to, H298R, R430C, E433K, N642H, Y665F, P702A, V712E, S715F, and combinations thereof.

In further aspects provided herein is an engineered T cell that does not express cyclin-dependent kinase inhibitor 2A (CDKN2A), cyclin-dependent kinase inhibitor 2B (CDKN2B), and/or S-methyl-5′-thioadenosine phosphorylase (MTAP), and comprises a transgene encoding a signal transducer and activator of transcription 5A (STAT5A) mutant and/or a signal transducer and activator of transcription 5B (STA5B) mutant, and TERT. In some embodiments, a STAT5A mutant can include, but is not limited to, H299R, N642H, Y665F, S711F, and combinations thereof. In some embodiments, a STAT5B mutant can include, but is not limited to, H298R, R430C, E433K, N642H, Y665F, P702A, V712E, S715F, and combinations thereof.

In further aspects provided herein is an engineered T cell expressing a transgene encoding a B-cell lymphoma-extra large (Bcl-XL) that does not express cyclin-dependent kinase inhibitor 2A (CDKN2A), cyclin-dependent kinase inhibitor 2B (CDKN2B), S-methyl-5′-thioadenosine phosphorylase (MTAP) and/or comprises a transgene encoding a signal transducer and activator of transcription 5A (STAT5A) mutant and/or a signal transducer and activator of transcription 5B (STA5B) mutant, and TERT. In some embodiments, a STAT5A mutant can include, but is not limited to, H299R, N642H, Y665F, S711F, and combinations thereof. In some embodiments, a STAT5B mutant can include, but is not limited to, H298R, R430C, E433K, N642H, Y665F, P702A, V712E, S715F, and combinations thereof.

In further aspects provided herein is an engineered T cell expressing a transgene encoding a B-cell lymphoma-extra large (Bcl-XL) that does not express cyclin-dependent kinase inhibitor 2A (CDKN2A), cyclin-dependent kinase inhibitor 2B (CDKN2B), S-methyl-5′-thioadenosine phosphorylase (MTAP) and/or comprises a transgene encodes a signal transducer and activator of transcription 5A (STAT5A) mutant and/or a signal transducer and activator of transcription 5B (STA5B) mutant, and MYC. In some embodiments, a STAT5A mutant can include, but is not limited to, H299R, N642H, Y665F, S711F, and combinations thereof. In some embodiments, a STAT5B mutant can include, but is not limited to, H298R, R430C, E433K, N642H, Y665F, P702A, V712E, S715F, and combinations thereof.

In further aspects provided herein is an engineered T cell expressing a transgene encoding a B-cell lymphoma-extra large (Bcl-XL) that does not express cyclin-dependent kinase inhibitor 2A (CDKN2A), cyclin-dependent kinase inhibitor 2B (CDKN2B), S-methyl-5′-thioadenosine phosphorylase (MTAP) and/or comprises a transgene encodes a signal transducer and activator of transcription 5A (STAT5A) mutant and/or a signal transducer and activator of transcription 5B (STA5B) mutant, TERT and MYC. In some embodiments, a STAT5A mutant can include, but is not limited to, H299R, N642H, Y665F, S711F, and combinations thereof. In some embodiments, a STAT5B mutant can include, but is not limited to, H298R, R430C, E433K, N642H, Y665F, P702A, V712E, S715F, and combinations thereof.

In some aspects, provided herein is an engineered T cell that does not express cyclin-dependent kinase inhibitor 2A (CDKN2A), cyclin-dependent kinase inhibitor 2B (CDKN2B), and S-methyl-5′-thioadenosine phosphorylase (MTAP), and also comprises an optional transgene encoding B-cell lymphoma-extra large (Bcl-XL) and a transgene encoding MYC.

In some embodiments, the engineered T cell further comprises a transgene encoding KRAS. In some embodiments, the KRAS is a mutant KRAS selected from G12C and A146V. In an embodiment, the mutant KRAS is A146V. In some embodiments, the engineered T cell further comprises a knockout or ablation of p53. In some embodiments, the engineered T cell further comprises a knockout or ablation of PTEN. In some embodiments, the engineered T cell further comprises a transgene encoding TERT.

In some aspects, provided herein is an engineered T cell that does not express cyclin-dependent kinase inhibitor 2A (CDKN2A), cyclin-dependent kinase inhibitor 2B (CDKN2B), S-methyl-5′-thioadenosine phosphorylase (MTAP), and does not express p53, and also comprises an optional transgene encoding B-cell lymphoma-extra large (Bcl-XL) and a transgene encoding MYC.

In some aspects, provided herein is an engineered T cell that does not express cyclin-dependent kinase inhibitor 2A (CDKN2A), cyclin-dependent kinase inhibitor 2B (CDKN2B), S-methyl-5′-thioadenosine phosphorylase (MTAP), and does not express PTEN, and also comprises an optional transgene encoding B-cell lymphoma-extra large (Bcl-XL) and a transgene encoding MYC.

In some aspects, provided herein is an engineered T cell that does not express cyclin-dependent kinase inhibitor 2A (CDKN2A), cyclin-dependent kinase inhibitor 2B (CDKN2B), and S-methyl-5′-thioadenosine phosphorylase (MTAP), and also comprises a transgene encoding TERT.

In further aspects, provided herein is an engineered T cell that does not express cyclin-dependent kinase inhibitor 2A (CDKN2A), cyclin-dependent kinase inhibitor 2B (CDKN2B), and S-methyl-5′-thioadenosine phosphorylase (MTAP), and also comprises a transgene encoding TERT, an optional transgene encoding B-cell lymphoma-extra large (Bcl-XL) and a transgene encoding MYC.

In some embodiments, the engineered T cell further comprises a transgene encoding KRAS. In some embodiments, the KRAS is a mutant KRAS selected from G12C and A146V. In an embodiment, the mutant KRAS is A146V. In some embodiments, the engineered T cell further comprises a knockout or ablation of p53. In some embodiments, the engineered T cell further comprises a knockout or ablation of PTEN.

In some embodiments, any of the engineered T cells as described herein further comprise a transgene encoding a signal transducer and activator of transcription 5A (STAT5A) mutant and/or a signal transducer and activator of transcription 5B (STA5B) mutant. In some embodiments, the STAT5A mutant can include, but is not limited to, H299R, N642H, Y665F, S711F, and combinations thereof. In some embodiments, the STAT5B mutant can include, but is not limited to, H298R, R430C, E433K, N642H, Y665F, P702A, V712E, S715F, and combinations thereof.

In some aspects, provided herein is an engineered NK cell that does not express cyclin-dependent kinase inhibitor 2A (CDKN2A), cyclin-dependent kinase inhibitor 2B (CDKN2B), and S-methyl-5′-thioadenosine phosphorylase (MTAP), and also comprises a transgene encoding TERT.

In further aspects, provided herein is an engineered NK cell that does not express cyclin-dependent kinase inhibitor 2A (CDKN2A), cyclin-dependent kinase inhibitor 2B (CDKN2B), and S-methyl-5′-thioadenosine phosphorylase (MTAP), and also comprises a transgene encoding TERT, an optional transgene encoding B-cell lymphoma-extra large (Bcl-XL) and a transgene encoding MYC.

In some embodiments, the engineered NK cell further comprises a transgene encoding KRAS. In some embodiments, the KRAS is a mutant KRAS selected from G12C and A146V. In an embodiment, the mutant KRAS is A146V. In some embodiments, the engineered NK cell further comprises a knockout or ablation of p53. In some embodiments, the engineered NK cell further comprises a knockout or ablation of PTEN.

In some embodiments, any of the engineered NK cells as described herein further comprise a transgene encoding a signal transducer and activator of transcription 5A (STAT5A) mutant and/or a signal transducer and activator of transcription 5B (STA5B) mutant. In some embodiments, the STAT5A mutant can include, but is not limited to, H299R, N642H, Y665F, S711F, and combinations thereof. In some embodiments, the STAT5B mutant can include, but is not limited to, H298R, R430C, E433K, N642H, Y665F, P702A, V712E, S715F, and combinations thereof.

In certain aspects, the engineered T cell as disclosed herein does not express of one or more endogenous immune related genes in the primary immune cells. In some aspects, the endogenous immune related gene is beta-2 microglobulin (B2M) or T-cell receptor α constant (TRAC).

In certain aspects, the engineered T cell as disclosed herein does not express p53.

In certain aspects, the engineered T cell as disclosed herein does not express cluster of differentiation 38 (CD38).

In further aspects the disclosure herein provides an engineered T cell that does not express cyclin-dependent kinase inhibitor 2A (CDKN2A), cyclin-dependent kinase inhibitor 2B (CDKN2B), S-methyl-5′-thioadenosine phosphorylase (MTAP), beta-2 microglobulin (B2M), T-cell receptor α constant (TRAC), cluster of differentiation 38 (CD38), and/or phosphatase and tensin homolog (PTEN).

In certain aspects, the engineered T cell as disclosed comprises a polynucleotide that encodes a chimeric antigen receptor (CAR). In some aspects, the CAR binds glypican 3 (GPC3), B-cell maturation antigen (BCMA), or human epidermal growth factor receptor 2 ((HER2); also known as Erb-B2 Receptor Tyrosine Kinase 2 (ERBB2)).

In certain aspects, the engineered T cell as disclosed herein is a CD8⁺ T cell, a CD4+ T cell, a gamma delta T cell, a mucosal associated invariant T (MAIT) T cell, a natural killer (NK) cell, a natural killer T (NKT) cell, or a combination thereof.

In some aspects, the engineered T cell is resistant to replicative senescence (RRS). In some aspects, the engineered T cell is a CD8⁺ T cell. In some aspects, the engineered T cell is a CD4⁺ T cell. In some aspects, the engineered T cell is human.

Expanded T cell populations disclosed herein are useful for cellular immunotherapies including, without limitation, T cell therapy, adoptive cell therapy (ACT), and CAR T cell therapy.

Expanded populations of T cells populations disclosed herein are useful for treating or preventing various disorders such as a cancer (e.g., a blood malignancy such as lymphoma or leukemia or solid tumors such as melanoma or kidney cancer), autoimmune diseases or an infectious disease such as HIV.

In one aspect, this disclosure provides for the use of an engineered T cell for the manufacture of a medicament for treating cancer in a patient, wherein the engineered T cell does not express cyclin-dependent kinase inhibitor 2A (CDKN2A), cyclin-dependent kinase inhibitor 2B (CDKN2B), and/or S-methyl-5′-thioadenosine phosphorylase (MTAP), and wherein the engineered T cell comprises a transgene encoding one or more STAT5A mutants and/or one or more STAT5B mutants.

In one aspect, this disclosure provides for the use of an engineered T cell for the manufacture of a medicament for treating cancer in a patient, wherein the engineered T cell does not express cyclin-dependent kinase inhibitor 2A (CDKN2A), cyclin-dependent kinase inhibitor 2B (CDKN2B), and/or S-methyl-5′-thioadenosine phosphorylase (MTAP), and wherein the engineered T cell comprises an optional transgene encoding B-cell lymphoma-extra large (Bcl-xL) and a transgene encoding MYC.

In one aspect, this disclosure provides for the use of an engineered T cell for the manufacture of a medicament for treating cancer in a patient, wherein the engineered T cell does not express cyclin-dependent kinase inhibitor 2A (CDKN2A), cyclin-dependent kinase inhibitor 2B (CDKN2B), and/or S-methyl-5′-thioadenosine phosphorylase (MTAP), and wherein the engineered T cell comprises a transgene encoding TERT.

In some embodiments of the use of the engineered T cells disclosed herein, the one or more STAT5A mutants can be H299R, N642H, Y665F, S711F, and combinations thereof, and/or wherein the one or more STAT5B mutants can be H298R, R430C, E433K, N642H, Y665F, P702A, V712E, S715F, and combinations thereof.

In some embodiments of the use of the engineered T cells disclosed herein, the engineered T cells further comprise a transgene encoding TERT in the population of primary immune cells.

In some embodiments of the use of the engineered T cells disclosed herein, the engineered T cells further comprise inhibited expression of one or more endogenous immune related genes in the population of primary immune cells. In some embodiments, the endogenous immune related gene is beta-2 microglobulin (B2M) and/or T-cell receptor α constant (TRAC).

In some embodiments of the use of the engineered T cells disclosed herein, the engineered T cells comprises one or more transgenes encoding an anti-apoptotic factor or a virally-derived factor into the primary immune cells. In some embodiments, the anti-apoptotic factor is either B-cell lymphoma-extra large (Bcl-xL) or B-cell lymphoma 2 (Bcl-2). In some embodiments, the virally-derived factor is any one of Saimiriine gammaherpesvirus 2 StpA A11, Herpesvirus saimiri StpC, Herpesvirus saimiri Tip, or a modified Herpesvirus Ateles-Epstein-Barr virus Tio-LMP1.

In some embodiments of the use of the engineered T cells disclosed herein, the engineered T cells further comprise inhibited expression of cluster of differentiation 38 (CD38) in the population of primary immune cells. In some embodiments of the use of the engineered T cells disclosed herein, the engineered T cells further comprise inhibited expression of phosphatase and tensin homolog (PTEN) in the population of primary immune cells. In some embodiments of the use of the engineered T cells disclosed herein, the engineered T cells further comprise inhibited expression of p53 in the population of primary immune cells. In some embodiments of the use of the engineered T cells disclosed herein, the engineered T cells further comprise a transgene encoding MYC in the population of primary immune cells.

In some embodiments of the use of the engineered T cells disclosed herein, the engineered T cells further comprise a transgene encoding KRAS in the population of primary immune cells. In some embodiments, KRAS is a mutant KRAS A146V.

In some embodiments of the use of the engineered T cells disclosed herein, the engineered T cells further comprise a polynucleotide that encodes a chimeric antigen receptor (CAR) in the population of primary immune cells.

In one aspect, this disclosure provides an engineered T cell for the treatment of cancer in a patient, wherein the engineered T cell does not express cyclin-dependent kinase inhibitor 2A (CDKN2A), cyclin-dependent kinase inhibitor 2B (CDKN2B), and/or S-methyl-5′-thioadenosine phosphorylase (MTAP), and wherein the engineered T cell comprises a transgene encoding one or more STAT5A mutants and/or one or more STAT5B mutants.

In one aspect, this disclosure provides an engineered T cell for the treatment of cancer in a patient, wherein the engineered T cell does not express cyclin-dependent kinase inhibitor 2A (CDKN2A), cyclin-dependent kinase inhibitor 2B (CDKN2B), and/or S-methyl-5′-thioadenosine phosphorylase (MTAP), and comprises an optional transgene encoding B-cell lymphoma-extra large (Bcl-xL) and a transgene encoding MYC.

In one aspect, this disclosure provides an engineered T cell for the treatment of cancer in a patient that does not express cyclin-dependent kinase inhibitor 2A (CDKN2A), cyclin-dependent kinase inhibitor 2B (CDKN2B), and/or S-methyl-5′-thioadenosine phosphorylase (MTAP), and comprises a transgene encoding TERT.

The terms “treatment” or “treat,” as used herein, refer to both therapeutic treatment and prophylactic or preventative measures. Those in need of treatment include subjects having cancer as well as those prone to having cancer or those in cancer is to be prevented. In some aspects, the methods, compositions, and combinations disclosed herein can be used for the treatment of cancer. In other aspects, those in need of treatment include subjects having a tumor as well as those prone to have a tumor or those in which a tumor is to be prevented. In certain aspects, the methods, compositions, and combinations disclosed herein can be used for the treatment of tumors. In other aspects, treatment of a tumor includes inhibiting tumor growth, promoting tumor reduction, or both inhibiting tumor growth and promoting tumor reduction.

In some cases, T cells obtained according to a method provided herein can be administered as a pharmaceutical composition comprising a therapeutically effective amount of T cells as a therapeutic agent (i.e., for therapeutic applications).

The terms “pharmaceutical composition” or “therapeutic composition,” as used herein, refer to a compound or composition capable of inducing a desired therapeutic effect when properly administered to a subject. In some aspects, the disclosure provides a pharmaceutical composition comprising a pharmaceutically acceptable carrier and a therapeutically effective amount of at least one immune cell of the disclosure.

The terms “pharmaceutically acceptable carrier” or “physiologically acceptable carrier,” as used herein, refer to one or more formulation materials suitable for accomplishing or enhancing the delivery of one or more immune cells of the disclosure.

The term “subject” is intended to include human and non-human animals, particularly mammals. In certain aspects, the subject is a human patient.

The terms “administration” or “administering,” as used herein, refer to providing, contacting, and/or delivering a compound or compounds by any appropriate route to achieve the desired effect. Administration may include, but is not limited to, oral, sublingual, parenteral (e.g., intravenous, subcutaneous, intracutaneous, intramuscular, intraarticular, intraarterial, intrasynovial, intrasternal, intrathecal, intralesional, or intracranial injection), transdermal, topical, buccal, rectal, vaginal, nasal, ophthalmic, via inhalation, and implants.

Without limiting the disclosure, a number of aspects of the disclosure are described herein for purpose of illustration.

### Embodiments

Embodiment 1. A method of generating a population of primary immune cells resistant to replicative senescence (RRS), comprising:
(a) inhibiting the expression of cyclin-dependent kinase inhibitor 2A (CDKN2A), cyclin-dependent kinase inhibitor 2B (CDKN2B), and S-methyl-5′-thioadenosine phosphorylase (MTAP) in the population of primary immune cells;
(b) introducing a transgene encoding one or more STAT5A mutants and/or one or more STAT5B mutants in the population of primary immune cells; and
(c) culturing the primary immune cells in a culture medium;
wherein the culturing induces proliferation of the primary immune cells to yield the population of primary immune cells resistant to replicative senescence (RRS).

Embodiment 2. The method of embodiment 1 further comprising stimulating the population of primary immune cells before performing step (a) and/or step (b) and/or step (c) in the population of primary immune cells.

Embodiment 3. The method of either embodiment 1 or embodiment 2 further comprising stimulating the population of primary immune cells after performing step (a) and/or step (b) and/or step (c) in the population of primary immune cells.

Embodiment 4. The method of any one of embodiments 1-3, wherein the one or more STAT5A mutants can be H299R, N642H, Y665F, S711F, and combinations thereof, and/or wherein the one or more STAT5B mutants can be H298R, R430C, E433K, N642H, Y665F, P702A, V712E, S715F, and combinations thereof.

Embodiment 5. The method of any one of embodiments 1-4 further comprising introducing a transgene encoding TERT in the population of primary immune cells.

Embodiment 6. The method of any one of embodiments 1-5 further comprising inhibiting the expression of one or more endogenous immune related genes in the population of primary immune cells.

Embodiment 7. The method of embodiment 6, wherein the endogenous immune related gene is beta-2 microglobulin (B2M) and/or T-cell receptor α constant (TRAC).

Embodiment 8. The method of any one of embodiments 1-7, further comprising introducing one or more transgenes encoding an anti-apoptotic factor or a virally-derived factor into the population of primary immune cells.

Embodiment 9. The method of embodiment 8, wherein the anti-apoptotic factor is either B-cell lymphoma-extra large (Bcl-xL) or B-cell lymphoma 2 (Bcl-2).

Embodiment 10. The method of embodiment 8, wherein the virally-derived factor is any one of Saimiriine gammaherpesvirus 2 StpA A11, Herpesvirus saimiri StpC, Herpesvirus saimiri Tip, or a modified Herpesvirus Ateles-Epstein-Barr virus Tio-LMP1.

Embodiment 11. The method of any one of embodiments 1-10 further comprising inhibiting the expression of cluster of differentiation 38 (CD38), inhibiting the expression of phosphatase and tensin homolog (PTEN), and/or inhibiting the expression of p53 in the population of primary immune cells.

Embodiment 12. The method of any one of embodiments 1-11 further comprising introducing a transgene encoding MYC and/or introducing a transgene encoding KRAS in the population of primary immune cells.

Embodiment 13. A method of generating a population of primary immune cells resistant to replicative senescence (RRS), comprising:
(a) inhibiting the expression of cyclin-dependent kinase inhibitor 2A (CDKN2A), cyclin-dependent kinase inhibitor 2B (CDKN2B), and S-methyl-5′-thioadenosine phosphorylase (MTAP) in the population of primary immune cells;
(b) introducing a transgene encoding MYC in the population of primary immune cells; and
(c) culturing the primary immune cells in a culture medium;
wherein the culturing induces proliferation of the primary immune cells to yield the population of primary immune cells resistant to replicative senescence (RRS).

Embodiment 14. The method of embodiment 13 further comprising introducing a transgene encoding B-cell lymphoma-extra large (Bcl-xL) in the population of primary immune cells.

Embodiment 15. The method of either embodiment 13 or embodiment 14 further comprising inhibiting the expression of p53 in the population of primary immune cells.

Embodiment 16. The method of any one of embodiments 13-15 further comprising introducing a transgene encoding KRAS in the population of primary immune cells.

Embodiment 17. The method of embodiment 16, wherein KRAS comprises a KRAS A146V mutation.

Embodiment 18. The method of any one of embodiments 13-17 further comprising inhibiting the expression of phosphatase and tensin homolog (PTEN) in the population of primary immune cells.

Embodiment 19. The method of embodiment 18, wherein PTEN expression is inhibited by a CRISPR/Cas system.

Embodiment 20. A method of generating a population of primary immune cells resistant to replicative senescence (RRS), comprising:
(a) inhibiting the expression of cyclin-dependent kinase inhibitor 2A (CDKN2A), cyclin-dependent kinase inhibitor 2B (CDKN2B), and S-methyl-5′-thioadenosine phosphorylase (MTAP) in the population of primary immune cells;
(b) introducing a transgene encoding TERT in the population of primary immune cells; and
(c) culturing the primary immune cells in a culture medium;
wherein the culturing induces proliferation of the primary immune cells to yield the population of primary immune cells resistant to replicative senescence (RRS).

Embodiment 21. The method of embodiment 20 further comprising introducing a transgene encoding B-cell lymphoma-extra large (Bcl-xL) and introducing a transgene encoding MYC in the population of primary immune cells.

Embodiment 22. The method of either embodiment 20 or embodiment 21 further comprising introducing a transgene encoding KRAS in the population of primary immune cells.

Embodiment 23. The method of embodiment 22, wherein KRAS comprises a KRAS A146V mutation.

Embodiment 24. The method of any one of embodiments 13-23 further comprising stimulating the population of primary immune cells before performing step (a) and/or step (b) and/or step (c) in the population of primary immune cells.

Embodiment 25. The method of any one of embodiments 13-23 further comprising stimulating the population of primary immune cells after performing step (a) and/or step (b) and/or step (c) in the population of primary immune cells.

Embodiment 26. The method of any one of embodiments 13-25 further comprising inhibiting the expression of one or more endogenous immune related genes in the primary immune cells in the population of primary immune cells.

Embodiment 27. The method of embodiment 26, wherein the endogenous immune related gene is beta-2 microglobulin (B2M) and/or T-cell receptor α constant (TRAC).

Embodiment 28. The method of any one of embodiments 13-27 further comprising introducing into the primary immune cells one or more transgenes encoding any one of Saimiriine gammaherpesvirus 2 StpA A11, Herpesvirus saimiri StpC Herpesvirus saimiri Tip, or a modified Herpesvirus Ateles-Epstein-Barr virus Tio-LMP1.

Embodiment 29. The method of any one of embodiments 13-28 further comprising inhibiting the expression of cluster of differentiation 38 (CD38) in the population of primary immune cells.

Embodiment 30. The method of any one of embodiments 1-29, wherein the population of primary immune cells comprises total T cells.

Embodiment 31. The method of any one of embodiments 1-29, wherein the population of primary immune cells comprises CD8+ T cells.

Embodiment 32. The method of any one of embodiments 1-29, wherein the population of primary immune cells comprises CD4+ T cells.

Embodiment 33. The method of any one of embodiments 1-32, wherein the population of primary immune cells comprises gamma-delta T cells, mucosal associated invariant T (MAIT) T cells, natural killer (NK) cells, and/or natural killer T (NKT) cells.

Embodiment 34. The method of any one of embodiments 1-33, wherein the population of primary immune cells is human.

Embodiment 35. The method of any one of embodiments 1-34, further comprising introducing a polynucleotide that encodes a chimeric antigen receptor (CAR) in the population of primary immune cells.

Embodiment 36. The method of any one of embodiments 1-35, wherein the population of primary immune cells can be cultured with or without TCR stimulation for at least 100 days.

Embodiment 37. The method of any one of embodiments 1-36, wherein the population of primary immune cells undergoes at least about a 10⁶-fold expansion during culturing.

Embodiment 38. The method of any one of embodiments 1-37, wherein the population of primary immune cells is cultured in a culture medium that does not include a primary immune cell stimulus.

Embodiment 39. The method of any one of embodiments 1-38 further comprising
(d) restimulating the population of primary immune cells.

Embodiment 40. The method of embodiment 39, wherein the population of primary immune cells undergoes at least about a 10⁸-fold expansion during culturing.

Embodiment 41. The method of any one of embodiments 1-40, wherein the transgene is introduced using a plasmid-based DNA transposon.

Embodiment 42. The method of any one of embodiments 1-40, wherein the transgene is introduced using a lentivirus platform.

Embodiment 43. The method of any one of embodiments 1-40, wherein the transgene is introduced using site specific integration via CRISPR.

Embodiment 44. A method of generating a population of primary immune cells resistant to replicative senescence (RRS), comprising:
(a) inhibiting the expression of one or more endogenous regulatory factors in the population of primary immune cells,
wherein the endogenous regulatory factor is cyclin-dependent kinase inhibitor 2A (CDKN2A), cyclin-dependent kinase inhibitor 2B (CDKN2B), or S-methyl-5′-thioadenosine phosphorylase (MTAP);
(b) inhibiting the expression of one or more endogenous immune related genes in the population of primary immune cells,
wherein the endogenous immune related gene is beta-2 microglobulin (B2M) and/or T-cell receptor α constant (TRAC);
(c) introducing a transgene encoding one or more STAT5A mutants and/or one or more STAT5B mutants in the population of primary immune cells; and
(d) culturing the population of primary immune cells in a culture medium;
wherein the culturing induces proliferation of the primary immune cells to yield the population of primary immune cells resistant to replicative senescence (RRS).

Embodiment 45. The method of embodiment 44 further comprising introducing a transgene encoding either B-cell lymphoma-extra large (Bcl-xL) or B-cell lymphoma 2 (Bcl-2) into the population of primary immune cells.

Embodiment 46. The method of either embodiment 44 or embodiment 45 further comprising stimulating the population of primary immune cells before performing step (a) and/or step (b) and/or step (c) and/or step (d) in the population of primary immune cells.

Embodiment 47. The method of any one of embodiments 44-46 further comprising stimulating the population of primary immune cells after performing step (a) and/or step (b) and/or step (c) and/or step (d) in the population of primary immune cells.

Embodiment 48. The method of any one of embodiments 44-47, wherein the one or more STAT5A mutants can be H299R, N642H, Y665F, S711F, and combinations thereof, and/or wherein the one or more STAT5B mutants can be H298R, R430C, E433K, N642H, Y665F, P702A, V712E, S715F, and combinations thereof.

Embodiment 49. The method of any one of embodiments 44-48 further comprising introducing a transgene encoding TERT in the population of primary immune cells.

Embodiment 50. The method of any one of embodiments 44-49 further comprising inhibiting the expression of cluster of differentiation 38 (CD38), inhibiting the expression of phosphatase and tensin homolog (PTEN), and/or inhibiting the expression of p53 in the population of primary immune cells.

Embodiment 51. The method of any one of embodiments 44-50 further comprising introducing a transgene encoding MYC and/or introducing a transgene encoding KRAS in the population of primary immune cells.

Embodiment 52. A method of generating a population of primary immune cells resistant to replicative senescence (RRS), comprising:
(a) inhibiting the expression of cyclin-dependent kinase inhibitor 2A (CDKN2A), cyclin-dependent kinase inhibitor 2B (CDKN2B), and S-methyl-5′-thioadenosine phosphorylase (MTAP) in the population of primary immune cells;
(b) inhibiting the expression of one or more endogenous immune related genes in the population of primary immune cells, wherein the endogenous immune related gene is beta-2 microglobulin (B2M) and/or T-cell receptor α constant (TRAC);
(c) introducing a transgene encoding MYC in the population of primary immune cells; and
(d) culturing the primary immune cells in a culture medium;
wherein the culturing induces proliferation of the primary immune cells to yield the population of primary immune cells resistant to replicative senescence (RRS).

Embodiment 53. The method of embodiment 52 further comprising introducing a transgene encoding B-cell lymphoma-extra large (Bcl-xL) in the population of primary immune cells.

Embodiment 54. The method of either embodiment 52 or embodiment 53 further comprising inhibiting the expression of p53 in the population of primary immune cells.

Embodiment 55. The method of any one of embodiments 52-54 further comprising introducing a transgene encoding KRAS in the population of primary immune cells.

Embodiment 56. The method of embodiment 55, wherein KRAS comprises a KRAS A146V mutation.

Embodiment 57. The method of any one of embodiments 52-56 further comprising inhibiting the expression of phosphatase and tensin homolog (PTEN) in the population of primary immune cells.

Embodiment 58. The method of embodiment 57, wherein PTEN expression is inhibited by a CRISPR/Cas system.

Embodiment 59. A method of generating a population of primary immune cells resistant to replicative senescence (RRS), comprising:
(a) inhibiting the expression of cyclin-dependent kinase inhibitor 2A (CDKN2A), cyclin-dependent kinase inhibitor 2B (CDKN2B), and S-methyl-5′-thioadenosine phosphorylase (MTAP) in the population of primary immune cells;
(b) inhibiting the expression of one or more endogenous immune related genes in the population of primary immune cells, wherein the endogenous immune related gene is beta-2 microglobulin (B2M) and/or T-cell receptor α constant (TRAC);
(c) introducing a transgene encoding TERT in the population of primary immune cells; and
(d) culturing the primary immune cells in a culture medium;
wherein the culturing induces proliferation of the primary immune cells to yield the population of primary immune cells resistant to replicative senescence (RRS).

Embodiment 60. The method of embodiment 59 further comprising introducing a transgene encoding B-cell lymphoma-extra large (Bcl-xL) and introducing a transgene encoding MYC in the population of primary immune cells.

Embodiment 61. The method of either embodiment 59 or embodiment 60 further comprising introducing a transgene encoding KRAS in the population of primary immune cells.

Embodiment 62. The method of embodiment 61, wherein KRAS comprises a KRAS A146V mutation.

Embodiment 63. The method of any one of embodiments 52-62 further comprising stimulating the population of primary immune cells before performing step (a) and/or step (b) and/or step (c) and/or step (d) in the population of primary immune cells.

Embodiment 64. The method of any one of embodiments 52-62 further comprising stimulating the population of primary immune cells after performing step (a) and/or step (b) and/or step (c) and/or step (d) in the population of primary immune cells.

Embodiment 65. The method of any one of embodiments 52-64 further comprising introducing into the primary immune cells one or more transgenes encoding any one of Saimiriine gammaherpesvirus 2 StpA A11, Herpesvirus saimiri StpC Herpesvirus saimiri Tip, or a modified Herpesvirus Ateles-Epstein-Barr virus Tio-LMP1.

Embodiment 66. The method of any one of embodiments 52-65 further comprising inhibiting the expression of cluster of differentiation 38 (CD38) in the population of primary immune cells.

Embodiment 67. The method of any one of embodiments 44-66, wherein the population of primary immune cells comprises total T cells.

Embodiment 68. The method of any one of embodiments 44-66, wherein the population of primary immune cells comprises CD8⁺ T cells.

Embodiment 69. The method of any one of embodiments 44-66, wherein the population of primary immune cells comprises CD4⁺ T cells.

Embodiment 70. The method of any one of embodiments 44-66, wherein the population of primary immune cells comprises gamma-delta T cells, mucosal associated invariant T (MAIT) T cells, natural killer (NK) cells, and/or natural killer T (NKT) cells.

Embodiment 71. The method of any one of embodiments 44-70, wherein the population of primary immune cells is human.

Embodiment 72. The method of any one of embodiments 44-71 further comprising introducing a polynucleotide that encodes a chimeric antigen receptor (CAR) in the population of primary immune cells.

Embodiment 73. The method of any one of embodiments 44-72, wherein the population of primary immune cells can be cultured for at least 100 days.

Embodiment 74. The method of any one of embodiments 44-73, wherein the population of primary immune cells undergoes at least about a 10⁶-fold expansion during culturing.

Embodiment 75. The method of any one of embodiments 44-74, wherein the population of primary immune cells is cultured in a culture medium that does not include a primary immune cell stimulus.

Embodiment 76. The method of any of one embodiments 44-75 further comprising (e) restimulating the population of primary immune cells.

Embodiment 77. The method of embodiment 76, wherein the population of primary immune cells undergoes at least about a 10⁸-fold expansion during culturing.

Embodiment 78. The method of any one of embodiments 44-77, wherein the transgene is introduced using a plasmid-based DNA transposon.

Embodiment 79. The method of any one of embodiments 44-78, wherein the transgene is introduced using a lentivirus platform.

Embodiment 80. The method of any one of embodiments 44-79, wherein the transgene is introduced using site specific integration via CRISPR.

Embodiment 81. A method of generating a population of primary immune cells resistant to replicative senescence (RRS), comprising:
(a) inhibiting expression of cyclin-dependent kinase inhibitor 2A (CDKN2A), cyclin-dependent kinase inhibitor 2B (CDKN2B), or S-methyl-5′-thioadenosine phosphorylase (MTAP) in the population of primary immune cells;
(b) introducing a transgene encoding one or more STAT5A mutants and/or one or more STAT5B mutants in the population of primary immune cells;
(c) introducing a transgene encoding TERT in the population of primary immune cells; and
(d) culturing the population of primary immune cells in a culture medium;
wherein the culturing induces proliferation of the primary immune cells to yield the population of primary immune cells resistant to replicative senescence (RRS).

Embodiment 82. The method of embodiment 81 further comprising stimulating the primary immune cells before performing step (a) and/or step (b) and/or step (c) and/or step (d) in the population of primary immune cells.

Embodiment 83. The method of either embodiment 81 or embodiment 82 further comprising stimulating the primary immune cells after performing step (a) and/or step (b) and/or step (c) and/or step (d) in the population of primary immune cells.

Embodiment 84. The method of any one of embodiments 81-83 further comprising inhibiting the expression of one or more endogenous immune related genes in the population of primary immune cells.

Embodiment 85. The method of any one of embodiments 81-84, wherein the one or more STAT5A mutants can be H299R, N642H, Y665F, S711F, and combinations thereof, and/or wherein the one or more STAT5B mutants can be H298R, R430C, E433K, N642H, Y665F, P702A, V712E, S715F, and combinations thereof.

Embodiment 86. The method of embodiment 84, wherein the endogenous immune related gene is beta-2 microglobulin (B2M) and/or T-cell receptor α constant (TRAC).

Embodiment 87. The method of any one of embodiments 81-86 further comprising inhibiting the expression of cluster of differentiation 38 (CD38), inhibiting the expression of phosphatase and tensin homolog (PTEN), and/or inhibiting the expression of p53 in the population of primary immune cells.

Embodiment 88. The method of any one of embodiments 81-87 further comprising introducing a transgene encoding MYC and/or introducing a transgene encoding KRAS in the population of primary immune cells.

Embodiment 89. A method of generating a population of primary immune cells resistant to replicative senescence (RRS), comprising:
(a) inhibiting the expression of cyclin-dependent kinase inhibitor 2A (CDKN2A), cyclin-dependent kinase inhibitor 2B (CDKN2B), and S-methyl-5′-thioadenosine phosphorylase (MTAP) in the population of primary immune cells;
(b) introducing a transgene encoding MYC in the population of primary immune cells;
(c) introducing a transgene encoding TERT in the population of primary immune cells; and
(d) culturing the primary immune cells in a culture medium;
wherein the culturing induces proliferation of the primary immune cells to yield the population of primary immune cells resistant to replicative senescence (RRS).

Embodiment 90. The method of embodiment 89 further comprising introducing a transgene encoding B-cell lymphoma-extra large (Bcl-xL) in the population of primary immune cells.

Embodiment 91. The method of either embodiment 89 or embodiment 90 further comprising inhibiting the expression of p53 in the population of primary immune cells.

Embodiment 92. The method of any one of embodiments 89-91 further comprising introducing a transgene encoding KRAS in the population of primary immune cells.

Embodiment 93. The method of embodiment 92, wherein KRAS comprises a KRAS A146V mutation.

Embodiment 94. The method of any one of embodiments 89-93 further comprising inhibiting the expression of phosphatase and tensin homolog (PTEN) in the population of primary immune cells.

Embodiment 95. The method of embodiment 94, wherein PTEN expression is inhibited by a CRISPR/Cas system.

Embodiment 96. The method of any one of embodiments 89-95 further comprising stimulating the population of primary immune cells before performing step (a) and/or step (b) and/or step (c) and/or step (d) in the population of primary immune cells.

Embodiment 97. The method of any one of embodiments 89-96 further comprising stimulating the population of primary immune cells after performing step (a) and/or step (b) and/or step (c) and/or step (d) in the population of primary immune cells.

Embodiment 98. The method of any one of embodiments 89-97 further comprising inhibiting the expression of one or more endogenous immune related genes in the primary immune cells in the population of primary immune cells.

Embodiment 99. The method of embodiment 98, wherein the endogenous immune related gene is beta-2 microglobulin (B2M) and/or T-cell receptor α constant (TRAC).

Embodiment 100. The method of any one of embodiments 89-99 further comprising introducing into the primary immune cells one or more transgenes encoding any one of Saimiriine gammaherpesvirus 2 StpA A11, Herpesvirus saimiri StpC, Herpesvirus saimiri Tip, or a modified Herpesvirus Ateles-Epstein-Barr virus Tio-LMP1.

Embodiment 101. The method of any one of embodiments 89-100 further comprising inhibiting the expression of cluster of differentiation 38 (CD38) in the population of primary immune cells.

Embodiment 102. The method of any one of embodiments 81-101, wherein the population of primary immune cells comprises total T cells.

Embodiment 103. The method of any one of embodiments 81-101, wherein the population of primary immune cells comprises CD8+ T cells.

Embodiment 104. The method of any one of embodiments 81-101, wherein the population of primary immune cells comprises CD4+ T cells.

Embodiment 105. The method of any one of embodiments 81-101, wherein the population of primary immune cells comprises gamma-delta T cells, mucosal associated invariant T (MAIT) T cells, natural killer (NK) cells, and/or natural killer T (NKT) cells.

Embodiment 106. The method of any one of embodiments 81-105, wherein the population of primary immune cells is human.

Embodiment 107. The method of any one of embodiments 81-106 further comprising introducing a polynucleotide that encodes a chimeric antigen receptor (CAR) in the population of primary immune cells.

Embodiment 108. The method of any one of embodiments 81-107, wherein the population of primary immune cells can be cultured for at least 100 days.

Embodiment 109. The method of any one of embodiments 81-108, wherein the population of primary immune cells undergoes at least about a 10⁶-fold expansion during culturing.

Embodiment 110. The method of any one of embodiments 81-109, wherein the population of primary immune cells is cultured in a culture medium that does not include a primary immune cell stimulus.

Embodiment 111. The method of any one of embodiments 81-110 further comprising (c) stimulating the population of primary immune cells.

Embodiment 112. The method of embodiment 111, wherein the population of primary immune cells undergoes at least about a 10⁸-fold expansion during culturing.

Embodiment 113. The method of any one of embodiments 81-112, wherein the transgene is introduced using a plasmid-based DNA transposon.

Embodiment 114. The method of any one of embodiments 81-112, wherein the transgene is introduced using a lentivirus platform.

Embodiment 115. The method of any one of embodiments 81-112, wherein the transgene is introduced using site specific integration via CRISPR.

Embodiment 116. An engineered immune cell population produced according to the method of any one of embodiments 1-115.

Embodiment 117. A pharmaceutical composition comprising the engineered immune cell population of embodiment 116 and a pharmaceutically acceptable carrier.

Embodiment 118. A method of treating a cancer in a subject in need thereof, comprising administering to the subject a therapeutically effective amount of the pharmaceutical composition of embodiment 117.

Embodiment 119. An engineered T cell that does not express cyclin-dependent kinase inhibitor 2A (CDKN2A), cyclin-dependent kinase inhibitor 2B (CDKN2B), and/or S-methyl-5′-thioadenosine phosphorylase (MTAP), wherein the engineered T cell comprises a transgene encoding one or more STAT5A mutants and/or one or more STAT5B mutants.

Embodiment 120. The engineered T cell of embodiment 119, wherein the one or more STAT5A mutants can be H299R, N642H, Y665F, S711F, and combinations thereof, and/or wherein the one or more STAT5B mutants can be H298R, R430C, E433K, N642H, Y665F, P702A, V712E, S715F, and combinations thereof.

Embodiment 121. The engineered T cell of either embodiment 119 or embodiment 120 further comprising introducing a transgene encoding TERT.

Embodiment 122. The engineered T cell of embodiment 119, wherein the engineered T cell further comprises a transgene encoding either B-cell lymphoma-extra large (Bcl-xL) or B-cell lymphoma 2 (Bcl-2).

Embodiment 123. The engineered T cell of either embodiment 119 or embodiment 120, wherein the engineered T cell does not express of one or more endogenous immune related genes.

Embodiment 124. The engineered T cell of embodiment 123, wherein the endogenous immune related gene is beta-2 microglobulin (B2M) and/or T-cell receptor α constant (TRAC).

Embodiment 125. The engineered T cell of any one of embodiments 119-124, wherein the engineered T cell does not express cluster of differentiation 38 (CD38), phosphatase and tensin homolog (PTEN), and/or p53.

Embodiment 126. The engineered T cell of any one of embodiments 119-125 further comprising a transgene encoding MYC and/or a transgene encoding KRAS.

Embodiment 127. An engineered T cell that does not express cyclin-dependent kinase inhibitor 2A (CDKN2A), cyclin-dependent kinase inhibitor 2B (CDKN2B), and/or S-methyl-5′-thioadenosine phosphorylase (MTAP).

Embodiment 128. The engineered T cell of embodiment 127 further comprising a transgene encoding B-cell lymphoma-extra large (Bcl-xL) and a transgene encoding MYC.

Embodiment 129. The engineered T cell of either embodiment 127 or embodiment 128, wherein the engineered T cell does not express p53.

Embodiment 130. The engineered T cell of any one of embodiments 127-129 further comprising a transgene encoding KRAS.

Embodiment 131. The engineered T cell of embodiment 130, wherein KRAS comprises a KRAS A146V mutation.

Embodiment 132. The engineered T cell of any one of embodiments 127-131, wherein the engineered T cell does not express phosphatase and tensin homolog (PTEN).

Embodiment 133. The engineered T cell of embodiment 132, wherein PTEN expression is inhibited by a CRISPR/Cas system.

Embodiment 134. An engineered T cell that does not express cyclin-dependent kinase inhibitor 2A (CDKN2A), cyclin-dependent kinase inhibitor 2B (CDKN2B), and/or S-methyl-5′-thioadenosine phosphorylase (MTAP), and comprises a transgene encoding TERT.

Embodiment 135. The engineered T cell of embodiment 134, wherein the engineered T cell comprises a transgene encoding B-cell lymphoma-extra large (Bcl-xL) and a transgene encoding MYC.

Embodiment 136. The engineered T cell of either embodiment 134 or embodiment 135 further comprising a transgene encoding KRAS.

Embodiment 137. The engineered T cell of embodiment 136, wherein KRAS comprises a KRAS A146V mutation.

Embodiment 138. The engineered T cell of any one of embodiments 127-138, wherein the engineered T cell does not express one or more endogenous immune related genes in the primary immune cells in the population of primary immune cells.

Embodiment 139. The engineered T cell of embodiment 138, wherein the endogenous immune related gene is beta-2 microglobulin (B2M) and/or T-cell receptor α constant (TRAC).

Embodiment 140. The engineered T cell of any one of embodiments 127-139, wherein the engineered T cell does not express cluster of differentiation 38 (CD38).

Embodiment 141. The engineered T cell of any one of embodiments 119-140 further comprising a polynucleotide that encodes a chimeric antigen receptor (CAR).

Embodiment 142. The engineered T cell of any one of embodiments 119-141, wherein the engineered T cell is a CD8+ T cell, a CD4+ T cell, a gamma-delta T cell, a mucosal associated invariant T (MAIT) T cell, a natural killer (NK) cell, a natural killer T (NKT) cell, or a combination thereof.

Embodiment 143. The engineered T cell of any one of embodiments 119-141, wherein the engineered T cell is a CD8+ T cell.

Embodiment 144. The engineered T cell of any one of embodiments 119-141, wherein the engineered T cell is a CD4+ T cell.

Embodiment 145. The engineered T cell of any one of embodiments 119-144, wherein the engineered T cell is human.

Embodiment 146. An engineered T cell that does not express cyclin-dependent kinase inhibitor 2A (CDKN2A), cyclin-dependent kinase inhibitor 2B (CDKN2B), S-methyl-5′-thioadenosine phosphorylase (MTAP), beta-2 microglobulin (B2M), and/or T-cell receptor α constant (TRAC), wherein the engineered T cell comprises a transgene encoding one or more STAT5A mutants and/or one or more STAT5B mutants, and wherein the engineered T cell comprises a transgene encoding TERT.

Embodiment 147. The engineered T cell of embodiment 146, wherein the engineered T cell does not express cluster of differentiation 38 (CD38), phosphatase and tensin homolog (PTEN), and/or p53.

Embodiment 148. The engineered T cell of either embodiment 146 or embodiment 147, wherein the engineered T cell further comprises a transgene encoding MYC and/or a transgene encoding KRAS.

Embodiment 149. The engineered T cell of any one of embodiments 146-148 further comprising a polynucleotide that encodes a chimeric antigen receptor (CAR).

Embodiment 150. The engineered T cell of any one of embodiments 146-149, wherein the engineered T cell is a gamma-delta T cell, a mucosal associated invariant T (MAIT) T cell, a natural killer (NK) cell, a natural killer T (NKT) cell, or a combination thereof.

Embodiment 151. The engineered T cell of any one of embodiments 146-149, wherein the engineered T cell is a CD8+ T cell.

Embodiment 152. The engineered T cell of any one of embodiments 146-149, wherein the engineered T cell is a CD4+ T cell.

Embodiment 153. The engineered T cell of any one of embodiments 146-152, wherein the engineered T cell is human.

Embodiment 154. An engineered T cell expressing a transgene encoding a B-cell lymphoma-extra large (Bcl-XL), wherein the engineered T cell does not express cyclin-dependent kinase inhibitor 2A (CDKN2A), cyclin-dependent kinase inhibitor 2B (CDKN2B), S-methyl-5′-thioadenosine phosphorylase (MTAP), and/or phosphatase and tensin homolog (PTEN), and wherein the engineered T cell comprises a transgene encoding one or more STAT5A mutants and/or one or more STAT5B mutants.

Embodiment 155. The engineered T cell of embodiment 154, wherein the one or more STAT5A mutants can be H299R, N642H, Y665F, S711F, and combinations thereof, and/or wherein the one or more STAT5B mutants can be H298R, R430C, E433K, N642H, Y665F, P702A, V712E, S715F, and combinations thereof.

Embodiment 156. The engineered T cell of either embodiment 154 or embodiment 155, wherein the engineered T cell does not express of one or more endogenous immune related genes.

Embodiment 157. The engineered T cell of any one of embodiments 154-156, wherein the endogenous immune related gene is beta-2 microglobulin (B2M) or T-cell receptor α

Embodiment 158. The engineered T cell of any one of embodiments 154-157, wherein the engineered T cell does not express cluster of differentiation 38 (CD38), phosphatase and tensin homolog (PTEN), and/or p53.

Embodiment 159. The engineered T cell of any one of embodiments 154-158, wherein the engineered T cell comprises a transgene encoding MYC and/or a transgene encoding KRAS.

Embodiment 160. The engineered T cell of any one of embodiments 154-159 further comprising a polynucleotide that encodes a chimeric antigen receptor (CAR).

Embodiment 161. The engineered T cell of any one of embodiments 154-160, wherein the engineered T cell is a CD8+ T cell, a CD4+ T cell, a delta gamma T cell, a mucosal associated invariant T (MAIT) T cell, a natural killer (NK) T cell, or a combination thereof.

Embodiment 162. The engineered T cell of any one of embodiments 154-160, wherein the engineered T cell is a CD8+ T cell.

Embodiment 163. The engineered T cell of any one of embodiments 154-160, wherein the engineered T cell is a CD4+ T cell.

Embodiment 164. The engineered T cell of any one of embodiments 154-163, wherein the engineered T cell is human.

Embodiment 165. Use of an engineered T cell for the manufacture of a medicament for treating cancer in a patient, wherein the engineered T cell does not express cyclin-dependent kinase inhibitor 2A (CDKN2A), cyclin-dependent kinase inhibitor 2B (CDKN2B), and/or S-methyl-5′-thioadenosine phosphorylase (MTAP), and wherein the engineered T cell comprises a transgene encoding one or more STAT5A mutants and/or one or more STAT5B mutants.

Embodiment 166. The use of embodiment 165, wherein the one or more STAT5A mutants can be H299R, N642H, Y665F, S711F, and combinations thereof, and/or wherein the one or more STAT5B mutants can be H298R, R430C, E433K, N642H, Y665F, P702A, V712E, S715F, and combinations thereof.

Embodiment 167. The use of either embodiment 165 or embodiment 166, wherein the engineered T cell further comprises a polynucleotide that encodes a chimeric antigen receptor (CAR).

Embodiment 168. The use of any one of embodiments 165-167, wherein the engineered T cell further comprises a transgene encoding either B-cell lymphoma-extra large (Bcl-xL) or B-cell lymphoma 2 (Bcl-2).

Embodiment 169. The use of any one of embodiments 165-168, wherein the engineered T cell further comprises a transgene encoding TERT.

Embodiment 170. The use of any one of embodiments 165-169, wherein the engineered T cell does not express of one or more endogenous immune related genes.

Embodiment 171. The use of embodiment 166, wherein the endogenous immune related gene is beta-2 microglobulin (B2M) and/or T-cell receptor α constant (TRAC).

Embodiment 172. The use of any one of embodiments 165-171, wherein the engineered T cell does not express cluster of differentiation 38 (CD38), phosphatase and tensin homolog (PTEN), and/or p53.

Embodiment 173. The use of any one of embodiments 165-172, wherein the engineered T cell comprises a transgene encoding MYC and/or a transgene encoding KRAS.

Embodiment 174. Use of an engineered T cell for the manufacture of a medicament for treating cancer in a patient, wherein the engineered T cell does not express cyclin-dependent kinase inhibitor 2A (CDKN2A), cyclin-dependent kinase inhibitor 2B (CDKN2B), and/or S-methyl-5′-thioadenosine phosphorylase (MTAP).

Embodiment 175. The use of embodiment 174, wherein the engineered T cell comprises a transgene encoding B-cell lymphoma-extra large (Bcl-xL) and a transgene encoding MYC.

Embodiment 176. The use of either embodiment 174 or embodiment 175, wherein the engineered T cell does not express p53.

Embodiment 177. The use of any one of embodiments 174-176, wherein the engineered T cell comprises a transgene encoding KRAS.

Embodiment 178. The use of embodiment 177, wherein KRAS comprises a KRAS A146V mutation.

Embodiment 179. The use of any one of embodiments 174-178, wherein the engineered T cell does not express phosphatase and tensin homolog (PTEN).

Embodiment 180. The use of embodiment 179, wherein PTEN expression is inhibited by a CRISPR/Cas system.

Embodiment 181. Use of an engineered T cell for the manufacture of a medicament for treating cancer in a patient, wherein the engineered T cell does not express cyclin-dependent kinase inhibitor 2A (CDKN2A), cyclin-dependent kinase inhibitor 2B (CDKN2B), and/or S-methyl-5′-thioadenosine phosphorylase (MTAP), and wherein the engineered T cell comprises a transgene encoding TERT.

Embodiment 182. The use of embodiment 181, wherein the engineered T cell comprises a transgene encoding B-cell lymphoma-extra large (Bcl-xL) and a transgene encoding MYC.

Embodiment 183. The use of either embodiment 181 or embodiment 182, wherein the engineered T cell comprises a transgene encoding KRAS.

Embodiment 184. The use of embodiment 183, wherein KRAS comprises a KRAS A146V mutation.

Embodiment 185. The use of any one of embodiments 181-184, wherein the engineered T cell further comprises a polynucleotide that encodes a chimeric antigen receptor (CAR).

Embodiment 186. The use of any one of embodiments 174-185, wherein the engineered T cell does not express one or more endogenous immune related genes in the primary immune cells in the population of primary immune cells.

Embodiment 187. The use of embodiment 186, wherein the endogenous immune related gene is beta-2 microglobulin (B2M) and/or T-cell receptor α constant (TRAC).

Embodiment 188. The use of any one of embodiments 174-187, wherein the engineered T cell does not express cluster of differentiation 38 (CD38).

Embodiment 189. The use of any one of embodiments 165-188, wherein the engineered T cell is a CD8+ T cell, a CD4+ T cell, a gamma-delta T cell, a mucosal associated invariant T (MAIT) T cell, a natural killer (NK) cell, a natural killer T (NKT) cell, or a combination thereof.

Embodiment 190. The use of any one of embodiments 165-188, wherein the engineered T cell is a CD8+ T cell.

Embodiment 191. The use of any one of embodiments 165-188, wherein the engineered T cell is a CD4+ T cell.

Embodiment 192. The use of any one of embodiments 165-191, wherein the engineered T cell is human.

Embodiment 193. An engineered T cell for the treatment of cancer in a patient, wherein the engineered T cell does not express cyclin-dependent kinase inhibitor 2A (CDKN2A), cyclin-dependent kinase inhibitor 2B (CDKN2B), and/or S-methyl-5′-thioadenosine phosphorylase (MTAP), and wherein the engineered T cell comprises a transgene encoding one or more STAT5A mutants and/or one or more STAT5B mutants.

Embodiment 194. The engineered T cell of embodiment 193, wherein the one or more STAT5A mutants can be H299R, N642H, Y665F, S711F, and combinations thereof, and/or wherein the one or more STAT5B mutants can be H298R, R430C, E433K, N642H, Y665F, P702A, V712E, S715F, and combinations thereof.

Embodiment 195. The engineered T cell of either embodiment 193 or embodiment 194, wherein the engineered T cell further comprises a transgene encoding either B-cell lymphoma-extra large (Bcl-xL) or B-cell lymphoma 2 (Bcl-2).

Embodiment 196. The engineered T cell of any one of embodiments 193-195, wherein the engineered T cell further comprises a transgene encoding TERT.

Embodiment 197. The engineered T cell of any one of embodiments 193-196, wherein the engineered T cell does not express of one or more endogenous immune related genes.

Embodiment 198. The engineered T cell of embodiment 197, wherein the endogenous immune related gene is beta-2 microglobulin (B2M) and/or T-cell receptor α constant (TRAC).

Embodiment 199. The engineered T cell of any one of embodiments 193-198, wherein the engineered T cell does not express cluster of differentiation 38 (CD38), PTEN, and/or p53.

Embodiment 200. The engineered T cell of any one of embodiments 193-199, wherein the engineered T cell comprises a transgene encoding MYC and/or a transgene encoding KRAS.

Embodiment 201. The engineered T cell of any one of embodiments 193-200, wherein the engineered T cell further comprises a polynucleotide that encodes a chimeric antigen receptor (CAR).

Embodiment 202. An engineered T cell for the treatment of cancer in a patient, wherein the engineered T cell does not express cyclin-dependent kinase inhibitor 2A (CDKN2A), cyclin-dependent kinase inhibitor 2B (CDKN2B), and/or S-methyl-5′-thioadenosine phosphorylase (MTAP).

Embodiment 203. The engineered T cell of embodiment 202, further comprising a transgene encoding B-cell lymphoma-extra large (Bcl-xL) and a transgene encoding MYC.

Embodiment 204. The engineered T cell of either embodiment 202 or embodiment 203, wherein the engineered T cell does not express p53.

Embodiment 205. The engineered T cell of any one of embodiments 202-204 further comprising a transgene encoding KRAS.

Embodiment 206. The engineered T cell of embodiment 205, wherein KRAS comprises a KRAS A146V mutation.

Embodiment 207. The engineered T cell of any one of embodiments 202-206, wherein the engineered T cell does not express phosphatase and tensin homolog (PTEN).

Embodiment 208. The engineered T cell of embodiment 207, wherein PTEN expression is inhibited by a CRISPR/Cas system.

Embodiment 209. An engineered T cell for the treatment of cancer in a patient that does not express cyclin-dependent kinase inhibitor 2A (CDKN2A), cyclin-dependent kinase inhibitor 2B (CDKN2B), and/or S-methyl-5′-thioadenosine phosphorylase (MTAP), and comprises a transgene encoding TERT.

Embodiment 210. The engineered T cell of embodiment 209, wherein the engineered T cell comprises a transgene encoding B-cell lymphoma-extra large (Bcl-xL) and a transgene encoding MYC.

Embodiment 211. The engineered T cell of either embodiment 209 or embodiment 210 further comprising a transgene encoding KRAS.

Embodiment 212. The engineered T cell of embodiment 211, wherein KRAS comprises a KRAS A146V mutation.

Embodiment 213. The engineered T cell of any one of embodiments 202-212, wherein the engineered T cell does not express one or more endogenous immune related genes in the primary immune cells in the population of primary immune cells.

Embodiment 214. The engineered T cell of embodiment 213, wherein the endogenous immune related gene is beta-2 microglobulin (B2M) and/or T-cell receptor α constant (TRAC).

Embodiment 215. The engineered T cell of any one of embodiments 202-214, wherein the engineered T cell does not express cluster of differentiation 38 (CD38).

Embodiment 216. The engineered T cell of any one of embodiments 202-215, wherein the engineered T cell further comprises a polynucleotide that encodes a chimeric antigen receptor (CAR).

Embodiment 217. The engineered T cell of any one of embodiments 193-216, wherein the engineered T cell is a CD8+ T cell, a CD4+ T cell, a gamma-delta T cell, a mucosal associated invariant T (MAIT) T cell, a natural killer (NK) cell, a natural killer T (NKT) cell, or a combination thereof.

Embodiment 218. The engineered T cell of any one of embodiments 193-216, wherein the engineered T cell is a CD8+ T cell.

Embodiment 219. The engineered T cell of any one of embodiments 193-216, wherein the engineered T cell is a CD4+ T cell.

Embodiment 220. The engineered T cell of any one of embodiments 193-219, wherein the engineered T cell is human.

### EXAMPLES

The Examples that follow are illustrative of specific aspects of the disclosure, and various uses thereof. They are set forth for explanatory purposes only and should not be construed as limiting the scope of the disclosure in any way.

### Example 1: Overexpression of Anti-Apoptotic or Virally-Derived Factors can Provide a Selective Survival Advantage to Transfected T Cells in Long-Term Culture

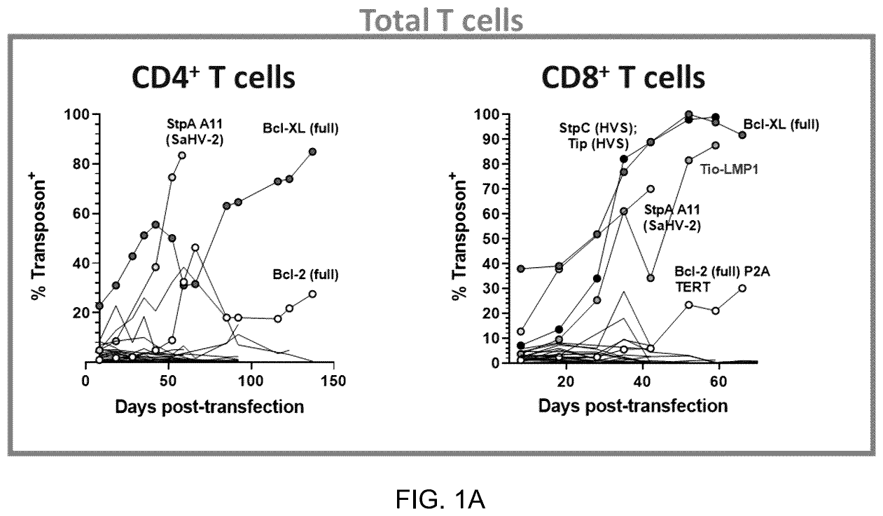

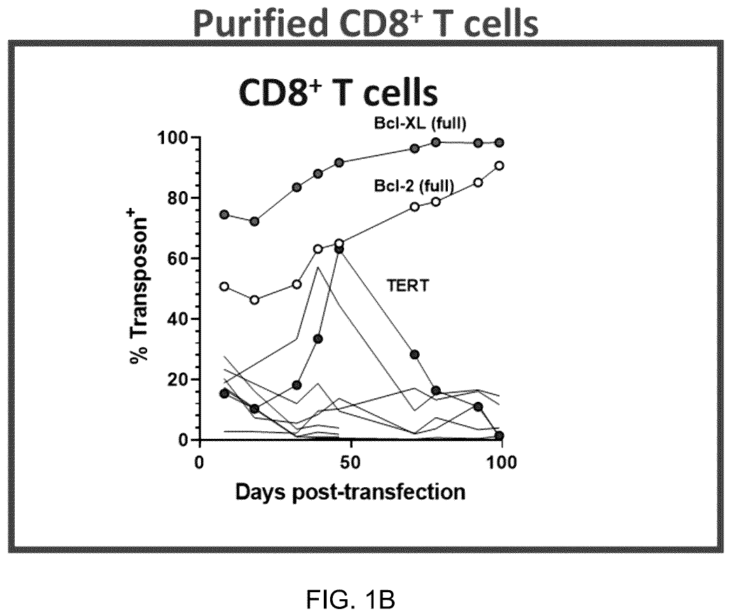

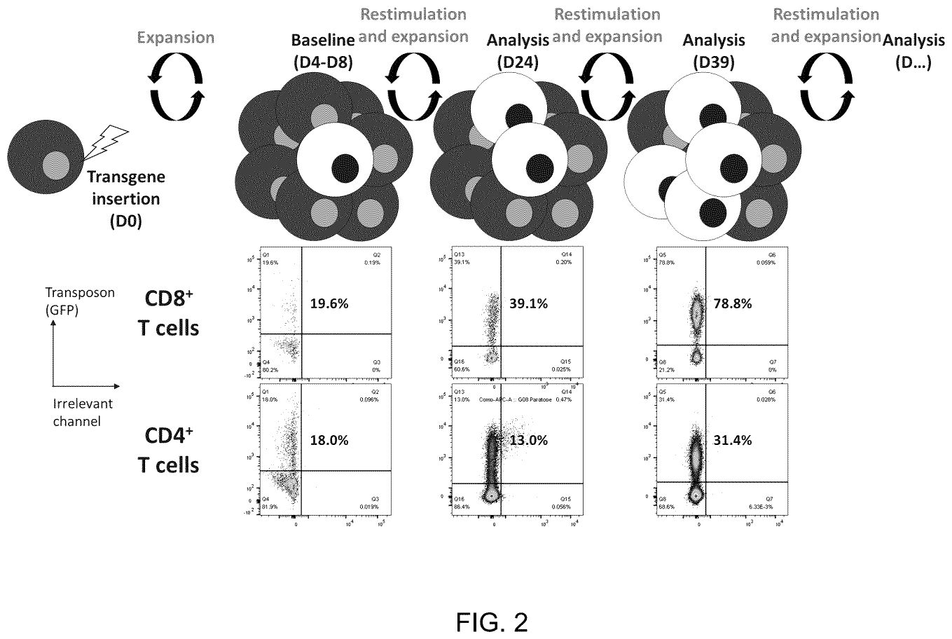

Transposon frequency was assessed in T cell subsets over a period of 66-137 days using flow cytometry. Total primary human T cells were isolated from the blood of healthy donors, activated for 72 hours with Dynabeads (Human T-Activator CD3/CD28) and then transfected with plasmids containing transposons encoding anti-apoptotic factors, virally-derived factors, mutant cytokine receptors, mutant signaling molecules, and/or mutant cell cycle regulatory molecules in addition to a fluorescent reporter. mRNA encoding a transposase was simultaneously transfected into cells to enable chromosomal integration of the transposable elements. In total, 52 transposon constructs were tested across various screens. Four to 8 days after transfection, cells were assessed for baseline transposon incorporation using flow cytometry. Cells were periodically restimulated with Dynabeads to drive them through proliferation and transposon enrichment was assessed using flow cytometry. Molecules that enhance the survival of expanding T cells would be expected to enrich over their starting frequency within the total T cell pool as demonstrated in the CD8⁺ and CD4⁺ T cells. (FIG. 1, FIG. 2). These screens revealed that the anti-apoptotic factor B-cell lymphoma-extra-large (Bcl-xL) consistently enriched in both CD4⁺ and CD8⁺ T cells that were driven through multiple rounds of proliferation over a period of extended in vitro culture, suggesting that this factor may act to enhance the survival of the mature T cells that overexpress it (FIG. 1). Other factors such Bcl-2 and the virally-derived proteins StpA A11 (Saimiriine gammaherpesvirus 2), StpC and Tip (Herpesvirus saimiri), and a modified Tio-LMP1 (Herpesvirus Ateles, Epstein-Barr virus) also demonstrated enrichment in T cell subsets (FIG. 1).

While cells expressing transgenes encoding endogenous anti-apoptotic factors (Bcl-2 and Bcl-xL) or virally-derived factors (StpA A11, StpC and Tip, and a modified Tio-LMP1) demonstrated an enhanced ability to survive in long-term culture with repeated stimulation through their TCRs relative to untransfected control cells in the same wells, these cells did not exhibit a large, sustained boost in their proliferative capacities. These data suggest that additional edits may be required to confer the desired phenotype of a T_{REX} cell.

### Example 2: Ablation of Expression of CDKN2A, CDKN2B, and MTAP Substantially Increases the Proliferative Capacity of Primary Human T Cells in Long-Term Culture

Patient-derived leukemic cell lines have been used for years in laboratories to conduct a variety of cellular assays. The transformed nature of these cells can be mapped on to a collection of mutations that are also frequently found in patients with T cell acute lymphoblastic leukemia (T-ALL) (Table 1). Mutations in T-ALL patients can be broken down into several large classes that each presumably contribute to the phenotype and generation of T-ALL cells: gain of activating signals, loss of signal suppressors, loss of cell cycle arrest regulators, as well as modification of pleiotropic factors such as transcription factors, epigenetic regulators, and other cellular machinery.

TABLE 1
Potential targets for T_{REX} cell development

| Biological | Pathway or | | General Strategy |
|---|---|---|---|
| Function | Target | Examples | for T_{REX} Cells |
| ACTIVATING | NOTCH | NOTCH1*, | AVOID genetic |
| signal | | FBXW7 | edits; significant, |
| | | | broad reaching |
| | | | effects of signaling. |
| | PI3K/mTOR/ | AKT*, | EDIT and if |
| | AKT | PI3KCA*, | effective express |
| | | mTOR* | in an inducible |
| | Cytokine | JAK1*, JAK3*, | manner to limit |
| | Signaling/Jak/ | IL7RA*, | cytokine- |
| | STAT | STAT5A*, | independence. |
| | | STAT5B* | |
| | Ras/MAPK | KRAS*, NRAS*, | |
| | | NF1 | |
| | Shared | MYC*; TERT* | |
| Signal | PI3K/mTOR/ | PTEN | EDIT to limit signal |
| SUPPRESSOR | AKT | | suppression. |
| | Other | PTPN2 | |
| Cell Cycle | CDKN2A/2B | CDKN2A/2B | EDIT to limit cell |
| ARREST | RB | RB1 | cycle arrest. |
| | P53 | TP53 | |
| Pleiotropic | Translocations | BCL11B, ETV6, | AVOID genetic |
| | | GATA3, | edits due to diverse |
| | | HOX11*, | range of biological |
| | | HOX11L2*, | effects. |
| | | HOXA* LEF1, | |
| | | LMO2*, MYB*, | |
| | | MYC* | |
| | | NKX2.1/ | |
| | | NKX2.2*, | |
| | | NUP214-ABL1/ | |
| | | ABL1 gain *, | |
| | | RUNX1, TAL1*, | |
| | | TLX1*, WT1 | |
| | Epigenetic | DNMT3A, EED, | |
| | Modifiers | EZH2, | |
| | Other | DNM2, CNOT3, | |
| | | RPL5, RPL10, | |
| | | RPL22 | |

*indicates gain of function for example through mutation of the protein, translocations, or mutation of the promoter/enhancer regions as may be the case for a protein such as TERT. Lack of an asterisk indicates loss of function (deletion, loss of function mutations, indels, truncations, etc.).

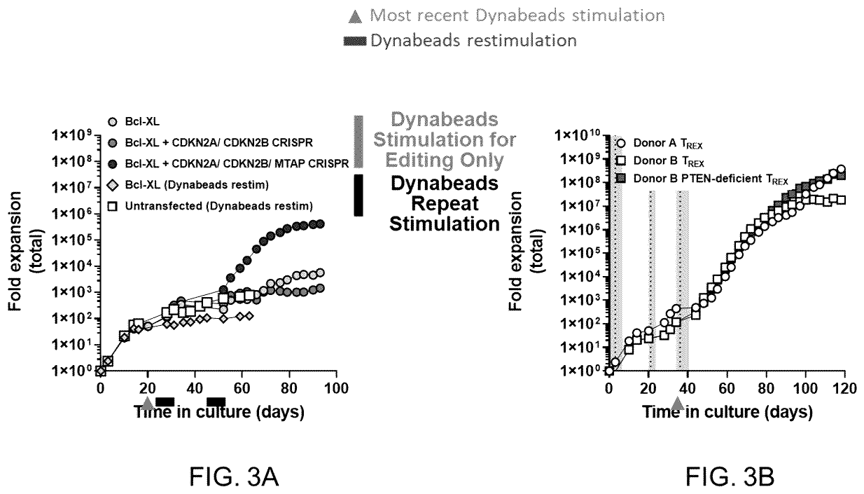

As stated above, it was hypothesized that in addition to providing a survival factor such as Bcl-XL, it may be necessary to modify T cell expression of a collection of the aforementioned genes in order to recreate the desired phenotype (Table 1). Therefore, total CD8⁺ T cells were isolated from the blood of healthy donors, activated using αCD3/αCD28 Dynabeads, and 72 hours later a transgene encoding Bcl-XL was inserted into the collective pool of purified CD8⁺ T cells. These cells were then expanded for a period of 17 days prior to reactivation using αCD3/αCD28 Dynabeads and subsequent ablation of expression of factors identified from leukemic T cell lines and patients with T-ALL. Increased proliferative capacity is one of the primary characteristics that was to be engineered into T_{REX} cells, therefore, effects of ablating expression of molecules from the “Cell Cycle ARREST” bin: cyclin-dependent kinase inhibitor 2A (CDKN2A) and CDKN2B were tested in these cells (FIG. 3A). S-methyl-5′-thioadenosine phosphorylase (MTAP) is chromosomally adjacent to CDKN2A and CDKN2B and is also frequently lost in patients with deletions of CDKN2A and CDKN2B. Accordingly, the effects of ablating expression of MTAP in conjunction with CDKN2A and CDKN2B (FIG. 3A) were tested. These Bcl-XL and CDKN2A/CDKN2B/MTAP-edited cells are subsequently referred to as “T_{REX}+Bcl-xL” cells (Table 2) and the CDKN2A/CDKN2B/MTAP edits (without Bcl-XL) are referred to as “T_{REX}” cells.

TABLE 2
Editing and population terminology

| Term | Specific Edits | Donor |
|---|---|---|
| T_{REX} + Bcl-xL | CDKN2A/CDKN2B/MTAP edits + | 40A30 |
| cells | Bcl-xL transgene insertion | (“Donor A”) |
| | | and 40B30 |
| | | (“Donor B”) |
| PTEN-deficient | CDKN2A/CDKN2B/MTAP edits + | 40B32 |
| T_{REX} cells | Bcl-xL transgene insertion + | (“Donor B-2”) |
| | ablation of PTEN expression | |

Bcl-xL-edited, Bcl-xL and CDKN2A/CDKN2B-edited, and T_{REX}+Bcl-xL cells were maintained in culture for nearly 100 days without additional stimulation through their TCRs and total fold expansion of each population was assessed (FIG. 3A). Proliferation of T_{REX}+Bcl-xL cells diverged from the other groups approximately 31 days after introducing these edits, and T_{REX}+Bcl-xL cell expansion was sustained in the absence of additional TCR stimulation, achieving a more than 400,000-fold expansion during this period. In contrast, Bcl-xL-edited and Bcl-xL/CDKN2A/CDKN2B-edited CD8⁺ T cells achieved 73-286-fold lower levels of expansion in this time (FIG. 3A, Table 3). Furthermore, even when unedited or Bcl-xL-edited cells were repeatedly restimulated through their TCRs using αCD3/αCD28 Dynabeads to drive proliferation, they achieved only low levels of total fold expansion (FIG. 1A, Table 3), well below those of the T_{REX}+Bcl-xL cells.

TABLE 3
CD8⁺ T cell proliferation

| | Total Fold | Total Fold |
|---|---|---|
| | Expansion | Expansion |
| Population | (Day 59) | (Day 93) |
| Bcl-xL cells | 554 | 5,677 |
| Bcl-xL + CDKN2A/CDKN2B | 926 | 1,459 |
| CRISPR cells | | |
| Bcl-xL + | 8,306 | 417,948 |
| CDKN2A/CDKN2B/MTAP | | |
| CRISPR (T_{REX} + Bcl-xL) cells | | |
| Bcl-xL cells (Dynabeads restim) | 121 | N/A |
| Untransfected cells | 689 | N/A |
| (Dynabeads restim) | | |

While T_{REX}+Bcl-xL cells demonstrated a substantially enhanced proliferative capacity relative to control CD8⁺ T cells from the same donor, further experiments were conducted to test whether these edits could confer a similar phenotype in other donors and whether it was possible to further enhance the T_{REX}+Bcl-xL phenotype by ablating expression of signal suppressors that are frequently mutated in patients with T-ALL (Table 1). The phosphatase and tensin homolog (PTEN) locus demonstrates frequent loss-of-function mutations in patient-derived leukemic cell lines and patients with T-ALL and is known to negatively regulate cell cycle progression (Table 1). T_{REX}+Bcl-xL cells were generated as above from two different donors (40A30 and 40B30) and expression of PTEN was ablated in one of these T_{REX}+Bcl-xL lines (40B32) approximately 2 weeks after “triplex” editing (FIG. 3B). Both sets of healthy donor-derived T_{REX} cells (lacking CDKN2A/CDKN2B/MTAP) exhibited substantial proliferative capacity in the absence of additional TCR stimulation, achieving >3.7e8 and >1.8e7 total fold expansion by day 118 in culture. Furthermore, in agreement with its established role in negatively regulating cell cycle progression through control of AKT signaling, ablation of PTEN expression in T_{REX}+Bcl-xL cells further enhanced the proliferative capacity of Donor B-2 T_{REX}+Bcl-xL cells, allowing these cells to reach >2.0e8 total fold expansion by day 118 in culture (FIG. 3B). The additive effects of ablation of PTEN expression took 49 days to emerge, reflecting low initial editing efficiency or a late competitive advantage of this edit after the cells have expanded >3e6 fold.

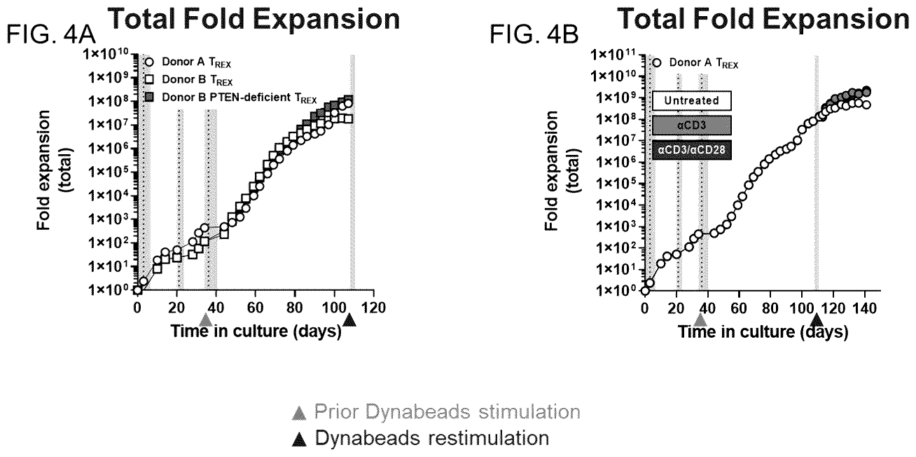

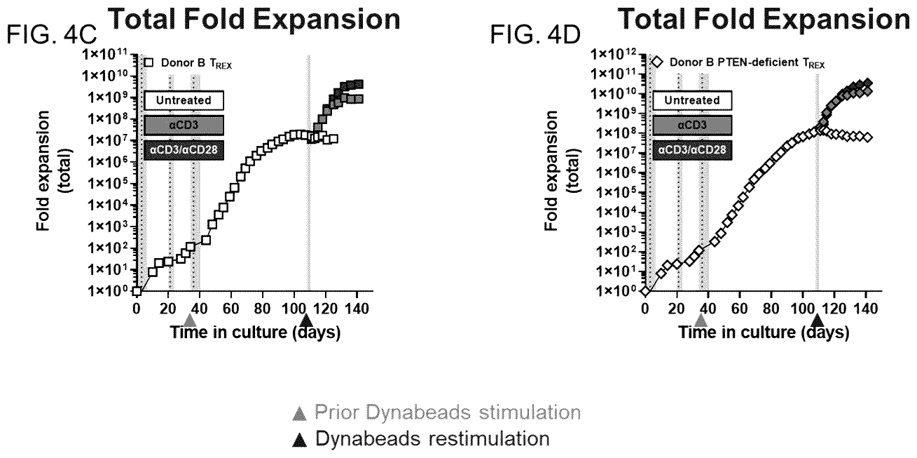

While T_{REX}+Bcl-xL cells with intact PTEN expression expanded dramatically in the absence of additional TCR stimulation, their proliferation ultimately slowed relative to T_{REX}+Bcl-xL cells deficient in PTEN expression (FIG. 3B). Furthermore, even PTEN-deficient T_{REX}+Bcl-xL cells eventually demonstrated decreased rates of proliferation (FIG. 3B). In order to determine if restimulation of T_{REX}+Bcl-xL cells in the presence or absence of co-stimulation could serve as a viable alternative or complementary approach to ablation of PTEN expression, it was tested whether αCD3 or αCD3/αCD28 Dynabeads could jumpstart cells back into cell cycle (FIG. 4). T_{REX}+Bcl-xL or PTEN-deficient T_{REX}+Bcl-xL cells were left untreated or restimulated with Dynabeads as above, debeaded, and total fold expansion of each population was tracked (FIG. 4). Restimulation substantially enhanced the ability of T_{REX}+Bcl-xL and PTEN-deficient T_{REX}+Bcl-xL lines to expand.

### Example 3: T_{REX}+Bcl-xL Cells Resemble Primary Human T Cells in Terms of Cytokine Dependence and Cell Phenotype

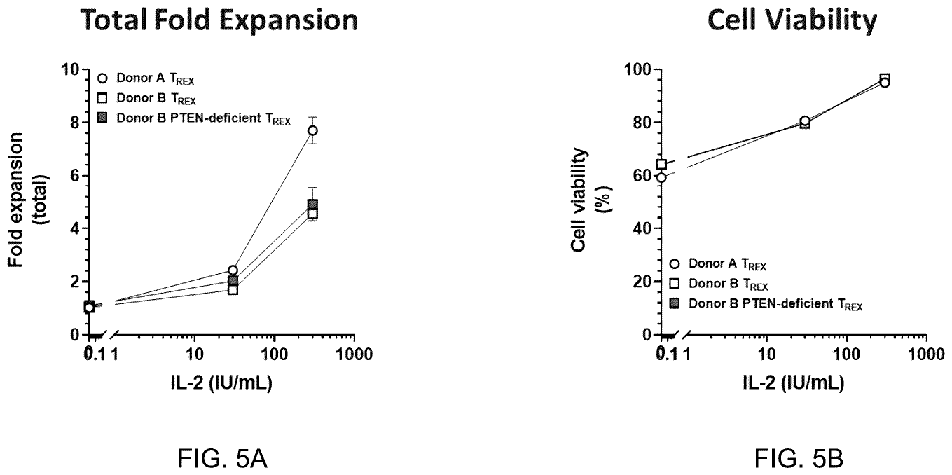

Primary T cells are dependent on cytokines such as IL-2 for survival and proliferation in vitro and in vivo, however, some leukemic cell lines grow independently of IL-2. T_{REX}+Bcl-xL cells and PTEN-deficient T_{REX}+Bcl-xL cells were generated in media containing IL-2. It was investigated whether these cells still resemble normal primary human T cells in regards to cytokine dependence by tracking cell proliferation and survival across a range of IL-2 concentrations over a period of 6 days in culture (FIG. 5). In line with normal T cells, T_{REX}+Bcl-xL cells and PTEN-deficient T_{REX}+Bcl-xL cells were highly dependent on IL-2 for both proliferation and survival.

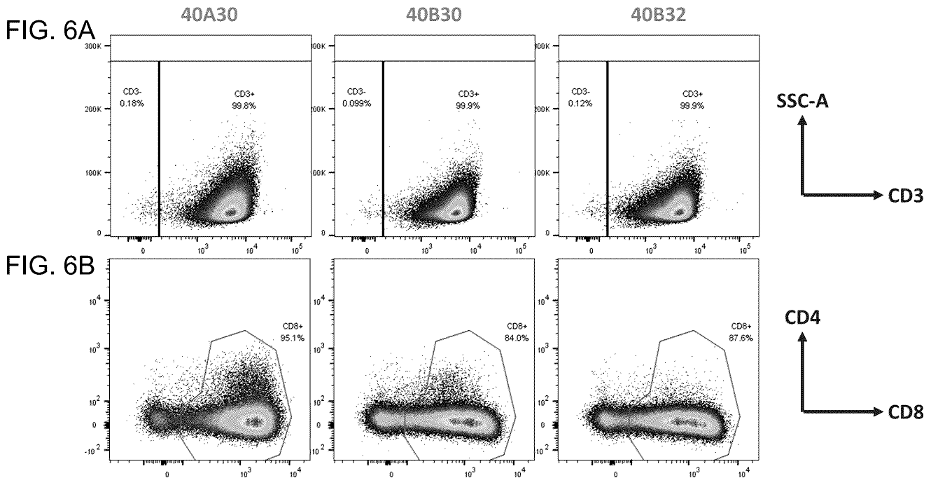

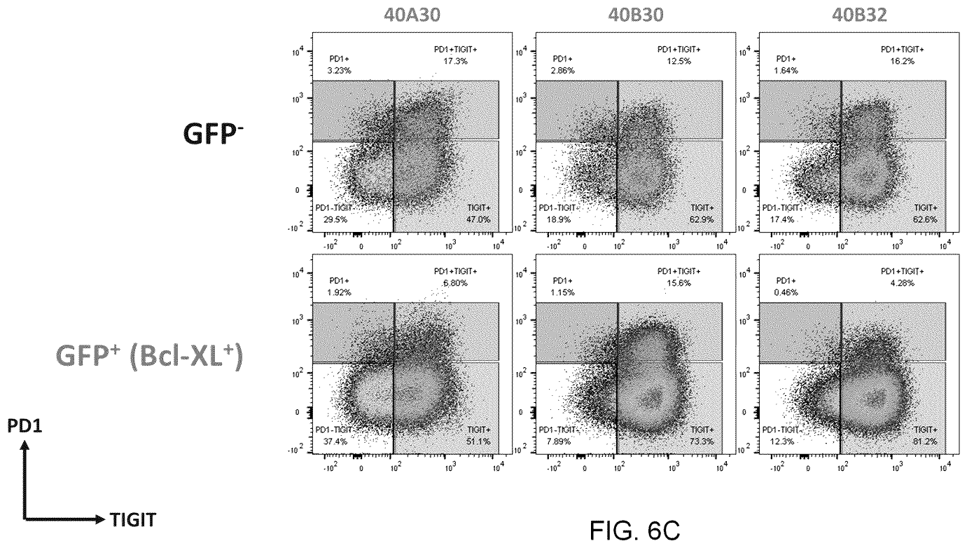

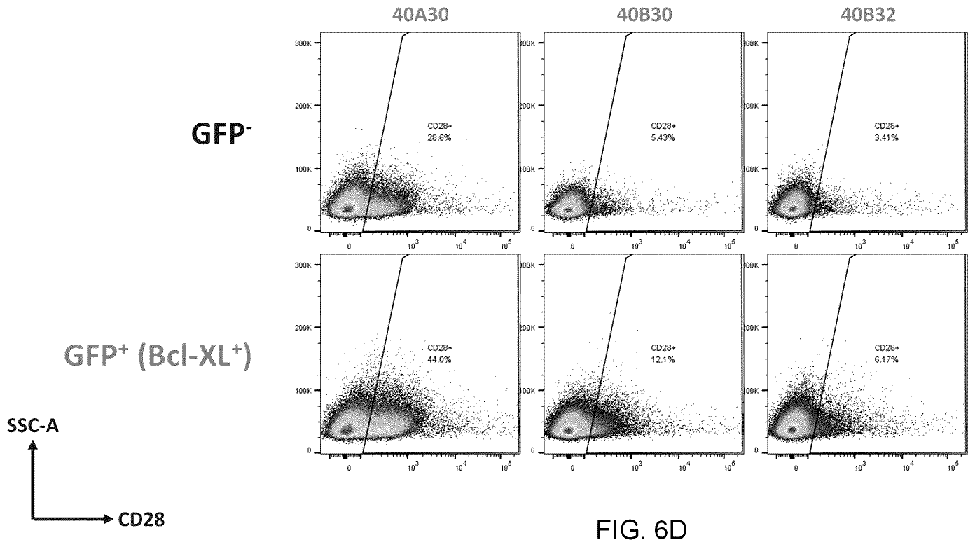

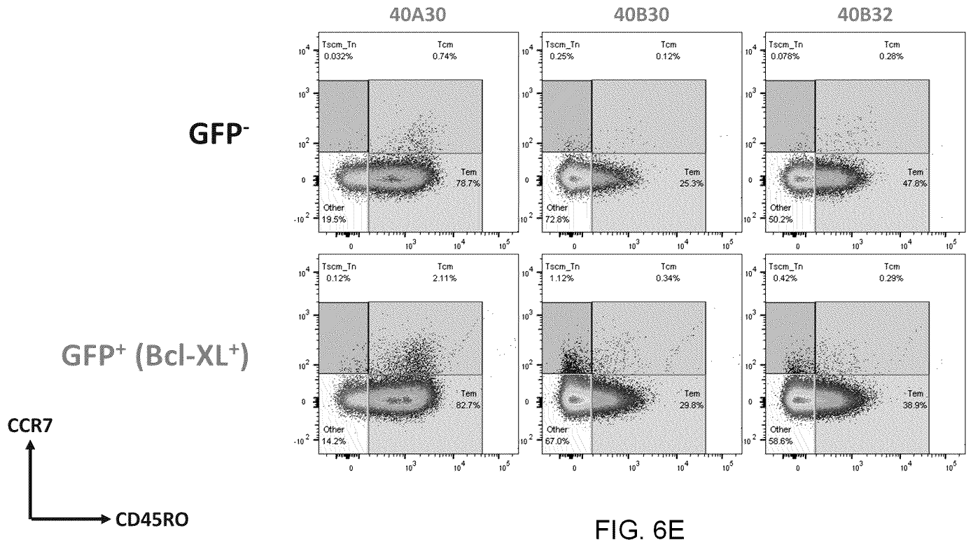

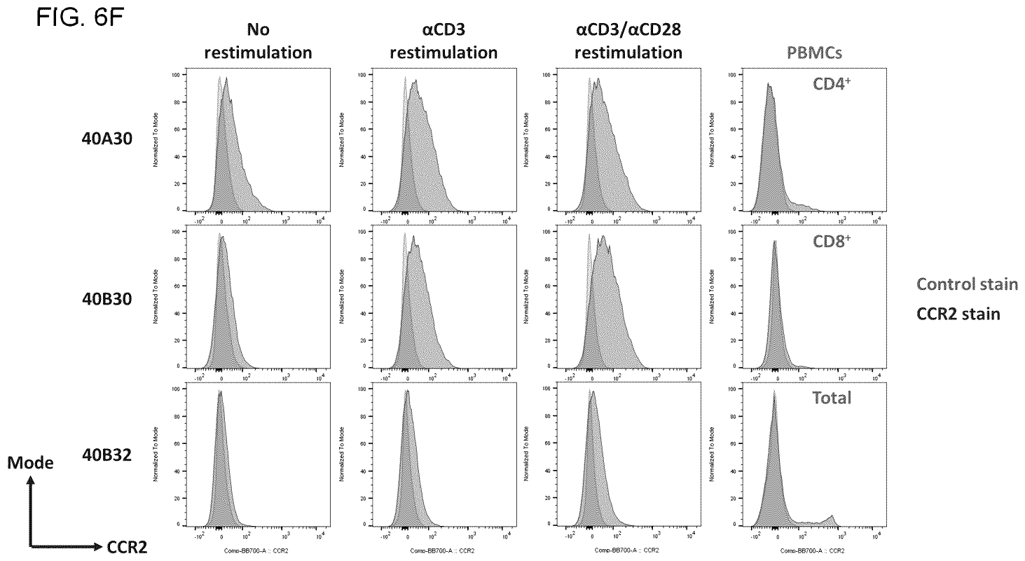

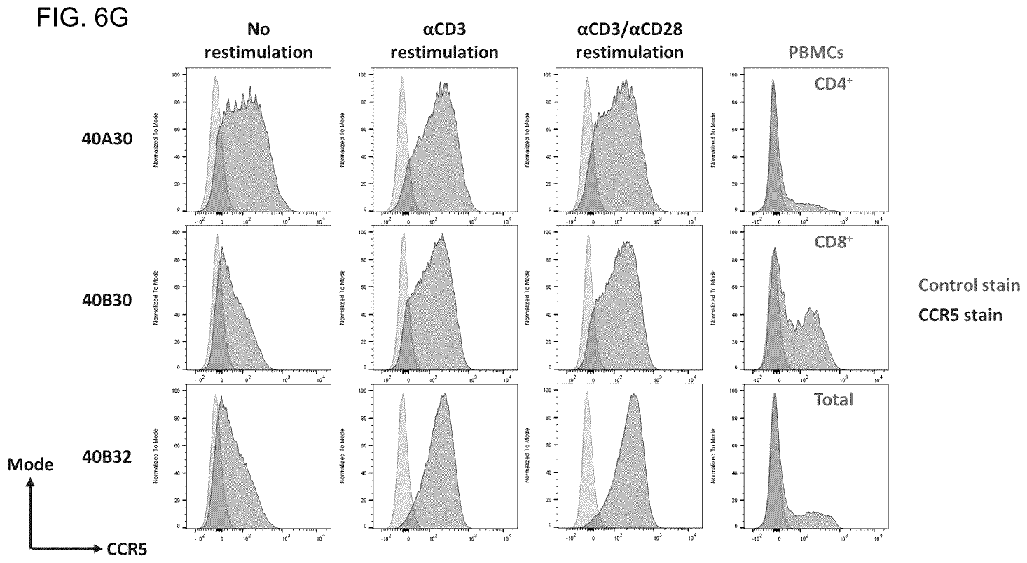

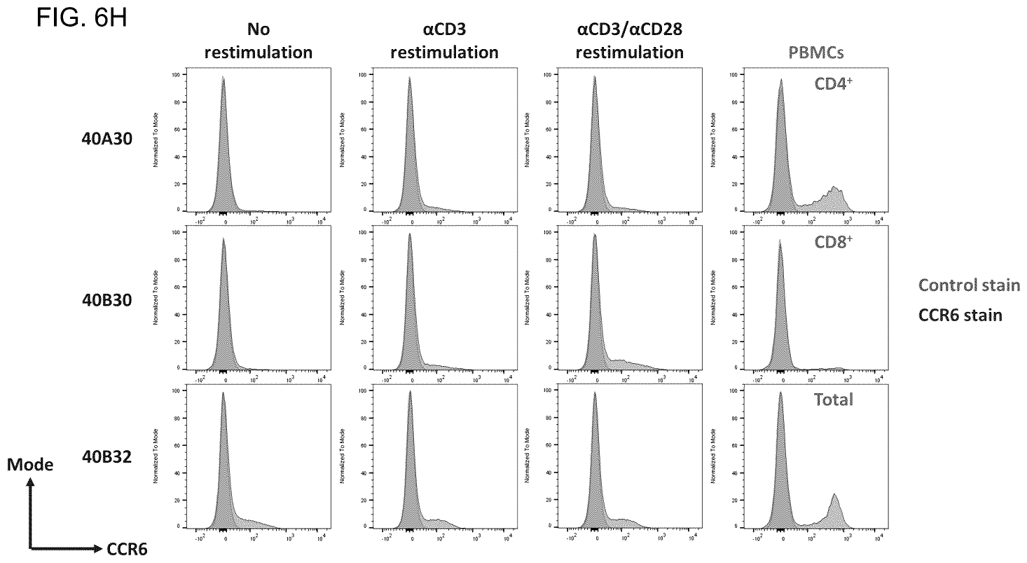

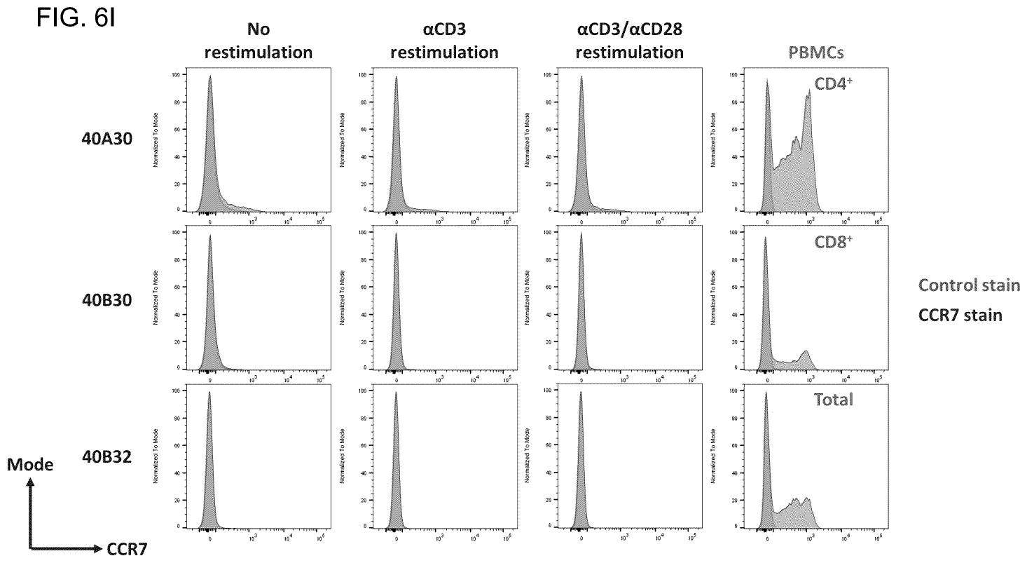

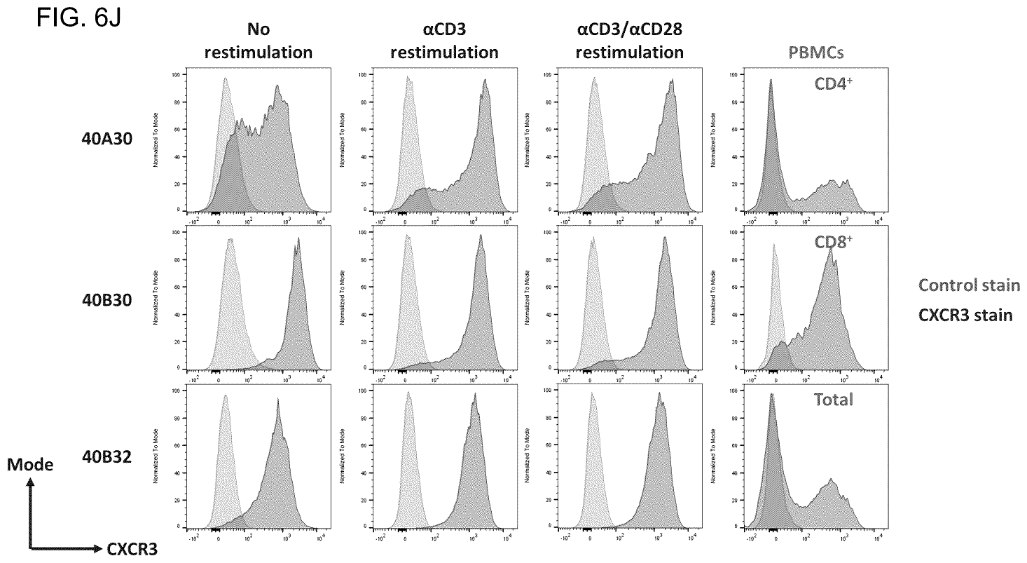

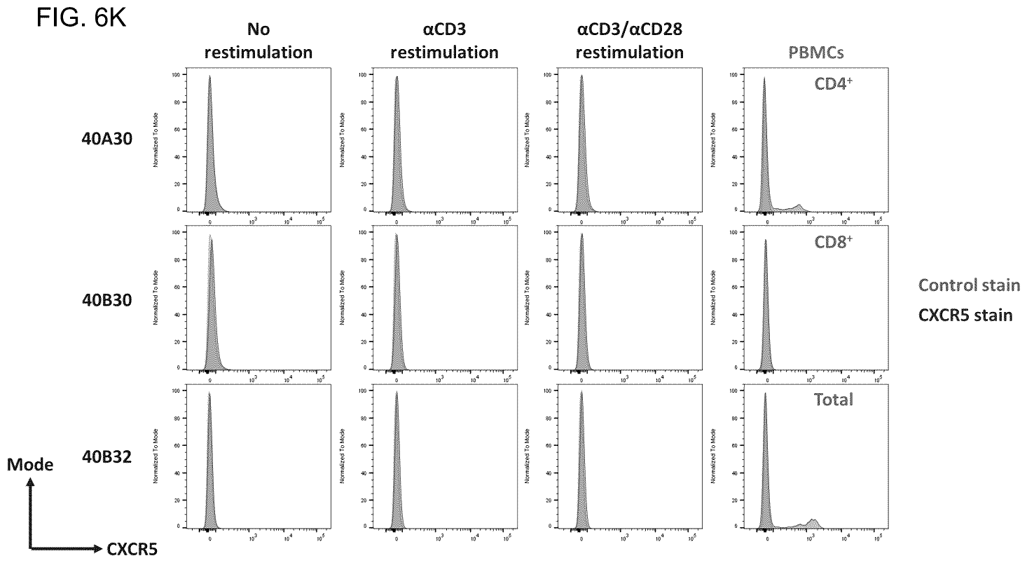

It was also tested whether T_{REX}+Bcl-xL cells and PTEN-deficient T_{REX}+Bcl-xL cells maintain phenotypes similar to normal T cells after modification and extended in vitro culture or whether these conditions drive T_{REX}+Bcl-xL cells to an exhausted phenotype (FIG. 6). All three T_{REX}+Bcl-xL lines maintained expression of cell surface CD3 and CD8 (FIGS. 6A and 6B). Furthermore, they expressed variable levels of activation markers such as PD1 and TIGIT (FIG. 6C) and maintained expression of CD28 in a donor-dependent manner (FIG. 6D). Finally, these T_{REX}+Bcl-xL lines exhibited differentiation phenotypes defined by surface expression of CD45RO and CCR7 that tracked in a donor-dependent manner (FIG. 6E). These data suggest that despite substantial proliferation and an extended duration of in vitro culture, T_{REX}+Bcl-xL cells resemble normal T cells and do not exhibit a surface phenotype associated with a dysfunctional state.

Chemokine receptors are important for trafficking of immune cells to sites of inflammation. Therefore, T_{REX}+Bcl-xL cells, PTEN-deficient T_{REX}+Bcl-xL cells, restimulated T_{REX}+Bcl-xL cells, and restimulated PTEN-deficient T_{REX}+Bcl-xL cells were analyzed for expression of the chemokine receptors CCR2, CCR5, CCR6, CCR7, CXCR3, and CXCR5 using flow cytometry (FIG. 6F-K). T_{REX}+Bcl-xL lines and PTEN-deficient T_{REX}+Bcl-xL lines demonstrated expression of CCR2 (FIG. 6F), CCR5 (FIG. 6G), and CXCR3 (FIG. 6J). Expression of CCR6 (FIG. 6H) was heterogenous, while expression of CCR7 (FIG. 6I) and CXCR5 (FIG. 6K) was low to absent. Therefore, T_{REX}+Bcl-xL cells and PTEN-deficient T_{REX}+Bcl-xL cells maintain expression of key chemokine receptors that will enable them to traffic to sites of inflammation.

### Example 4: T_{REX}+Bcl-xL Cells are Cytotoxic

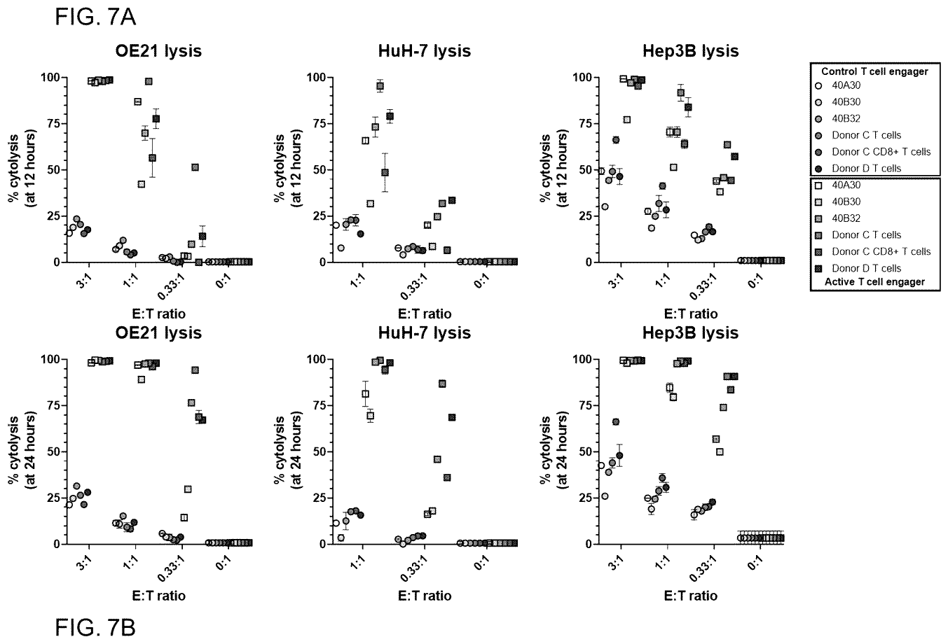

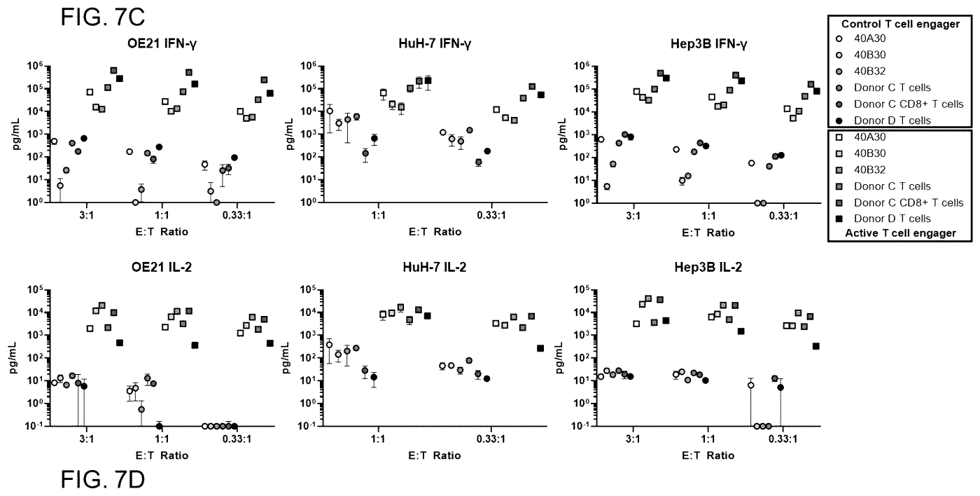

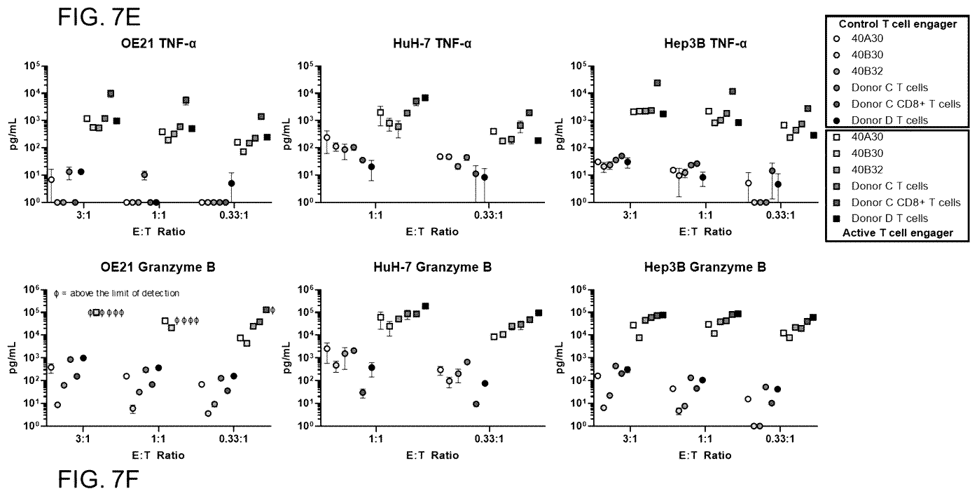

Having established that T_{REX}+Bcl-xL lines resemble normal primary human T cells, it was determined whether T_{REX}+Bcl-xL cells maintain potent cytotoxic function after long-term culture and expansion. A T cell engager was used in the presence of target tumor cells and the impedance-based xCELLigence platform to quantify T_{REX}+Bcl-xL cell cytotoxic function (FIG. 7). Day 80 T_{REX}+Bcl-xL lines demonstrated a comparable ability to lyse target tumor cells in the presence of the T cell engager as unmodified primary total T cells and unmodified primary CD8⁺ T cells (FIGS. 7A and 7B). Supernatants from these co-cultures were collected 72 hours after the addition of effector cells and the active T cell engager or control T cell engager molecule and analyzed for the presence of interferon γ (IFN-γ), IL-2, tumor necrosis factor α (TNF-α), and granzyme B (FIG. 7C-7F). T_{REX}+Bcl-xL lines produced lower levels of these cytokines relative to unmodified primary T cells despite a similar capacity to lyse target cells in an antigen-dependent manner. These data indicate that even after 80 days in culture and substantial expansion, T_{REX}+Bcl-xL cells are not functionally exhausted and maintain their cytotoxic potential.

### Example 5: T_{REX}+Bcl-xL Cells can Produce Functional CAR-T_{REX} Cells

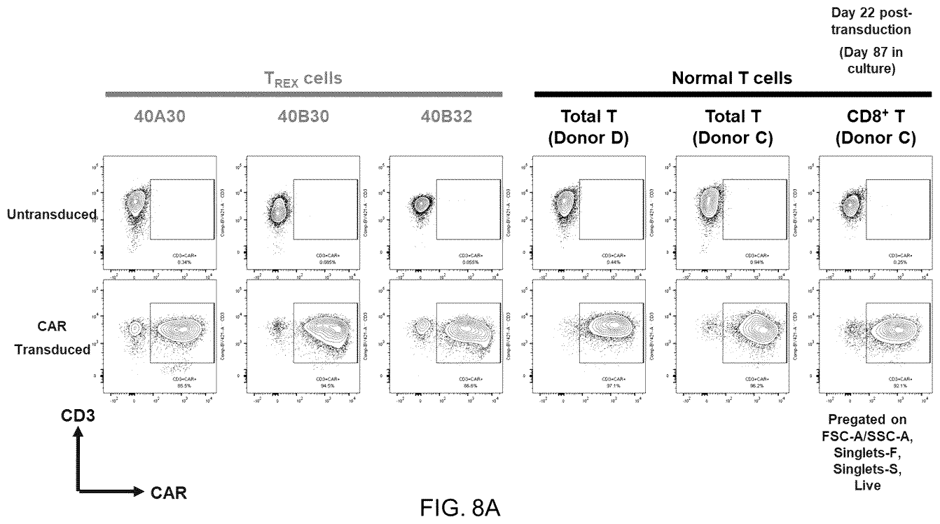

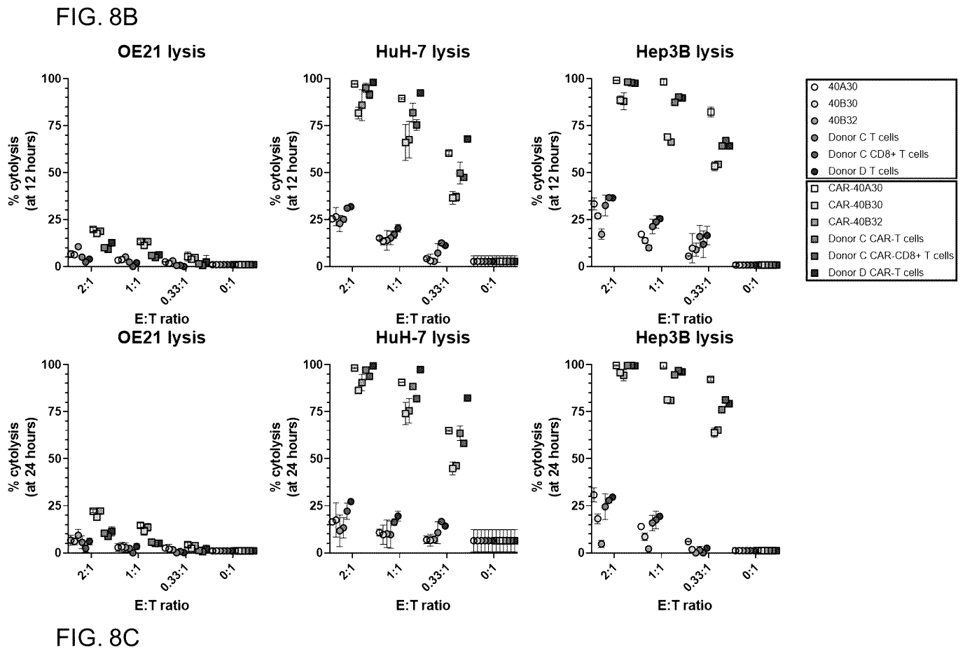

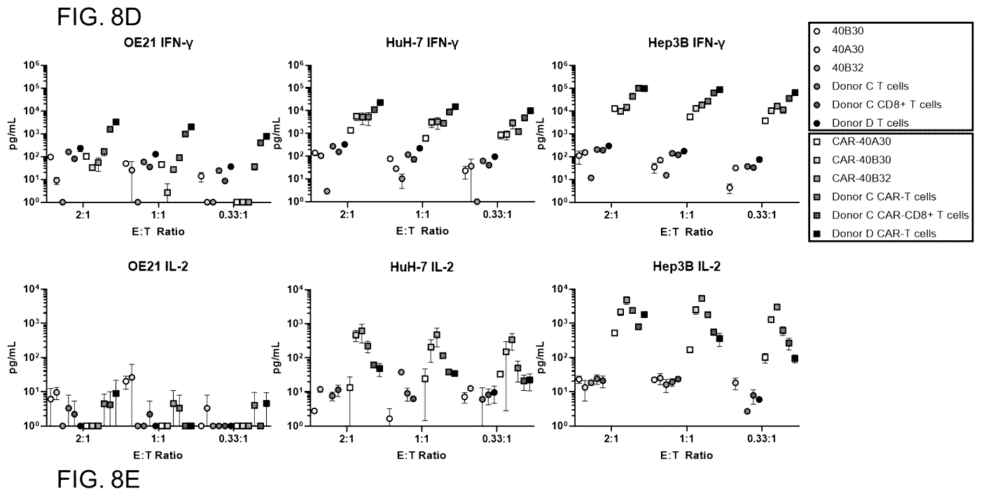

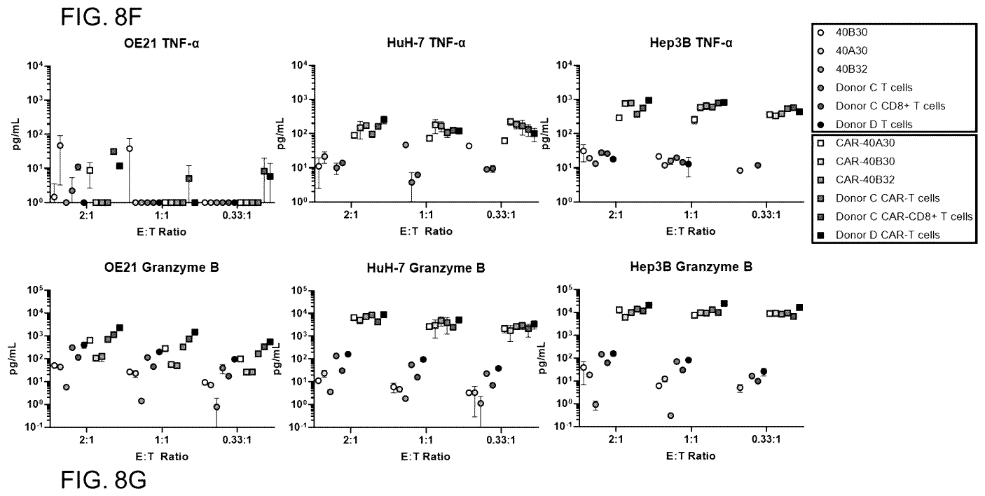

In order to develop T_{REX}+Bcl-xL cells into a potential cellular therapy these cells must be capable of expressing a targeting molecule such as a chimeric antigen receptor (CAR) to direct their cytotoxic function. The three T_{REX}+Bcl-xL cell lines generated as described above were transduced with a lentivirus encoding a CAR that recognizes glypican 3 (GPC3). Surface expression of the GPC3 CAR was subsequently measured using flow cytometry (FIG. 8A). Each T_{REX}+Bcl-xL line was found to successfully express the GPC3 CAR at levels similar to normal primary total T cells and normal primary CD8⁺ T cells (FIG. 8A).

CAR-directed cytotoxic function of T_{REX}+Bcl-xL cells was assessed by performing impedance-based xCELLigence assays using target tumor cells with varying degrees of antigen expression: OE21 (antigen-negative), HuH-7 (antigen-intermediate), and Hep3B (antigen-high) (FIGS. 8B and 8C). T_{REX}+Bcl-xL cells rapidly lysed target tumor cells in a CAR- and antigen-specific manner at levels similar to normal CAR-T cells and normal CAR-CD8⁺ T cells (FIGS. 8B and 8C). Supernatants were harvested from these co-cultures 72 hours after T cell addition and subsequently analyzed for secretion of IFN-γ, IL-2, TNF-α, and granzyme B (FIG. 8D-8G). In agreement with their potent cytotoxic function, CAR-T_{REX}+Bcl-xL cells demonstrated a comparable ability to secrete effector cytokines as normal CAR-T cells and normal CAR-CD8⁺ T cells (FIG. 8D-8G). However, in general, IFN-γ and TNF-α levels were found to be lower in CAR-T_{REX}+Bcl-xL cells.

These data confirm that T_{REX}+Bcl-xL and PTEN-deficient T_{REX}+Bcl-xL cells are capable of expressing a CAR and carrying out CAR-directed cytotoxic function in an antigen-dependent manner even after significant in vitro expansion.

### Example 6: T_{REX} Cells Traffic to Similar Locations as Primary CD8⁺ T Cells and are Responsive to IL-2 In Vivo

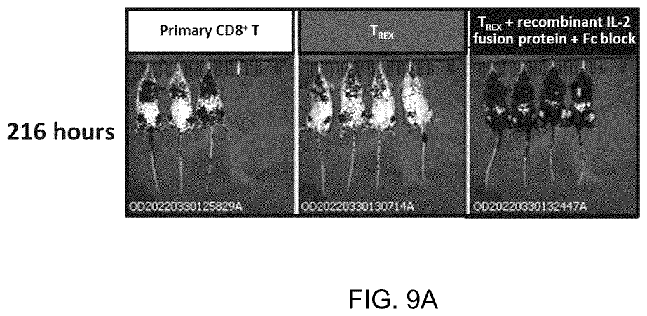

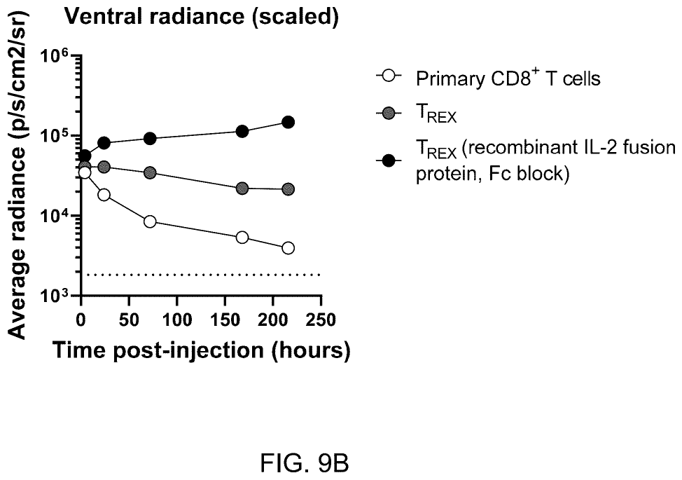

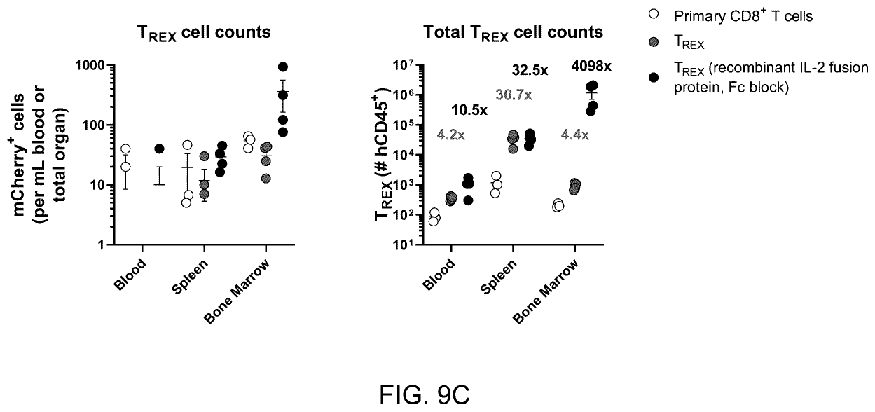

Cytokine cues can be used to modulate activity and expansion of human and murine T cells (Zhang et al., Science Translational Medicine, 22 Dec. 2021, Vol 13, Issue 625; Aspuria et al., Science Translational Medicine, 22 Dec. 2021, Vol 13, Issue 625) therefore T_{REX} cells were assessed for their capacity to respond to different human cytokines in vivo. Briefly, primary human CD8⁺ T cells or 278-day old T_{REX} cells were labeled with a luciferase reporter and 3E6 luciferase-expressing cells were infused into NSG mice with or without supplementation with a recombinant human IL-2 fusion protein. Mice were imaged using an IVIS Optical Imaging system to detect luciferase-expressing T cells (FIGS. 9A and 9B). As shown in FIG. 9A, imaging at 216 hours indicated similar localization of primary human CD8⁺ T cells and T_{REX} cells in mice. Further, mice supplemented with a recombinant human IL-2 fusion protein demonstrated enhanced proliferation of T_{REX} cells (FIG. 9A, right). Ventral radiance was graphed over time (FIG. 9B) and similarly demonstrates the ability of T_{REX} cells to respond to exogenously supplemented IL-2 in vivo. Mice were sacrificed 10 days after adoptive cell transfer and their blood, spleens, and bone marrow were assessed for the presence of primary CD8⁺ T cells or T_{REX} cells (FIG. 9C). While T_{REX} cells were found in similar organs as primary CD8⁺ T cells (FIG. 9C), they demonstrated slower decay kinetics and administration of a recombinant human IL-2 fusion protein could further enhance T_{REX} cell numbers in the blood and bone marrow of treated mice. These data suggest that T_{REX} cells home to similar sites as primary CD8⁺ T cells and maintain responsiveness to exogenous cytokine cues.

### Example 7: CAR-T_{REX} Cells Respond to IL-2 and IL-15 In Vivo

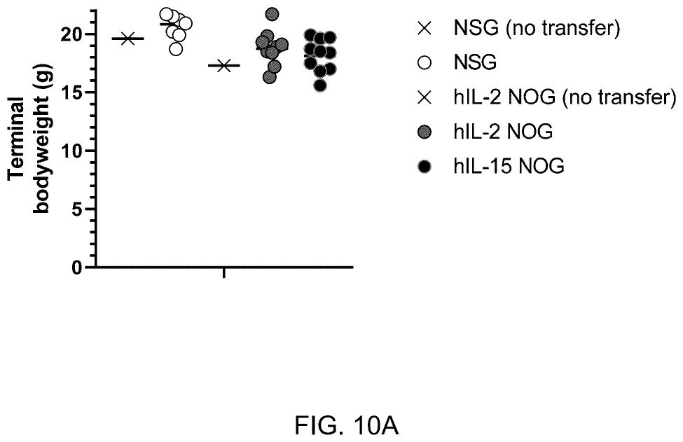

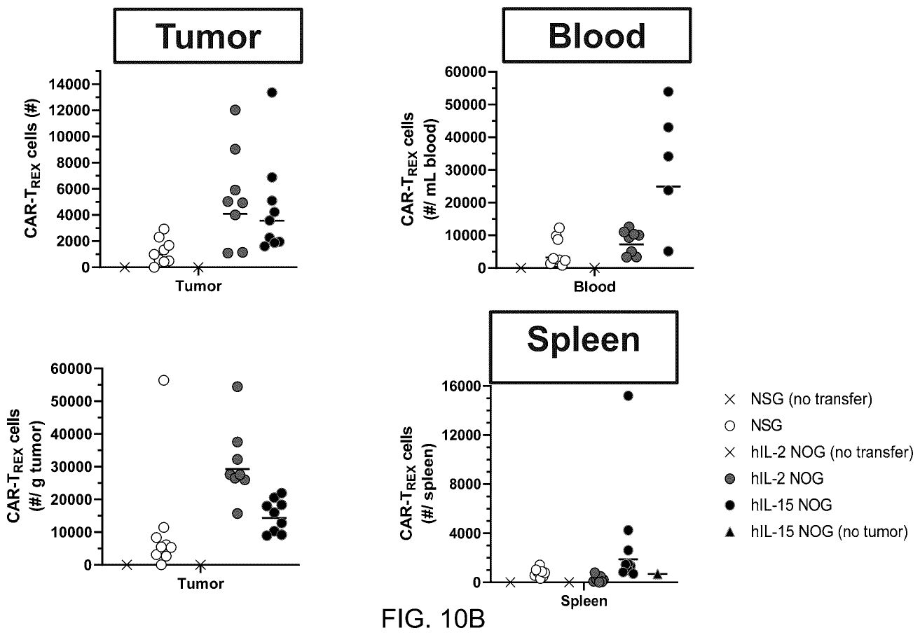

GPC3 targeting CAR-T_{REX}+Bcl-xL cells were assessed for their capacity to respond to different human cytokines in vivo. NSG, hIL-2 NOG, or hIL-15 NOG mice were inoculated with GPC3 expressing Hep3B tumor cells. Once tumors were established, mice were left untreated or were treated with 10E6 GPC3 targeting CAR-T_{REX}+Bcl-xL cells. CAR-T_{REX}+Bcl-xL cells were 121 days at the time of infusion. Mice were sacrificed 8 days after CAR-T_{REX}+Bcl-xL cell infusion and bodyweights were measured (FIG. 10A) with no discernable differences observed indicating lack of toxicity. Tumors, blood, and spleens were harvested and analyzed for the presence of CAR-T_{REX}+Bcl-xL cells (FIG. 10B). CAR-T_{REX}+Bcl-XL cell numbers were enhanced in tumor-bearing hIL-2 NOG and hIL-15 NOG mice indicating that CAR-T_{REX}+Bcl-xL cells are capable of responding to exogenous cytokine cues in vivo. Expansion profiles were specific to the particular cytokine support that was provided (FIG. 10B).

### Example 8: CAR-T_{REX} Cells Target Solid Tumors In Vivo

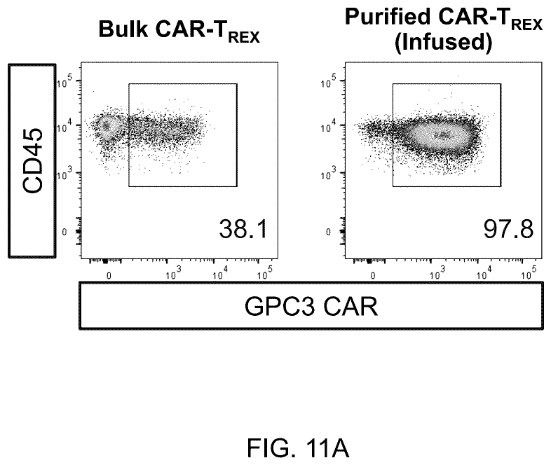

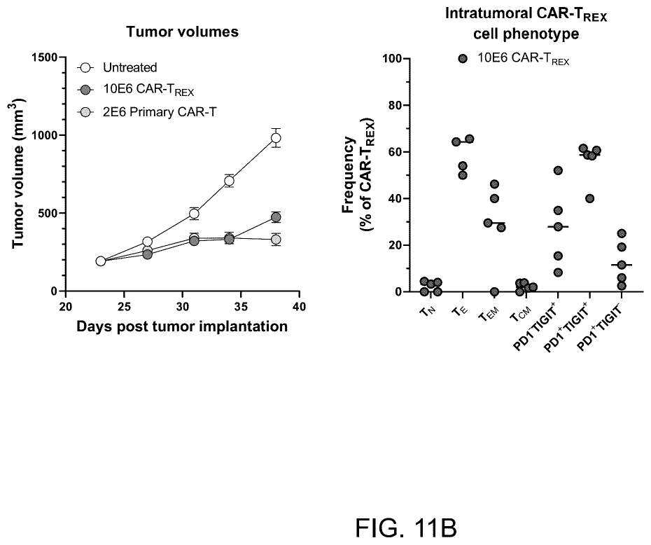

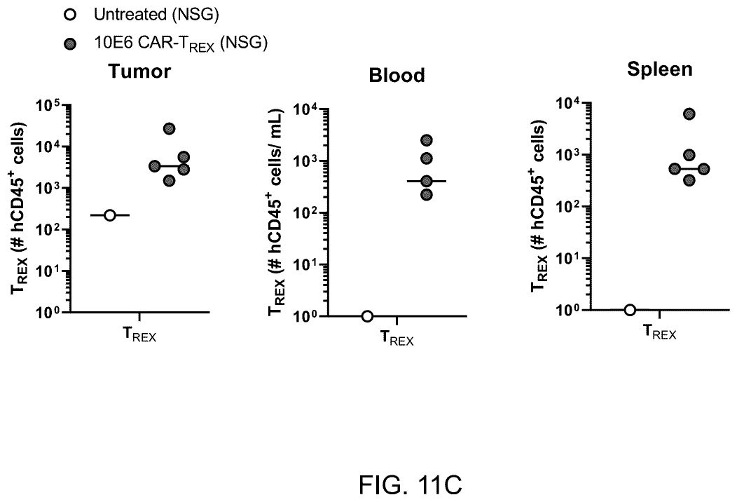

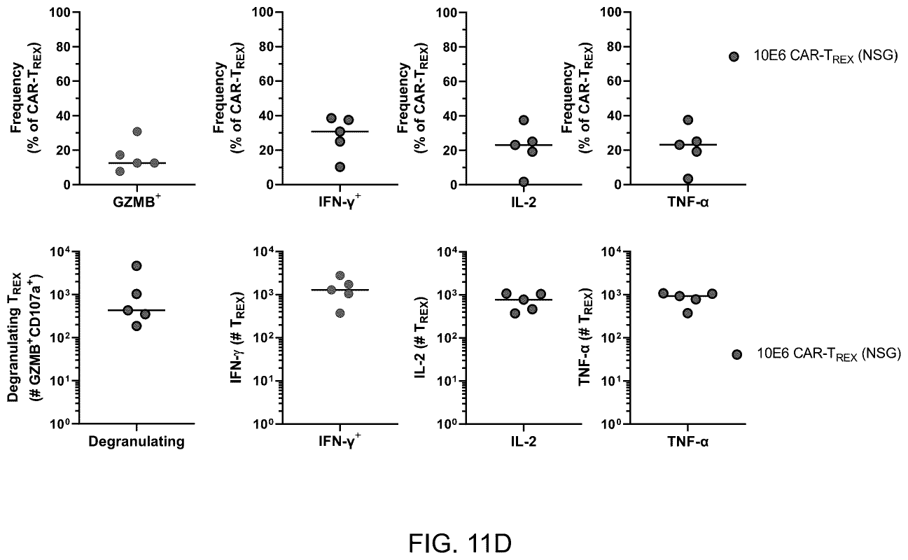

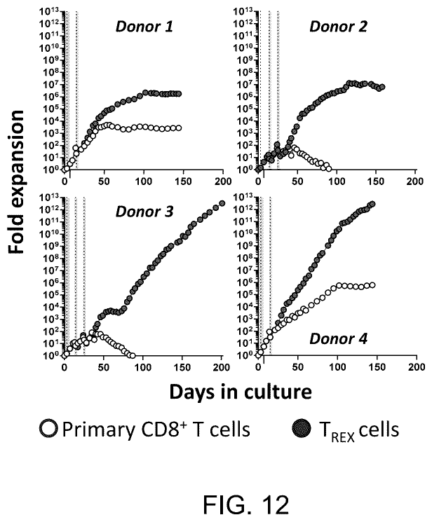

The capacity of GPC3 targeting CAR-T_{REX} cells to control solid tumors was determined. Hep3B tumors were established in NSG mice and then 92 day old purified CAR-T_{REX} cells (FIG. 11A) were infused into mice. 10E6 CAR-T_{REX} cells or 2E6 CAR-T cells were infused and tumor volumes were then measured and graphed over time (FIG. 11B, left). CAR-T_{REX} cells exhibited tumor growth inhibition and control of Hep3B tumors. 18 days post-CAR-T_{REX} cell transfer, mice were sacrificed and tumors, blood, and spleen were harvested for further analysis (FIGS. 11B, 11C, and 11D). Intratumor CAR-T_{REX} cell phenotypes were examined (FIG. 11B), and CAR-T_{REX} cell numbers were determined in these different tissues (FIG. 11C). CAR-T_{REX} cells were found in highest numbers in the tumors of mice and these cells demonstrated an activated phenotype where they were actively secreting effector cytokines and degranulating (FIG. 11D). T_{REX} cells additionally demonstrated an enhanced capacity to proliferate in vitro and can be expanded millions of fold and maintained in culture for more than 100 days without additional stimulation through their TCRs (FIG. 12). Furthermore, when the aforementioned target genes were simultaneously edited in healthy donor CD8⁺ T cells using CRISPR/Cas9, these edits reproducibly conferred a REX phenotype across different donors (FIG. 12).

### Example 9: Additional Genetic Edits for Clinical Profile

While the REX edits confer enhanced proliferation of the T_{REX} cell product, additional editing at the B2M and CD38 loci was performed to delay rejection of the T_{REX} cell product by a patient's immune system. B2M is a protein of 119 amino acids that is encoded by a gene on chromosome 15 in humans. It is also a component of major histocompatibility class (MHC) I molecules and also associates with non-classical, MHC I like molecules such as CD1, MR1, the neonatal Fc receptor, and Qa-1. Though it is located outside of the MHC locus, B2M is required for the successful expression of classical and non-classical MHC I molecules on the surfaces of nucleated cells. By eliminating B2M expression in T_{REX} cells using CRISPR/Cas9, the cells will be shielded from patient CD8+ T cells. Additionally, ablation of B2M expression and consequently MHC-I expression by the T_{REX} cell product will sensitize it to rejection by patient NK cells. Knockout of B2M was carried out simultaneously with knock in of a targeting a CAR (e.g. GPC3, HER2, BCMA) in the T_{REX} cell product.

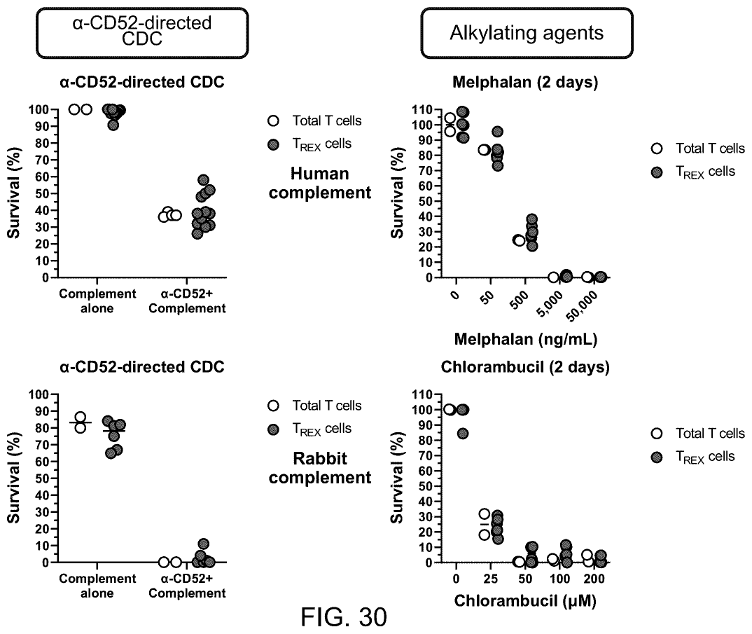

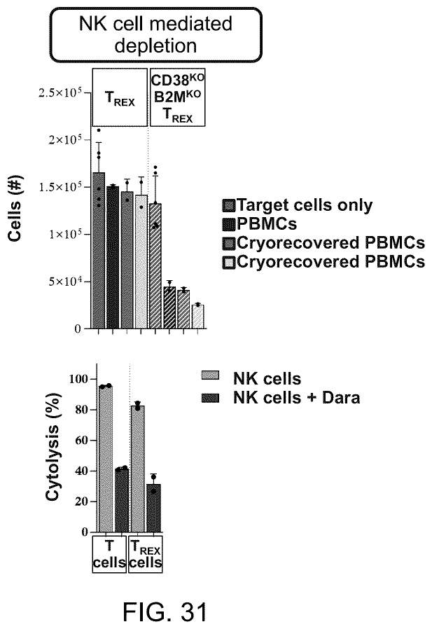

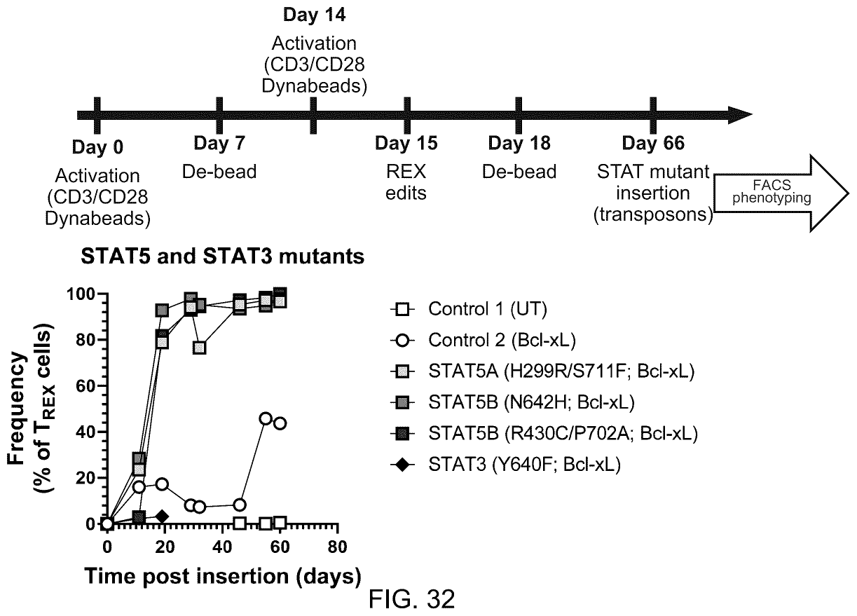

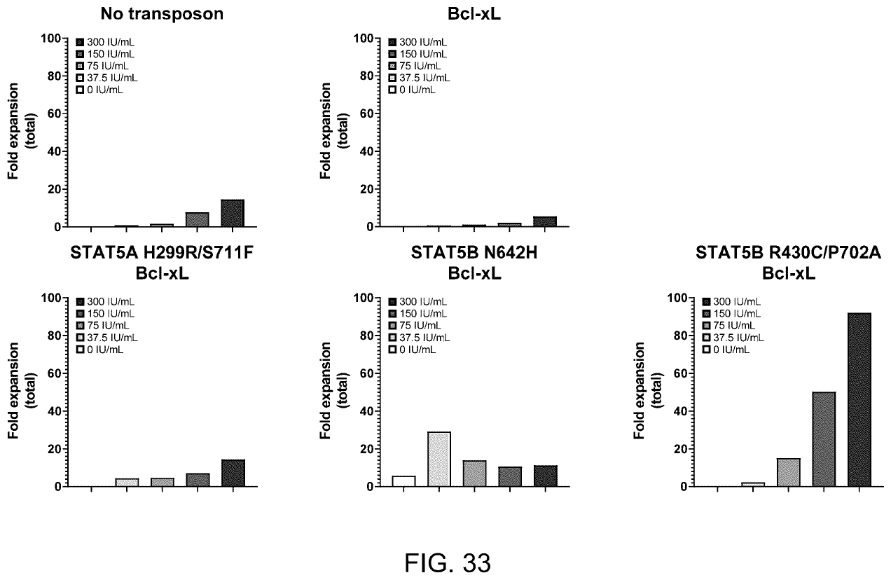

NK cells express high levels of CD38 and are depleted in certain cancer patients, e.g., multiple myeloma patients, receiving anti-CD38 monoclonal antibodies such as daratumumab and isatuximab. In order to prolong the persistence of this allogeneic cell population in patients, CD38 was knocked out of T_{REX} cells using CRISPR/Cas9 and daratumumab or isatuximab can be co-administered with T_{REX} cells (see FIGS. 30 and 31).

As an allogeneic CD8⁺ T cell population, T_{REX} cells are expected to be capable of targeting the HLA-mismatched patient's healthy cells through their TCRs, resulting in GvHD. In order to prevent the development of this pathology, the T_{REX} cell population was edited at the T Cell Receptor Alpha Constant (TRAC) locus, which encodes the TCR a chain. CRISPR/Cas9 editing of TRAC leads to loss of expression of the TCR a chain, which in turn prevents surface expression of the TCR by T_{REX} cells.

TABLE 4
Summary of cell edits

| Gene | Type of edit | Purpose |
|---|---|---|
| CDKN2A | CRISPR/Cas9 deletion | Resistance to replicative senescence |
| CDKN2B | CRISPR/Cas9 deletion | Resistance to replicative senescence |
| MTAP | CRISPR/Cas9 deletion | Resistance to replicative senescence |
| B2M | CRISPR/Cas9 deletion | Limit HvG |
| TRAC | CRISPR/Cas9 deletion | Avoid GvHD |
| CD38 | CRISPR/Cas9 deletion | Resistance to daratumumab |

### Example 10: Anti BCMA-T_{REX} Allogeneic Cell Therapy

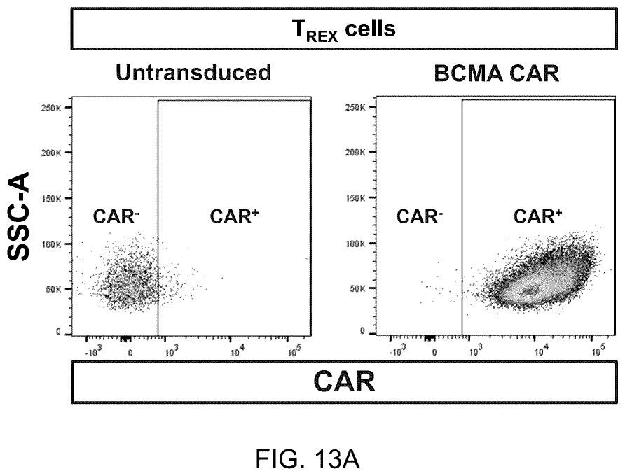

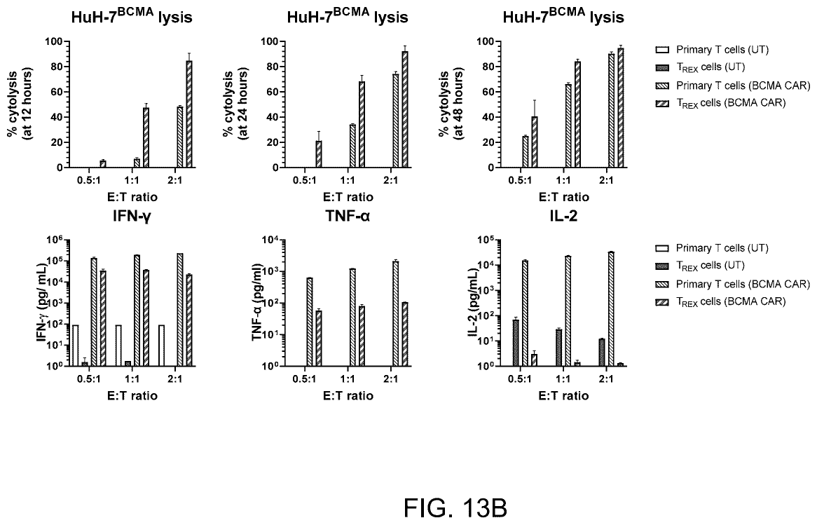

A BCMA targeting CAR was expressed in T_{REX} cells (i.e., cells lacking CDKN2A/CDKN2B/MTAP) (FIG. 13A). The genome of the anti-BCMA-T_{REX} cells was further edited to ablate expression of Human Leukocyte Antigen (HLA) class I and the αβ T cell receptor (TCR) by inactivation of the B2M and TRAC genes, respectively, to minimize host-versus-graft (HvG) and graft-versus-host (GvH) allogeneic responses, respectively. Additionally, the CD38 gene was inactivated in anti-BCMA-T_{REX} cells using CRISPR/Cas9 to render the cells resistant to anti-CD38 depleting monoclonal antibodies. The inactivation of these three genes enhances the total in vitro expansion potential of peripheral blood CD8⁺ T cells such that downstream cell population numbers far exceed those achievable with unedited peripheral blood CD8⁺ T cells. The cells retain hallmark proliferative characteristics of primary T cells (dependence on both anti-CD3 stimulation prior to TRAC inactivation and IL-2 for expansion/survival) but with greater potential for expansion. Anti-BCMA-T_{REX} cells maintain cytotoxic function but display reduced cytokine release compared to conventional CAR-T cell preparations composed of mixed CD4⁺ and CD8⁺ T cell populations.

Assessment of anti-BCMA-T_{REX} cells indicates that these cells are likely to control BCMA-expressing tumors similarly to primary anti-BCMA-CAR-T cells while exhibiting a potentially improved safety profile in the form of diminished cytokine release and potentially reduced risk of CRS (FIG. 13B). Briefly, anti-BCMA-T_{REX} cells and anti-BCMA-CAR-T cells were cultured with BCMA-expressing tumor cells. Tumor cell lysis was measured at varying effector:target cell ratios at different timepoints following initiation of co-culture (FIG. 13B, top row). Supernatants were collected 72 hours after start of co-culture and levels of IFN-γ, TNF-α, and IL-2 were determined (FIG. 13B, bottom row) by MSD.

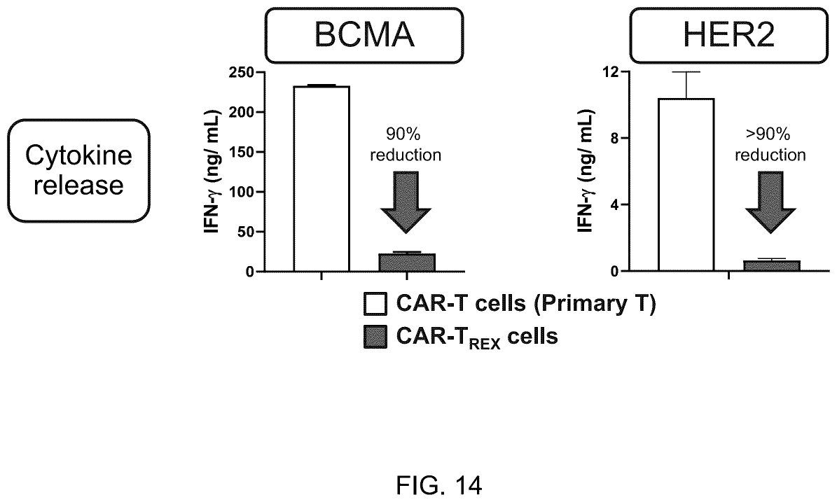

Anti-BCMA-T_{REX} cells (82 days in culture) or anti-BCMA-CAR-T cells were cultured with BCMA-expressing tumor cells. Supernatants were collected 72 hours after initiation of co-culture and assessed for levels of IFN-γ using MSD kits (FIG. 14, left). Data demonstrate a 90% reduction in IFN-γ levels in co-cultures with anti-BCMA-T_{REX} cells (83 days in culture) than in co-cultures with anti-BCMA-CAR-T cells despite similar control of tumor cells. These data demonstrate that CAR-T_{REX} cells exhibit a cytokine secretion profile that may confer lower risk of CRS than CAR-T cells.

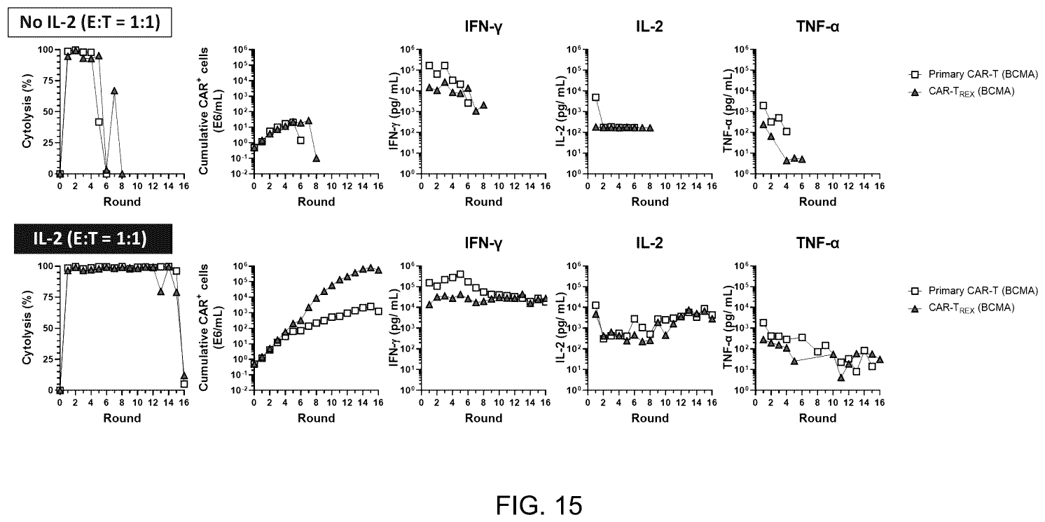

Anti-BCMA-T_{REX} cells (112 days in culture) were assessed for their ability to persist in a serial kill assay with or without IL-2 support. Briefly, anti-BCMA-T_{REX} cells or anti-BCMA-CAR-T cells were serially cultured with BCMA-expressing JJN3 cells at an effector:target cell ratio of 1:1. Tumor cell control (% cytolysis), effector cell numbers, and effector cytokine secretion was measured after each round of co-culture and graphed (FIG. 15). Anti-BCMA-T_{REX} cells persisted a comparable number of rounds in this serial kill assay as anti-BCMA-CAR-T cells and inclusion of IL-2 in the cell culture medium further increased the number of rounds for which anti-BCMA-T_{REX} cells and anti-BCMA-CAR-T cells could control tumor cell growth. Anti-BCMA-T_{REX} cells and anti-BCMA-CAR-T cells demonstrated enhanced proliferation in response to IL-2 and effector cytokine secretion was sustained for a longer duration in co-cultures in which IL-2 was included in the culture medium (FIG. 15 top versus bottom rows). These data indicate that anti-BCMA-T_{REX} cells demonstrate similar cytotoxicity to anti-BCMA-CAR-T cells in vitro and also exhibit a similar capacity to respond to exogenous IL-2. Further, anti-BCMA-T_{REX} cells secreted lower levels of effector cytokines following CAR-engagement than anti-BCMA-CAR-T cells despite comparable tumor control.

### Example 11: Anti HER2-T_{REX} Allogeneic Cell Therapy

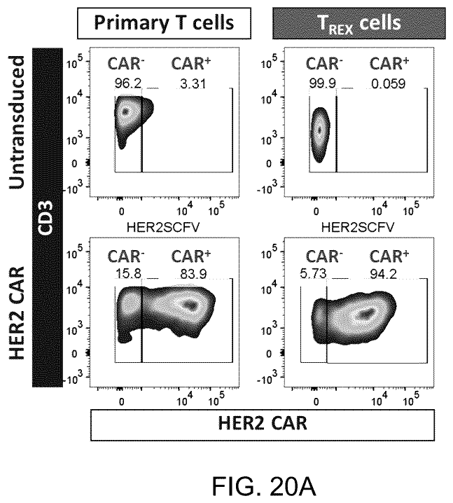

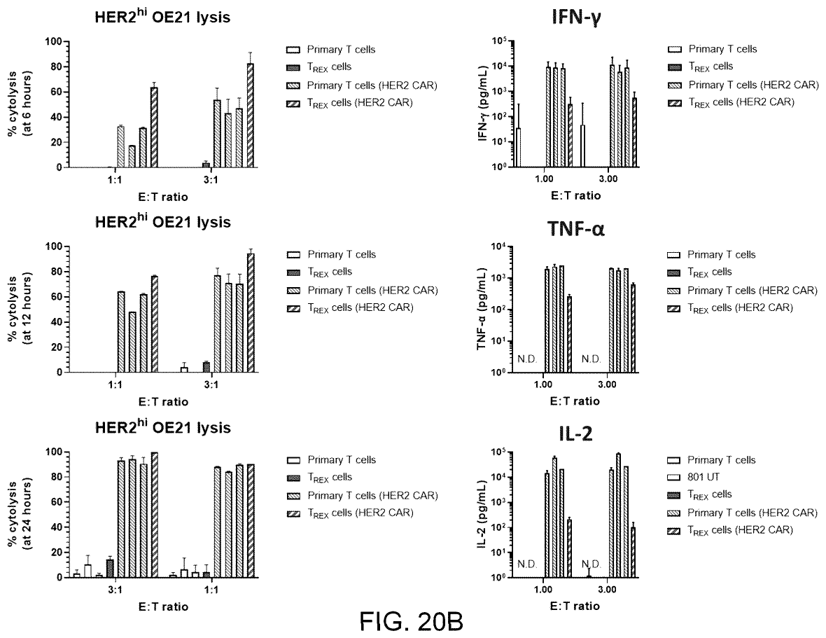

A HER2 targeting CAR was expressed in T_{REX} cells or Primary T cells (FIG. 20A) to generate CAR-T_{REX} cells and CAR-T cells. Anti-HER2-T_{REX} cells and anti-HER2-CAR-T cells were assessed for their ability to target HER2 overexpressing OE21 cells at varying effector:target cell ratios (FIG. 20B, left). Anti-HER-T_{REX} cells demonstrated comparable or improved control of HER2-expressing tumor cells relative to anti-HER2-CAR-T cells generated from three different Primary T cell donors. Supernatants were collected 72 hours after initiation of co-culture and subsequently examined for the presence of effector cytokines (FIG. 20B, right). As was previously observed, despite comparable or improved tumor cell control, anti-HER2-T_{REX} cells secreted lower levels of cytokines (IFN-γ, TNF-α, and IL-2) than anti-HER2-CAR-T cells, suggesting that CAR-T_{REX} cells may have a lower propensity to cause CRS in patients. Additionally, as shown above for anti-BCMA-T_{REX} cells, reduced secretion of IFN-γ was also observed in supernatants taken from co-cultures of HER2-expressing tumor cells and anti-HER2-T_{REX} cells as compared with supernatants from co-cultures with anti-HER2-CAR-T cells (FIG. 14, right). These data again demonstrate that CAR-T_{REX} cells exhibit a cytokine secretion profile that may confer lower risk of CRS than CAR-T cells.

### Example 12: T_{REX} Cell Phenotype can be Generated Using Different Combinations of Edits

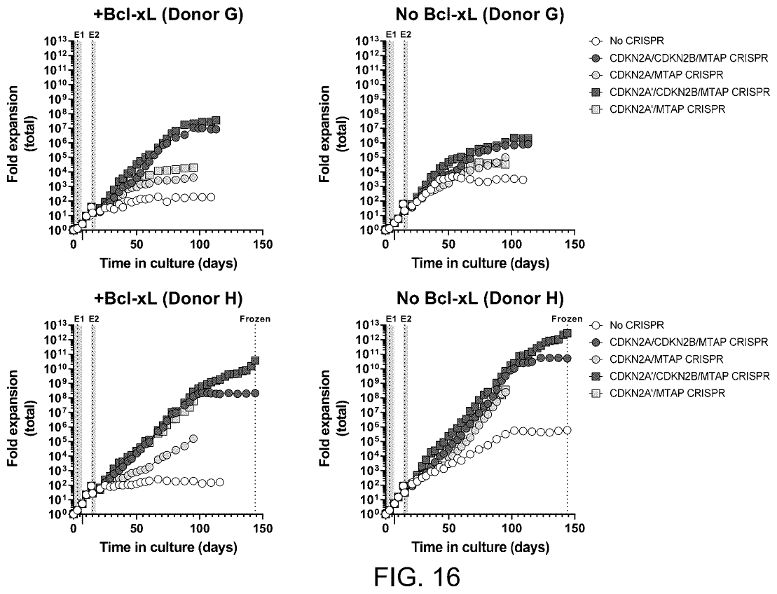

Requirements for overexpression of Bcl-xL and the various REX target genes to confer the REX phenotype were assessed in isolated CD8⁺ T cells from two donors (denoted as G and H). Briefly, CD8⁺ T cells were negatively selected and then activated with αCD3/αCD28 Dynabeads for 3 days. Bcl-xL was introduced into some cells while other cells were cultured and various combinations of the REX target genes were knocked out using CRISPR/Cas9 (FIG. 16). Cell expansion was monitored and graphed over time. (CDKN2A and CDKN2A′ reflect targeting of single versus multiple isoforms). Bcl-xL was found to be dispensable for the REX phenotype while the three REX target genes yielded a consistent phenotype across donors (FIG. 16, right).

### Example 13: T_{REX} Cells are Edited at the Targeted Loci

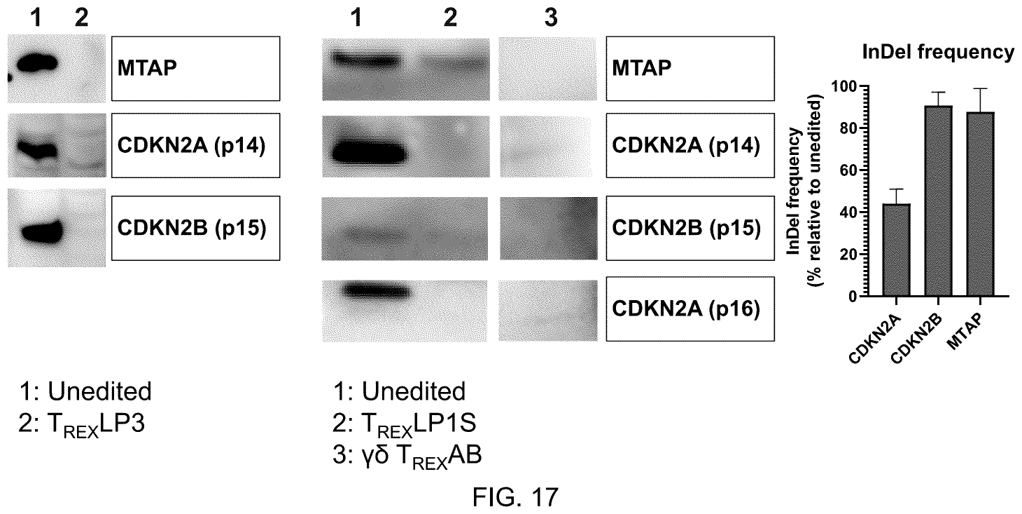

T_{REX} cells and γδ T_{REX} cells were examined for ablation of expression of the REX target genes by Western Blot analysis (FIG. 17, left and center panels). As expected these cells demonstrated loss of expression of MTAP, CDKN2A (p14), CDKN2A (p16), and CDKN2B (p15). In contrast, expression of these genes was maintained in donor-matched, unedited control cells. Further, Sanger Sequencing data indicate a high prevalence of InDels at these three loci in edited T_{REX} cells (FIG. 17, right).

### Example 14: T_{REX} Cells Demonstrate Enrichment in Cell Cycle-Associated Gene Signatures

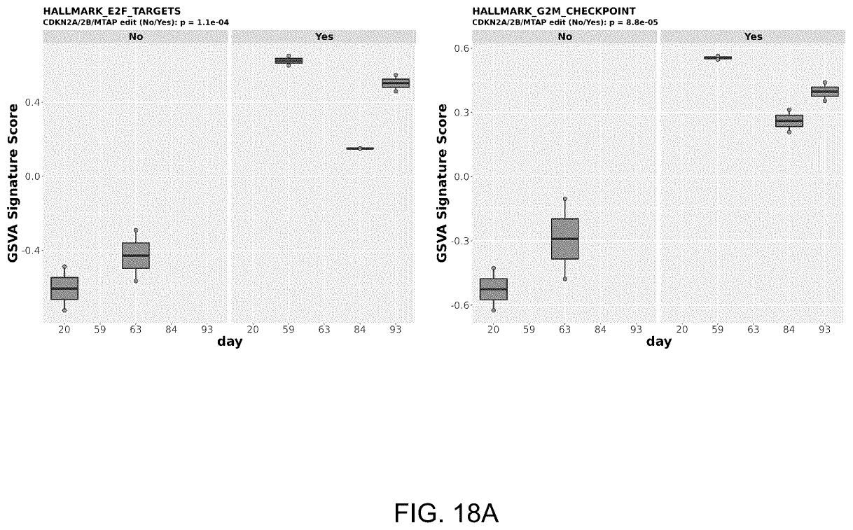

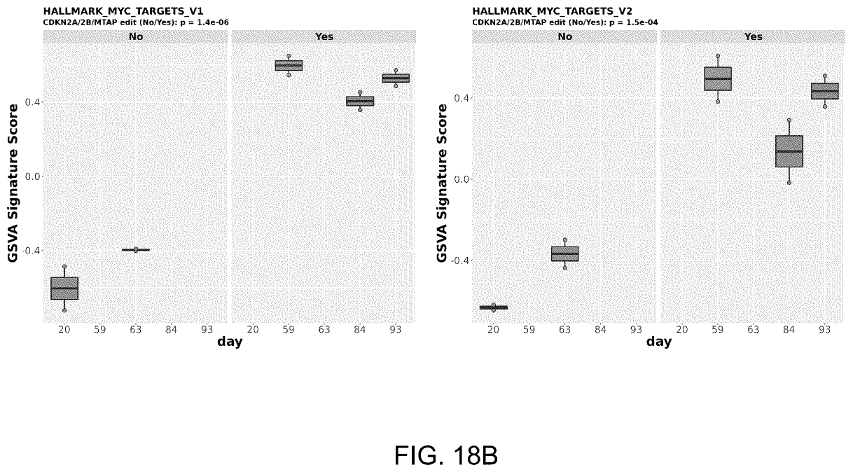

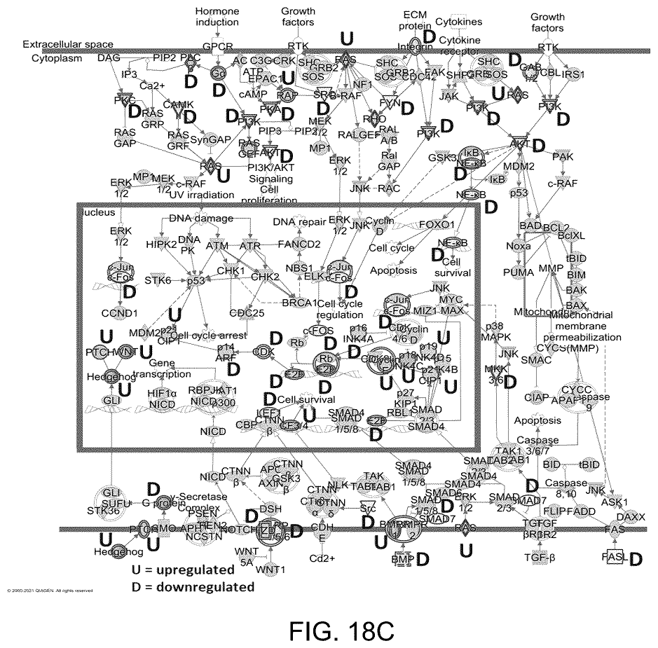

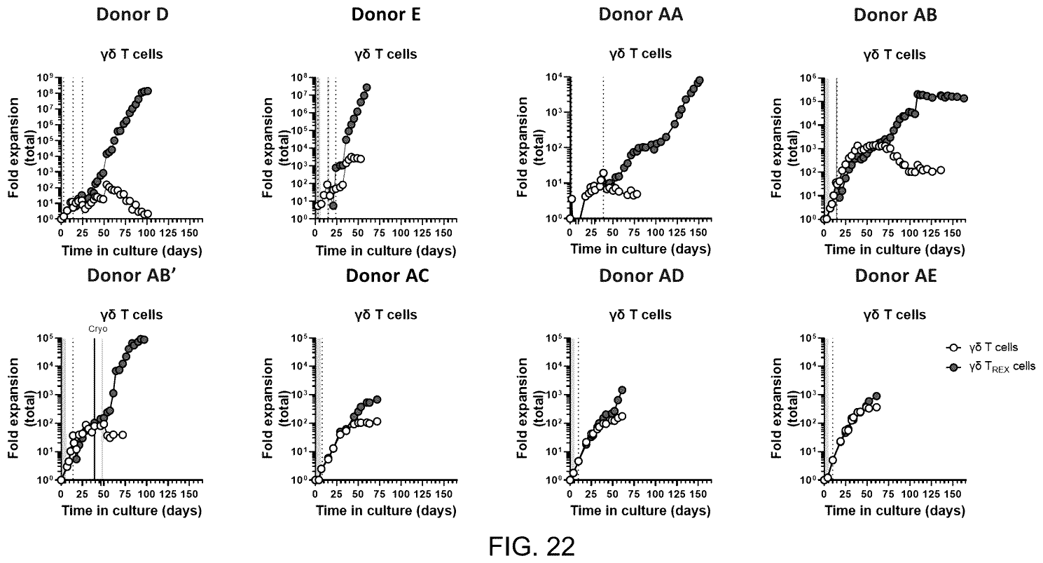

Bcl-xL overexpressing T_{REX} cells and donor-matched unedited control CD8⁺ T cells were cultured over time. RNAseq analysis was performed on cell pellets generated at various points and gene signatures were assessed in Bcl-XL T_{REX} cells and control cells; as expected, T_{REX} cells showed enrichment in gene signatures associated with cell cycle such as E2F target genes and G2M checkpoint target genes (FIG. 18A). Bcl-xL T_{REX} cells also showed higher levels of expression of MYC target genes (FIG. 18B) in agreement with the observed proliferation rates of these cells. Further, Bcl-XL T_{REX} cells showed modulation of expression of multiple cell-cycle-associated genes (FIG. 22C). These data confirm that the REX phenotype is associated with cell cycle progression and increased proliferation.

### Example 15: T_{REX} Cells are Dependent on IL-2 for Survival and Proliferation

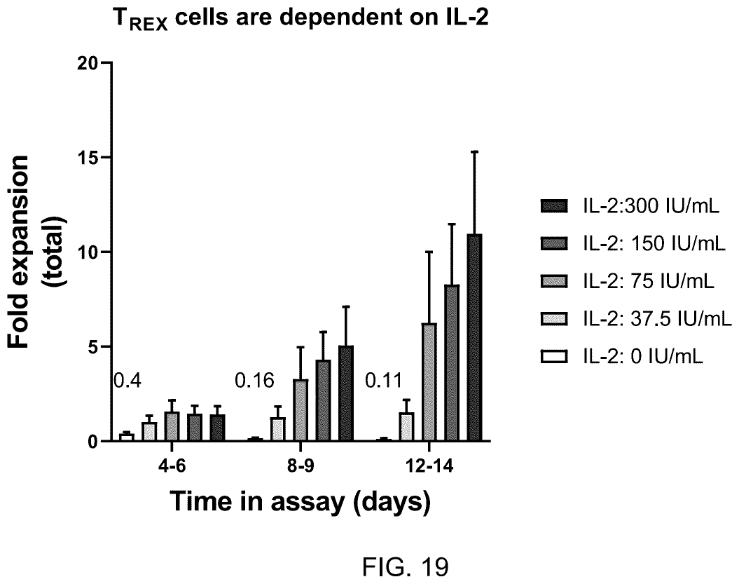

T_{REX} cells and were cultured with varying amounts of IL-2 for a period of 12-14 days. Cell expansion was tracked and graphed over this time (FIG. 19). As shown above for Bcl-XL T_{REX} cells (see, e.g., FIG. 5), T_{REX} cells are highly dependent on IL-2 for proliferation and survival in vitro. T_{REX} cells exhibited dose-dependent proliferation in response to IL-2; in the absence of IL-2, T_{REX} cells showed a rapid decline in survival with more than 60% of T_{REX} cells being eliminated within the first 4 days.

### Example 17: REX Edits Bolster the Proliferative Capacity of CD4⁺ T_{REX} Cells

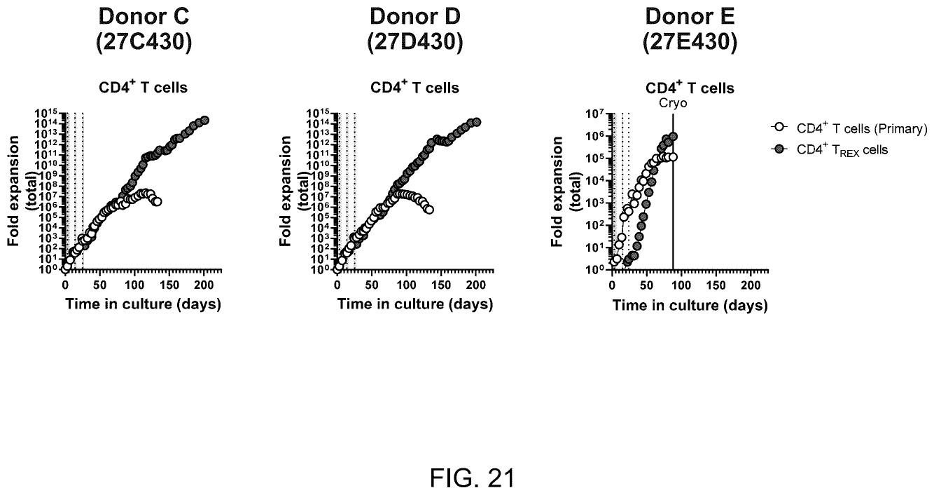

REX edits reproducibly confer an enhanced resistance to replicative senescence in CD8⁺ T cells. The capacity of ablation of expression of the REX target genes (CDKN2A, CDKN2B, and MTAP) to enhance CD4⁺ T cell resistance to replicative senescence was determined. CD4⁺ T cells were isolated from three healthy donors, stimulated using αCD3/αCD28 Dynabeads and then edited at these loci. Proliferation of CD4⁺ T_{REX} cells and donor-matched unedited CD4⁺ T cell controls was tracked over time and graphed. (FIG. 21). As was previously demonstrated with CD8⁺ T cells, targeting the REX genes in CD4⁺ T cells reproducibly bolstered the proliferative capacity of these cells and rendered them resistant to replicative senescence.

### Example 18: γδ T_{REX} Cells can be Generated Using REX Edits

γδ T cells are another cytotoxic subset of T cells. The ability of REX edits to confer a T_{REX} cell phenotype in γδ T cells was investigated using γδ cells from eight different donors (FIG. 22). γδ T cells were isolated and stimulated with αCD3/αCD28 Dynabeads or αCD3 antibody and subsequently edited at the REX loci using CRISPR/Cas9. γδ cell and γδ T_{REX} cell proliferation was monitored and graphed over time. REX edits reproducibly enhanced γδ cell resistance to replicative senescence and led to generation of a γδ T_{REX} cell phenotype.

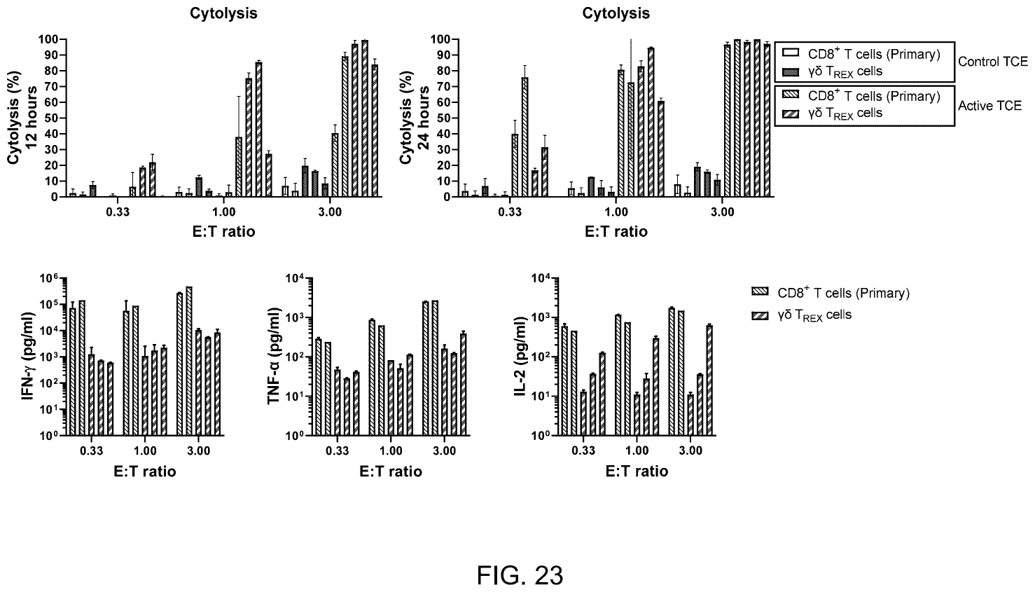

Having established that γδ T_{REX} cell lines demonstrate enhanced resistance to replicative senescence, it was determined whether γδ T_{REX} cells maintain potent cytotoxic function after long-term culture and expansion. A T cell engager was used in the presence of target tumor cells and the impedance-based xCELLigence platform to quantify γδ T_{REX} cell cytotoxic function (FIG. 23). Day 79 and Day 88 γδ T_{REX} cell lines demonstrated a comparable ability to lyse target tumor cells in the presence of the T cell engager as unmodified primary CD8⁺ T cells (FIG. 23, top). Supernatants from these co-cultures were collected 72 hours after the addition of effector cells and the active T cell engager or control T cell engager molecule and analyzed for the presence of IFN-γ, IL-2, and TNF-α (FIG. 23, bottom). γδ T_{REX} cell lines produced lower levels of these cytokines than unmodified primary CD8⁺ T cells despite a similar ability to lyse target cells in an antigen-dependent manner. These data indicate that even after 79 days in culture and substantial expansion, γδ T_{REX} cells are not functionally exhausted and maintain their cytotoxic potential.

γδ cells are typically comprised of multiple subsets including Vδ1, Vδ2, Vδ3, and Vδ5, among others (Lawand et al., Front. Immunol., 30 Jun. 2017). In humans the Vδ1 and Vδ2 make up the majority of γδ T cells with Vδ2 cells being found primarily in the blood and Vδ1 cells being found in tissues.

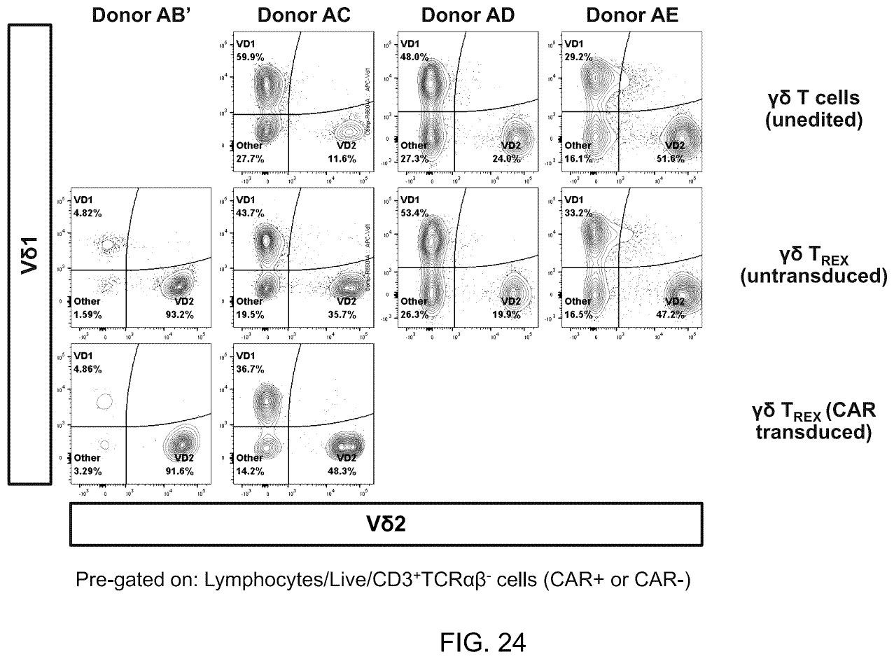

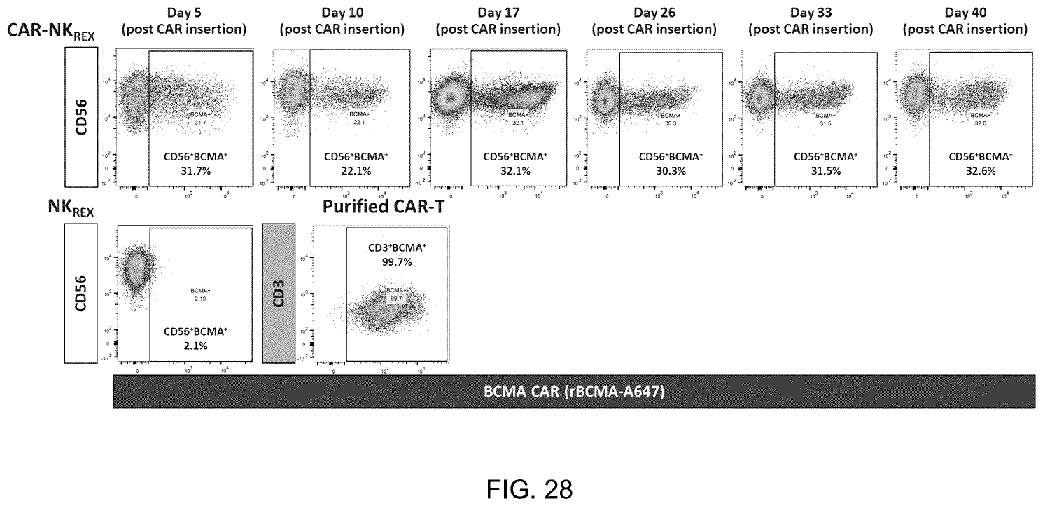

γδ T_{REX} cells, γδ CAR-T_{REX} cells, and donor-matched, unedited γδ cells were stained and analyzed for expression of Vδ1 and Vδ2 (FIG. 24). FACS analysis revealed that γδ T_{REX} cells were comprised of multiple γδ cell subtypes (Vδ1, Vδ2, and Vδ1⁻Vδ2⁻), indicating that REX edits can enhance resistance to replicative senescence for multiple γδ T cell subtypes. Further, diversity of γδ cell subtypes was maintained in γδ CAR-T_{REX} cells (FIG. 28, bottom).

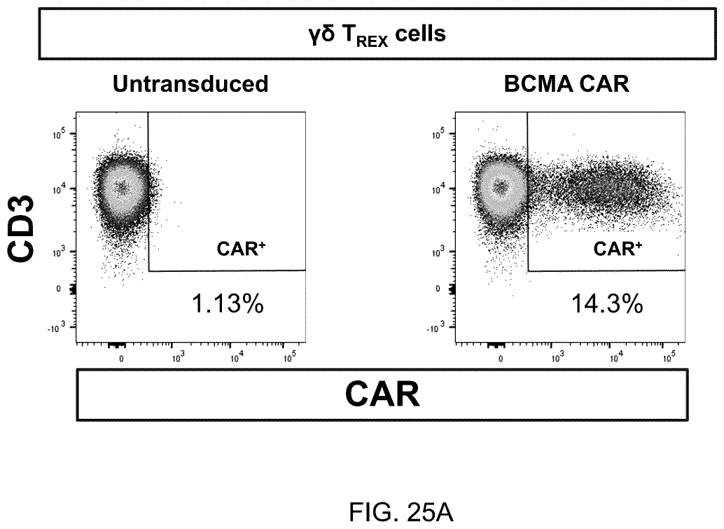

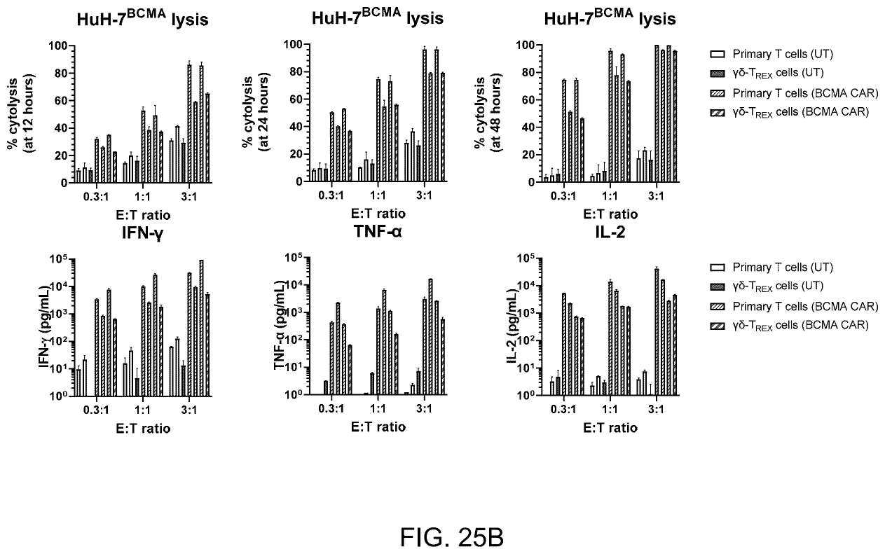

γδ T_{REX} cells were next investigated for their ability take instruction from a tumor-targeting moiety such as a BCMA-targeting CAR (FIG. 25). γδ T_{REX} cells were transduced to express a BCMA-targeting CAR (FIG. 25A), and these cells were co-cultured with BCMA-expressing tumor cells at various effector:target cell ratios. Tumor cell lysis was monitored over time using the xCELLigence platform (FIG. 25B) and supernatants were harvested 72 hours after initiation of co-culture. γδ T_{REX} cells demonstrated similar ability to control BCMA-expressing tumor cells as primary CAR-T cell controls, however, they generally secreted lower levels of effector cytokines including IFN-γ, TNF-α, and IL-2 (FIG. 25B, bottom). These data indicate γδ CAR-T_{REX} cells are capable of taking direction from a tumor-targeting CAR and may also be less likely than Primary CAR-T cells to cause CRS.

### Example 19: REX Edits in NK Cells Support an NK_{REX} Cell Phenotype

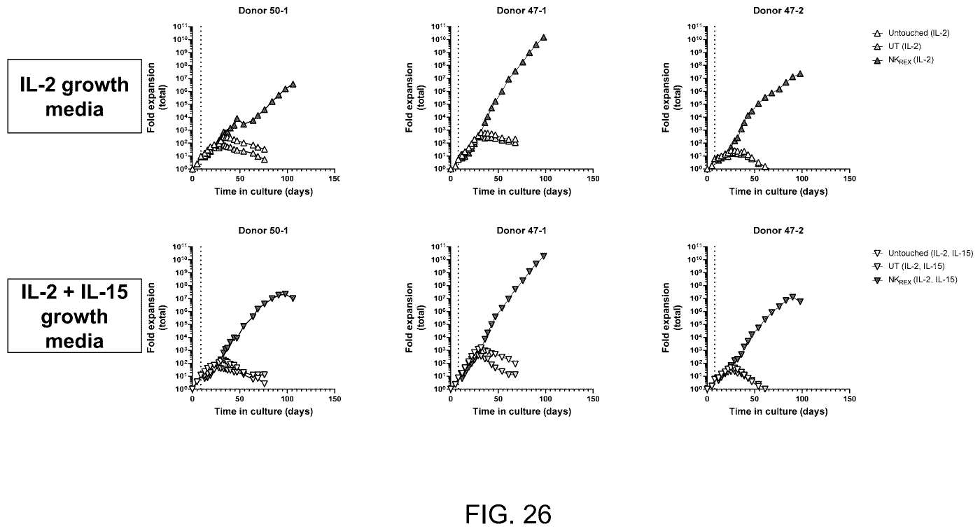

REX edits enhance T cell resistance to replicative senescence, however it was unclear whether they would support an NK_{REX} cell phenotype. Therefore, NK cells were isolated from three different donors and cultured in media containing IL-2 or a combination of IL-2 and IL-15. NK cells were then edited at the REX loci using CRISPR/Cas9 and proliferation of NK_{REX} and donor-matched unedited NK cells was monitored over time (FIG. 26). In all donors and cytokine conditions, REX edits were reproducibly able to enhance NK_{REX} cell resistance to replicative senescence (FIG. 26). NK_{REX} cells could be cultured for over 90 days, expanding >10⁶->10¹⁰ fold while unedited NK cells failed to expand and died within 80 days.

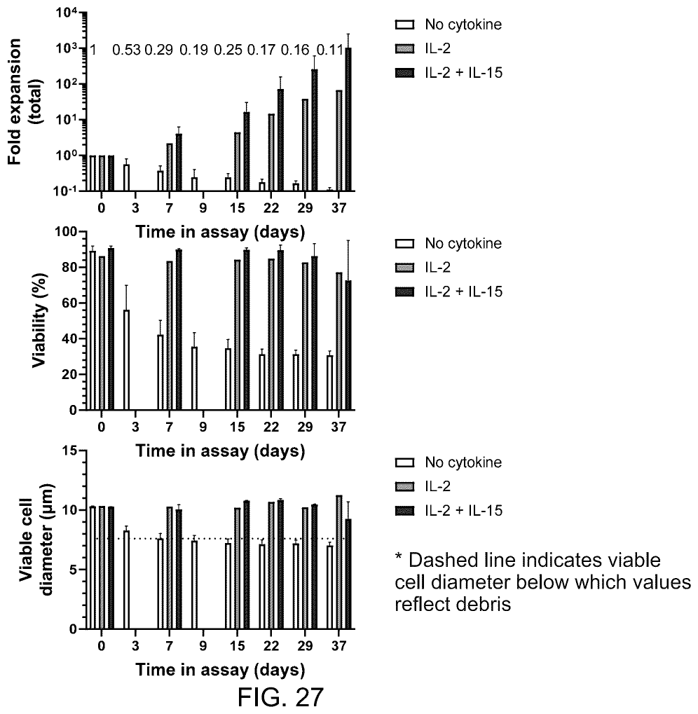

Given the enhancement in resistance to replicative senescence, it was important to determine whether NK_{REX} cells maintained their dependence on cytokine support. NK_{REX} cells were generated in media containing IL-2 or a combination of IL-2 and IL-15. NK_{REX} cell dependency on these cytokines was determined in an experiment in which cytokines were withdrawn from the growth media and NK_{REX} cell numbers were monitored for a period of 37 days. NK_{REX} cells failed to proliferate following cytokine withdrawal and these cells demonstrated a rapid drop off in cell viability and viable cell diameter, reinforcing their dependency on cytokine support despite editing of the REX genes (FIG. 27).

NK_{REX} cells were transduced to express a BCMA-targeting CAR to determine whether these cells were capable of stably expressing a tumor-targeting CAR (FIG. 28). CAR expression was maintained in CAR-NK_{REX} cells over time and levels of expression (mean fluorescence intensity, MFI) was similar to that in purified CAR-T cells. These data indicate that CAR-NK_{REX} cells can stably express a CAR and that levels of expression are comparable to that of standard CAR-T cells.

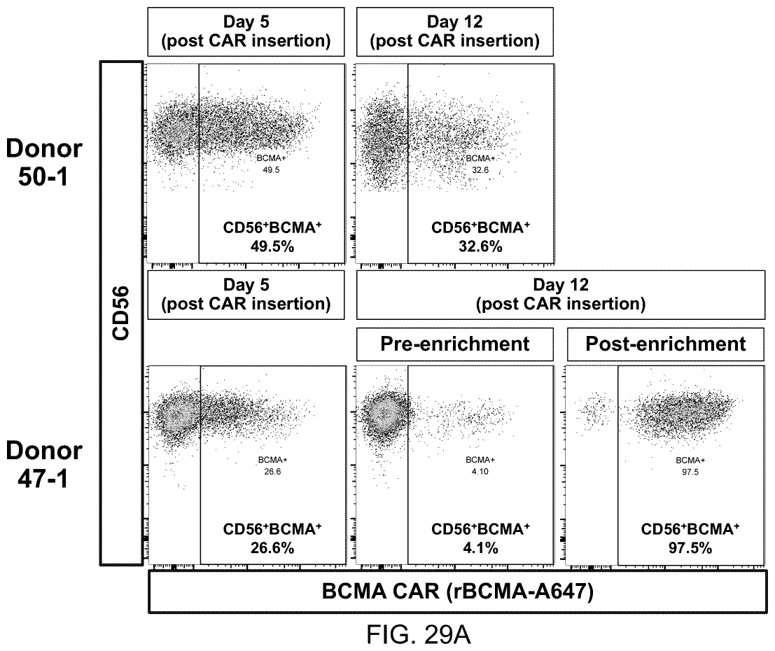

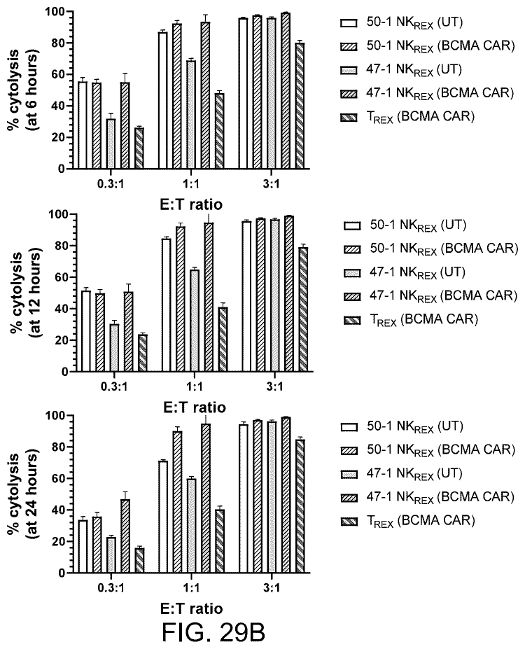

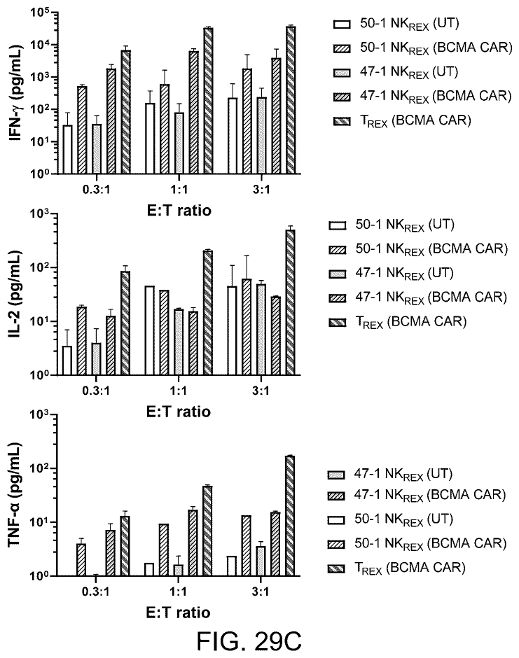

While NK_{REX} cells could be expanded in culture for long periods of time, it was unclear: 1) whether their cytotoxic potential was maintained following sustained proliferation; and 2) whether they could take direction a tumor-targeting CAR. Therefore, CAR-NK_{REX} cells were generated from two different NK_{REX} lines (FIG. 29A). One of these CAR-NK_{REX} lines was purified based on CAR expression to generate a >95% CAR⁺ CAR-NK_{REX} line (FIG. 29A, bottom right). NK_{REX} and CAR-NK_{REX} lines from donors 50-1 and 47-1 were tested for their ability to lyse BCMA-expressing tumor cells in an xCELLigence assay (FIG. 29B). Even after 78 and 86 days in culture, NK_{REX} and CAR-NK_{REX} cells were potently cytotoxic. These lines rapidly lysed BCMA-expressing target cells, achieving a higher level of control more rapidly than CAR-T_{REX} cells (FIG. 29B). While NK_{REX} cells were able to lyse tumor cells independently of CAR expression, likely due to engagement of activating receptors on NK_{REX} and CAR-NK_{REX} cells, at lower effector:target cell ratios, contributions of CAR-directed cytotoxicity could be observed for both donors 50-1 and 47-1. Supernatants were collected after 48 hours of co-culture and levels of IFN-γ, IL-2, and TNF-α were determined using MSD (FIG. 29C). NK_{REX} cells secreted lower levels of these cytokines than CAR-NK_{REX} cells, which CAR-T_{REX} cells secreted the highest levels of these factors (FIG. 29C). These data indicate that CAR-NK_{REX} cells are capable of stably expressing and taking direction from a tumor-targeting CAR. Further, these cells rapidly lyse tumor cells and accumulate lower levels of IFN-γ, IL-2, and TNF-α in co-culture supernatants.

### Example 20: T_{REX} Cells are Sensitive to T Cell Depleting Agents and Chemotherapies

T_{REX} cells have been modified to increase their resistance to replicative senescence. However, these cells have displayed hallmarks of normal T cells. In order to better understand the ability to control T_{REX} cells, their susceptibility to standard T cell depleting agents and chemotherapies was determined relative to unedited total T cells that were activated to enter cell cycle (FIG. 30). Unedited, recently activated total T cells or T_{REX} cells were incubated with 10 μg/mL anti-CD52 and 10% human complement (FIG. 30, top left) or 10% rabbit complement (FIG. 30, bottom left). After 3 hours, cell survival was assessed using a Cell Titer Glo assay. Unedited, recently activated total T cells or T_{REX} cells were also incubated with indicated quantities of melphalan (FIG. 30, top right) or chlorambucil (FIG. 30, bottom right) and cell survival was measured after 2 days using a Cell Titer Glo assay. In all cases, T_{REX} cells demonstrated comparable susceptibility to these agents as unedited, recently activated total T cells.

### Example 21: B2M^{KO} T_{REX} Cells are Sensitive to NK Cell Mediated Depletion and this can be Modulated Using Anti-CD38 Antibodies

As an allogenic cell product, T_{REX} cells are modified at the B2M locus, increasing their susceptibility to NK cell mediated depletion. These cells can be further modified at the CD38 locus to limit their depletion by anti-CD38 antibodies. T_{REX} cell lines and CD38^{KO}B2M^{KO} T_{REX} cell lines were generated using CRISPR/Cas9. T_{REX} cells and CD38^{KO}B2M^{KO} T_{REX} cells were co-cultured with PBMCs isolated from healthy donors. T_{REX} cells did not exhibit a drop in number when co-cultured with PBMCs while CD38^{KO}B2M^{KO} T_{REX} cells were susceptible to NK cell mediated lysis as expected (FIG. 31, top). NK cells express high levels of CD38 and when NK cells were preincubated with the CD38 targeting antibody Daratumamab (Dara) prior to co-culture with CD38^{KO}B2M^{KO} total T cells or CD38^{KO}B2M^{KO} T_{REX} cells, this conferred a >50% reduction in cytolysis. These data indicate that CD38^{KO}B2M^{KO} T_{REX} cells are susceptible to NK cell mediated lysis and that this sensitivity to depletion can be regulated through the administration of anti-CD38 antibodies.

### Example 22: STAT5A and STAT5B Mutants Enrich in REX Edited CD8⁺ T Cells In Vitro

T_{REX} cells (REX edit containing CD8⁺ T cells) were further modified to modulate their dependence on exogenous cytokines. T_{REX} cells are highly dependent on IL-2 for proliferation and survival and can respond to both IL-2 and IL-15 in vivo. The IL-2 and IL-15 pathways signal through STAT3 and STAT5 family members, and mutant forms of these molecules have been identified to extend the duration of signaling through these pathways. In order to test whether overexpression of mutant forms of STAT5 and STAT3 can confer a selective advantage to T_{REX} cells, one STAT5A mutant, two STAT5B mutants, and one STAT3 mutant were overexpressed with Bcl-xL in T_{REX} cells as per the timeline shown (FIG. 32 top). Enrichment of STAT mutants was followed over time using a fluorescent reporter (FIG. 32 bottom). For these experiments, cells were cultured in the presence of 300 IU/mL IL-2 and monitored for reporter positivity at indicated timepoints. STAT5A mutant and STAT5B mutant containing T_{REX} cells demonstrated enrichment throughout the culture process while the STAT3 mutant and Bcl-xL alone groups showed little to no selective advantage. These results indicate that T_{REX} cells that overexpress STAT5A H299R/S711F, STAT5B N642H, or STAT5B R430C/P702A exhibit a selective advantage over T_{REX} cells and T_{REX}+Bcl-xL cells even when cultured in the presence of IL-2.

TABLE 5
STAT mutants

| Construct | STAT | Mutation | Additional genes |
|---|---|---|---|
| STAT5A H299R/S711F* | STAT5A* | H299R/S711F | REX^{KO}, Bcl-xL |
| STAT5B N642H* | STAT5B* | N642H | REX^{KO}, Bcl-xL |
| STAT5B R430C/P702A* | STAT5B* | R430C/P702A | REX^{KO}, Bcl-xL |
| STAT5B N642H* | STAT5B* | N642H | REX^{KO} |
| STAT5B R430C/P702A* | STAT5B* | R430C/P702A | REX^{KO} |
| STAT5B V712E* | STAT5B* | V712E | REX^{KO} |
| STAT5A N642H^{Δ} | STAT5A^{Δ} | N642H | REX^{KO} |
| STAT5A Y665F^{Δ} | STAT5A^{Δ} | Y665F | REX^{KO} |
| STAT5B N642H^{Δ} | STAT5B^{Δ} | N642H | REX^{KO} |
| STAT5B Y665F^{Δ} | STAT5B^{Δ} | Y665F | REX^{KO} |
| STAT5B H298R/S715F^{Δ} | STAT5B^{Δ} | H298R/S715F | REX^{KO} |
| STAT3 Y640F* | STAT3* | Y640F | REX^{KO} , Bcl-xL |

STAT mutants were introduced into T_{REX} cells (REX edit containing CD8⁺ T cells, signified here as REX^{KO}) using different modalities. In some instances, STAT mutants were overexpressed with the survival factor Bcl-xL.
*Signifies transposon
^{Δ}Signifies lentivirus

### Example 23: STAT5A and STAT5B Mutants Exhibit Varying Degrees of IL-2 Independence In Vitro

T_{REX} cells that overexpress STAT5A H299R/S711F, STAT5B N642H, or STAT5B R430C/P702A exhibit a selective advantage over T_{REX} cells and T_{REX}+Bcl-xL cells even when cultured in the presence of IL-2 (FIG. 32). Not all STAT5A and STAT5B mutants are functionally equivalent, therefore, it was next determined whether overexpression of STAT5A and STAT5B mutants decreases T_{REX} cell dependence on exogenous IL-2. One STAT5A mutant and two STAT5B mutants were overexpressed with Bcl-XL in T_{REX} cells as per FIG. 32. STAT5 mutant T_{REX} cells and control cells (T_{REX} cells (“No transposon”) and Bcl-xL overexpressing T_{REX} cells) were cultured in decreasing amounts of IL-2 and cell expansion was followed over time. Both control groups, the STAT5A H299R/S711F mutant group, and the STAT5B R430C/P702A group exhibited dependency on IL-2 while the STAT5B N642H mutant group grew independently of IL-2 over a period of 18 days in culture (FIG. 33, white bars). As shown in FIG. 33, while the STAT5B R430C/P702A mutant exhibited IL-2 dependence, this mutation dramatically enhances T_{REX} cell expansion in the presence of even small amounts of IL-2. These data indicate that while all STAT5A and STAT5B mutants investigated in this example provide a selective advantage to T_{REX} cells, they confer varying degrees of T_{REX} cell independence from exogenous IL-2.

### Example 24: STAT Mutant Expressing T_{REX} Cells Retain Functionality in a T Cell Engager Assay in Accordance with Observed Surface CD3 Expression

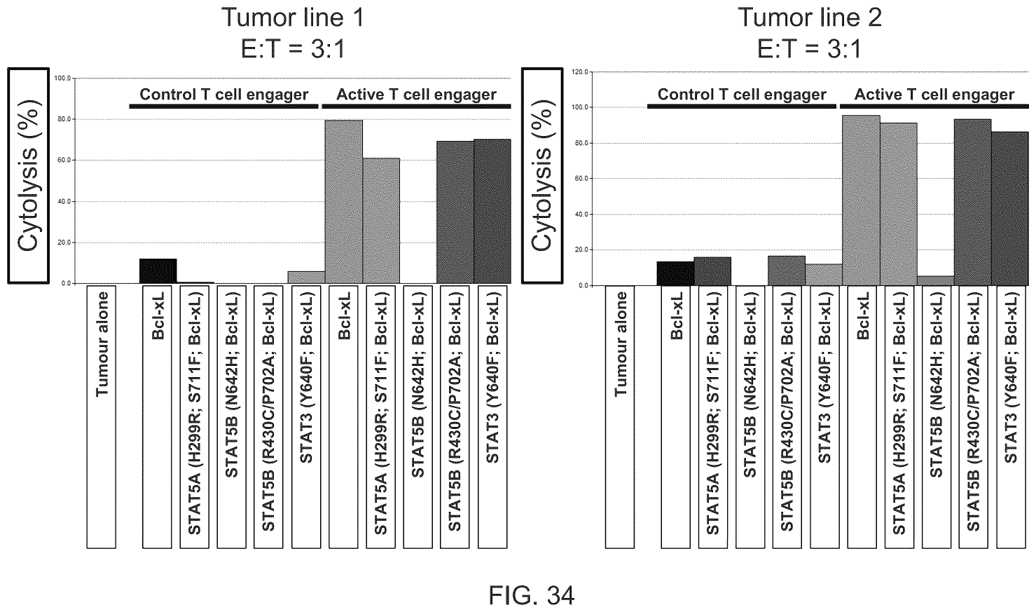

Having established that STAT5A mutant and STAT5B mutant overexpressing T_{REX} cells demonstrate enhanced growth capacity and cytokine independence relative to standard T_{REX} cells, it was next determined whether these cells maintain potent cytotoxic function. One STAT5A mutant, two STAT5B mutants, and one STAT3 mutant were overexpressed with Bcl-xL in T_{REX} cells as per FIG. 32. Cytotoxic function of STAT mutant containing T_{REX} cells was then assessed using a control (non-targeting) or active (tumor targeting) T cell engager using the impedance-based xCELLigence platform. Percent cytolysis was determined 6 hours post addition of T cell engagers. T_{REX} cells were co-cultured with two different antigen-expressing tumor lines (left and right) at an effector:target cell ratio (E:T) of 3:1. As shown in FIG. 34, STAT5A H299R/S711F, STAT5B R430C/P702A, and STAT3 Y640F transfected T_{REX} cells exhibited comparable cytotoxicity to control T_{REX}+Bcl-xL cells. STAT5B N642H mutant containing T_{REX} cells demonstrated low cytotoxicity in this assay due to low surface expression of CD3. STAT mutant overexpression did not promote non-specific cytotoxicity in this assay suggesting that STAT mutant overexpressing T_{REX} cells maintain normal functionality.

### Example 25: STAT Mutant CAR-T_{REX} Cells Maintain Cytotoxic Function in a CAR-Directed Manner In Vitro

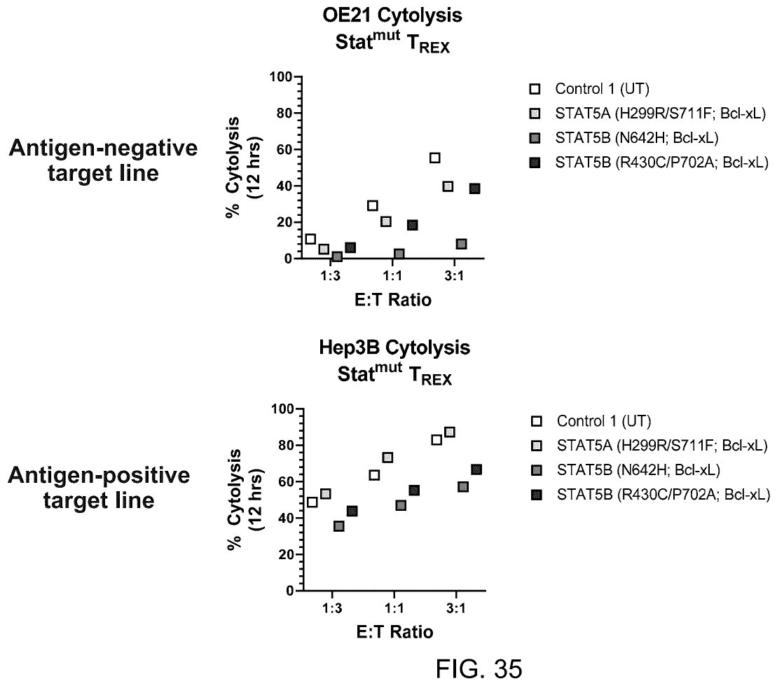

In order to function as a potential chassis, STAT mutant overexpressing T_{REX} cells must be capable of expressing a targeting molecule such as a chimeric antigen receptor (CAR) to direct their cytotoxic function. Therefore, one STAT5A mutant and two STAT5B mutants were overexpressed with Bcl-XL in T_{REX} cells as per FIG. 32. Once these cells enriched to 100% STAT mutant expression, a GPC3 targeting CAR was introduced. Cytotoxic function of STAT mutant containing CAR-T_{REX} cells was assessed using the impedance-based xCELLigence platform. Percent cytolysis was determined 12 hours post initiation of co-culture of T_{REX} cells and target cells at various effector:target (E:T) cell ratios. As shown in FIG. 35, STAT5A and STAT5B mutant T_{REX} cells retained functionality as indicated by their capacity to specifically lyse antigen-expressing tumor cells following engagement of the CAR.

### Example 26: Additional STAT5A and STAT5B Mutants Enrich in REX Edited CD8⁺ T Cells In Vitro

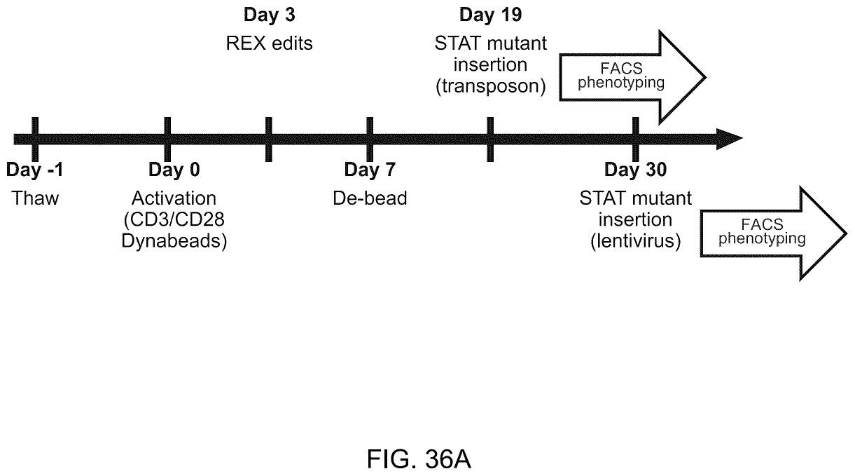

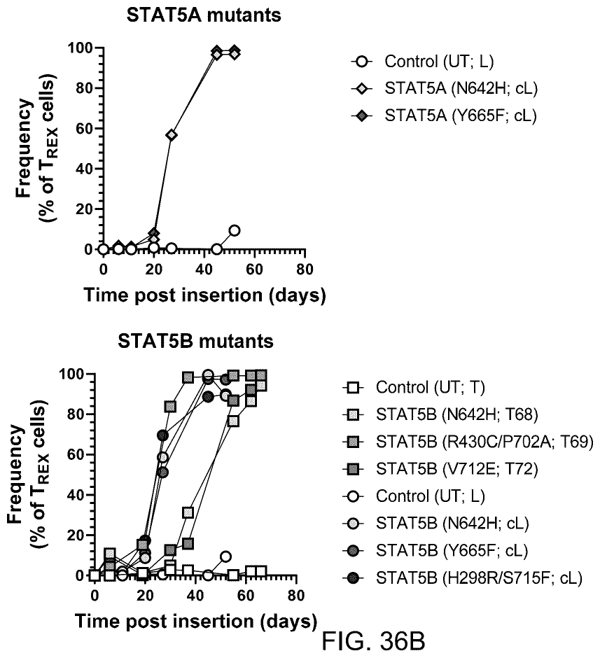

While the above studies established the functional enhancements resulting from overexpression of STAT5A H299R/S711F, STAT5B N642H, and STAT5B R430C/P702A in T_{REX} cells, these studies also incorporated overexpression of the anti-apoptotic factor Bcl-xL. In this study, the list of STAT5 mutants characterized in T_{REX} cells was expanded, and the role of the Bcl-xL transgene in conferring the enhanced T_{REX} cell phenotype was assessed. To that end, two STAT5A mutants and five STAT5B mutants were overexpressed in T_{REX} cells (in the absence of the Bcl-xL transgene) as per the timeline shown (FIG. 36A). STAT mutants were introduced using transposons (T) or lentiviruses (L) as indicated. Enrichment of STAT mutants was again followed over time using a fluorescent reporter and graphed (FIG. 36B). As shown in FIG. 36, STAT5A and STAT5B mutant expressing T_{REX} cells enriched during the cell culture process regardless of the mode of transgene introduction. These data indicate that STAT5A mutant and STAT5B mutant overexpression in T_{REX} cells confers a selective advantage to T_{REX} cells without requiring the Bcl-XL transgene. Further, these studies expand the collection of STAT5A and STAT5B mutants that promote this enhanced phenotype.

### Example 27: Expression of STAT5A and STAT5B Mutants Enhances REX Edited CD8⁺ T Cell Expansion In Vitro

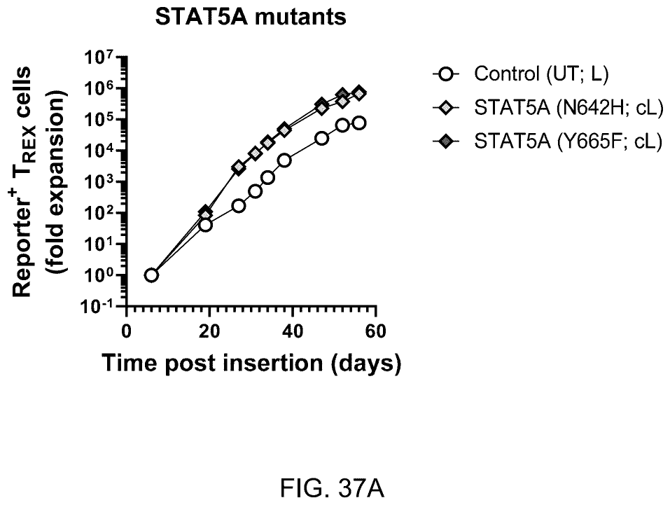

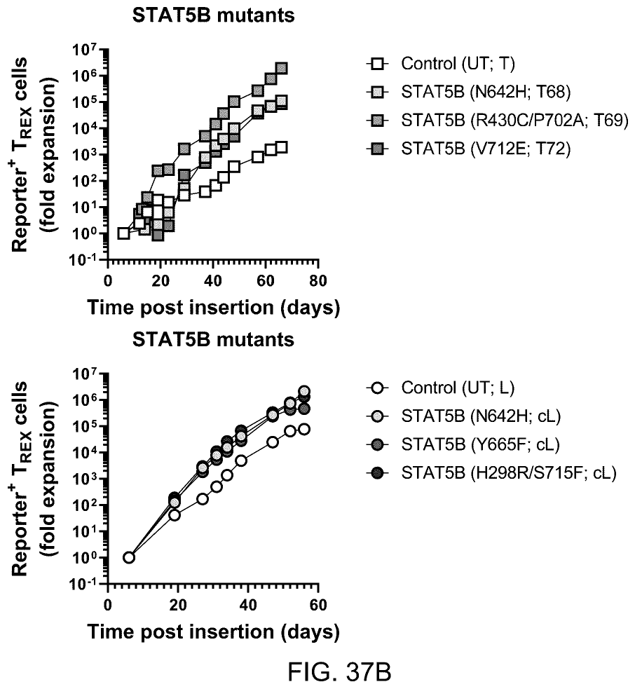

Proliferation of STAT5A mutant T_{REX} cells, STAT5B mutant T_{REX} cells, and control T_{REX} cells generated in FIG. 36 was tracked over time. As shown in FIG. 37, STAT5A and STAT5B mutant T_{REX} cells demonstrated enhanced expansion relative to unedited control T_{REX} cells. While most STAT5A and STAT5B mutants produced similar enhancement in proliferative capacity relative to control T_{REX} cells under standard in vitro cell culture conditions, STAT5A R430C/P702A overexpressing T_{REX} cells again exhibited the largest degree enhancement in T_{REX} cell expansion (FIG. 37B, top). Therefore, STAT5A and STAT5B mutant overexpression in T_{REX} cells boosts the expansion capacity of these cells under standard cell culture conditions.

### Example 28: STAT5A and STAT5B Mutants are Functional in T_{REX} Cells, Leading to Upregulation of CD25 Expression

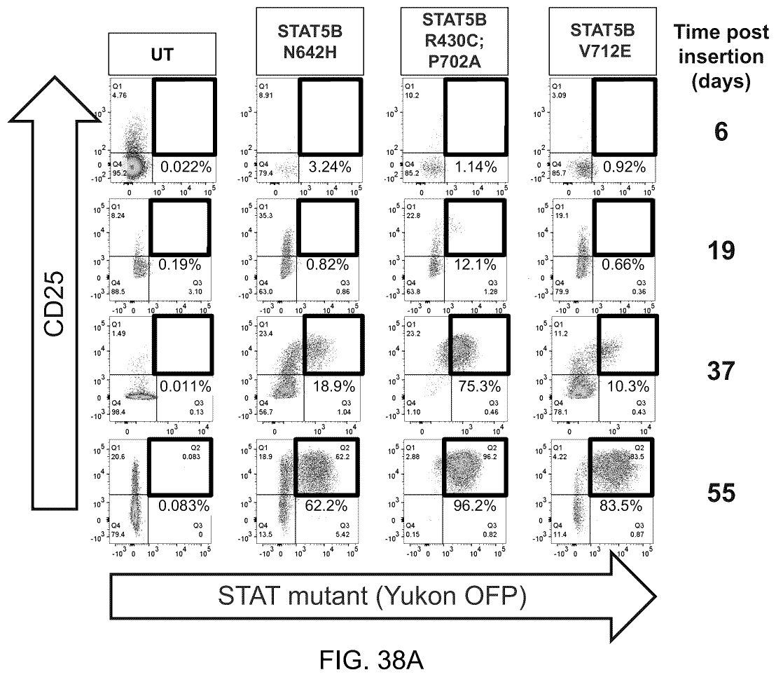

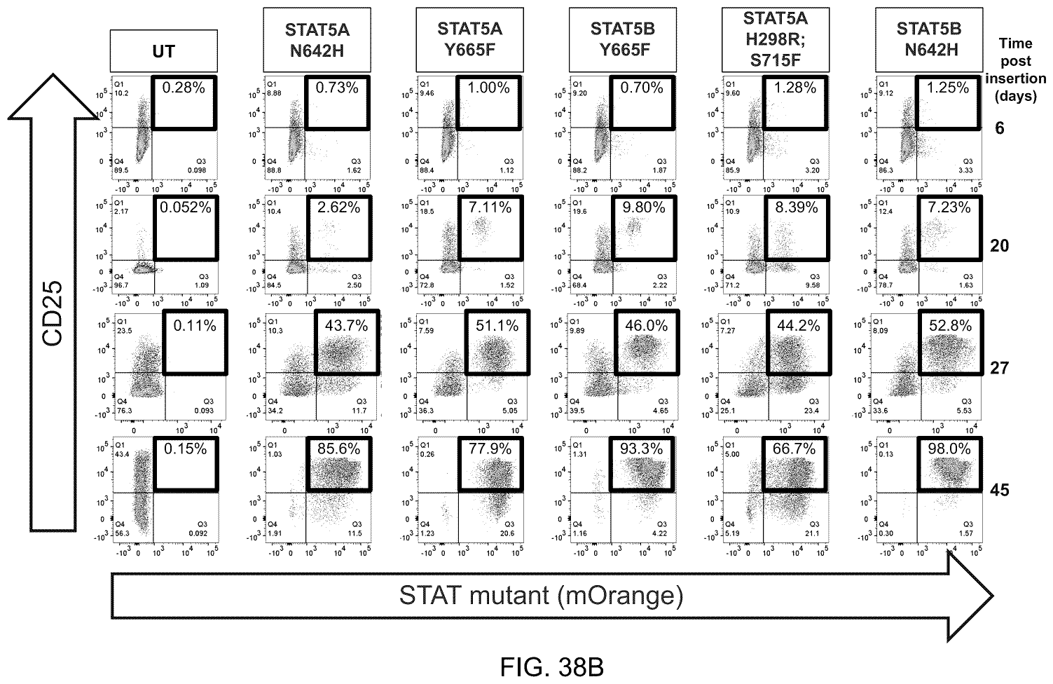

The preceding experiments suggest that overexpression of STAT5A and STAT5B mutants in T_{REX} cells enhances the phenotype of these cells and modulates their dependence on exogenous cytokines such as IL-2. To further validate the functionality of the STAT5A and STAT5B mutants that were overexpressed in these cells, two STAT5A mutants and five STAT5B mutants were transfected in T_{REX} cells (in the absence of the Bcl-XL transgene) as per the timeline shown (FIG. 36A). Signaling through the IL-2 pathway in T cells leads to upregulation CD25 (IL-2Ra) and formation of the high affinity IL-2 receptor; comprised of CD25, IL-2RB (CD122), and IL-2Ry (CD132); enhancing the ability of these cells to utilize IL-2. If these STAT5A and STAT5B mutants are functional in T_{REX} cells, then reporter⁺ cells should demonstrate increased expression of cell surface CD25 relative to control T_{REX} cells. Surface expression of CD25 was assessed in control T_{REX} cells (UT) and STAT5A and STAT5B mutant containing T_{REX} cells over extended cell culture. STAT mutants were introduced using transposons (FIG. 38A), or lentiviruses (FIG. 38B). As shown in FIG. 38, STAT5A and STAT5B mutant expressing T_{REX} cells demonstrated enhanced expression of cell surface CD25, and the frequency of CD25⁺ STAT5A mutant and CD25⁺ STAT5B mutant T_{REX} cells increased throughout the culture process. These data confirm that the STAT5A and STAT5B mutants investigated in these studies are functional and that overexpression of these molecules in T_{REX} cells boosts downstream signaling leading to upregulation of cell surface CD25, which in turn would be expected to further sensitize these cells to IL-2.

### Example 29: Generation of Alternative T_{REX} and NK_{REX} Cells

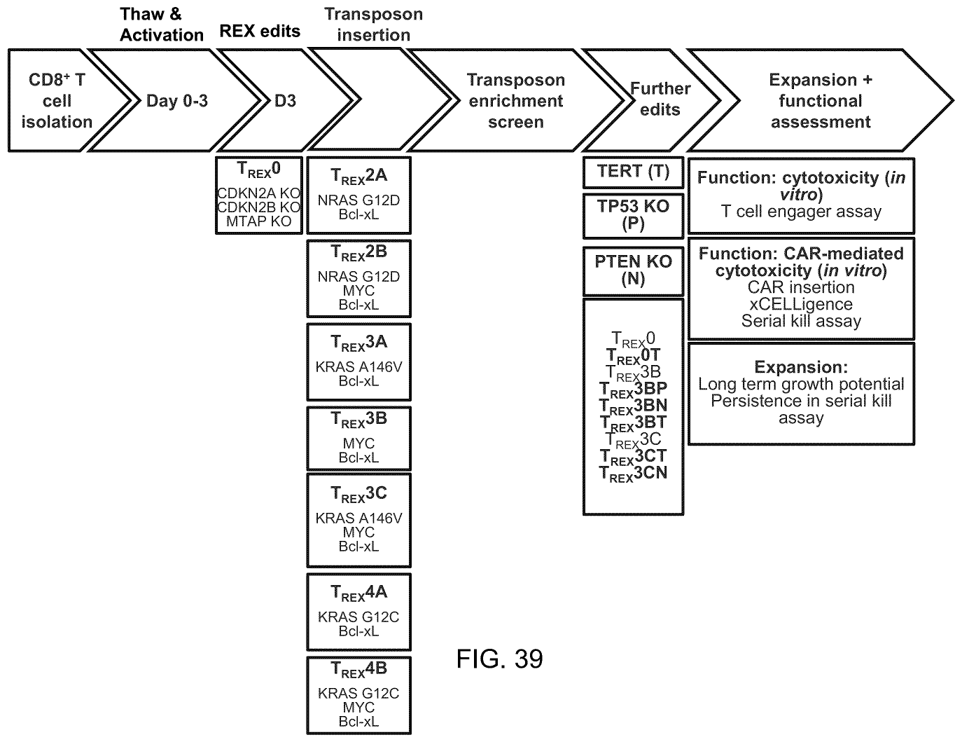

Additional combinations of edits were investigated in T_{REX} cell variants and NK_{REX} cell variants to determine whether it was possible to further enhance T_{REX} cell and NK_{REX} cell resistance to replicative senescence (Table 6). In some instances, cells were additionally modified to express a CAR. T_{REX} cell variants were generated, screened, and functionally assessed according to the workflow described in FIG. 39. Briefly, T_{REX} cells were generated and then further modified to overexpress specific genes of interest (transposon insertion) prior to an enrichment screen. In some instances, these cells were further modified to overexpress or ablate expression of other genes of interest (further edits). Engineered T_{REX} cells were assessed for long term growth potential and functionality using a variety of assays.

TABLE 6
Additional potential combinations of edits.

| Abbreviations | Cell type | Edits |
|---|---|---|
| T_{REX}0 | CD8⁺ T | REX^{KO} (CDKN2A CRISPR, CDKN2B |
| | | CRISPR, MTAP CRISPR) |
| T_{REX}2A | CD8⁺ T | REX^{KO}; +NRAS G12D, +Bcl-xL |
| T_{REX}2B | CD8⁺ T | REX^{KO}; +NRAS G12D, +Bcl-xL, |
| | | +MYC |
| T_{REX}3A | CD8⁺ T | REX^{KO}; +KRAS A146V, +Bcl-xL |
| T_{REX}3B, T_{REX}3B_3 | CD8⁺ T | REX^{KO}; +Bcl-xL, +MYC |
| T_{REX}3C, T_{REX}3C_3 | CD8⁺ T | REX^{KO}; +KRAS A146V, +Bcl-xL, |
| | | +MYC |
| T_{REX}3BP | CD8⁺ T | REX^{KO}; +Bcl-xL, +MYC; TP53 |
| | | CRISPR |
| T_{REX}3BN | CD8⁺ T | REX^{KO}; +Bcl-xL, +MYC; PTEN |
| | | CRISPR |
| T_{REX}3CN | CD8⁺ T | REX^{KO}; +KRAS A146V, +Bcl-xL, |
| | | +MYC; PTEN CRISPR |
| T_{REX}0T | CD8⁺ T | REX^{KO}; +TERT |
| T_{REX}3BT | CD8⁺ T | REX^{KO}; +Bcl-xL, +MYC; +TERT |
| T_{REX}3CT | CD8⁺ T | REX^{KO}; +KRAS A146V, +Bcl-xL, |
| | | +MYC; +TERT |
| NK_{REX} | NK | REX^{KO} |
| NK_{REX} TERT | NK | REX^{KO}; +TERT |

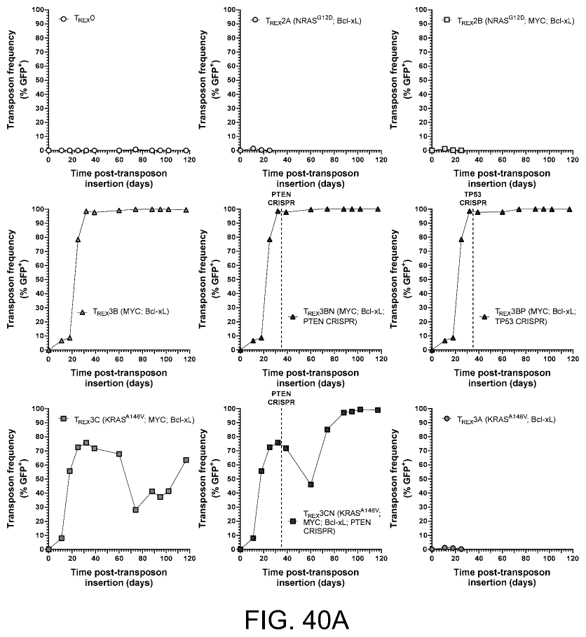

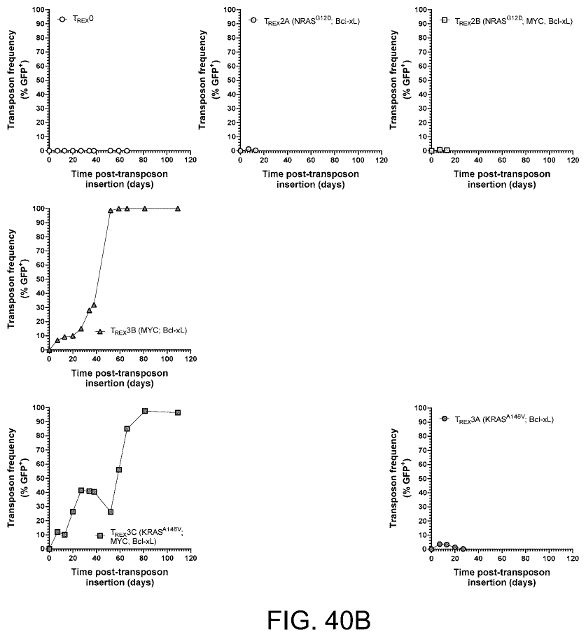

In order to determine whether additional engineering of T_{REX} cells could further enhance T_{REX} cell resistance to replicative senescence, T_{REX} cells were generated and then further modified as in FIG. 39. Engineered T_{REX} cell variants were assessed for enrichment of edit combinations over time using flow cytometry (FIG. 40A is round 1 and FIG. 40B is round 2). Five edit combinations were reassessed at a later timepoint to confirm reproducibility of earlier results (FIG. 40B, round 2). As shown in FIG. 40, specific edit combinations enriched in T_{REX} cells indicating that these T_{REX} cell variants possess a selective advantage relative to the basal T_{REX} cell phenotype. Importantly, not all edit combinations investigated in T_{REX} cells conferred this selective advantage, highlighting the importance of the specific modifications.

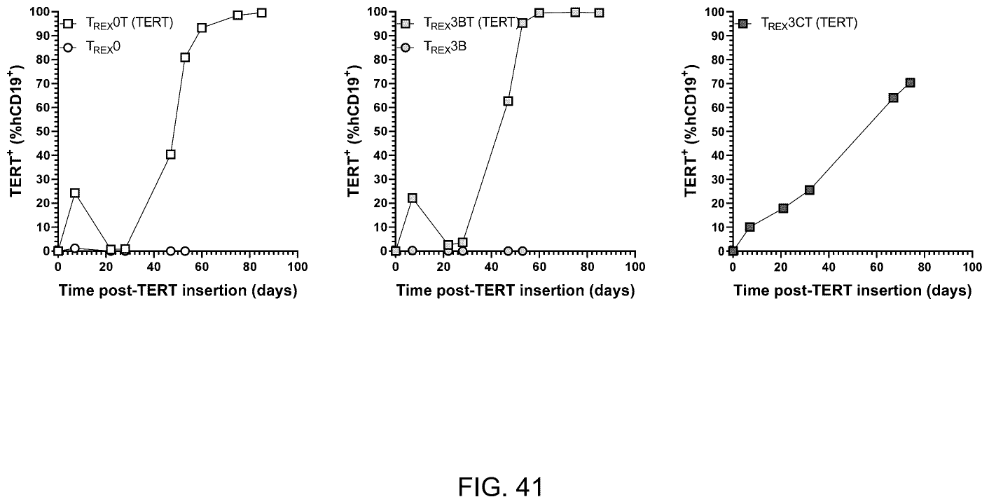

Cellular senescence can occur as a result of telomere shortening. Telomere reverse transcriptase (TERT) functions as part of a complex that extends telomeres. Accordingly, this study examined whether overexpression of TERT in T_{REX}0 cells (REX edits), T_{REX}3B cells (REX edits; MYC; Bcl-xL), and T_{REX}3C cells (REX edits; KRAS A146V; MYC; Bcl-xL), generated as per FIG. 39, could further bolster the resistances of these cells to replicative senescence. Enrichment of TERT overexpressing T_{REX}0 cells (T_{REX}0T), T_{REX}3B cells (T_{REX}3BT), and T_{REX}3C cells (T_{REX}3CT) was tracked over time (FIG. 41). As shown in FIG. 41, TERT expressing T_{REX}0, T_{REX}3B, and T_{REX}3C cells, enrich in culture, demonstrating a selective advantage conferred by overexpression of this transgene.

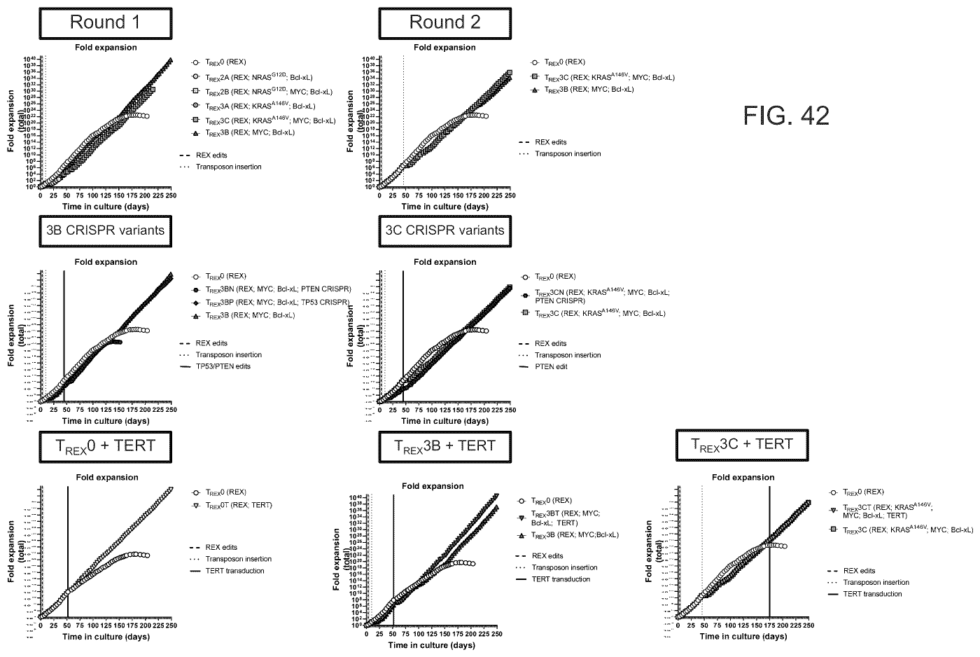

The results from FIGS. 40 and 41 indicate that further engineering of T_{REX} cells can confer a selective advantage as evidenced by enrichment of these T_{REX} cell variants in cell culture. In order to better understand whether these edit combinations also enhance T_{REX} cell resistance to replicative senescence, long term expansion of T_{REX} cells (T_{REX}0) and T_{REX} cell variants, generated as in FIGS. 39-41, was tracked (FIG. 42). Specific edit combinations (3B, 3C, 3BP, 3CN, 0T, 3BT, and 3CT) were found to substantially enhance the longevity and proliferative capacity of T_{REX} cell variants relative to T_{REX} (T_{REX}0) cells, allowing these T_{REX} cell variants to persist in culture for greater than 245 days and enhancing expansion of these cells by 6.14×10¹¹-fold to 9.82×10¹⁷-fold over maximal T_{REX} cell expansion at the time at which this experiment was ended. However, in some instances edit combinations (2A, 2B, 3A, 3BN) impaired T_{REX} cell longevity and proliferation in cell culture. These data indicate that further engineering of T_{REX} cells can enhance their resistance to replicative senescence, and that the specific combinations of these edits are critical to this phenotypic enhancement.

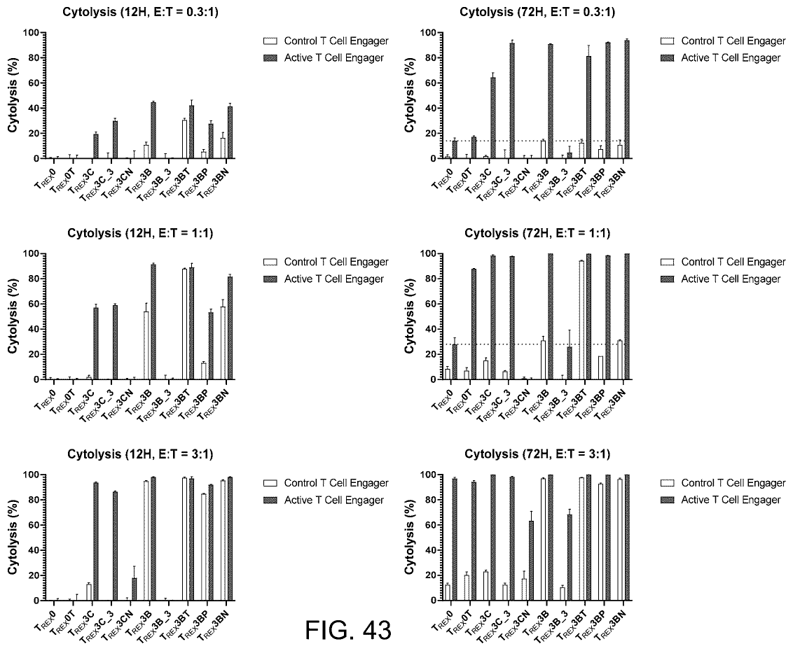

Having established that further engineering of T_{REX} cells can boost their resistance to replicative senescence, the cytotoxic potential of these T_{REX} cell variants was examined after extended cell culture. In particular, the cytotoxic potential of T_{REX} cells (T_{REX}0) and T_{REX} cell variants was assessed using a control (non-targeting) or active (tumor targeting) T cell engager using the impedance-based xCELLigence platform. Percent cytolysis was determined 12 and 72 hours post addition of T cell engagers (FIG. 43). T_{REX} effector cells were co-cultured with target cells at different effector:target (E:T) cell ratios for T_{REX}0 cells (REX edits), T_{REX}0T cells (REX edits; TERT), T_{REX}3C cells (REX edits; KRAS A146V; MYC, Bcl-xL), T_{REX}3C_3 cells (REX edits; KRAS A146V; MYC; Bcl-xL), T_{REX}3CN cells (REX edits; KRAS A146V; MYC; Bcl-xL; PTEN CRISPR), T_{REX}3B cells (REX edits; MYC; Bcl-xL), T_{REX}3B_3 cells (REX edits; MYC; Bcl-XL), T_{REX}3BT cells (REX edits; MYC; Bcl-xL; TERT), T_{REX}3BP cells (REX edits; MYC; Bcl-xL; TP53 CRISPR), and T_{REX}3BN cells (REX edits; MYC; Bcl-xL; PTEN CRISPR). Dashed line signifies cytotoxicity of T_{REX} (T_{REX}0) cells at 72 hours. As shown in FIG. 43, most T_{REX} cell variants not only maintain cytotoxic function but also exhibit increased potency relative to T_{REX}0 in this T cell engager assay, as demonstrated by accelerated killing kinetics and increased tumor cell lysis at low E:T ratios.

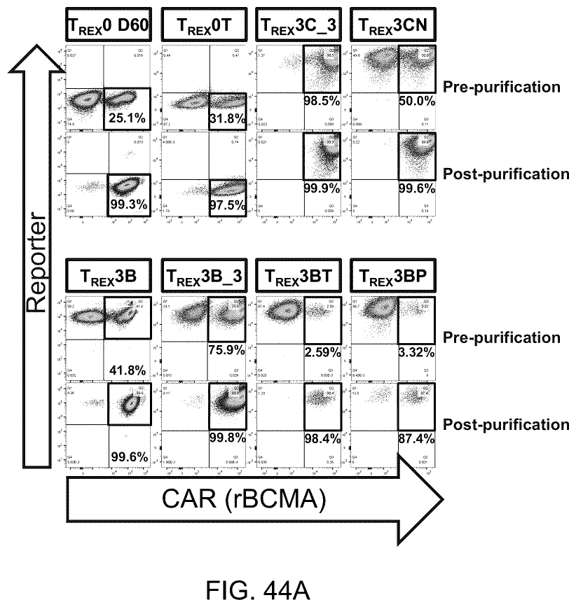

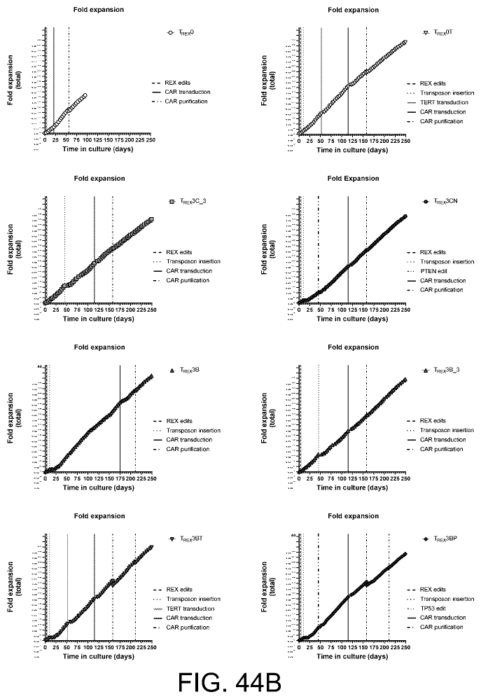

Having confirmed that T_{REX} cell variants maintain cytotoxic potential in a T cell engager assay, the ability of these cells to express a CAR and expand following CAR expression and purification was then assessed. A BCMA-targeting CAR was introduced into young (day 21) T_{REX}0 cells as a benchmark, or into long-term expanded T_{REX} cell variants (T_{REX}0T, T_{REX}3C_3, T_{REX}3CN, T_{REX}3B, T_{REX}3B_3, T_{REX}3BT, and T_{REX}3BP), and CAR expression was measured by flow cytometry 39-43 days after introduction of the CAR (FIG. 44A). Day 60 CAR-T_{REX}0 cells and day 158 to day 214 CAR-T_{REX} cell variants were further enriched to high purity prior to functional assessment (FIG. 44A). Expansion of T_{REX} cells, T_{REX} cell variants, CAR-T_{REX} cells, and CAR-T_{REX} cell variants was tracked throughout the editing, transduction, and enrichment process for each group (FIG. 44B). FIG. 44B demonstrates that CAR-T_{REX} cell variants expand robustly during the culture process.

### Example 30: CAR-T_{REX} Cell Variants Remain Functional Even after More than 200 Days in Culture

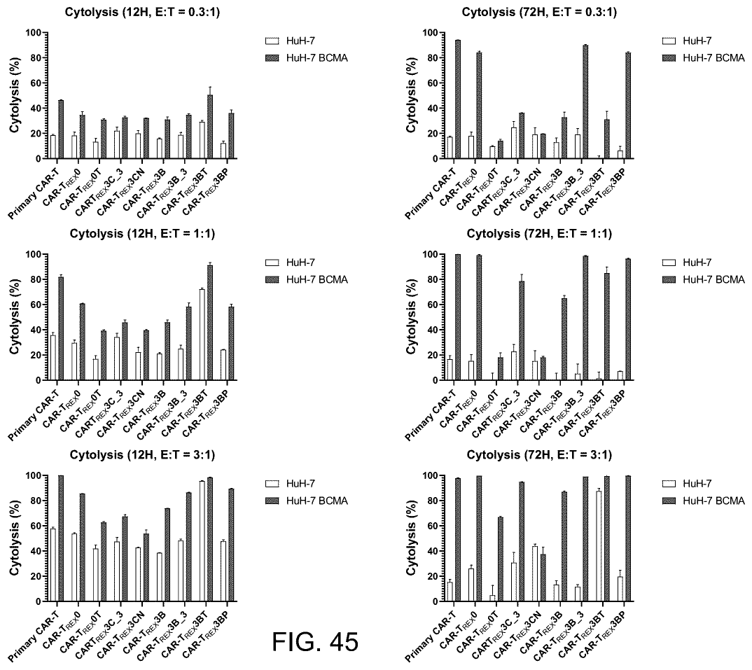

Having confirmed the capacity of T_{REX} cell variants to express a BCMA-targeting CAR, the ability of these CAR-T_{REX} cell variants to lyse tumor cells in a CAR-and-antigen-directed manner was then investigated. A BCMA targeting CAR was introduced into total primary T cells, young T_{REX} cells (T_{REX}0 D60 at time of assay) and T_{REX} cell variants (T_{REX}0T, T_{REX}3C_3, T_{REX}3CN, T_{REX}3B, T_{REX}3B_3, T_{REX}3BT, and T_{REX}3BP) as shown in FIG. 44. Cytotoxicity of CAR-expressing cells was measured using the impedance-based xCELLigence platform (FIG. 45). Percent cytolysis was determined 12 hours and 72 hours post initiation of co-culture of effector cells and target cells at various effector:target (E:T) cell ratios. Antigen-negative (HuH-7) and antigen-positive (HuH-7 BCMA) target cells were used in this experiment. The results shown in FIG. 45 confirm that CAR-T_{REX} cell variants remain functional even after more than 200 days in culture.

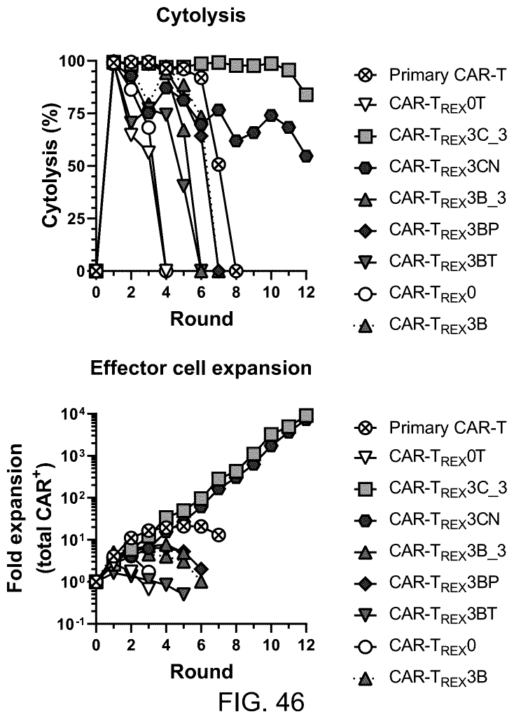

Having established that long-term expanded CAR-T_{REX} cell variants generally retained cytotoxic function through a single round of tumor cell lysis (FIG. 45), the persistence and cytotoxic capacity of these cells through multiple rounds of tumor cell lysis was then evaluated. A BCMA targeting CAR was introduced into total primary T cells, young T_{REX} cells (T_{REX}0 D67 at time of assay) and T_{REX} cell variants (T_{REX}0T, T_{REX}3C_3, T_{REX}3CN, T_{REX}3B, T_{REX}3B_3, T_{REX}3BT, and T_{REX}3BP). Cytotoxicity and persistence of CAR-expressing cells were measured over multiple rounds of co-culture with BCMA-expressing JJN3 target cells at an effector:target cell ratio of 1:1. Following each round of co-culture, percent cytolysis (top) and effector cell expansion (bottom) were assessed (FIG. 46). As shown in FIG. 46, most CAR-T_{REX} cell variants persist similarly to Primary CAR-T cells in a serial kill assay and these 221-day old CAR-T_{REX} cell variants are at least as functional as young CAR-T_{REX} cell benchmarks. CAR-T_{REX}3C_3 and CAR-T_{REX}3CN variants persisted substantially longer than Primary CAR-T cells, young CAR-T_{REX} cell benchmarks, and other CAR-T_{REX} cell variants in this assay.

### Example 31: TERT Overexpression Confers an Advantage to NK_{REX} and CAR-NK_{REX} Cells

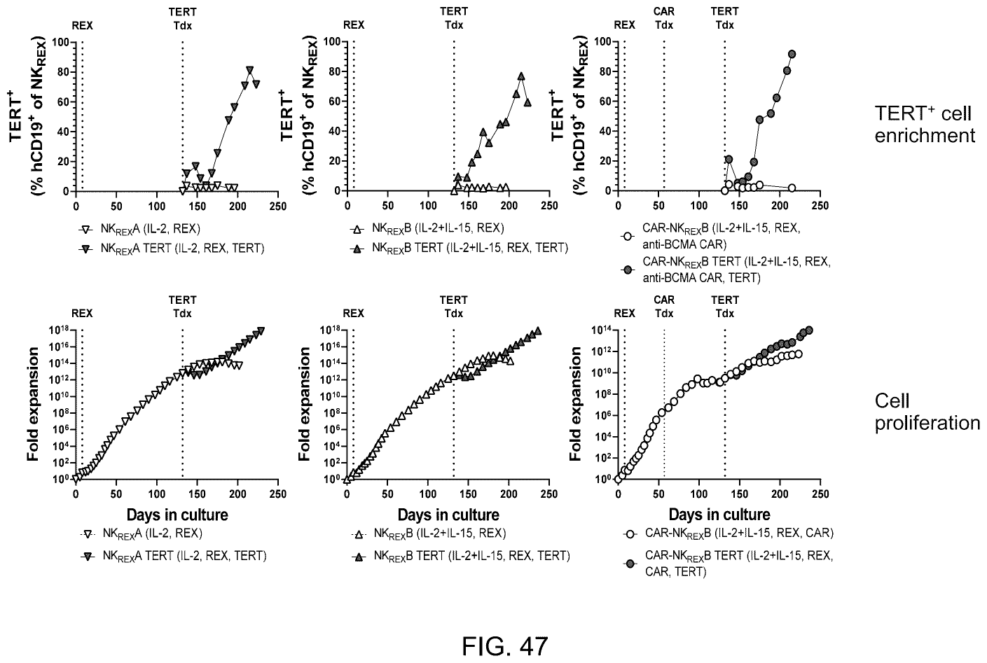

Having established that overexpression of TERT in T_{REX} cells and T_{REX} cell variants can further enhance their resistance to replicative senescence, these studies were further extended to NK_{REX} cells (REX edit containing NK cells) and CAR-NK_{REX} cells. NK_{REX} cells were generated and in some instances modified to express a BCMA-targeting CAR. NK_{REX} and CAR-NK_{REX} cells were then further engineered to overexpress TERT (FIG. 47). All groups were monitored for enrichment of TERT-expressing NK_{REX} and CAR-NK_{REX} cells over time (FIG. 47 top). Proliferation of all groups was also measured throughout the culture process (FIG. 47 bottom). Cells were grown under indicated cytokine conditions (IL-2 or IL-2+IL-15). As shown in FIG. 47, TERT-expressing NK_{REX} cells and TERT-expressing CAR-NK_{REX} cells demonstrated enrichment throughout the cell culture process (FIG. 47 top). Further, these TERT-expressing variants exhibited enhanced resistance to replicative senescence as measured by increased longevity and expansion in cell culture relative to NK_{REX} and CAR-NK_{REX} controls (FIG. 47 bottom). CAR-NK_{REX} TERT IL-2 IL-15 cells also continued to expand (eventually reaching 10\hat{ }30 cells) after cryo-preservation and re-culture.

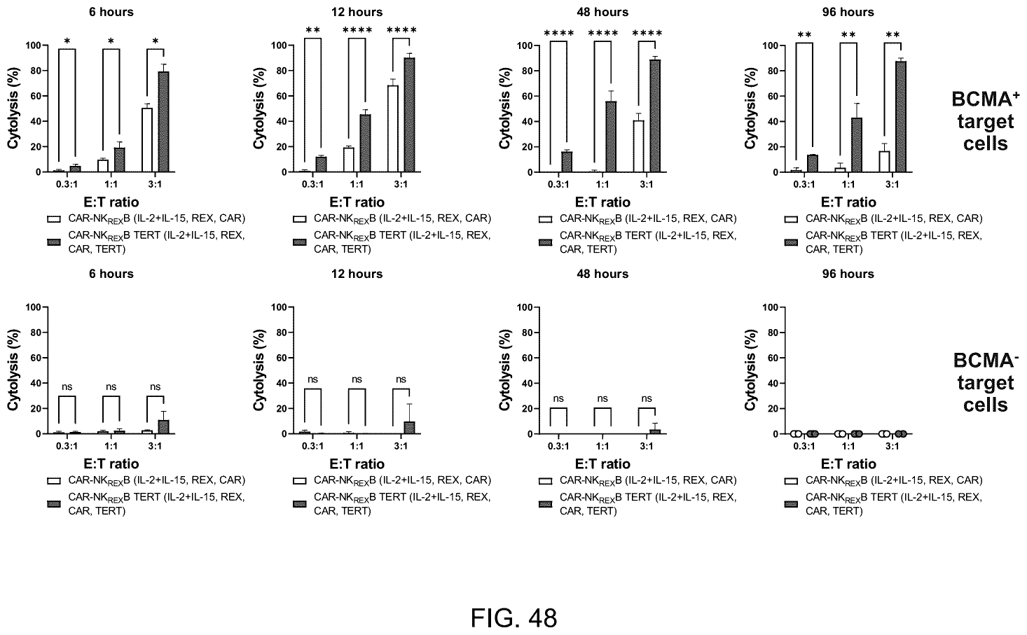

While overexpression of TERT in NK_{REX} cells and CAR-NK_{REX} cells enhanced the resistance of these cells to replicative senescence, it was unclear whether these variants maintained their cytotoxic function. Therefore, CAR-NK_{REX} cells (CAR-NK_{REX}B) and TERT overexpressing CAR-NK_{REX} cells (CAR-NK_{REX}B TERT) were generated as described above and the cytotoxicity of CAR-expressing cells was measured using the impedance-based xCELLigence platform. Percent cytolysis was determined 6, 12, 48, and 96 hours post initiation of co-culture of effector cells and target cells at various effector:target (E:T) cell ratios (FIG. 48). Antigen-negative and antigen-positive target cells were used in this experiment. CAR-NK_{REX} cells were grown under indicated cytokine conditions (IL-2+IL-15) prior to the co-culture experiment in which no cytokine support was provided. As shown in FIG. 48, TERT overexpressing CAR-NK_{REX} cells (CAR-NK_{REX} B TERT) show improved function relative to CAR-NK_{REX} cells (CAR-NK_{REX}B) as evidenced by accelerated killing kinetics and maximal cytotoxicity observed at each E:T ratio. Further, these results demonstrate that enhancement in cytotoxicity was CAR-mediated, as no increase in lysis of antigen-negative tumor cells was observed.

### Example 32: T_{REX} Cells Demonstrate Enrichment in Combined Edits

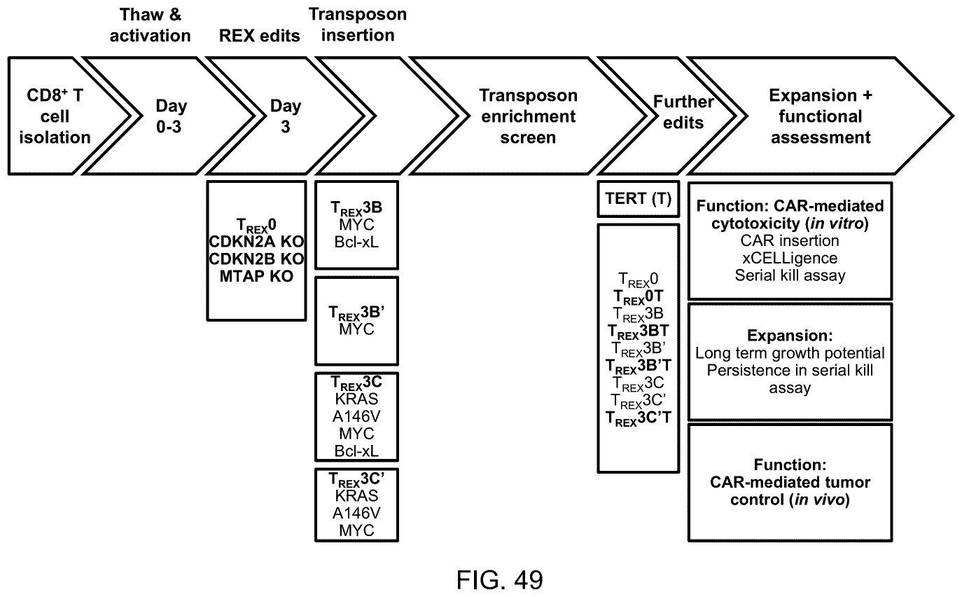

T_{REX} cells (REX edit containing CD8⁺ T cells) were generated and then further modified to overexpress specific genes of interest (transposon insertion) prior to an enrichment screen. In some instances, these cells were further modified to overexpress other genes of interest (further edits) (see FIG. 49). Engineered T_{REX} cells were assessed for long term growth potential and functionality using different assays.

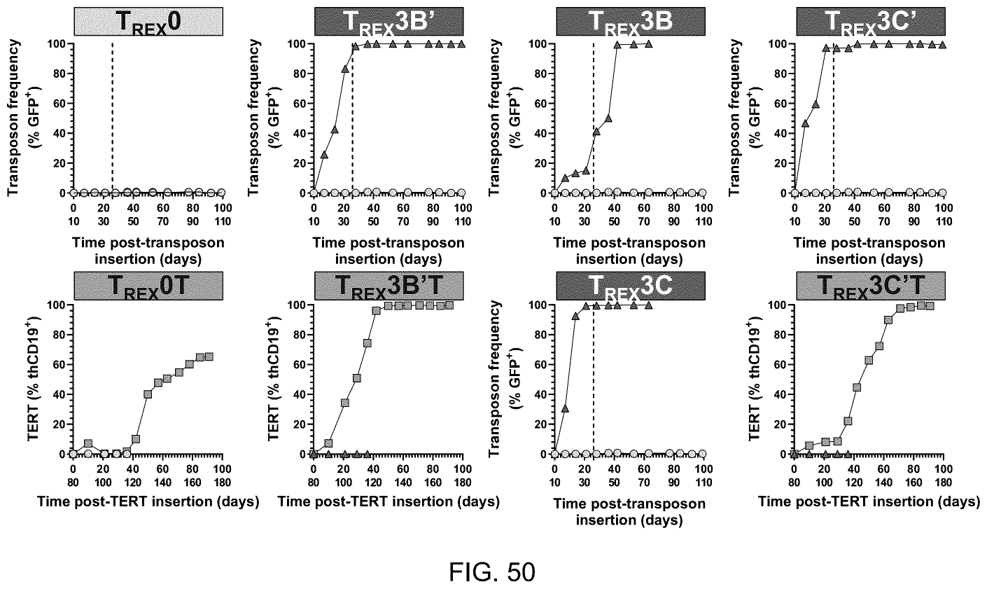

T_{REX}0T cells, T_{REX}3B′ cells, T_{REX}3B′T cells, T_{REX}3B cells, T_{REX}3C cells, T_{REX}3C′ cells, and T_{REX}3C′T cells were assessed for enrichment of edit combinations over time, as shown in FIG. 50. Each of these further edit combinations, detailed in Table 7, enriched within the REX edited CD8⁺ T cell pool (T_{REX}), indicating that they conferred a further selective advantage throughout the cell culture process, beyond that observed with the REX edits.

TABLE 7
Additional potential combinations of edits.

| Abbreviations | Cell type | Edits |
|---|---|---|
| T_{REX}0 | CD8⁺ T | REX^{KO} (CDKN2A CRISPR, CDKN2B |
| | | CRISPR, MTAP CRISPR) |
| T_{REX}3B | CD8⁺ T | REX^{KO}; +Bcl-xL, +MYC |
| T_{REX}3B′ | CD8⁺ T | REX^{KO}; +MYC |
| T_{REX}3C | CD8⁺ T | REX^{KO}; +KRAS A146V, +Bcl-xL, +MYC |
| T_{REX}3C′ | CD8⁺ T | REX^{KO}; +KRAS A146V, +MYC |
| T_{REX}0T | CD8⁺ T | REX^{KO}; +TERT |
| T_{REX}3BT | CD8⁺ T | REX^{KO}; +Bcl-xL, +MYC; +TERT |
| T_{REX}3B′T | CD8⁺ T | REX^{KO}; +MYC; +TERT |
| T_{REX}3C′T | CD8⁺ T | REX^{KO}; +KRAS A146V, +MYC; +TERT |

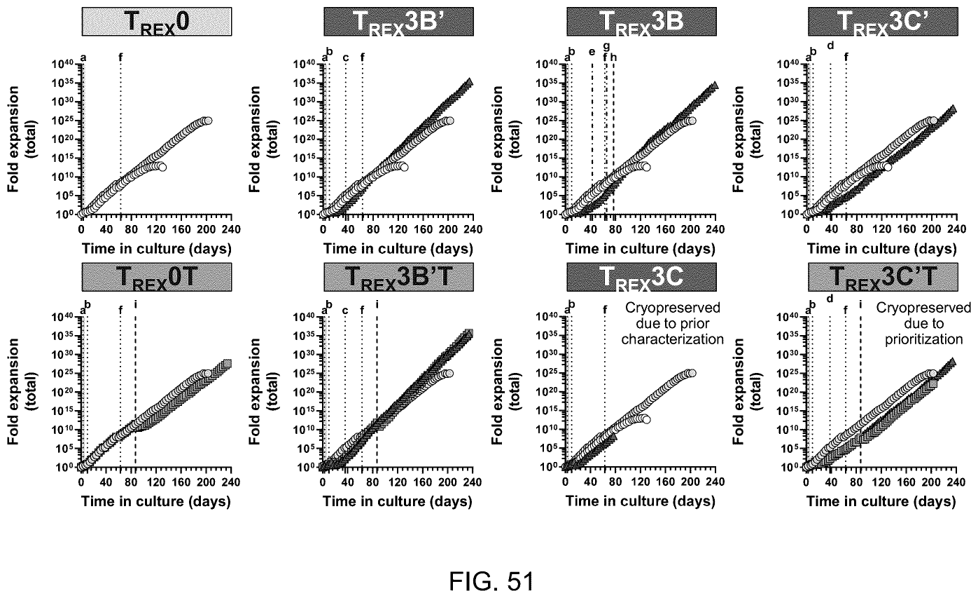

T_{REX} cell variants were generated as described in FIG. 49. Cell expansion was tracked over time and T_{REX} cell variants (T_{REX}0T, T_{REX}3B′, T_{REX}3B′T, T_{REX}3B, T_{REX}3C*, T_{REX}3C′, and T_{REX}3C′T*) exhibited enhanced longevity and proliferative capacity relative to T_{REX} (T_{REX}0) cells (see FIG. 51). While T_{REX}0 showed improved proliferation (max expansion=9.92E+024, max culture=203 days) relative to unedited, donor-matched CD8⁺ T cells (max expansion=1.05E+013, max culture=130 days), T_{REX}0 plateaued by approximately day 190 in culture. In contrast to T_{REX}0, the T_{REX} cell variants investigated in this example continued proliferating in a log-linear fashion comparable to cell lines. T_{REX} cell variants were generally cultured for approximately 240 days at which point they were cryopreserved for further use and characterization. In some instances, T_{REX} cell variants (T_{REX}3C, T_{REX}3C′T) were cryopreserved early due to prioritization. However, these cells exhibited enhanced, cell-line like growth. Excluding the two groups that were cryo-preserved early due to prioritization, T_{REX} cell variants achieved 4.08E+027 to 5.17E+035 fold expansion prior to cryo-preservation. In some instances, Bcl-xL was not required for the cells to exhibit advantageous enrichment and growth characteristics (for example compare T_{REX}3B′ vs. T_{REX}3B and T_{REX}3C′ vs. T_{REX}3C in FIG. 51).

### Example 33: T_{REX} Cell Variants can be Single-Cell Cloned

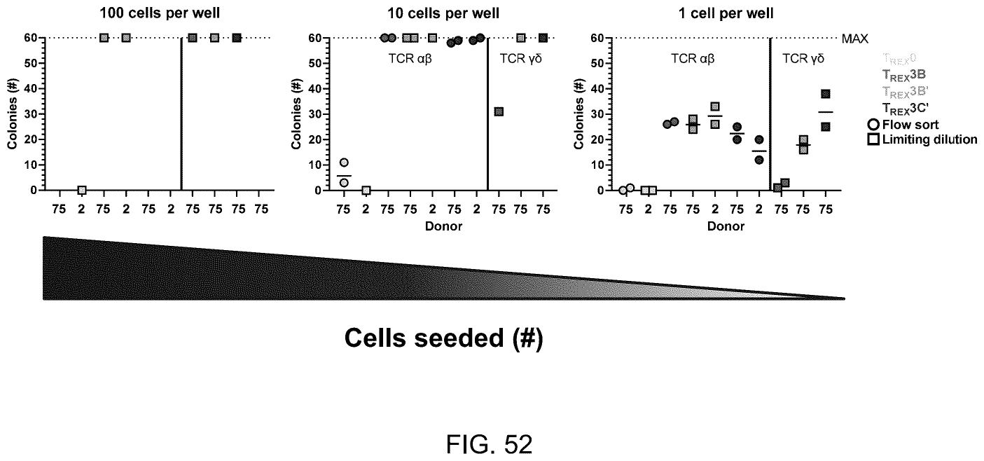

T_{REX} cells (REX edit containing CD8⁺ T cells) were generated and then further modified as in FIG. 49 to produce T_{REX}3B′ cells, T_{REX}3B cells, and T_{REX}3C′ cells. Single-cell clonability of engineered T_{REX} cells was assessed using flow cytometry based cell sorting or limiting dilution analysis. Engineered cells were seeded at 1, 10, or 100 cells per well and cultured in the presence of IL-2 containing media for 2 weeks. Plates were imaged to assess colony formation. A maximum of 60 colonies per plate were obtained. In contrast to T_{REX}0 cells which could not be single cell cloned, T_{REX}3B′ cells, T_{REX}3B cells, and T_{REX}3C′ cells could produce colonies at varying frequencies. Similar results were obtained for T_{REX}0T, T_{REX}3B, and T_{REX}3CN cells (see FIG. 52).

### Example 34: T_{REX} Cell Variants Expand Robustly During the Culture Process

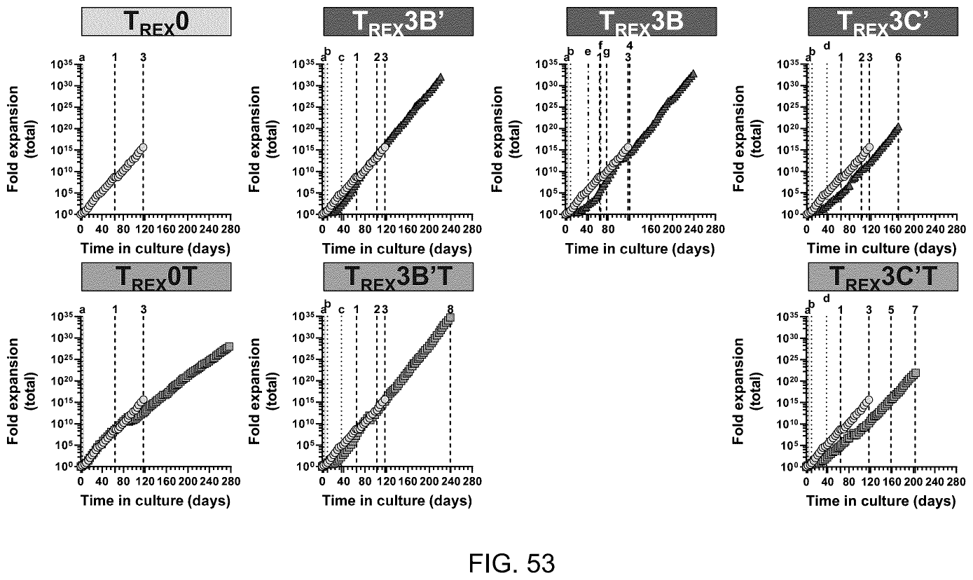

A BCMA targeting CAR was introduced into young T_{REX} cells (T_{REX}0) and T_{REX} cell variants (T_{REX}0T, T_{REX}3B′, T_{REX}3B′T, T_{REX}3B, T_{REX}3C′, and T_{REX}3C′T). CAR-expressing cells were further enriched to high purity prior to functional assessment. Expansion of CAR-T_{REX} cells and CAR-T_{REX} cell variants was tracked throughout the editing, transduction, and enrichment process for each group. While T_{REX}0 CAR-T cells were cryo-preserved at the peak of their expansion and functionality for use in subsequent studies (4.30E+015, day 117), T_{REX} cell variant CAR-T cells demonstrated a capacity to continue proliferating throughout the culture process, achieving the following degrees of expansion at the indicated times of cryo-preservation: T_{REX}0T (1.05E+028, day 277), T_{REX}3B′ (1.17E+032, day 221), T_{REX}3B′T (5.86E+034, day 239), T_{REX}3B (7.48E+032, day 239), T_{REX}3C′ (3.06E+020, day 171), and T_{REX}3C′T (8.35E+021, day 204). Even groups that were cryo-preserved early demonstrated a consistent pattern of proliferation and had not yet begun to plateau in cell culture. Results demonstrate that T_{REX} cell variants expand robustly during the culture process (see FIG. 53). In some instances, Bcl-xL was not required for the cells to exhibit advantageous enrichment and growth characteristics (for example T_{REX}3B′ and T_{REX}3B have similar growth characteristics).

### Example 35: CAR-T_{REX} Cell Variants are Cytotoxic and Persist in Serial Kill Assays

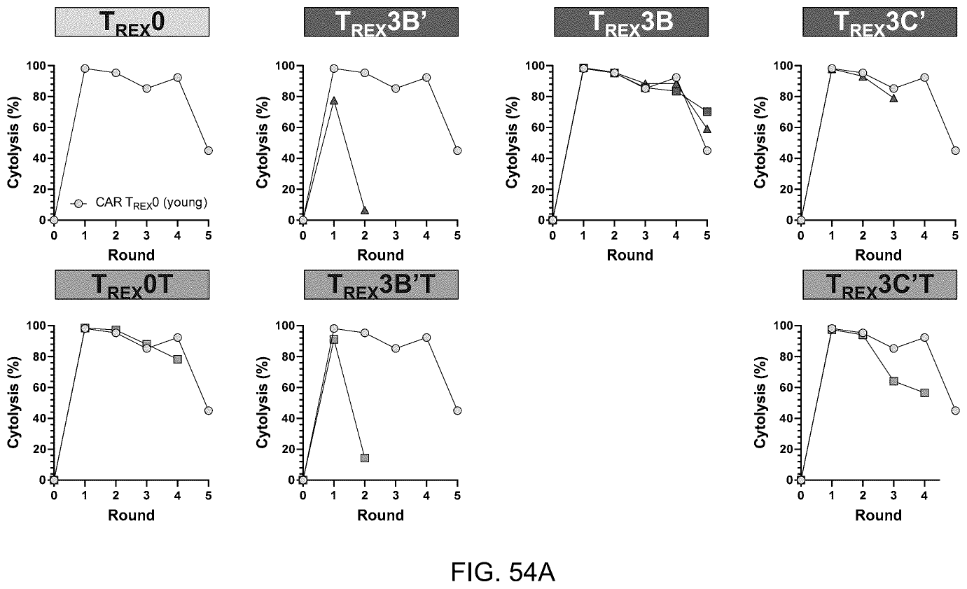

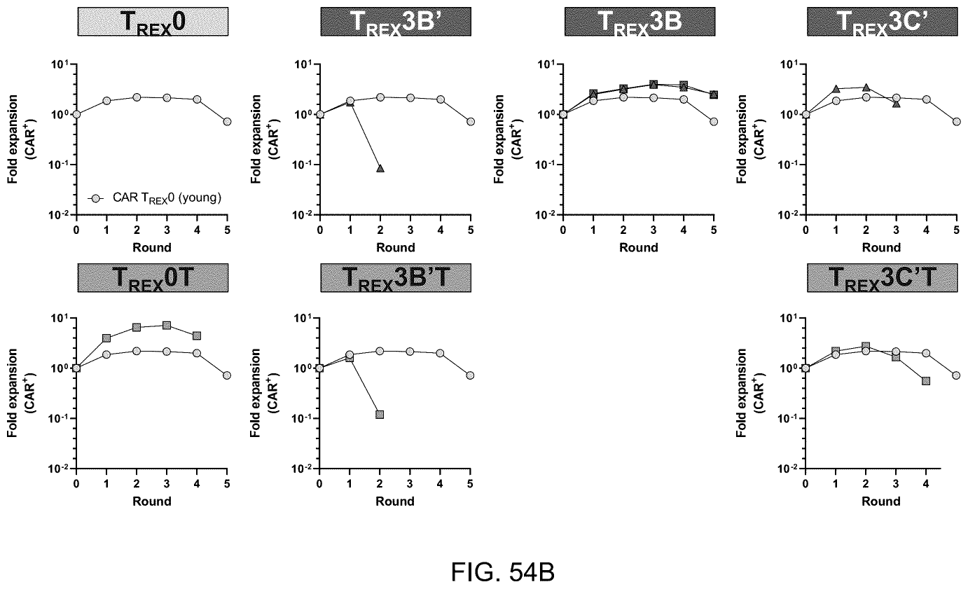

A BCMA targeting CAR was introduced into total primary T cells, young T_{REX} cells (T_{REX}0-D88) and T_{REX} cell variants (T_{REX}0T, T_{REX}3B′, T_{REX}3B′T, T_{REX}3B, T_{REX}3C′, and T_{REX}3C′T-D126-182). Cytotoxicity (FIG. 54A) and persistence (FIG. 54B) of CAR-expressing cells were measured over multiple rounds of co-culture with BCMA-expressing JJN3 target cells at an effector:target cell ratio of 1:1. Following each round of co-culture, percent cytolysis (FIG. 54A) and effector cell expansion (FIG. 54B) were assessed. Unmodified CAR-T_{REX} cells (T_{REX}0 (young)) at the peak of function completed 5 rounds of serial kill, achieving >30% cytolysis. CAR-T_{REX} cell variants completed 1-5 rounds of serial kill at >30% cytolysis as seen in FIG. 54A. Activity of CAR-T_{REX}0T and CAR-T_{REX}3B cells was most potent in this assay with cells completing 4-5 rounds of serial kill. CAR-T_{REX}0T (max expansion: 7.13 fold) and CAR-T_{REX}3B cells (max expansion: 4.00 fold) also exhibited enhanced proliferation relative to CAR-T_{REX}0 cells (max expansion: 2.19 fold) in this assay as seen in FIG. 54B.

### Example 36: CAR-T_{REX} Cell Variants are at Least as Functional as Young CAR-T_{REX} Cell Benchmarks In Vivo

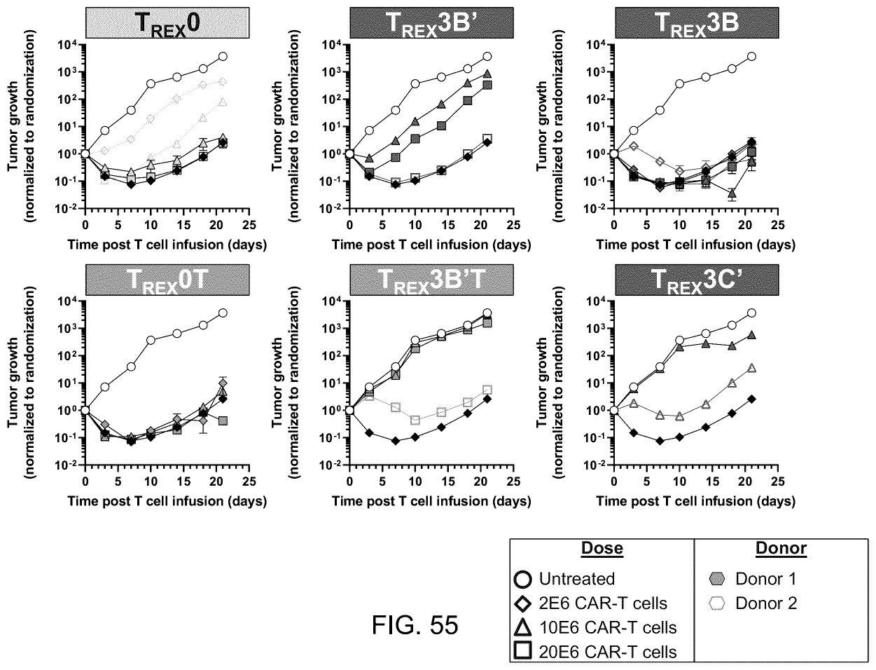

NSG mice were inoculated with 10E6 MMIS-luciferase tumor cells. 3 days later, Primary CAR-T cells (D14), CAR-T_{REX} cells (T_{REX}0, D99-102), or CAR-T_{REX} cell variants (T_{REX}0T, T_{REX}3B′, T_{REX}3B′T, T_{REX}3B, and T_{REX}3C′-D137-200) were dosed at 2E6, 10E6, or 20E6 cells per mouse. Tumor burden was monitored twice weekly using IVIS imaging. All CAR-T_{REX} cell variants demonstrated in vivo functionality, despite higher degrees of in vitro expansion (see Table 8 below, and FIG. 55). In some cases, CAR-T_{REX} cell variants were even more efficacious than young CAR-T_{REX} cells (T_{REX}0) at low doses (T_{REX}3B and T_{REX}0T were capable of controlling tumor in vivo at a 2E6 cell dose while T_{REX}0 cells were unable to control disease under these conditions. At a 2E6 cell dose, CAR-T_{REX}0 cells were unable to clear tumor as evidenced by 246±116.6% tumor growth at day 3 post-effector-infusion. In contrast at this time point, 2E6 CAR-T_{REX}3B cells cleared 47.3%±20.2% and 94.3%±0.58% of tumor depending on the donor line while 2E6 CAR-T_{REX}0 cells cleared 93%±1.7% of tumor in mice).

TABLE 8
In vivo functional CAR-T_{REX} cells

| | Age at infusion | Fold Expansion |
|---|---|---|
| Groups | (days in culture) | at infusion |
| Primary (2E6) | 14 | 1.35E+02 |
| Donor 1 T_{REX}0 (10E6, 20E6) | 99 | 1.41E+13 |
| Donor 2 T_{REX}0 (2E6, 10E6) | 102 | 4.45E+12 |
| Donor 1 T_{REX}3B′ (10E6, 20E6) | 137 | 8.17E+19 |
| Donor 2 T_{REX}3B′ (20E6) | 148 | 1.62E+22 |
| Donor 1 T_{REX}3B (2E6) | 162 | 9.26E+20 |
| Donor 1 T_{REX}3B (10E6, 20E6) | 155 | 1.01E+20 |
| Donor 2 T_{REX}3B (2E6, 10E6) | 158 | 4.35E+23 |
| Donor 1 T_{REX}0T (2E6) | 200 | 5.28E+21 |
| Donor 1 T_{REX}0T (10E6, 20E6) | 193 | 8.30E+20 |
| Donor 1 T_{REX}3B′T (10E6, 20E6) | 193 | 4.53E+27 |
| Donor 2 T_{REX}3B′T (20E6) | 200 | 2.54E+31 |
| Donor 1 T_{REX}3C′ (10E6) | 144 | 1.99E+16 |
| Donor 2 T_{REX}3C′ (10E6) | 148 | 9.60E+19 |

### Example 37: Cryo-Recovered TERT Overexpressing CAR-NK_{REX} Cells are Cytotoxic and Persist Better than in Serial Kill Assays than Younger Cryo-Recovered CAR-NK_{REX} Cells

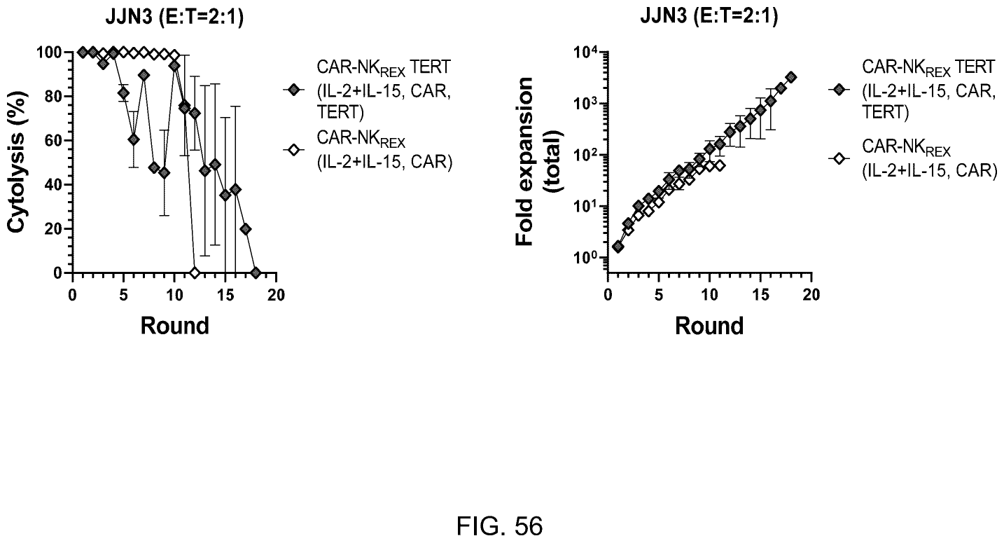

BCMA targeting CAR-NK_{REX} cells and TERT overexpressing CAR-NK_{REX} cells were generated as described in FIG. 47. Cells were cryo-preserved on days 84 and 256, respectively. Cells were cryo-recovered and cultured to days 89 and 304, respectively, at time of initiation of the serial kill assay. Cytotoxicity and persistence of CAR-expressing cells were measured over multiple rounds of co-culture with BCMA-expressing JJN3 target cells at an effector:target cell ratio of 2:1 in the presence of IL-2. Following each round of co-culture, percent cytolysis and effector cell expansion were assessed (see FIG. 56). TERT overexpressing CAR-NK_{REX} cells expanded for more than 450 days in culture, reaching >10\hat{ }30-fold expansion. Despite extended additional culture (CAR-NK_{REX}=10\hat{ }9 fold expansion at assay start, TERT overexpressing CAR-NK_{REX}=10\hat{ }26 fold expansion at assay start) TERT overexpressing CAR-NK_{REX} cells demonstrated enhanced functionality and persistence in this assay, completing 16 rounds of serial kill with at least 30% cytolysis. In contrast, despite being younger and less expanded, CAR-NK_{REX} cells only completed 11 rounds under these conditions. Similarly, While CAR-NK_{REX} cells only achieved a maximum of 69 fold expansion in this study, TERT overexpressing CAR-NK_{REX} cells reached 3236 fold expansion in this assay.

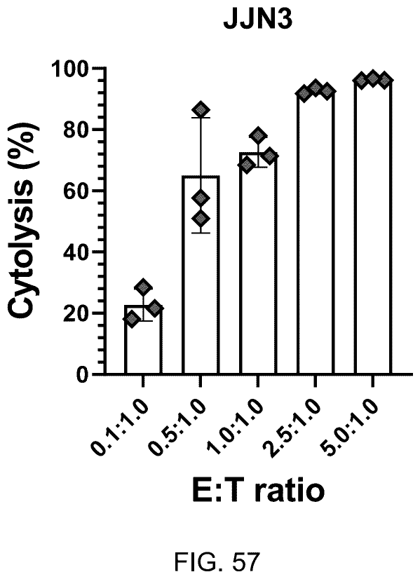

TERT overexpressing CAR-NK_{REX} cells were generated as in FIG. 47. Cells were cryo-preserved on day 256 and then cryo-recovered and expanded to day 414 before assay initiation. TERT overexpressing CAR-NK_{REX} cells were co-cultured with BCMA expressing JJN3 cells at various effector:target cell ratios for a period of 3 days. Percent cytolysis was computed at the end of the co-culture. TERT overexpressing CAR-NK_{REX} cells maintain cytotoxic potential following cryo-recovery and expansion to more than 400 days in culture (see FIG. 57).

### Example 38: Generation of TERT Overexpressing NK_{REX} and CAR-NK_{REX} Cells

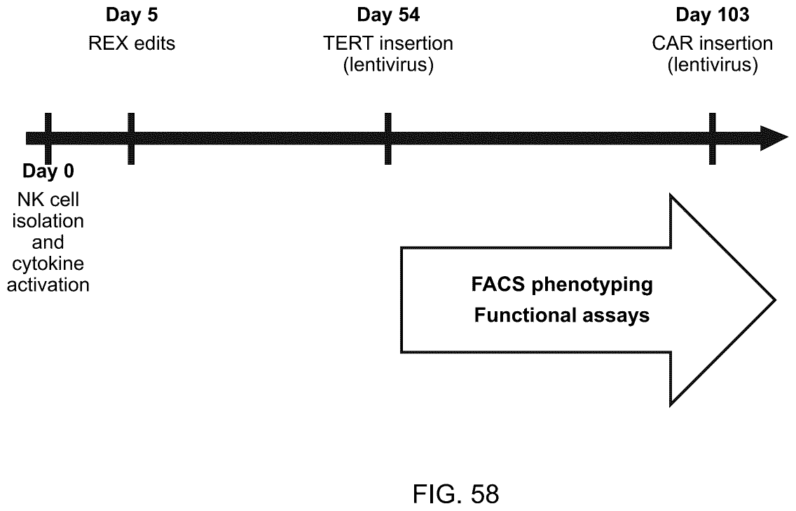

In the preceding studies, CAR-NK_{REX} cells were modified to overexpress TERT. In order to investigate the ability of TERT to enhance NK_{REX} cell and CAR-NK_{REX} cell expansion and functionality, NK_{REX} cells were first generated and then modified to overexpress TERT. TERT overexpressing NK_{REX} cells were then engineered to express a BCMA targeting CAR (see FIG. 58).

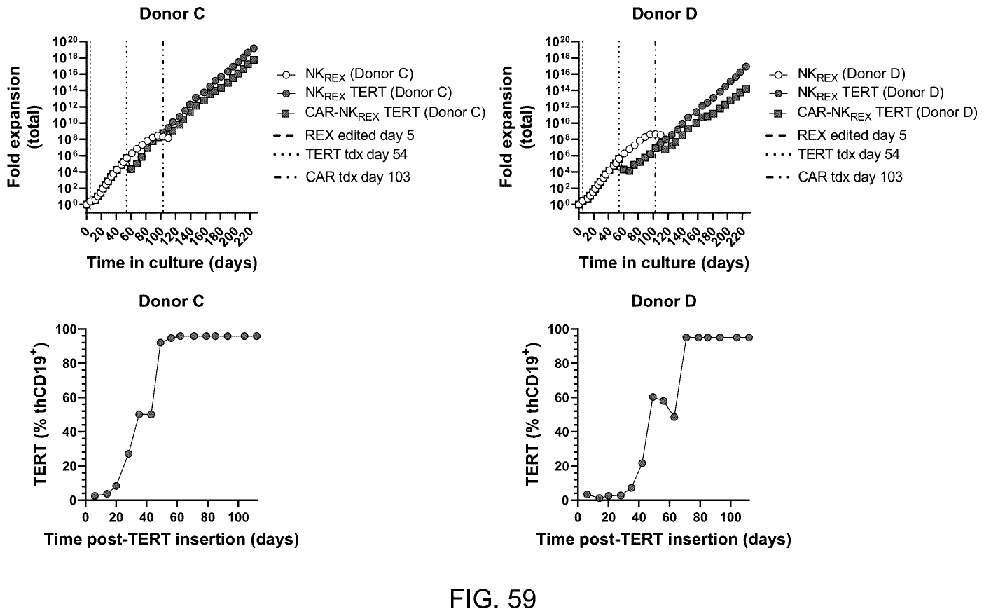

NK_{REX} cells (REX edit containing NK cells) were generated from two additional donors and further engineered to overexpress TERT. In some instances, these cells were modified to express a BCMA-targeting CAR. All groups were monitored for expansion throughout the engineering and culture process. Enrichment of TERT-expressing NK_{REX} cells was also assessed over time. Consistent with previous results, NK_{REX} cells Donors C and D showed enrichment of the TERT transgene over time with cells reaching nearly 100% transgene expression as seen in the bottom panel of FIG. 59. Further, NK_{REX} cells from Donors C and D plateaued at days 96 and 103 in culture, achieving a maximum expansion of 2.88E+008 fold and 4.36E+008 fold respectively. NK_{REX} TERT cells from these donors continued proliferating through the time of cryo-preservation at day 225 achieving 1.47E+019 fold (donor C) and 8.53E+016 fold (donor D) expansion. CAR-NK_{REX} TERT cells from these donors also continued proliferating through the time of cryo-preservation on day 225, achieving 5.65E+017 fold (donor C) and 1.7E+014 fold (donor D) expansion. These results demonstrate that TERT overexpression reproducibly confers an advantage to NK_{REX} and CAR-NK_{REX} cells (see FIG. 59).

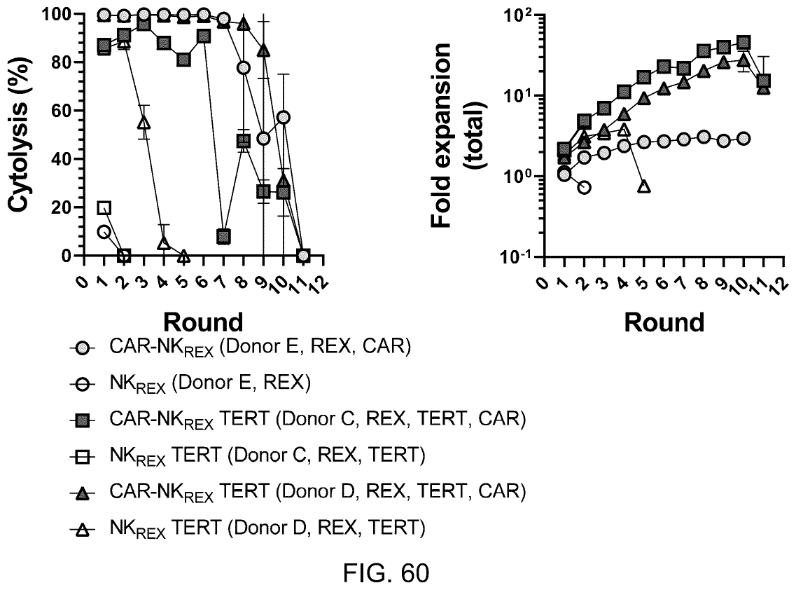

NK_{REX} cells, TERT overexpressing NK_{REX} cells, BCMA targeting CAR-NK_{REX} cells and TERT overexpressing CAR-NK_{REX} cells were generated. Cytotoxicity and persistence of effector cells were measured over multiple rounds of co-culture with BCMA-expressing JJN3 target cells at an effector:target cell ratio of 2:1 in the presence of IL-2. Following each round of co-culture, percent cytolysis and effector cell expansion were assessed. At assay initiation NK_{REX} and CAR-NK_{REX} cells were 119 days in culture while TERT overexpressing NK_{REX} and TERT overexpressing CAR-NK_{REX} were in culture for 139 days. CAR expression was necessary for tumor cell lysis and persistence in this assay for both NK_{REX} cells and TERT overexpressing NK_{REX} cells. CAR-negative groups persisted for only 2 (Donor E NK_{REX}), 2 (Donor C NK_{REX} TERT), and 5 rounds (Donor D NK_{REX} TERT); and completed 0 (Donor E NK_{REX}), 0 (Donor C NK_{REX} TERT), and 3 (Donor D NK_{REX} TERT) rounds of serial kill in which they achieved at least 30% cytolysis. In line with poor function and persistence of these CAR-deficient groups, they showed limited maximum expansion in this assay: 1.1 fold (Donor E NK_{REX}), 4.6 fold (Donor C NK_{REX} TERT), and 3.8 fold (Donor D NK_{REX} TERT). In contrast, CAR-NK_{REX} cells (Donor E) completed 10 rounds of serial kill in which they achieved at least 30% cytolysis and they reached a maximum expansion of 3.07 fold. CAR NK_{REX} TERT cells from Donor C completed 8 rounds of serial kill with 30% cytolysis but continued to kill tumor cells for an additional 2 rounds at a level of approximately 25%. CAR NK_{REX} TERT cells from Donor C achieved a maximum expansion of 45.8 fold. Finally, CAR NK_{REX} TERT cells from Donor D completed 10 rounds of serial kill with 30% cytolysis and reached a maximal expansion of 27.7 fold. Therefore, while CAR-NK_{REX} cells and TERT overexpressing NK_{REX} cells completed a similar number of rounds of tumor cell lysis, TERT overexpression enhanced the overall expansion of CAR-NK_{REX} cells. The results demonstrate that TERT overexpressing CAR-NK_{REX} cells are cytotoxic and expand better than in serial kill assays than younger CAR-NK_{REX} cells (see FIG. 60).

### Example 39: STAT5A and STAT5B Mutants Enrich in REX Edited CD8⁺ T Cells In Vitro

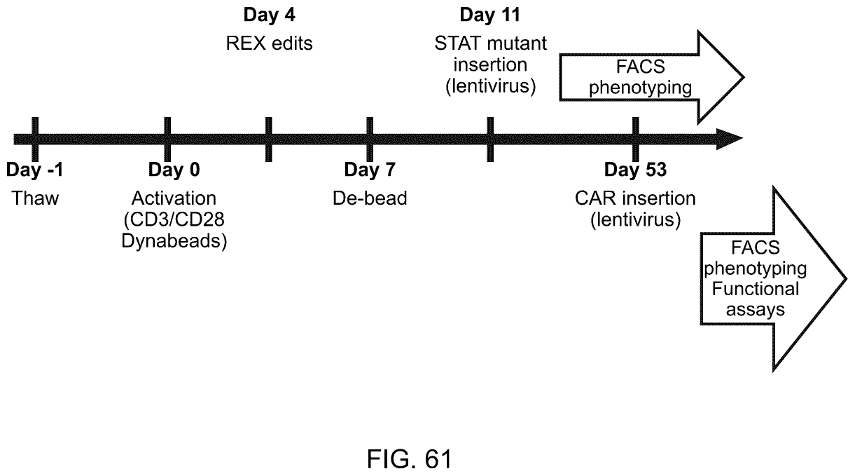

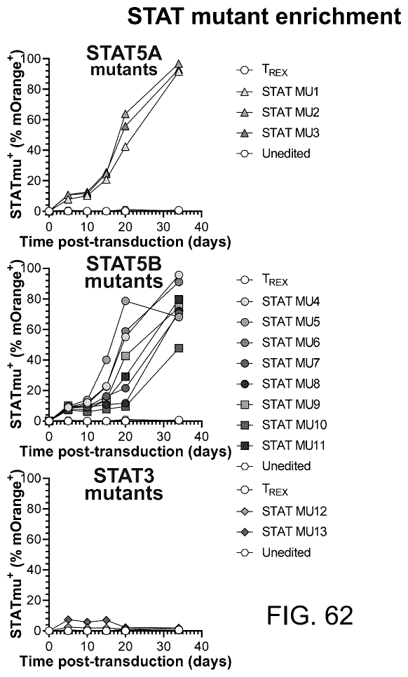

Three STAT5A mutants, eight STAT5B mutants, and two STAT3 mutants were overexpressed in T_{REX} cells (REX edit containing CD8⁺ T cells) as per the timeline shown in FIG. 61. Table 9 below lists all STAT mutants tested in this study as well as a description of signaling pathways regulated by these molecules. Enrichment of STAT mutants was followed over time using a fluorescent reporter. STAT5A and STAT5B mutant expressing T_{REX} cells enriched during the cell culture process while the STAT3 mutant alone groups showed no selective advantage. FIG. 62 shows STAT mutant enrichment.

TABLE 9
STAT mutants.

| Key | STAT | Shared Mutations | | Cytokine Pathway | |
|---|---|---|---|---|---|
| STAT MUT 1 | STAT5A | 1/2 | (H299R/S711F) | 1. | Common γ |
| (STAT MU1) | | | | | chain family: |
| STAT MUT 2 | STAT5A | 3 | (N642H) | | IL-2, IL-4, |
| (STAT MU2) | | | | | IL-7, IL-9, |
| STAT MUT 3 | STAT5A | 4 | (Y665F) | | IL-15, and |
| (STAT MU3) | | | | | IL-21 |
| STAT MUT 4 | STAT5B | 1/2 | (H298R/S715F) | 2. | IL-3 family: |
| (STAT MU4) | | | | | IL-3, IL-5, |
| STAT MUT 5 | STAT5B | 3 | (N642H) | | GM-CSF |
| (STAT MU5) | | | | 3. | Single chain |
| STAT MUT 6 | STAT5B | 4 | (Y665F) | | receptors: EPO, |
| (STAT MU6) | | | | | TPO, GH, |
| STAT MUT 7 | STAT5B | 5/6 | (R430C/P702A) | | Prolactin, |
| (STAT MU7) | | | | | G-CSF |
| STAT MUT 8 | STAT5B | 7/6 | (E433K/P702A) | 4. | Growth factor |
| (STAT MU8) | | | | | receptors: SCF, |
| STAT MUT 9 | STAT5B | 1/8 | (H298R/V712E) | | PDGF, EGF |
| (STAT MU9) | | | | | |
| STAT MUT 10 | STAT5B | 7/8 | (E433K/V712E) | | |
| (STAT MU10) | | | | | |
| STAT MUT 11 | STAT5B | 8 | (V712E) | | |
| (STAT MU11) | | | | | |
| STAT MUT 12 | STAT3 | 9 | (Y640F) | 1. | IL-2 family: |
| (STAT MU12) | | | | | IL-2, IL-7, |
| STAT MUT 13 | STAT3 | 10 | (D661Y) | | IL-21 |
| (STAT MU13) | | | | 2. | IL-6 family: |
| | | | | | IL-6, IL-11, |
| | | | | | LIF |
| | | | | 3. | IL-10 |
| | | | | 4. | GPCRs and |
| | | | | | growth factor |
| | | | | | receptors |

### Example 40: STAT5A and STAT5B Mutants are Functional in T_{REX} Cells

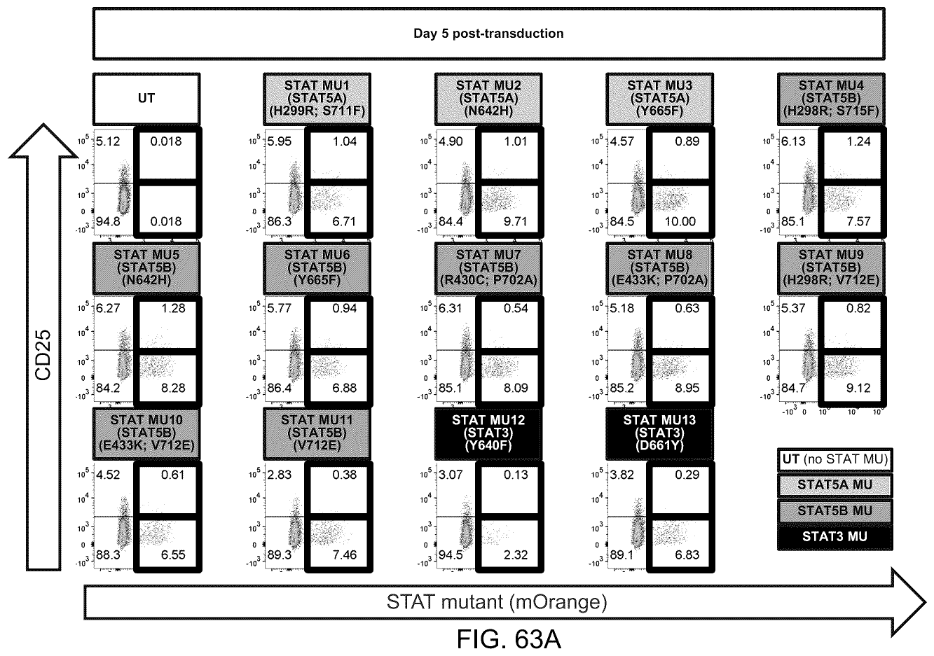

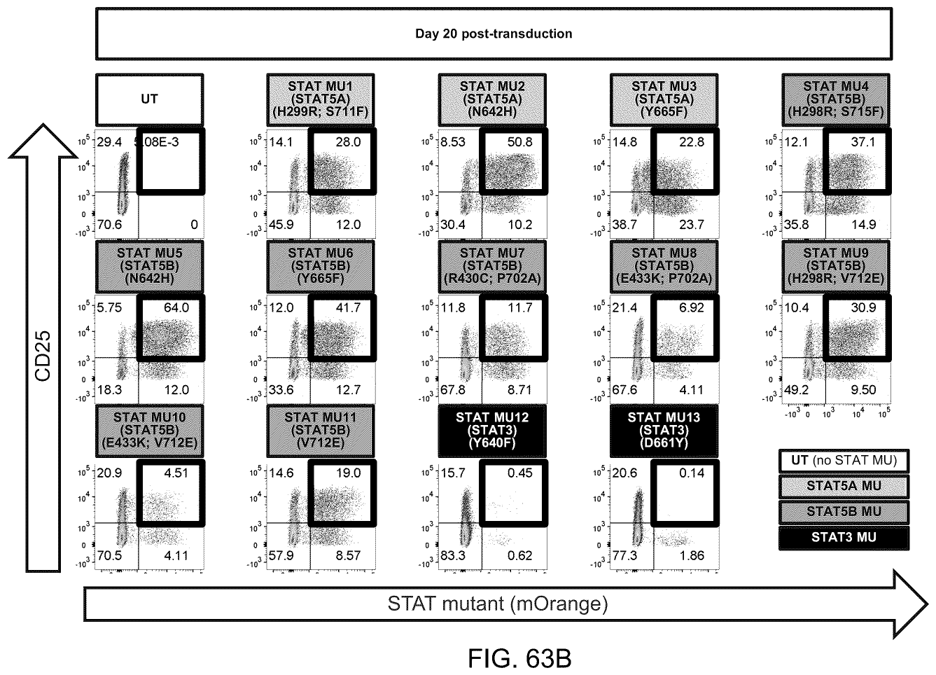

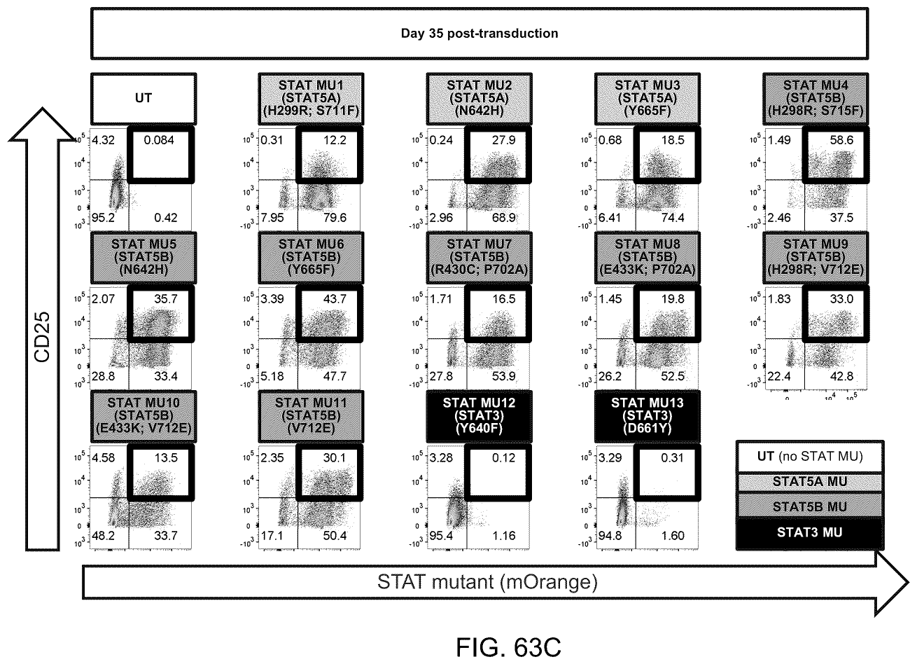

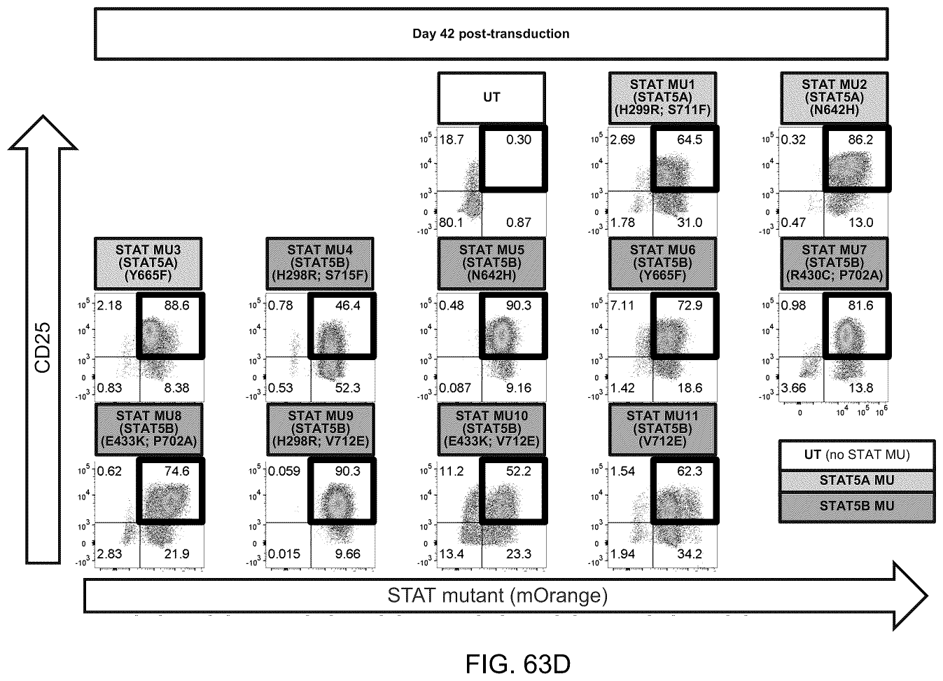

Surface expression of CD25 was assessed in control T_{REX} cells (UT) and STAT5A, STAT5B, and STAT3 mutant containing T_{REX} cells over extended cell culture. STAT5A and STAT5B mutants are functional in T_{REX} cells, leading to upregulation of CD25 expression 5 days after transduction (see FIG. 63A).

Surface expression of CD25 was assessed in control T_{REX} cells (UT) and STAT5A, STAT5B, and STAT3 mutant containing T_{REX} cells over extended cell culture. STAT5A and STAT5B mutants are functional in T_{REX} cells, leading to upregulation of CD25 expression 20 days after transduction (see FIG. 63B).

Surface expression of CD25 was assessed in control T_{REX} cells (UT) and STAT5A, STAT5B, and STAT3 mutant containing T_{REX} cells over extended cell culture. STAT5A and STAT5B mutants are functional in T_{REX} cells, leading to upregulation of CD25 expression 35 days after transduction (see FIG. 63C).

Surface expression of CD25 was assessed in control T_{REX} cells (UT) and STAT5A, STAT5B, and STAT3 mutant containing T_{REX} cells over extended cell culture. STAT5A and STAT5B mutants are functional in T_{REX} cells, leading to upregulation of CD25 expression 42 days after transduction (see FIG. 63D).

### Example 41: STAT5A and STAT5B Mutant T_{REX} Cells can Express a CAR and Expand Robustly During the Culture Process

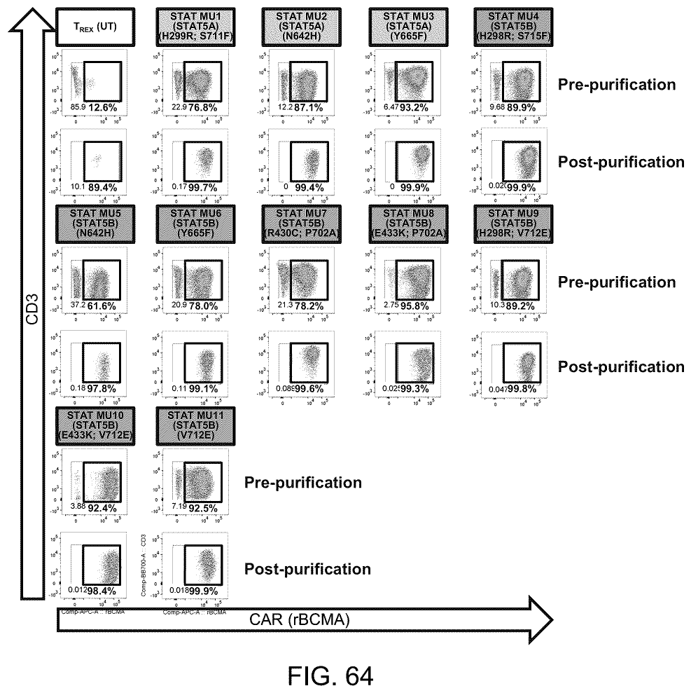

A BCMA targeting CAR was introduced into unmodified T_{REX} cells (T_{REX} (UT)) and STAT mutant T_{REX} cells (STAT MU1, STAT MU2, STAT MU3, STAT MU4, STAT MU5, STAT MU6, STAT MU7, STAT MU8, STAT MU9, STAT MU10, and STAT MU11). CAR-expressing cells were further enriched to high purity prior to functional assessment, and expansion of STAT mutant CAR-T_{REX} cells was tracked throughout the editing, transduction, and enrichment process (see FIG. 64).

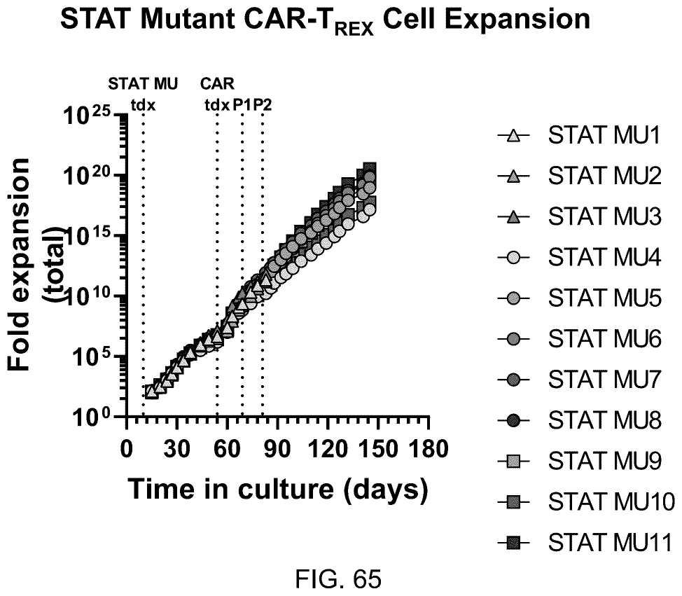

Expansion of STAT mutant CAR-T_{REX} cells was tracked throughout the editing, transduction, and enrichment process, and as shown in FIG. 65 STAT mutant CAR-T_{REX} cell expand robustly during the culture process.

### Example 42: STAT5A and STAT5B Mutants Confer Varying Degrees of IL-2 Independence In Vitro

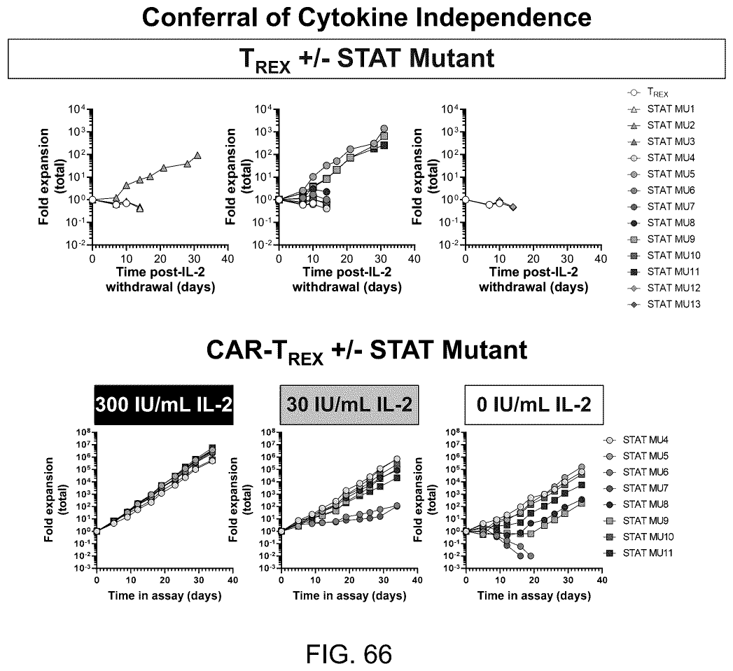

Three STAT5A mutants, eight STAT5B mutants, and two STAT3 mutants were overexpressed in T_{REX} cells (REX edit containing CD8⁺ T cells) as per FIG. 61. STAT5A mutant, STAT5B mutant, and STAT3 mutant T_{REX} cells and control T_{REX} cells were cultured in media in the absence of IL-2 and expansion was followed over time. In the absence of a CAR, multiple groups showed IL-2 dependence (control T_{REX} cells, STAT MU1, STAT MU3, STAT MU4, STAT MU6, STAT MU7, STAT MU8, STAT MU10, STAT MU12, STAT MU13) while others grew independently of IL-2 (STAT MU2, STAT MU5, STAT MU9, and STAT MU11). Despite showing IL-2 dependency, the following STAT mutants produced higher than baseline (control T_{REX}) T_{REX} cell expansion at early timepoints following IL-2 withdrawal: STAT MU6, STAT MU8. Some STAT mutant T_{REX} cells were further engineered to express a CAR as per FIG. 61. Cells were grown in the presence of decreasing amounts of IL-2 and expansion was followed over time. The combination of CAR and STAT mutant expression enhanced the expansion of multiple groups. STAT MU6 and STAT MU7 CAR-T_{REX} cells maintained dependence on IL-2 (though these groups proliferated under low-IL-2 conditions). STAT MU8 and STAT MU9 CAR-T_{REX} cells demonstrated a capacity to proliferate in the absence of IL-2, while the greatest degree of IL-2 independence was observed in STAT MU4, STAT MU5, STAT MU10 and STAT MU11 CAR-T_{REX} cells (see FIG. 66).

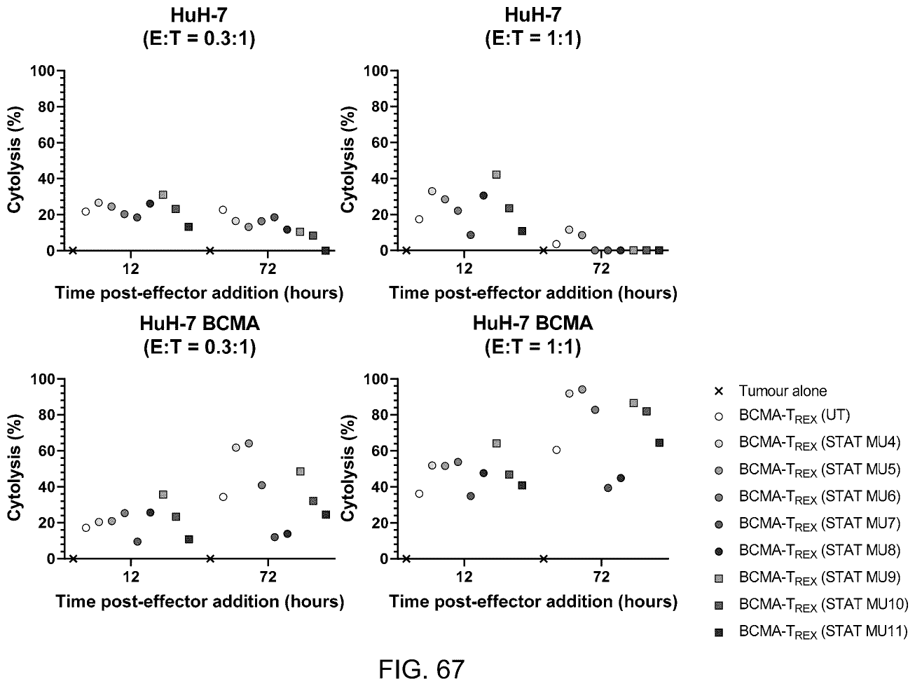

A BCMA targeting CAR was introduced into young T_{REX} cells (BCMA-T_{REX} (UT); 99 days in culture) and STAT mutant T_{REX} cells (STAT MU4, STAT MU5, STAT MU6, STAT MU7, STAT MU8, STAT MU9, STAT MU10, STAT MU11; all 155 days in culture). Cytotoxicity of CAR-expressing cells was measured using the impedance-based xCELLigence platform. Percent cytolysis was determined 12 hours and 72 hours post initiation of co-culture of effector cells and target cells at various effector:target (E:T) cell ratios. Antigen-negative (HuH-7) and antigen-positive (HuH-7 BCMA) target cells were used in this experiment. STAT5B mutant CAR-T_{REX} cell variants remain functional even after more than 150 days in culture (see FIG. 67).

### Example 43: STAT5B Mutant CAR-T_{REX} Cells Demonstrate Enhanced Persistence and Functionality Relative to Young CAR-T_{REX} Cell Benchmarks in a Serial Kill Assay

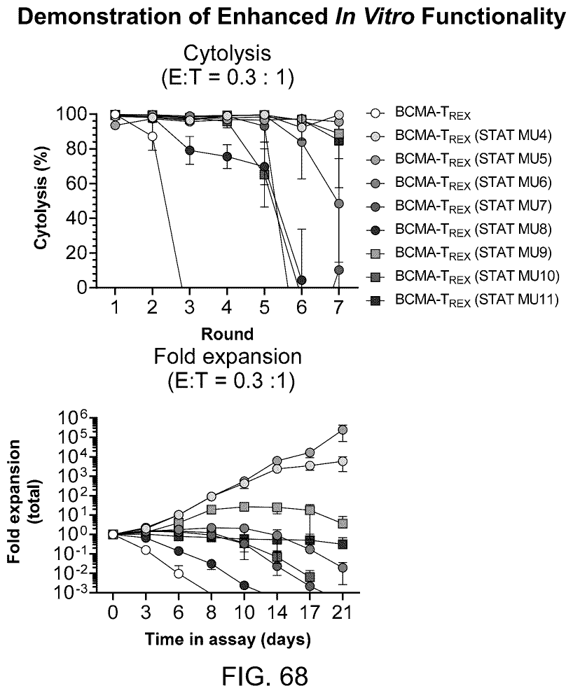

A BCMA targeting CAR was introduced into T_{REX} cells (BCMA-T_{REX}) and STAT5B mutant expressing T_{REX} cells (STAT MU4, STAT MU5, STAT MU6, STAT MU7, STAT MU8, STAT MU9, STAT MU10, STAT MU11). Cytotoxicity and persistence of CAR-expressing cells were measured over multiple rounds of co-culture with BCMA-expressing JJN3 target cells at an effector:target cell ratio of 0.3:1. Following each round of co-culture, percent cytolysis and effector cell expansion were assessed. All STAT5B mutants enhanced CAR-T_{REX} cell persistence and cytotoxicity to varying degrees (see FIG. 68). BCMA-T_{REX} cells could complete only 2 rounds of this assay in which they achieved at least 30% cytolysis and they effectively perished after 3 rounds, failing to expand and persist. All STAT5B mutant containing CAR-T_{REX} cells completed at least 5 rounds of serial kill in which they exhibited at least 30% cytolysis. Further, some STAT5B mutant containing BCMA-T_{REX} cells achieved >80% cytolysis at assay termination (STAT MU9 BCMA-T_{REX} cells and STAT MU11 BCMA-T_{REX} cells) and others even >95% cytolysis at the end of round 7 (STAT MU4 BCMA-T_{REX} cells and STAT MU5 BCMA-T_{REX} cells). All STAT5B mutant containing CAR-T_{REX} cells (max expansion=2.45E+05 observed in STAT MU5 BCMA-T_{REX} cells) expanded more robustly than BCMA-T_{REX} cells (max expansion=1 fold).

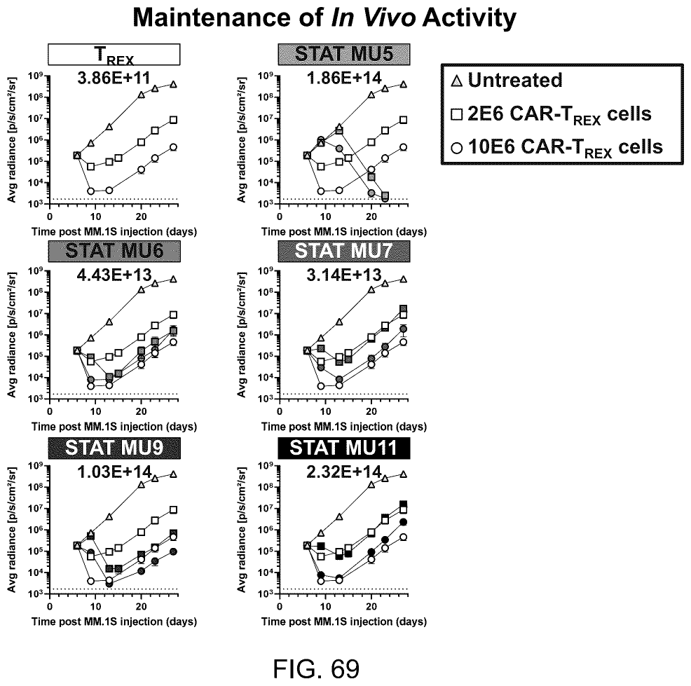

NSG mice were inoculated with 2E6 MMIS-luciferase tumor cells. Six days later, BCMA-T_{REX} cells (T_{REX}, day 99) or STAT mutant BCMA-T_{REX} cells (STAT MU5, STAT MU6, STAT MU7, STAT MU9, and STAT MU11-all day 112) were dosed at 2E6 or 10E6 cells per mouse. Tumor burden was monitored twice weekly using IVIS imaging. All STAT mutant T_{REX} cells demonstrated in vivo functionality, despite higher degrees of in vitro expansion (numbers shown under group title). In some cases, STAT mutant BCMA-T_{REX} cells were even more efficacious than control BCMA-T_{REX} cells at low doses (STAT MU5, STAT MU6, STAT MU9). For these groups, STAT5B mutant BCMA-T_{REX} cells dosed at 2E6 cells cleared tumor comparably or more effectively than BCMA-T_{REX} cells dosed at 10E6 cells. Therefore, STAT5B mutant CAR-T_{REX} cells are at least as functional as young CAR-T_{REX} cell benchmarks in vivo (see FIG. 69).

### Example 44: STAT5B and STAT3 Mutants Enrich in REX Edited NK Cells In Vitro

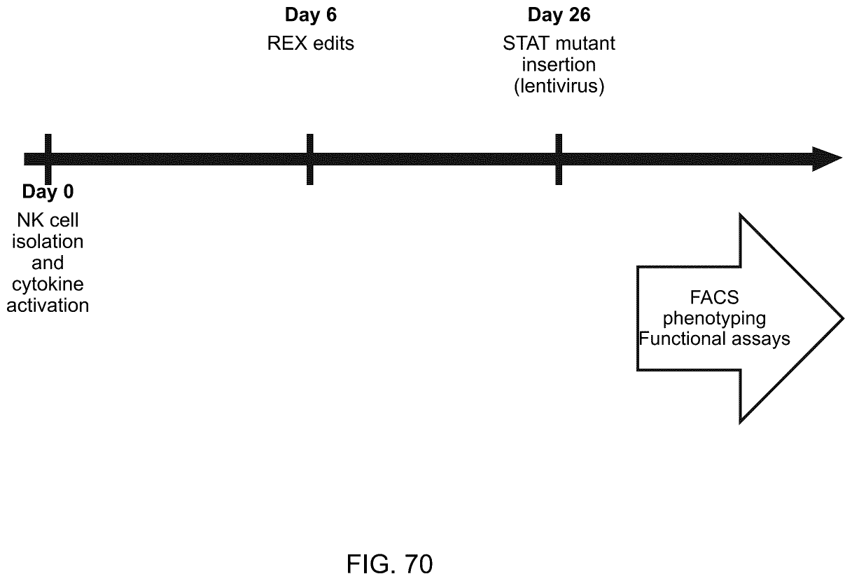

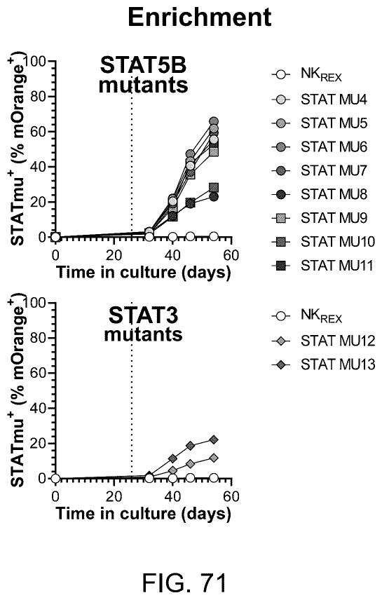

Eight STAT5B mutants and two STAT3 mutants were overexpressed in NK_{REX} cells (REX edit containing NK cells) as per the timeline shown (see FIG. 70). Table 10 lists all STAT mutants tested in this study as well as a description of signaling pathways regulated by these molecules. Enrichment of STAT mutants was followed over time using a fluorescent reporter. STAT5B and STAT3 mutants were found to enrich in REX edited NK cells in vitro suggesting that overexpression of these mutant proteins may enhance the NK_{REX} cell phenotype (see FIG. 71).

TABLE 10
STAT5B and STAT3 mutants enrich in REX edited NK cells in vitro.

| Key | STAT | Shared Mutations | | Cytokine Pathway | |
|---|---|---|---|---|---|
| STAT MUT 4 | STAT5B | 1/2 | (H298R/S715F) | 1. | Common γ |
| (STAT MU 4) | | | | | chain family: |
| STAT MUT 5 | STAT5B | 3 | (N642H) | | IL-2, IL-4, |
| (STAT MU 5) | | | | | IL-7, IL-9, |
| STAT MUT 6 | STAT5B | 4 | (Y665F) | | IL-15, |
| (STAT MU 6) | | | | | and IL-21 |
| STAT MUT 7 | STAT5B | 5/6 | (R430C/P702A) | 2. | IL-3 family: |
| (STAT MU 7) | | | | | IL-3, IL-5, |
| STAT MUT 8 | STAT5B | 7/6 | (E433K/P702A) | | GM-CSF |
| (STAT MU 8) | | | | 3. | Single chain |
| STAT MUT 9 | STAT5B | 1/8 | (H298R/V712E) | | receptors: EPO, |
| (STAT MU 9) | | | | | TPO, GH, |
| STAT MUT 10 | STAT5B | 7/8 | (E433K/V712E) | | Prolactin, |
| (STAT MU 10) | | | | | G-CSF |
| STAT MUT 11 | STAT5B | 8 | (V712E) | 4. | Growth factor |
| (STAT MU 11) | | | | | receptors: SCF, |
| | | | | | PDGF, EGF |
| STAT MUT 12 | STAT3 | 9 | (Y640F) | 1. | IL-2 family: |
| (STAT MU 12) | | | | | IL-2, IL-7, |
| STAT MUT 13 | STAT3 | 10 | (D661Y) | | IL-21 |
| (STAT MU 13) | | | | 2. | IL-6 family: |
| | | | | | IL-6, IL-11, |
| | | | | | LIF |
| | | | | 3. | IL-10 |
| | | | | 4. | GPCRs |
| | | | | | and growth |
| | | | | | factor receptors |

### Example 45: STAT5B and STAT3 Mutant Expression Enhances Proliferation of NK_{REX} Cells

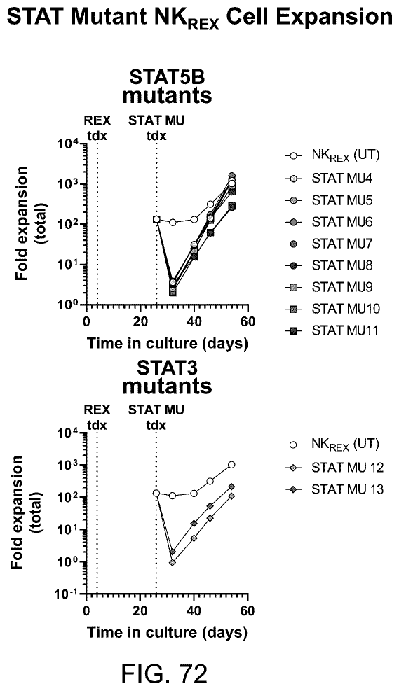

Eight STAT5B mutants and two STAT3 mutants were overexpressed in NK_{REX} cells (REX edit containing NK cells) as per FIG. 70. Expansion of control NK_{REX} cells (REX-edited NK cells) and STAT mutant containing NK_{REX} cells was assessed throughout the culture process. STAT5B mutant NK_{REX} cells and STAT3 mutant NK_{REX} cells proliferated at a faster rate than donor matched unmodified NK_{REX} cells as evidenced by steeper growth curves following introduction of the STAT mutants. STAT5B and STAT3 mutant expression enhances proliferation of NK_{REX} cells (see FIG. 72).

### Example 46: STAT Mutant Expressing NK_{REX} Cells Retain Functionality in an In Vitro Cytotoxicity Assay

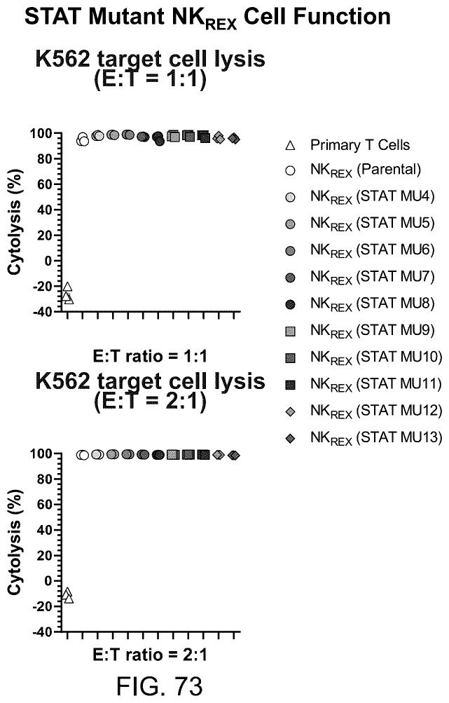

Eight STAT5B mutants and two STAT3 mutants were overexpressed in NK_{REX} cells (REX edit containing NK cells) as per FIG. 70. Cytotoxic function of STAT mutant containing NK_{REX} cells was assessed through co-culture of control (unmodified) NK_{REX} and STAT mutant NK_{REX} cells with K562-luciferase cells. Primary T cells were used as a control as they exhibit no cytotoxicity against K562 cells in this assay. Percent cytolysis was determined 24 hours post initiation of the co-cultures. Effector cells were co-cultured with K562 cells at 2 different effector:target cell ratios (1:1-top and 2:1-bottom). STAT5B mutant and STAT3 mutant expressing NK_{REX} cells demonstrated similar cytotoxic activity to unmodified NK_{REX} cells. Primary T cell controls did not lyse K562-luciferase cells in this assay. Therefore, STAT mutant expressing NK_{REX} cells retain functionality in an in vitro cytotoxicity assay (see FIG. 73).

The embodiments described herein can be practiced in the absence of any element or elements, limitation or limitations that are not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the embodiments claimed. Thus, it should be understood that although the present description has been specifically disclosed by embodiments, optional features, modification and variation of the concepts herein disclosed may be resorted to by those skilled in the art, and that such modifications and variations are considered to be within the scope of these embodiments as defined by the description and the appended claims. Although some aspects of the present disclosure can be identified herein as particularly advantageous, it is contemplated that the present disclosure is not limited to these particular aspects of the disclosure.

Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group.

It should it be understood that, in general, where the disclosure, or aspects of the disclosure, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein.

All patents and publications mentioned in this specification are herein incorporated by reference to the same extent as if each independent patent and publication was specifically and individually indicated to be incorporated by reference. Citation or identification of any reference in any section of this application shall not be construed as an admission that such reference is available as prior art to the present disclosure.

1. A method of generating a population of primary immune cells resistant to replicative senescence (RRS), comprising:
(a) inhibiting the expression of cyclin-dependent kinase inhibitor 2A (CDKN2A), cyclin-dependent kinase inhibitor 2B (CDKN2B), and S-methyl-5′-thioadenosine phosphorylase (MTAP) in the population of primary immune cells;
(b) introducing a transgene encoding one or more STAT5A mutants and/or one or more STAT5B mutants in the population of primary immune cells; and
(c) culturing the primary immune cells in a culture medium;
wherein the culturing induces proliferation of the primary immune cells to yield the population of primary immune cells resistant to replicative senescence (RRS).

2-12. (canceled)

13. A method of generating a population of primary immune cells resistant to replicative senescence (RRS), comprising:
(a) inhibiting the expression of cyclin-dependent kinase inhibitor 2A (CDKN2A), cyclin-dependent kinase inhibitor 2B (CDKN2B), and S-methyl-5′-thioadenosine phosphorylase (MTAP) in the population of primary immune cells;
(b) introducing a transgene encoding MYC in the population of primary immune cells; and
(c) culturing the primary immune cells in a culture medium;
wherein the culturing induces proliferation of the primary immune cells to yield the population of primary immune cells resistant to replicative senescence (RRS).

14-19. (canceled)

20. A method of generating a population of primary immune cells resistant to replicative senescence (RRS), comprising:
(a) inhibiting the expression of cyclin-dependent kinase inhibitor 2A (CDKN2A), cyclin-dependent kinase inhibitor 2B (CDKN2B), and S-methyl-5′-thioadenosine phosphorylase (MTAP) in the population of primary immune cells;
(b) introducing a transgene encoding TERT in the population of primary immune cells; and
(c) culturing the primary immune cells in a culture medium;
wherein the culturing induces proliferation of the primary immune cells to yield the population of primary immune cells resistant to replicative senescence (RRS).

21-118. (canceled)

119. An engineered T cell that does not express cyclin-dependent kinase inhibitor 2A (CDKN2A), cyclin-dependent kinase inhibitor 2B (CDKN2B), and/or S-methyl-5′-thioadenosine phosphorylase (MTAP), wherein the engineered T cell comprises a transgene encoding one or more STAT5A mutants and/or one or more STAT5B mutants.

120. The engineered T cell of claim 119, wherein the one or more STAT5A mutants can be H299R, N642H, Y665F, S711F, and combinations thereof, and/or wherein the one or more STAT5B mutants can be H298R, R430C, E433K, N642H, Y665F, P702A, V712E, S715F, and combinations thereof.

121. The engineered T cell of claim 119 further comprising introducing a transgene encoding TERT.

122. The engineered T cell of claim 119, wherein the engineered T cell further comprises a transgene encoding either B-cell lymphoma-extra large (Bcl-xL) or B-cell lymphoma 2 (Bcl-2).

123. The engineered T cell of claim 119, wherein the engineered T cell does not express of one or more endogenous immune related genes.

124. The engineered T cell of claim 123, wherein the endogenous immune related gene is beta-2 microglobulin (B2M) and/or T-cell receptor α constant (TRAC).

125. The engineered T cell of claim 119, wherein the engineered T cell does not express cluster of differentiation 38 (CD38), phosphatase and tensin homolog (PTEN), and/or p53.

126. The engineered T cell of claim 119 further comprising a transgene encoding MYC and/or a transgene encoding KRAS.

127. An engineered T cell that does not express cyclin-dependent kinase inhibitor 2A (CDKN2A), cyclin-dependent kinase inhibitor 2B (CDKN2B), and/or S-methyl-5′-thioadenosine phosphorylase (MTAP), wherein the engineered T cell comprises a transgene encoding MYC.

128. The engineered T cell of claim 127 further comprising a transgene encoding B-cell lymphoma-extra large (Bcl-xL).

129. The engineered T cell of claim 127, wherein the engineered T cell does not express p53.

130. The engineered T cell of claim 127 further comprising a transgene encoding KRAS.

131. The engineered T cell of claim 130, wherein KRAS comprises a KRAS A146V mutation.

132. The engineered T cell of claim 127, wherein the engineered T cell does not express phosphatase and tensin homolog (PTEN).

133. The engineered T cell of claim 132, wherein PTEN expression is inhibited by a CRISPR/Cas system.

134. An engineered T cell that does not express cyclin-dependent kinase inhibitor 2A (CDKN2A), cyclin-dependent kinase inhibitor 2B (CDKN2B), and/or S-methyl-5′-thioadenosine phosphorylase (MTAP), and comprises a transgene encoding TERT.

135. The engineered T cell of claim 134, wherein the engineered T cell comprises a transgene encoding MYC.

136. The engineered T cell of claim 134 further comprising a transgene encoding KRAS.

137. The engineered T cell of claim 136, wherein KRAS comprises a KRAS A146V mutation.

138. The engineered T cell of claim 127, wherein the engineered T cell does not express one or more endogenous immune related genes in the primary immune cells in the population of primary immune cells.

139. The engineered T cell of claim 138, wherein the endogenous immune related gene is beta-2 microglobulin (B2M) and/or T-cell receptor α constant (TRAC).

140. The engineered T cell of claim 127, wherein the engineered T cell does not express cluster of differentiation 38 (CD38).

141. The engineered T cell of claim 119 further comprising a polynucleotide that encodes a chimeric antigen receptor (CAR).

142. The engineered T cell of claim 119, wherein the engineered T cell is a CD8⁺ T cell, a CD4⁺ T cell, a gamma-delta T cell, a mucosal associated invariant T (MAIT) T cell, a natural killer (NK) cell, a natural killer T (NKT) cell, or a combination thereof.

143. The engineered T cell of claim 119, wherein the engineered T cell is a CD8⁺ T cell.

144. The engineered T cell of claim 119, wherein the engineered T cell is a CD4⁺ T cell.

145. The engineered T cell of claim 119, wherein the engineered T cell is human.

146-164. (canceled)

165. A method of treating cancer in a subject in need thereof, comprising administering to the subject a composition comprising an engineered T cell, wherein the engineered T cell does not express cyclin-dependent kinase inhibitor 2A (CDKN2A), cyclin-dependent kinase inhibitor 2B (CDKN2B), and/or S-methyl-5′-thioadenosine phosphorylase (MTAP), and wherein the engineered T cell comprises a transgene encoding one or more STAT5A mutants and/or one or more STAT5B mutants.

166-173. (canceled)

174. A method of treating cancer in a subject in need thereof, comprising administering to the subject a composition comprising an engineered T cell, wherein the engineered T cell does not express cyclin-dependent kinase inhibitor 2A (CDKN2A), cyclin-dependent kinase inhibitor 2B (CDKN2B), and/or S-methyl-5′-thioadenosine phosphorylase (MTAP), and wherein the engineered T cell comprises a transgene encoding MYC.

175-180. (canceled)

181. A method of treating cancer in a subject in need thereof, comprising administering to the subject a composition comprising an engineered T cell, wherein the engineered T cell does not express cyclin-dependent kinase inhibitor 2A (CDKN2A), cyclin-dependent kinase inhibitor 2B (CDKN2B), and/or S-methyl-5′-thioadenosine phosphorylase (MTAP), and wherein the engineered T cell comprises a transgene encoding TERT.

182-220. (canceled)
